(19) United States
(12) Patent Application Publication
(10) Pub. No.: US 2025/0084160 A1
(43) Pub. Date: Mar. 13, 2025

(54) ANTIBODIES TARGETING CDH19 FOR MELANOMA

(71) Applicant: Amgen Inc., Thousand Oaks, CA (US)

(72) Inventors: Shouhua Xiao, Foster City, CA (US); Zheng Pan, Fremont, CA (US); Dineli Wickramasinghe, San Francisco, CA (US); M. Shawn Jeffries, Indianapolis, IN (US); Chadwick Terence King, North Vancouver (CA); Brian Mingtung Chan, Port Coquitlam (CA)

(21) Appl. No.: 18/610,640

(22) Filed: Mar. 20, 2024

Related U.S. Application Data
(63) Continuation of application No. 17/326,717, filed on May 21, 2021, now abandoned.
Application No. 17/326,717 is a division of application No. 15/878,047, filed on Jan. 23, 2018, now Pat. No. 11,053,311.
Application No. 15/878,047 is a division of application No. 14/762,053, filed on Jul. 20, 2015, now Pat. No. 9,920,121.
(60) Provisional application No. 61/756,977, filed on Jan. 25, 2013.
(60) Provisional application No. 61/785,119, filed on Mar. 14, 2013.

(51) Int. Cl. C07K 16/28; A61K 39/00; A61K 47/68; C07K 16/30

(52) U.S. Cl. CPC C07K 16/28; A61K 47/68033; A61K 47/6849; A61K 47/6865; C07K 16/3053; A61K 2039/505; C07K 2317/21; C07K 2317/33; C07K 2317/34; C07K 2317/55; C07K 2317/73; C07K 2317/77; C07K 2317/92

(57) ABSTRACT

The present disclosure provides a human antibody or antigen binding fragment thereof or an antibody construct comprising a human binding domain or antigen binding fragment thereof capable of binding to human CDH19 on the surface of a target cell. The disclosure relates to a nucleic acid sequence encoding the antibody or antigen binding fragment thereof contained in the antibody construct, a vector comprising the nucleic acid sequence and a host cell transformed or transfected with the vector. Furthermore, the disclosure relates to a process for the production of the antibody construct of the disclosure, a medical use or a method of treatment using the antibody construct and a kit comprising the antibody or antigen binding fragment thereof or the antibody construct.

### RELATED APPLICATIONS

This application is related to a U.S. provisional application entitled “Antibody constructs for CDH19 and CD3,” filed on Mar. 15, 2013, the same day as the present application is filed. This related application is incorporated in its entirety by reference.

### INCORPORATION BY REFERENCE OF THE SEQUENCE LISTING

This application includes a sequence listing submitted electronically as I-49735C_SeqListing.xml; Size: 1,442,361 bytes; Created: Mar. 19, 2024 and is incorporated herein by reference.

### FIELD OF THE INVENTION

The present invention relates to compositions of antigen binding proteins including antibodies capable of binding to human CDH19 on the surface of a target cell, as well as related methods. Moreover, the invention provides a nucleic acid sequence encoding the antibody construct, a vector comprising the nucleic acid sequence and a host cell transformed or transfected with the nucleic acid sequence or a vector comprising the nucleic acid sequence. Furthermore, the invention provides a process for the production of the antibody of the invention, a method of treatment using the antibody and a kit comprising the antibody.

### BACKGROUND OF THE INVENTION

Melanoma is a skin cancer that is caused by the oncogenic transformation of melanocytes, which are pigment producing skin cells. As of 2009, Melanoma had a prevalence of more than 870,000 cases in the US alone (US National Institutes of Health). Each year, over 75,000 new cases of melanoma are diagnosed in the US, and approximately 25% of patients have advanced disease at the time of diagnosis. Despite the fact that cases of primary melanoma can be cured by surgery if they are detected early enough, melanoma is the leading cause of death from skin disease in the US, responsible for about 10,000 deaths per year in the US. Once the disease has spread and became metastatic, the prognosis is poor, with a 5 year relative survival of 15%.

There are four basic types of melanomas. Three types are found in the top layers of the skin and the fourth one is invasive and has penetrated deeper into the skin and may have spread to other areas of the body.

Superficial spreading melanoma is the most common type of melanoma which accounts for about 70% of all cases. It grows along the top layer of the skin for a fairly long time before penetrating more deeply. It first appears as a flat or slightly raised discolored patch that has irregular borders and may be somewhat asymmetrical in form. The color varies, and you may see areas of tan, brown, black, red, blue or white. This type of melanoma can occur in a previously benign mole and is found most often in young people.

Lentigo maligna is similar to the superficial spreading type, as it also remains close to the skin surface for quite a while, and usually appears as a flat or mildly elevated mottled tan, brown or dark brown discoloration. It is found most often in the elderly. When this cancer becomes invasive, it is referred to as lentigo maligna melanoma.

Acral lentiginous melanoma also spreads superficially before penetrating more deeply. It is quite different from the others, though, as it usually appears as a black or brown discoloration under the nails or on the soles of the feet or palms of the hands. This type of melanoma is sometimes found on dark-skinned people, and can often advance more quickly than superficial spreading melanoma and lentigo maligna.

Nodular melanoma is usually invasive at the time it is first diagnosed. The malignancy is recognized when it becomes a bump. It is usually black, but occasionally is blue, gray, white, brown, tan, red or skin tone. This is the most aggressive of the melanomas, and is found in 10 to 15 percent of cases.

Common treatments for metastatic melanoma include chemotherapy, targeted therapies for eligible patients (e.g. BRAF inhibitor treatment for patients with BRAF mutations) and immunotherapy. Metastatic melanoma is a tumor type where immunotherapy has been demonstrated to not only slow disease progression, but to lead to cures in late stage patients. Interleukin-2 was approved for the use in metastatic melanoma in 1998, and in 2011 an antibody targeting CTLA4, a member of a new generation of immune checkpoint inhibitors, gained approval by the FDA.

CDH19 is a type II cadherin transmembrane protein of unknown function. The human gene was cloned in 2000 based on its sequence similarity to CDH7 (Kools, P. et al. Genomics. 2000). Expressed Sequence Tags (ESTs) for CDH19 were isolated from melanocyte cDNA libraries, indicating that expression of CDH19 may be limited to cells of neural crest origin (Kools, P. et al. Genomics. 2000). In support of this notion, rat CDH19 was found to be expressed primarily in nerve ganglia and in Schwann cells during rat embryonic development (Takahashi, M. and Osumi, O. Devl Dynamics. 2005.).

Diagnostic antibodies detecting CDH19 in Western Blot, immunohistochemitstry or flow cytometry are known in the art and commercially available. Those antibodies comprise poly- and monoclonal antibodies generated in animal hosts.

### SUMMARY OF THE INVENTION

The present invention provides an isolated human antibody or antigen binding fragment thereof capable of binding to human CDH19 on the surface of a target cell. In a preferred embodiment the antibody or antigen binding fragment thereof comprises a monoclonal antibody or a fragment thereof.

In one embodiment the human antibody or antigen binding fragment thereof of the invention comprises a human binding domain or antigen binding fragment thereof comprising a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 52, CDR-H2 as depicted in SEQ ID NO: 53, CDR-H3 as depicted in SEQ ID NO: 54, CDR-L1 as depicted in SEQ ID NO: 220, CDR-L2 as depicted in SEQ ID NO: 221 and CDR-L3 as depicted in SEQ ID NO: 222, CDR-H1 as depicted in SEQ ID NO: 82, CDR-H2 as depicted in SEQ ID NO: 83, CDR-H3 as depicted in SEQ ID NO: 84, CDR-L1 as depicted in SEQ ID NO: 250, CDR-L2 as depicted in SEQ ID NO: 251 and CDR-L3 as depicted in SEQ ID NO: 252, CDR-H1 as depicted in SEQ ID NO: 82, CDR-H2 as depicted in SEQ ID NO: 83, CDR-H3 as depicted in SEQ ID NO: 84, CDR-L1 as depicted in SEQ ID NO: 250, CDR-L2 as depicted in SEQ ID NO: 251 and CDR-L3 as depicted in SEQ ID NO: 927, CDR-H1 as depicted in SEQ ID NO: 82, CDR-H2 as depicted in SEQ ID NO: 83, CDR-H3 as depicted in SEQ ID NO: 909, CDR-L1 as depicted in SEQ ID NO: 250, CDR-L2 as depicted in SEQ ID NO: 251 and CDR-L3 as depicted in SEQ ID NO: 927, CDR-H1 as depicted in SEQ ID NO: 52, CDR-H2 as depicted in SEQ ID NO: 53, CDR-H3 as depicted in SEQ ID NO: 54, CDR-L1 as depicted in SEQ ID NO: 220, CDR-L2 as depicted in SEQ ID NO: 221 and CDR-L3 as depicted in SEQ ID NO: 926, and CDR-H1 as depicted in SEQ ID NO: 52, CDR-H2 as depicted in SEQ ID NO: 53, CDR-H3 as depicted in SEQ ID NO: 904, CDR-L1 as depicted in SEQ ID NO: 220, CDR-L2 as depicted in SEQ ID NO: 221 and CDR-L3 as depicted in SEQ ID NO: 926;
(b) CDR-H1 as depicted in SEQ ID NO: 124, CDR-H2 as depicted in SEQ ID NO: 125, CDR-H3 as depicted in SEQ ID NO: 126, CDR-L1 as depicted in SEQ ID NO: 292, CDR-L2 as depicted in SEQ ID NO: 293 and CDR-L3 as depicted in SEQ ID NO: 294, CDR-H1 as depicted in SEQ ID NO: 130, CDR-H2 as depicted in SEQ ID NO: 131, CDR-H3 as depicted in SEQ ID NO: 132, CDR-L1 as depicted in SEQ ID NO: 298, CDR-L2 as depicted in SEQ ID NO: 299 and CDR-L3 as depicted in SEQ ID NO: 300, CDR-H1 as depicted in SEQ ID NO: 136, CDR-H2 as depicted in SEQ ID NO: 137, CDR-H3 as depicted in SEQ ID NO: 138, CDR-L1 as depicted in SEQ ID NO: 304, CDR-L2 as depicted in SEQ ID NO: 305 and CDR-L3 as depicted in SEQ ID NO: 306, CDR-H1 as depicted in SEQ ID NO: 142, CDR-H2 as depicted in SEQ ID NO: 143, CDR-H3 as depicted in SEQ ID NO: 144, CDR-L1 as depicted in SEQ ID NO: 310, CDR-L2 as depicted in SEQ ID NO: 311 and CDR-L3 as depicted in SEQ ID NO: 312, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 318, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 336, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 294, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 928, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 929, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 336, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 942, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 943, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 318, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 937, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 938, CDR-H1 as depicted in SEQ ID NO: 148, CDR-H2 as depicted in SEQ ID NO: 149, CDR-H3 as depicted in SEQ ID NO: 919, CDR-L1 as depicted in SEQ ID NO: 316, CDR-L2 as depicted in SEQ ID NO: 317 and CDR-L3 as depicted in SEQ ID NO: 938, CDR-H1 as depicted in SEQ ID NO: 142, CDR-H2 as depicted in SEQ ID NO: 143, CDR-H3 as depicted in SEQ ID NO: 144, CDR-L1 as depicted in SEQ ID NO: 310, CDR-L2 as depicted in SEQ ID NO: 311 and CDR-L3 as depicted in SEQ ID NO: 935, CDR-H1 as depicted in SEQ ID NO: 142, CDR-H2 as depicted in SEQ ID NO: 143, CDR-H3 as depicted in SEQ ID NO: 918, CDR-L1 as depicted in SEQ ID NO: 310, CDR-L2 as depicted in SEQ ID NO: 311 and CDR-L3 as depicted in SEQ ID NO: 935, CDR-H1 as depicted in SEQ ID NO: 142, CDR-H2 as depicted in SEQ ID NO: 143, CDR-H3 as depicted in SEQ ID NO: 918, CDR-L1 as depicted in SEQ ID NO: 310, CDR-L2 as depicted in SEQ ID NO: 311 and CDR-L3 as depicted in SEQ ID NO: 936, CDR-H1 as depicted in SEQ ID NO: 136, CDR-H2 as depicted in SEQ ID NO: 137, CDR-H3 as depicted in SEQ ID NO: 138, CDR-L1 as depicted in SEQ ID NO: 304, CDR-L2 as depicted in SEQ ID NO: 305 and CDR-L3 as depicted in SEQ ID NO: 933, CDR-H1 as depicted in SEQ ID NO: 136, CDR-H2 as depicted in SEQ ID NO: 137, CDR-H3 as depicted in SEQ ID NO: 917, CDR-L1 as depicted in SEQ ID NO: 304, CDR-L2 as depicted in SEQ ID NO: 305 and CDR-L3 as depicted in SEQ ID NO: 934, CDR-H1 as depicted in SEQ ID NO: 130, CDR-H2 as depicted in SEQ ID NO: 131, CDR-H3 as depicted in SEQ ID NO: 132, CDR-L1 as depicted in SEQ ID NO: 298, CDR-L2 as depicted in SEQ ID NO: 299 and CDR-L3 as depicted in SEQ ID NO: 930, CDR-H1 as depicted in SEQ ID NO: 130, CDR-H2 as depicted in SEQ ID NO: 131, CDR-H3 as depicted in SEQ ID NO: 916, CDR-L1 as depicted in SEQ ID NO: 298, CDR-L2 as depicted in SEQ ID NO: 299 and CDR-L3 as depicted in SEQ ID NO: 931, and CDR-H1 as depicted in SEQ ID NO: 130, CDR-H2 as depicted in SEQ ID NO: 131, CDR-H3 as depicted in SEQ ID NO: 916, CDR-L1 as depicted in SEQ ID NO: 298, CDR-L2 as depicted in SEQ ID NO: 299 and CDR-L3 as depicted in SEQ ID NO: 932;
(c) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 100, CDR-H2 as depicted in SEQ ID NO: 101, CDR-H3 as depicted in SEQ ID NO: 102, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 118, CDR-H2 as depicted in SEQ ID NO: 119, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, CDR-H1 as depicted in SEQ ID NO: 154, CDR-H2 as depicted in SEQ ID NO: 155, CDR-H3 as depicted in SEQ ID NO: 156, CDR-L1 as depicted in SEQ ID NO: 322, CDR-L2 as depicted in SEQ ID NO: 323 and CDR-L3 as depicted in SEQ ID NO: 324, CDR-H1 as depicted in SEQ ID NO: 100, CDR-H2 as depicted in SEQ ID NO: 101, CDR-H3 as depicted in SEQ ID NO: 912, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 100, CDR-H2 as depicted in SEQ ID NO: 101, CDR-H3 as depicted in SEQ ID NO: 913, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 94, CDR-H2 as depicted in SEQ ID NO: 95, CDR-H3 as depicted in SEQ ID NO: 910, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 94, CDR-H2 as depicted in SEQ ID NO: 95, CDR-H3 as depicted in SEQ ID NO: 911, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 118, CDR-H2 as depicted in SEQ ID NO: 119, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, CDR-H1 as depicted in SEQ ID NO: 118, CDR-H2 as depicted in SEQ ID NO: 914, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, and CDR-H1 as depicted in SEQ ID NO: 154, CDR-H2 as depicted in SEQ ID NO: 155, CDR-H3 as depicted in SEQ ID NO: 920, CDR-L1 as depicted in SEQ ID NO: 322, CDR-L2 as depicted in SEQ ID NO: 323 and CDR-L3 as depicted in SEQ ID NO: 324;
(d) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 172, CDR-L2 as depicted in SEQ ID NO: 173 and CDR-L3 as depicted in SEQ ID NO: 174, CDR-H1 as depicted in SEQ ID NO: 10, CDR-H2 as depicted in SEQ ID NO: 11, CDR-H3 as depicted in SEQ ID NO: 12, CDR-L1 as depicted in SEQ ID NO: 178, CDR-L2 as depicted in SEQ ID NO: 179 and CDR-L3 as depicted in SEQ ID NO: 180, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 196, CDR-L2 as depicted in SEQ ID NO: 197 and CDR-L3 as depicted in SEQ ID NO: 198, CDR-H1 as depicted in SEQ ID NO: 34, CDR-H2 as depicted in SEQ ID NO: 35, CDR-H3 as depicted in SEQ ID NO: 36, CDR-L1 as depicted in SEQ ID NO: 202, CDR-L2 as depicted in SEQ ID NO: 203 and CDR-L3 as depicted in SEQ ID NO: 204, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 214, CDR-L2 as depicted in SEQ ID NO: 215 and CDR-L3 as depicted in SEQ ID NO: 216, CDR-H1 as depicted in SEQ ID NO: 58, CDR-H2 as depicted in SEQ ID NO: 59, CDR-H3 as depicted in SEQ ID NO: 60, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 64, CDR-H2 as depicted in SEQ ID NO: 65, CDR-H3 as depicted in SEQ ID NO: 66, CDR-L1 as depicted in SEQ ID NO: 232, CDR-L2 as depicted in SEQ ID NO: 233 and CDR-L3 as depicted in SEQ ID NO: 234, CDR-H1 as depicted in SEQ ID NO: 70, CDR-H2 as depicted in SEQ ID NO: 71, CDR-H3 as depicted in SEQ ID NO: 72, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 328, CDR-L2 as depicted in SEQ ID NO: 329 and CDR-L3 as depicted in SEQ ID NO: 330, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 902, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 903, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 925, CDR-L2 as depicted in SEQ ID NO: 215 and CDR-L3 as depicted in SEQ ID NO: 216, CDR-H1 as depicted in SEQ ID NO: 70, CDR-H2 as depicted in SEQ ID NO: 907, CDR-H3 as depicted in SEQ ID NO: 72, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 70, CDR-H2 as depicted in SEQ ID NO: 907, CDR-H3 as depicted in SEQ ID NO: 908, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 28, CDR-H2 as depicted in SEQ ID NO: 901, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, CDR-H1 as depicted in SEQ ID NO: 58, CDR-H2 as depicted in SEQ ID NO: 905, CDR-H3 as depicted in SEQ ID NO: 906, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 58, CDR-H2 as depicted in SEQ ID NO: 905, CDR-H3 as depicted in SEQ ID NO: 60, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 939, CDR-L2 as depicted in SEQ ID NO: 329 and CDR-L3 as depicted in SEQ ID NO: 330, CDR-H1 as depicted in SEQ ID NO: 160, CDR-H2 as depicted in SEQ ID NO: 921, CDR-H3 as depicted in SEQ ID NO: 162, CDR-L1 as depicted in SEQ ID NO: 939, CDR-L2 as depicted in SEQ ID NO: 329 and CDR-L3 as depicted in SEQ ID NO: 940, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 941, CDR-L2 as depicted in SEQ ID NO: 329 and CDR-L3 as depicted in SEQ ID NO: 330, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 196, CDR-L2 as depicted in SEQ ID NO: 197 and CDR-L3 as depicted in SEQ ID NO: 923, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, CDR-H1 as depicted in SEQ ID NO: 28, CDR-H2 as depicted in SEQ ID NO: 901, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, and CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 939, CDR-L2 as depicted in SEQ ID NO: 329 and CDR-L3 as depicted in SEQ ID NO: 330; and
(e) CDR-H1 as depicted in SEQ ID NO: 76, CDR-H2 as depicted in SEQ ID NO: 77, CDR-H3 as depicted in SEQ ID NO: 78, CDR-L1 as depicted in SEQ ID NO: 244, CDR-L2 as depicted in SEQ ID NO: 245 and CDR-L3 as depicted in SEQ ID NO: 246, CDR-H1 as depicted in SEQ ID NO: 88, CDR-H2 as depicted in SEQ ID NO: 89, CDR-H3 as depicted in SEQ ID NO: 90, CDR-L1 as depicted in SEQ ID NO: 256, CDR-L2 as depicted in SEQ ID NO: 257 and CDR-L3 as depicted in SEQ ID NO: 258, CDR-H1 as depicted in SEQ ID NO: 106, CDR-H2 as depicted in SEQ ID NO: 107, CDR-H3 as depicted in SEQ ID NO: 108, CDR-L1 as depicted in SEQ ID NO: 274, CDR-L2 as depicted in SEQ ID NO: 275 and CDR-L3 as depicted in SEQ ID NO: 276, CDR-H1 as depicted in SEQ ID NO: 112, CDR-H2 as depicted in SEQ ID NO: 113, CDR-H3 as depicted in SEQ ID NO: 114, CDR-L1 as depicted in SEQ ID NO: 280, CDR-L2 as depicted in SEQ ID NO: 281 and CDR-L3 as depicted in SEQ ID NO: 282, and CDR-H1 as depicted in SEQ ID NO: 106, CDR-H2 as depicted in SEQ ID NO: 107, CDR-H3 as depicted in SEQ ID NO: 108, CDR-L1 as depicted in SEQ ID NO: 274, CDR-L2 as depicted in SEQ ID NO: 275 and CDR-L3 as depicted in SEQ ID NO: 276.

In a further embodiment of the human antibody or antigen binding fragment thereof of the invention the human binding domain or antigen binding fragment thereof comprises a VH region selected from the group consisting of VH regions
(a) as depicted in SEQ ID NO: 362, SEQ ID NO: 364, SEQ ID NO: 485, SEQ ID NO: 486, SEQ ID NO: 487, SEQ ID NO: 492, SEQ ID NO: 493, SEQ ID NO: 494, and SEQ ID NO: 495;
(b) as depicted in SEQ ID NO: 342, SEQ ID NO: 366, SEQ ID NO: 370, SEQ ID NO: 344, SEQ ID NO: 372, SEQ ID NO: 368, SEQ ID NO: 496, SEQ ID NO: 497, SEQ ID NO: 498, SEQ ID NO: 499, SEQ ID NO: 500, SEQ ID NO: 508, SEQ ID NO: 509, SEQ ID NO: 510, SEQ ID NO: 511, SEQ ID NO: 512, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, and SEQ ID NO: 538;
(c) as depicted in SEQ ID NO: 338, SEQ ID NO: 354, SEQ ID NO: 378, SEQ ID NO: 356, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 501, SEQ ID NO: 502, SEQ ID NO: 503, SEQ ID NO: 504, SEQ ID NO: 505, SEQ ID NO: 506, SEQ ID NO: 517, and SEQ ID NO: 518;
(d) as depicted in SEQ ID NO: 352, SEQ ID NO: 360, SEQ ID NO: 388, SEQ ID NO: 386, SEQ ID NO: 340, SEQ ID NO: 346, SEQ ID NO: 374, SEQ ID NO: 348, SEQ ID NO: 390, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 488, SEQ ID NO: 489, SEQ ID NO: 490, SEQ ID NO: 491, SEQ ID NO: 513, SEQ ID NO: 514, SEQ ID NO: 515, SEQ ID NO: 516, SEQ ID NO: 540, SEQ ID NO: 541, SEQ ID NO: 542, and SEQ ID NO: 543; and
(e) as depicted in SEQ ID NO: 376, SEQ ID NO: 392, SEQ ID NO: 358, SEQ ID NO: 350, and SEQ ID NO: 507.

In another embodiment the human antibody or antigen binding fragment thereof of the invention comprises the human binding domain or antigen binding fragment thereof comprising a VL region selected from the group consisting of VL regions (a) as depicted in SEQ ID NO: 418, SEQ ID NO: 420, SEQ ID NO: 580, SEQ ID NO: 581, SEQ ID NO: 582, SEQ ID NO: 587, SEQ ID NO: 588, SEQ ID NO: 589, and SEQ ID NO: 590;
(b) as depicted in SEQ ID NO: 398, SEQ ID NO: 422, SEQ ID NO: 426, SEQ ID NO: 400, SEQ ID NO: 428, SEQ ID NO: 424, SEQ ID NO: 591, SEQ ID NO: 592, SEQ ID NO: 593, SEQ ID NO: 594, SEQ ID NO: 595, SEQ ID NO: 603, SEQ ID NO: 604, SEQ ID NO: 605, SEQ ID NO: 606, SEQ ID NO: 607, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, and SEQ ID NO: 633;
(c) as depicted in SEQ ID NO: 394, SEQ ID NO: 410, SEQ ID NO: 434, SEQ ID NO: 412, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 596, SEQ ID NO: 597, SEQ ID NO: 598, SEQ ID NO: 599, SEQ ID NO: 600, SEQ ID NO: 601, SEQ ID NO: 612, and SEQ ID NO: 613;
(d) as depicted in SEQ ID NO: 408, SEQ ID NO: 416, SEQ ID NO: 444, SEQ ID NO: 442, SEQ ID NO: 396, SEQ ID NO: 402, SEQ ID NO: 430, SEQ ID NO: 404, SEQ ID NO: 446, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 583, SEQ ID NO: 584, SEQ ID NO: 585, SEQ ID NO: 586, SEQ ID NO: 608, SEQ ID NO: 609, SEQ ID NO: 610, SEQ ID NO: 611, SEQ ID NO: 635, SEQ ID NO: 636, SEQ ID NO: 637, and SEQ ID NO: 638; and
(e) as depicted in SEQ ID NO: 432, SEQ ID NO: 448, SEQ ID NO: 414, SEQ ID NO: 406, and SEQ ID NO: 602.

The invention further provides an embodiment of the human antibody or antigen binding fragment thereof of the invention, wherein the human binding domain or antigen binding fragment thereof comprises a VH region and a VL region selected from the group consisting of:
(1) pairs of a VH region and a VL region as depicted in SEQ ID NOs: 362+418, SEQ ID NOs: 364+420, SEQ ID NOs: 485+580, SEQ ID NOs: 486+581, SEQ ID NOs: 487+582, SEQ ID NOs: 492+587, SEQ ID NOs: 493+588, SEQ ID NOs: 494+589, and SEQ ID NOs: 495+590;
(2) pairs of a VH region and a VL region as depicted in SEQ ID NOs: 342+398, SEQ ID NOs: 366+422, SEQ ID NOs: 370+426, SEQ ID NOs: 344+400, SEQ ID NOs: 372+428, SEQ ID NOs: 368+424, SEQ ID NOs: 496+591, SEQ ID NOs: 497+592, SEQ ID NOs: 498+593, SEQ ID NOs: 499+594, SEQ ID NOs: 500+595, SEQ ID NOs: 508+603, SEQ ID NOs: 509+604, SEQ ID NOs: 510+605, SEQ ID NOs: 511+606, SEQ ID NOs: 512+607, SEQ ID NOs: 519+614, SEQ ID NOs: 520+615, SEQ ID NOs: 521+616, SEQ ID NOs: 522+617, SEQ ID NOs: 523+618, SEQ ID NOs: 524+619, SEQ ID NOs: 525+620, SEQ ID NOs: 526+621, SEQ ID NOs: 527+622, SEQ ID NOs: 528+623, SEQ ID NOs: 529+624, SEQ ID NOs: 530+625, SEQ ID NOs: 531+626, SEQ ID NOs: 532+627, SEQ ID NOs: 533+628, SEQ ID NOs: 534+629, SEQ ID NOs: 535+630, SEQ ID NOs: 536+631, SEQ ID NOs: 537+632, and SEQ ID NOs: 538+633;
(3) pairs of a VH region and a VL region as depicted in SEQ ID NOs: 338+394, SEQ ID NOs: 354+410, SEQ ID NOs: 378+434, SEQ ID NOs: 356+412, SEQ ID NOs: 476+571, SEQ ID NOs: 477+572, SEQ ID NOs: 478+573, SEQ ID NOs: 479+574, SEQ ID NOs: 480+575, SEQ ID NOs: 481+576, SEQ ID NOs: 482+577, SEQ ID NOs: 483+578, SEQ ID NOs: 484+579, SEQ ID NOs: 501+596, SEQ ID NOs: 502+597, SEQ ID NOs: 503+598, SEQ ID NOs: 504+599, SEQ ID NOs: 505+600, SEQ ID NOs: 506+601, SEQ ID NOs: 517+612, and SEQ ID NOs: 518+613;
(4) pairs of a VH region and a VL region as depicted in SEQ ID NOs: 352+408, SEQ ID NOs: 360+416, SEQ ID NOs: 388+444, SEQ ID NOs: 386+442, SEQ ID NOs: 340+396, SEQ ID NOs: 346+402, SEQ ID NOs: 374+430, SEQ ID NOs: 348+404, SEQ ID NOs: 390+446, SEQ ID NOs: 463+558, SEQ ID NOs: 464+559, SEQ ID NOs: 465+560, SEQ ID NOs: 466+561, SEQ ID NOs: 467+562, SEQ ID NOs: 468+563, SEQ ID NOs: 469+564, SEQ ID NOs: 470+565, SEQ ID NOs: 471+566, SEQ ID NOs: 472+567, SEQ ID NOs: 473+568, SEQ ID NOs: 474+569, SEQ ID NOs: 475+570, SEQ ID NOs: 488+583, SEQ ID NOs: 489+584, SEQ ID NOs: 490+585, SEQ ID NOs: 491+586, SEQ ID NOs: 513+608, SEQ ID NOs: 514+609, SEQ ID NOs: 515+610, SEQ ID NOs: 516+611, SEQ ID NOs: 540+635, SEQ ID NOs: 541+636, SEQ ID NOs: 542+637, and SEQ ID NOs: 543+638; and
(5) pairs of a VH region and a VL region as depicted in SEQ ID NOs: 376+432, SEQ ID NOs: 392+448, SEQ ID NOs: 358+414, SEQ ID NOs: 350+406, and SEQ ID NOs: 507+602.

In a further embodiment the human binding domain or antigen binding fragment thereof comprises the groups of heavy and light chains having an amino acid sequence selected from the group consisting of
(1) a heavy and light chain as depicted in SEQ ID NOs: 644+680, SEQ ID NOs: 650+686, SEQ ID NOs: 747+842, SEQ ID NOs: 748+843, SEQ ID NOs: 749+844, SEQ ID NOs: 754+849, SEQ ID NOs: 755+850, SEQ ID NOs: 756+851, and SEQ ID NOs: 757+852;
(2) a heavy and light chain as depicted in SEQ ID NOs: 660+696, SEQ ID NOs: 662+698, SEQ ID NOs: 668+704, SEQ ID NOs: 674+710, SEQ ID NOs: 672+708, SEQ ID NOs: 658+694, SEQ ID NOs: 758+853, SEQ ID NOs: 759+854, SEQ ID NOs: 760+855, SEQ ID NOs: 761+856, SEQ ID NOs: 762+857, SEQ ID NOs: 770+865, SEQ ID NOs: 771+866, SEQ ID NOs: 772+867, SEQ ID NOs: 773+868, SEQ ID NOs: 774+869, SEQ ID NOs: 781+876, SEQ ID NOs: 782+877, SEQ ID NOs: 783+878, SEQ ID NOs: 784+879, SEQ ID NOs: 785+880, SEQ ID NOs: 786+881, SEQ ID NOs: 787+882, SEQ ID NOs: 788+883, SEQ ID NOs: 789+884, SEQ ID NOs: 790+885, SEQ ID NOs: 791+886, SEQ ID NOs: 792+887, SEQ ID NOs: 793+888, SEQ ID NOs: 794+889, SEQ ID NOs: 795+890, SEQ ID NOs: 796+891, SEQ ID NOs: 797+892, SEQ ID NOs: 798+893, SEQ ID NOs: 799+894, and SEQ ID NOs: 800+895;
(3) a heavy and light chain as depicted in SEQ ID NOs: 656+692, SEQ ID NOs: 654+690, SEQ ID NOs: 664+700, SEQ ID NOs: 670+706, SEQ ID NOs: 738+833, SEQ ID NOs: 739+834, SEQ ID NOs: 740+835, SEQ ID NOs: 741+836, SEQ ID NOs: 742+837, SEQ ID NOs: 743+838, SEQ ID NOs: 744+839, SEQ ID NOs: 745+840, SEQ ID NOs: 746+841, SEQ ID NOs: 763+858, SEQ ID NOs: 764+859, SEQ ID NOs: 765+860, SEQ ID NOs: 766+861, SEQ ID NOs: 767+862, SEQ ID NOs: 768+863, SEQ ID NOs: 779+874, and SEQ ID NOs: 780+875;
(4) a heavy and light chain as depicted in SEQ ID NOs: 640+676, SEQ ID NOs: 642+678, SEQ ID NOs: 646+682, SEQ ID NOs: 648+684, SEQ ID NOs: 666+702, SEQ ID NOs: 725+820, SEQ ID NOs: 726+821, SEQ ID NOs: 727+822, SEQ ID NOs: 728+823, SEQ ID NOs: 729+824, SEQ ID NOs: 730+825, SEQ ID NOs: 731+826, SEQ ID NOs: 732+827, SEQ ID NOs: 733+828, SEQ ID NOs: 734+829, SEQ ID NOs: 735+830, SEQ ID NOs: 736+831, SEQ ID NOs: 737+832, SEQ ID NOs: 750+845, SEQ ID NOs: 751+846, SEQ ID NOs: 752+847, SEQ ID NOs: 753+848, SEQ ID NOs: 775+870, SEQ ID NOs: 776+871, SEQ ID NOs: 777+872, SEQ ID NOs: 778+873, SEQ ID NOs: 802+897, SEQ ID NOs: 803+898, SEQ ID NOs: 804+899, and SEQ ID NOs: 805+900; and
(5) a heavy and light chain as depicted in SEQ ID NOs: 652+688, and SEQ ID NOs: 769+864.

In another embodiment the invention is directed to an antibody construct comprising the human antibody or antigen binding fragment thereof capable of binding to human CDH19 on the surface of a target cell as described above that is conjugated to a chemotherapeutic agent.

In one embodiment of the antibody construct of the invention a linker conjugates the chemotherapeutic agent to the human antibody or antigen binding fragment thereof. In a preferred embodiment of the antibody construct of the invention the linker is a non-cleavable linker.

It is also preferred that the linker in the antibody construct of the invention comprises MCC. In a further embodiment of the antibody construct of the invention the chemotherapeutic agent is conjugated to one or more lysines contained in the human antibody or antigen binding fragment thereof.

In one embodiment of the antibody construct of the invention the chemotherapeutic agent is DM1.

In a preferred embodiment of the antibody construct of the invention the average number of DM1 molecules per antibody construct is between 1 and 10.

It is also preferred for the antibody construct of the invention that the average number of DM1 molecules per antibody construct is between 3 and 7.

Moreover, it is preferred for the antibody construct of the invention that the average number of DM1 molecules per antibody construct is between 4 and 6.

In a further alternative embodiment of the antibody construct of the invention the average number of DM1 molecules per antibody construct is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0.

The invention further provides an isolated nucleic acid molecule or sequence encoding a human antibody or antigen binding fragment thereof of the invention.

Furthermore, the invention provides a vector comprising a nucleic acid sequence of the invention. Moreover, the invention provides a host cell transformed or transfected with the nucleic acid sequence of the invention or with a vector comprising the nucleic acid molecule.

In a further embodiment the invention provides a process for the production of a human antibody or an antigen binding fragment thereof of the invention, said process comprising culturing a host cell of the invention under conditions allowing the expression of the human antibody or antigen binding fragment thereof of the invention and recovering the produced antibody or antigen binding fragment thereof from the culture.

In a further embodiment the invention provides a process for the production of an antibody consturct comprising a human antibody or an antigen binding fragment thereof of the invention, said process comprising culturing a host cell of the invention under conditions allowing the expression of the human antibody or antigen binding fragment thereof of the invention and recovering the produced antibody or antigen binding fragment thereof from the culture, and conjugating a chemotherapeutic agent to the recovered antibody or antigen binding fragment thereof to produce the antibody conjugate.

Moreover, the invention provides a pharmaceutical composition comprising a human antibody or antigen binding fragment thereof of the invention or an antibody construct of the invention or produced according to the process of the invention in admixture with a pharmaceutically acceptable carrier thereof.

In one embodiment the invention provides the human antibody or antigen binding fragment thereof of the invention, the antibody construct of the invention, or produced according to the process of the invention for use in the prevention, treatment or amelioration of a melanoma disease or metastatic melanoma disease. Preferably, the melanoma disease or metastatic melanoma disease is selected from the group consisting of superficial spreading melanoma, lentigo maligna, lentigo maligna melanoma, acral lentiginous melanoma and nodular melanoma.

The invention also provides a method for the treatment or amelioration of a melanoma disease or metastatic melanoma disease, comprising the step of administering to a subject in need thereof the antibody or antigen binding fragment thereof of the invention, the antibody construct of the invention, an antibody or antigen binding fragment thereof of the invention or the antibody construct of the invention produced according to the process of the invention or a pharmaceutical composition of the invention.

In a preferred embodiment method the invention the melanoma disease or metastatic melanoma disease is selected from the group consisting of superficial spreading melanoma, lentigo maligna, lentigo maligna melanoma, acral lentiginous melanoma and nodular melanoma.

In a further embodiment, the invention provides a kit comprising an antibody or antigen binding fragment thereof of the invention, an antibody construct of the invention, an antibody or antigen binding fragment thereof of the invention or the antibody construct produced according to the process of the invention, a vector of the invention, and/or a host cell of the invention.

### BRIEF DESCRIPTION OF THE DRAWINGS

FIG. 1 depicts cell viability data of Colo-699 cells that have been treated with fully human anti-CDH19 antibodies and a high concentration of a goat anti-human Fc monovalent Fab conjugated with DM1 (DM1-Fab) at a drug-antibody ratio (DAR) (˜1.3).

FIG. 2 depicts the average cell viability data from a CHL-1 assay plotted against the average cell viability data from the Colo-699 assay.

FIG. 3 shows the relative expression of CDH19 mRNA in metastatic and primary melanoma samples.

FIG. 4 shows the expression of CDH19 protein in human tumor samples by IHC.

FIG. 5 shows the results of the analysis of tumor cell lines by flow cytometry and IHC to identify model systems with CDH19 expression similar to human tumors based on the number of CDH19 receptors present on the cell surface.

FIG. 6 shows in vitro activity of a CDH19 ADC against the model tumor cell lines.

FIG. 7 shows in vitro activity of a CDH19 ADC in model tumor cell lines at varying DAR ratios.

FIG. 8 shows in vivo activity of CDH19 ADCs in a xenograft mouse model as compared to naked CDH19 antibodies.

FIG. 9 shows in vivo activity of CDH19 ADCs in a xenograft mouse model. 4B10-DM1 Moderately Inhibited Tumor Growth at 182 μg/kg DM1 in CHL-1 Xenografts

FIG. 10 shows in vivo activity of CDH19 ADCs in a xenograft mouse model. Increasing the DAR Did Not Increase Tumor Growth Inhibition in CHL-1 Xenografts

FIG. 11 shows in vivo activity of CDH19 ADCs in a xenograft mouse model. Anti-CDH19 ADCs Moderately Inhibited Tumor Growth in COL0699 Xenografts

### DETAILED DESCRIPTION OF THE INVENTION

### Definitions

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

The definition of the term “antibody” includes embodiments such as monoclonal, chimeric, single chain, humanized and human antibodies, as well as antibody fragments, like, inter alia, Fab fragments. Antibody fragments or derivatives further comprise F(ab′)₂, Fv, scFv fragments or single domain antibodies such as domain antibodies or nanobodies, single variable domain antibodies or immunoglobulin single variable domain comprising merely one variable domain, which might be VHH, VH or VL, that specifically bind an antigen or epitope independently of other V regions or domains; see, for example, Harlow and Lane (1988) and (1999), loc. cit.; Kontermann and Dubel, Antibody Engineering, Springer, 2nd ed. 2010 and Little, Recombinant Antibodies for Immunotherapy, Cambridge University Press 2009. Such immunoglobulin single variable domain encompasses not only an isolated antibody single variable domain polypeptide, but also larger polypeptides that comprise one or more monomers of an antibody single variable domain polypeptide sequence.

In line with this definition all above described embodiments of the term antibody can be subsumed under the term “antibody construct”. Said term also includes diabodies or Dual-Affinity Re-Targeting (DART) antibodies. Further envisaged are (bispecific) single chain diabodies, tandem diabodies (Tandab's), “minibodies” exemplified by a structure which is as follows: (VH-VL-CH3)₂, (scFv-CH3)₂ or (scFv-CH3-scFv)₂, “Fc DART” antibodies and, “IgG DART” antibodies, and multibodies such as triabodies. Immunoglobulin single variable domains encompass not only an isolated antibody single variable domain polypeptide, but also larger polypeptides that comprise one or more monomers of an antibody single variable domain polypeptide sequence.

Various procedures are known in the art and may be used for the production of such antibody constructs (antibodies and/or fragments). Thus, (antibody) derivatives can be produced by peptidomimetics. Further, techniques described for the production of single chain antibodies (see, inter alia, U.S. Pat. No. 4,946,778, Kontermann and Dubel (2010), loc. cit. and Little(2009), loc. cit.) can be adapted to produce single chain antibodies specific for elected polypeptide(s). Also, transgenic animals may be used to express humanized antibodies specific for polypeptides and fusion proteins of this invention. For the preparation of monoclonal antibodies, any technique, providing antibodies produced by continuous cell line cultures can be used. Examples for such techniques include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of a target polypeptide, such as CDH19 (Schier, Human Antibodies Hybridomas 7 (1996), 97-105; Malmborg, J. Immunol. Methods 183 (1995), 7-13). It is also envisaged in the context of this invention that the term “antibody” comprises antibody constructs, which may be expressed in a host as described herein below, e.g. antibody constructs which may be transfected and/or transduced via, inter alia, viruses or plasmid vectors.

Furthermore, the term “antibody” as employed in the invention also relates to derivatives or variants of the antibodies described herein which display the same specificity as the described antibodies. Accordingly, the term “antibody” also subsumes antibody constructs such as different types of fragments of antibodies, which still are characterized by the feature of specific binding for CDH19.

The terms “antigen-binding domain”, “antigen-binding fragment” and “antibody binding region” when used herein refer to a part of an antibody molecule that comprises amino acids responsible for the specific binding between antibody and antigen. The part of the antigen that is specifically recognized and bound by the antibody is referred to as the “epitope” as described herein above. As mentioned above, an antigen-binding domain may typically comprise an antibody light chain variable region (VL) and an antibody heavy chain variable region (VH); however, it does not have to comprise both. Fd fragments, for example, have two VH regions and often retain some antigen-binding function of the intact antigen-binding domain. Examples of antigen-binding fragments of an antibody include (1) a Fab fragment, a monovalent fragment having the VL, VH, CL and CH1 domains; (2) a F(ab′)2 fragment, a bivalent fragment having two Fab fragments linked by a disulfide bridge at the hinge region; (3) a Fd fragment having the two VH and CH1 domains; (4) a Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv). Although the two domains of the Fv fragment, VL and VH are coded for by separate genes, they can be joined, using recombinant methods, by a synthetic linker that enables them to be made as a single protein chain in which the VL and VH regions pair to form monovalent molecules (known as single chain Fv (scFv); see e.g., Huston et al. (1988) Proc. Natl. Acad. Sci USA 85:5879-5883). These antibody fragments are obtained using conventional techniques known to those with skill in the art, and the fragments are evaluated for function in the same manner as are intact antibodies.

The term “monoclonal antibody” as used herein refers to an antibody obtained from a population of substantially homogeneous antibodies, i.e., the individual antibodies comprising the population are identical except for possible naturally occurring mutations and/or post- translation modifications (e.g., isomerizations, amidations) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

The term “human antibody” includes antibodies having variable and constant regions corresponding substantially to human germline immunoglobulin sequences known in the art, including, for example, those described by Kabat et al (See Kabat et al. (1991) loc. cit.). The human antibodies of the invention may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence. It is emphasized that the definition of human antibodies as used herein also contemplates fully human antibodies, which include only non-artificially and/or genetically altered human sequences of antibodies as those can be derived by technologies using systems such as the Xenomice.

Examples of “antibody variants” include humanized variants of non- human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function (s) (see, e.g., U.S. Pat. No. 5,648,260, Kontermann and Dubel (2010), loc. cit. and Little(2009), loc. cit.).

As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term thus preferably excludes sequences generated by genomic rearrangement in an immune cell.

The pairing of a VH and VL together forms a single antigen-binding site. The CH domain most proximal to VH is designated as CH1. Each L chain is linked to an H chain by one covalent disulfide bond, while the two H chains are linked to each other by one or more disulfide bonds depending on the H chain isotype. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR 1, CDR2, and CDR3. Accordingly, CDR constituents on the heavy chain are referred to as H1, H2, and H3, while CDR constituents on the light chain are referred to as L1, L2, and L3.

The term “variable” refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies; it is concentrated in sub-domains of each of the heavy and light chain variable regions. These sub-domains are called “hypervariable” regions or “complementarity determining regions” (CDRs). The more conserved (i.e., non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.). The constant domains are not directly involved in antigen binding, but exhibit various effector functions, such as, for example, antibody-dependent, cell-mediated cytotoxicity and complement activation.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable region (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum (Kabat et al., loc. cit.; Chothia et al., J. Mol. Biol, 1987, 196: 901; and MacCallum et al., J. Mol. Biol, 1996, 262: 732). However, the numbering in accordance with the so-called Kabat system is preferred. The CDR3 of the light chain and, particularly, CDR3 of the heavy chain may constitute the most important determinants in antigen binding within the light and heavy chain variable regions. In some antibodies, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen.

In one embodiment, the antibody of the invention may comprise from one to six of the exemplary CDRs described herein. The antibodies of the invention may be of any type including IgM, IgG (including IgG1, IgG2, IgG3, IgG4), IgD, IgA, or IgE antibody. In a specific embodiment the antigen binding protein is an IgG type antibody, e.g., a IgG1 antibody. In one embodiment, the antibody of the invention may be a mutlispecific antibody, and notably a bispecfic antibody, also sometimes referred to as “diabodies.” These are antibodies that bind to two or more different antigens or different epitopes on a single antigen. In certain embodiments, a bispecific antibody binds CDH19 and an antigen on a human effector cell (e.g., T cell). Such antibodies are useful in targeting an effector cell response against a CDH19 expressing cells, such as a tumor cell. In preferred embodiments, the human effector cell antigen is CD3 (see corresponding formats e.g. in WO 2008/119567. Methods of making bispecific antibodies are known in the art. One such method involves engineering the Fc portion of the heavy chains such as to create “knobs” and “holes” which facilitate heterodimer formation of the heavy chains when co-expressed in a cell. U.S. Pat. No. 7,695,963. Another method also involves engineering the Fc portion of the heavy chain but uses electrostatic steering to encourage heterodimer formation while discouraging homodimer formation of the heavy chains when co-expressed in a cell. WO 2009/089004, which is incorporated herein by reference in its entirety.

In one embodiment, antibody of the invention is a minibody. Minibodies are minimized antibody-like proteins comprising a scFv joined to a CH3 domain. Hu et al., 1996, Cancer Res. 56:3055-3061.

In one embodiment, the antibody of the invention is a domain antibody; see, for example U.S. Pat. No. 6,248,516. Domain antibodies (dAbs) are functional binding domains of antibodies, corresponding to the variable regions of either the heavy (VH) or light (VL) chains of human antibodies. dABs have a molecular weight of approximately 13 kDa, or less than one-tenth the size of a full antibody. dABs are well expressed in a variety of hosts including bacterial, yeast, and mammalian cell systems. In addition, dAbs are highly stable and retain activity even after being subjected to harsh conditions, such as freeze-drying or heat denaturation. See, for example, U.S. Pat. Nos. 6,291,158; 6,582,915; 6,593,081; 6,172,197; US Serial No. 2004/0110941; European Patent 0368684; U.S. Pat. No. 6,696,245, WO04/058821, WO04/003019 and WO03/002609.

In one embodiment, the antibody of the invention is an antibody fragment, that is a fragment of any of the antibodies outlined herein that retain binding specificity to CDH19. In various embodiments, the antibody binding proteins comprise, but are not limited to, a F(ab), F(ab′), F(ab′)2, Fv, or a single chain Fv fragments. At a minimum, an antibody, as meant herein, comprises a polypeptide that can bind specifically to CDH19 comprising all or part of a light or heavy chain variable region, such as one or more CDRs.

Naturally occurring antibodies typically include a signal sequence, which directs the antibody into the cellular pathway for protein secretion and which is typically not present in the mature antibody. A polynucleotide encoding an antibody of the invention may encode a naturally occurring a signal sequence or a heterologous signal sequence as described below.

“Consisting essentially of” means that the amino acid sequence can vary by about 1, 2, 3, 4, 5, 6, 7, 8, 9, 10, 11, 12, 13, 14, or 15% relative to the recited SEQ ID NO: sequence and still retain biological activity, as described herein.

In some embodiments, the antibodies of the invention are isolated proteins or substantially pure proteins. An “isolated” protein is unaccompanied by at least some of the material with which it is normally associated in its natural state, for example constituting at least about 5%, or at least about 50% by weight of the total protein in a given sample. It is understood that the isolated protein may constitute from 5 to 99.9% by weight of the total protein content depending on the circumstances. For example, the protein may be made at a significantly higher concentration through the use of an inducible promoter or high expression promoter, such that the protein is made at increased concentration levels. The definition includes the production of an antigen binding protein in a wide variety of organisms and/or host cells that are known in the art.

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CAB/OS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=ll. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs are at least 80% to the sequences depicted herein, and more typically with preferably increasing homologies or identities of at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antigen binding protein. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs and the nucleotide sequences depicted herein are at least 80%, and more typically with preferably increasing homologies or identities of at least 80%, 81%, 82%, 83%, 84%, 85%, 86%, 87%, 88%, 89%, 90%, 91%, 92%, 93%, 94%, 95%, 96%, 97%, 98%, or 99%, and almost 100%.

Thus, a “variant CDR” is one with the specified homology, similarity, or identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities, such as CDH19 binding.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

The term “hypervariable region” (also known as “complementarity determining regions” or CDRs) when used herein refers to the amino acid residues of an antibody which are (usually three or four short regions of extreme sequence variability) within the V-region domain of an immunoglobulin which form the antigen-binding site and are the main determinants of antigen specificity. There are at least two methods for identifying the CDR residues: (1) An approach based on cross-species sequence variability (i. e., Kabat et al., loc. cit.); and (2) An approach based on crystallographic studies of antigen-antibody complexes (Chothia, C. et al., J. Mol. Biol. 196: 901-917 (1987)). However, to the extent that two residue identification techniques define regions of overlapping, but not identical regions, they can be combined to define a hybrid CDR. However, in general, the CDR residues are preferably identified in accordance with the so-called Kabat (numbering) system.

The term “framework region” refers to the art-recognized portions of an antibody variable region that exist between the more divergent (i.e., hypervariable) CDRs. Such framework regions are typically referred to as frameworks 1 through 4 (FR1, FR2, FR3, and FR4) and provide a scaffold for the presentation of the six CDRs (three from the heavy chain and three from the light chain) in three dimensional space, to form an antigen-binding surface.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800, each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues. The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al and/or revealed by other techniques, for example, crystallography and two or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature.

CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of immunological Interest, US Department of Health and Human Services (1991), eds. Kabat et al. Another standard for characterizing the antigen binding site is to refer to the hypervariable loops as described by Chothia. See, e.g., Chothia, et al. (1987; J. Mol. Biol. 227:799-817); and Tomlinson et al. (1995) EMBO J. 14: 4628-4638. Still another standard is the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, CA, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “binding molecule” or “antibody construct” in the sense of the present disclosure indicates any molecule capable of (specifically) binding to, interacting with or recognizing the target molecule CDH19. Such molecules or constructs may include proteinaceous parts and non-proteinaceous parts (e.g. chemical linkers or chemical cross-linking agents such as glutaraldehyde).

The term “multispecific” as used herein refers to a binding molecule which is an antibody construct and comprises at least a first and a second binding domain, wherein the first binding domain is capable of binding to one antigen or target, and the second binding domain is capable of binding to another antigen or target. Accordingly, antibody constructs according to the invention comprise at least a specificity for CDH19. The “antibody construct” of the invention also comprises multispecific binding molecules such as e.g. trispecific binding molecules, the latter ones including three binding domains.

It is also envisaged that the antibody construct of the invention has, in addition to its function to bind to the target molecules CDH19 and CD3, a further function. In this format, the antibody construct is a bi-, tri- or multifunctional antibody construct by targeting plasma cells through binding to CDH19, mediating cytotoxic T cell activity through CD3 binding and providing a further function such as a fully functional Fc constant domain mediating antibody-dependent cellular cytotoxicity through recruitment of effector cells like NK cells, a label (fluorescent etc.), a therapeutic agent such as, e.g. a toxin or radionuclide, and/or means to enhance serum half-life, etc.

The term “binding domain” characterizes in connection with the present invention a domain which is capable of specifically binding to/interacting with a given target epitope or a given target site on the target molecule CDH19.

Binding domains can be derived from a binding domain donor such as for example an antibody. It is envisaged that a binding domain of the present invention comprises at least said part of any of the aforementioned binding domains that is required for binding to/interacting with a given target epitope or a given target site on the target molecule CDH19.

It is envisaged that the binding domain of the aforementioned binding domain donors is characterized by that part of these donors that is responsible for binding the respective target, i.e. when that part is removed from the binding domain donor, said donor loses its binding capability. “Loses” means a reduction of at least 50% of the binding capability when compared with the binding donor. Methods to map these binding sites are well known in the art-it is therefore within the standard knowledge of the skilled person to locate/map the binding site of a binding domain donor and, thereby, to “derive” said binding domain from the respective binding domain donors.

The term “epitope” refers to a site on an antigen to which a binding domain, such as an antibody or immunoglobulin or derivative or fragment of an antibody or of an immunoglobulin, specifically binds. An “epitope” is antigenic and thus the term epitope is sometimes also referred to herein as “antigenic structure” or “antigenic determinant”. Thus, the binding domain is an “antigen-interaction-site”. Said binding/interaction is also understood to define a “specific recognition”. In one example, said binding domain which (specifically) binds to/interacts with a given target epitope or a given target site on the target molecule CDH19 is an antibody or immunoglobulin, and said binding domain is a VH and/or VL region of an antibody or of an immunoglobulin.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigen for one of the binding domains is comprised within the CDH19 protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. Moreover, the provided examples describe a further method to characterize a given binding domain by way of binning, which includes a test whether the given binding domain binds to one or more epitope cluster(s) of a given protein, in particular CDH19.

As used herein, the term “epitope cluster” denotes the entirety of epitopes lying in a defined contiguous stretch of an antigen. An epitope cluster can comprise one, two or more epitopes. The concept of epitope cluster is also used in the characterization of the features of the antibody or antigen binding fragment thereof of the invention.

The terms “(capable of) binding to”, “specifically recognizing”, “directed to” and “reacting with” mean in accordance with this invention that a binding domain is capable of specifically interacting with one or more, preferably at least two, more preferably at least three and most preferably at least four amino acids of an epitope.

As used herein, the terms “specifically interacting”, “specifically binding” or “specifically bind(s)” mean that a binding domain exhibits appreciable affinity for a particular protein or antigen and, generally, does not exhibit significant reactivity with proteins or antigens other than CDH19. “Appreciable affinity” includes binding with an affinity of about 10⁻⁶M (KD) or stronger. Preferably, binding is considered specific when binding affinity is about 10⁻¹² to 10 ⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than CDH19. Preferably, a binding domain of the invention does not essentially bind or is not capable of binding to proteins or antigens other than CDH19.

The term “does not essentially bind”, or “is not capable of binding” means that a binding domain of the present invention does not bind another protein or antigen other than CDH19, i.e., does not show reactivity of more than 30%, preferably not more than 20%, more preferably not more than 10%, particularly preferably not more than 9%, 8%, 7%, 6% or 5% with proteins or antigens other than CDH19, whereby binding to CDH19, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-site with its specific antigen may result in a simple binding of said site to the antigen. Moreover, the specific interaction of the antigen-interaction-site with its specific antigen may alternatively or additionally result in the initiation of a signal, e.g. due to the induction of a change of the conformation of the antigen, an oligomerization of the antigen, etc.

Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a hereteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. A “polypeptide” when referred to herein may also be chemically modified such as pegylated. Such modifications are well known in the art.

“Isolated” when used to describe the antibody or antigen binding fragment thereof or antibody construct disclosed herein, refers to the antibody or antigen binding fragment thereof or antibody construct disclosed herein that has been identified, separated and/or recovered from a component of its production environment. Preferably, the isolated the antibody or antigen binding fragment thereof or antibody construct disclosed herein is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody or antigen binding fragment thereof or antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

Amino acid sequence modifications of the antibody or antigen binding fragment thereof or antibody construct described herein are contemplated. For example, it may be desirable to improve the binding affinity and/or other biological properties of the antibody. Amino acid sequence variants of the antibody or antigen binding fragment thereof or antibody construct disclosed herein are prepared by introducing appropriate nucleotide changes into the antibody or antigen binding fragment thereof or antibody construct nucleic acid, or by peptide synthesis.

Such modifications include, for example, deletions from, and/or insertions into, and/or substitutions of, residues within the amino acid sequences of the antibody or antigen binding fragment thereof or antibody construct disclosed herein. Any combination of deletion, insertion, and substitution is made to arrive at the final construct, provided that the final construct possesses the desired characteristics. The amino acid changes also may alter post-translational processes of the antibody or antigen binding fragment thereof or antibody construct disclosed herein, such as changing the number or position of glycosylation sites. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs). The substitutions are preferably conservative substitutions as described herein. Additionally or alternatively, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs.

A useful method for identification of certain residues or regions of the antibody or antigen binding fragment thereof or antibody construct disclosed herein that are preferred locations for mutagenesis is called “alanine scanning mutagenesis” as described by Cunningham and Wells in Science, 244: 1081-1085 (1989). Here, a residue or group of target residues within the antibody or antigen binding fragment thereof or antibody construct disclosed herein is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions then are refined by introducing further or other variants at, or for, the sites of substitution. Thus, while the site for introducing an amino acid sequence variation is predetermined, the nature of the mutation per se needs not to be predetermined. For example, to analyze the performance of a mutation at a given site, ala scanning or random mutagenesis is conducted at a target codon or region and the expressed the antibody or antigen binding fragment thereof or antibody construct disclosed herein variants are screened for the desired activity.

Preferably, amino acid sequence insertions include amino- and/or carboxyl-terminal fusions ranging in length from 1, 2, 3, 4, 5, 6, 7, 8, 9 or 10 residues to polypeptides containing a hundred or more residues, as well as intrasequence insertions of single or multiple amino acid residues. An insertional variant of the antibody or antigen binding fragment thereof or antibody construct disclosed herein includes the fusion to the N- or C-terminus of the antibody to an enzyme or a fusion to a polypeptide which increases the serum half-life of the antibody.

Another type of variant is an amino acid substitution variant. These variants have preferably at least 1, 2, 3, 4, 5, 6, 7, 8, 9 or 10 amino acid residues in the antibody or antigen binding fragment thereof or antibody construct disclosed herein replaced by a different residue. The sites of greatest interest for substitutional mutagenesis include the CDRs of the heavy and/or light chain, in particular the hypervariable regions, but FR alterations in the heavy and/or light chain are also contemplated.

For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs of the heavy and/or light chain, it is preferred that the then-obtained “substituted” sequence is at least 60%, more preferably 65%, even more preferably 70%, particularly preferably 75%, more particularly preferably 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody or antigen binding fragment thereof or antibody construct disclosed herein may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 1, below) is envisaged as long as the antibody or antigen binding fragment thereof or antibody construct retains its capability to bind to CDH19 v and/or its CDRs have an identity to the then substituted sequence (at least 60%, more preferably 65%, even more preferably 70%, particularly preferably 75%, more particularly preferably 80% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino Acid Substitutions

| | | Preferred |
|---|---|---|
| Original | Exemplary Substitutions | Substitutions |
| Ala (A) | val, leu, ile | val |
| Arg (R) | lys, gin, asn | lys |
| Asn (N) | gin, his, asp, lys, arg | gin |
| Asp (D) | glu, asn | glu |
| Cys (C) | ser, ala | ser |
| Gln (Q) | asn, glu | asn |
| Glu (E) | asp, gin | Asp |
| Gly (G) | ala | Ala |
| His (H) | asn, gin, lys, arg | Arg |
| Ile (I) | leu, val, met, ala, phe | Leu |
| Leu (L) | norleucine, ile, val, met, ala | Ile |
| Lys (K) | arg, gin, asn | Arg |
| Met (M) | leu, phe, ile | Leu |
| Phe (F) | leu, val, ile, ala, tyr | Tyr |
| Pro (P) | ala | Ala |
| Ser (S) | thr | Thr |
| Thr (T) | ser | Ser |
| Trp (W) | tyr, phe | Tyr |
| Tyr (Y) | trp, phe, thr, ser | Phe |
| Val (V) | ile, leu, met, phe, ala | Leu |

Substantial modifications in the biological properties of the antibody or antigen binding fragment thereof or antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody or antigen binding fragment thereof or antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e. g. 6-7 sites) are mutated to generate all possible amino acid substitutions at each site. The antibody variants thus generated are displayed in a monovalent fashion from filamentous phage particles as fusions to the gene Ill product of M13 packaged within each particle. The phage-displayed variants are then screened for their biological activity (e. g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human CDH19. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

Other modifications of the antibody or antigen binding fragment thereof or antibody construct are contemplated herein. For example, the antibody or antigen binding fragment thereof or antibody construct may be linked to one of a variety of non-proteinaceous polymers, e.g., polyethylene glycol, polypropylene glycol, polyoxyalkylenes, or copolymers of polyethylene glycol and polypropylene glycol. The antibody or antigen binding fragment thereof or antibody construct may also be entrapped in microcapsules prepared, for example, by coacervation techniques or by interfacial polymerization (for example, hydroxymethylcellulose or gelatine-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16th edition, Oslo, A., Ed., (1980).

The antibody or antigen binding fragment thereof or antibody construct disclosed herein may also be formulated as immuno-liposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); U.S. Pat. Nos. 4,485,045 and 4,544,545; and WO 97/38731 published Oct. 23, 1997. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81 (19) 1484 (1989).

When using recombinant techniques, the antibody, antigen binding fragment thereof or antibody construct can be produced intracellularly, in the periplasmic space, or directly secreted into the medium. If the antibody, antigen binding fragment thereof or antibody construct is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli.

The antibody, antigen binding fragment thereof or antibody construct composition prepared from the cells can be purified using, for example, hydroxylapatite chromatography, gel electrophoresis, dialysis, and affinity chromatography, with affinity chromatography being the preferred purification technique.

The term “agent” is used herein to denote a chemical compound, a mixture of chemical compounds, a biological macromolecule, or an extract made from biological materials.

The term “nucleic acid” is well known to the skilled person and encompasses DNA (such as cDNA) and RNA (such as mRNA). The nucleic acid can be double stranded and single stranded, linear and circular. Said nucleic acid molecule is preferably comprised in a vector which is preferably comprised in a host cell. Said host cell is, e.g. after transformation or transfection with the nucleic acid sequence of the invention, capable of expressing the antibody or antigen binding fragment thereof or antibody construct disclosed herein. For that purpose the nucleic acid molecule is operatively linked with control sequences.

A vector is a nucleic acid molecule used as a vehicle to transfer (foreign) genetic material into a cell. The term “vector” encompasses- but is not restricted to-plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences such as a promoter sequence that drives expression of the transgene. Insertion of a vector into the target cell is usually called “transformation” for bacteria, “transfection” for eukaryotic cells, although insertion of a viral vector is also called “transduction”.

As used herein, the term “host cell” is intended to refer to a cell into which a nucleic acid encoding the antibody or antigen binding fragment thereof or antibody construct of the invention is introduced by way of transformation, transfection and the like. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “expression” includes any step involved in the production of a the antibody or antigen binding fragment thereof or antibody construct of the invention including, but not limited to, transcription, post-transcriptional modification, translation, post-translational modification, and secretion.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The terms “host cell,” “target cell” or “recipient cell” are intended to include any individual cell or cell culture that can be or has/have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacteria, yeast cells, animal cells, and mammalian cells, e.g., murine, rat, macaque or human.

Suitable host cells include prokaryotes and eukaryotic host cells including yeasts, fungi, insect cells and mammalian cells.

The antibody or antigen binding fragment thereof or antibody construct of the invention can be produced in bacteria. After expression, the antibody or antigen binding fragment thereof or antibody construct of the invention, preferably the antibody or antigen binding fragment thereof or antibody construct is isolated from the E. coli cell paste in a soluble fraction and can be purified through, e.g., affinity chromatography and/or size exclusion. Final purification can be carried out similar to the process for purifying antibody expressed e. g, in CHO cells.

In addition to prokaryotes, eukaryotic microbes such as filamentous fungi or yeast are suitable cloning or expression hosts for the antibody or antigen binding fragment thereof or antibody construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as, e.g., K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia(EP 244 234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated the antibody or antigen binding fragment thereof or antibody construct of the invention, preferably antibody derived antibody constructs are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e. g., the L−1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be utilized as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36: 59 (1977)); baby hamster kidney cells (BHK, ATCC CCL 10); Chinese hamster ovary cells/-DHFR (CHO, Urlaub et al., Proc. Natl. Acad. Sci. USA 77: 4216 (1980)); mouse sertoli cells (TM4, Mather, Biol. Reprod. 23: 243-251 (1980)); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells (Mather et al., Annals N. Y Acad. Sci. 383: 44-68 (1982)); MRC 5 cells; FS4 cells; and a human hepatoma line (Hep G2).

When using recombinant techniques, the antibody or antigen binding fragment thereof or antibody construct of the invention can be produced intracellularly, in the periplasmic space, or directly secreted into the medium. If the antibody or antigen binding fragment thereof or antibody construct is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody or antigen binding fragment thereof or antibody construct of the invention prepared from the host cells can be purified using, for example, hydroxylapatite chromatography, gel electrophoresis, dialysis, and affinity chromatography, with affinity chromatography being the preferred purification technique.

The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody or antigen binding fragment thereof or antibody construct of the invention comprises a CH3 domain, the Bakerbond ABXMresin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromato-focusing, SDS-PAGE, and ammonium sulfate precipitation are also available depending on the antibody to be recovered.

The term “culturing” refers to the in vitro maintenance, differentiation, growth, proliferation and/or propagation of cells under suitable conditions in a medium.

As used herein, the term “pharmaceutical composition” relates to a composition for administration to a patient, preferably a human patient. The particular preferred pharmaceutical composition of this invention comprises the antibody or antigen binding fragment thereof or antibody construct of the invention. Preferably, the pharmaceutical composition comprises suitable formulations of carriers, stabilizers and/or excipients. In a preferred embodiment, the pharmaceutical composition comprises a composition for parenteral, transdermal, intraluminal, intraarterial, intrathecal and/or intranasal administration or by direct injection into tissue. It is in particular envisaged that said composition is administered to a patient via infusion or injection. Administration of the suitable compositions may be effected by different ways, e.g., by intravenous, intraperitoneal, subcutaneous, intramuscular, topical or intradermal administration. In particular, the present invention provides for an uninterrupted administration of the suitable composition. As a non-limiting example, uninterrupted, i.e. continuous administration may be realized by a small pump system worn by the patient for metering the influx of therapeutic agent into the body of the patient. The pharmaceutical composition comprising the antibody or antigen binding fragment thereof or antibody construct of the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of these antibody or antigen binding fragment thereof or antibody constructs of the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

The inventive compositions may further comprise a pharmaceutically acceptable carrier. Examples of suitable pharmaceutical carriers are well known in the art and include solutions, e.g. phosphate buffered saline solutions, water, emulsions, such as oil/water emulsions, various types of wetting agents, sterile solutions, liposomes, etc. Compositions comprising such carriers can be formulated by well known conventional methods. Formulations can comprise carbohydrates, buffer solutions, amino acids and/or surfactants. Carbohydrates may be non-reducing sugars, preferably trehalose, sucrose, octasulfate, sorbitol or xylitol. In general, as used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counter-ions, such as sodium, polyhydric sugar alcohols; amino acids, such as alanine, glycine, asparagine, 2-phenylalanine, and threonine; sugars or sugar alcohols, such as trehalose, sucrose, octasulfate, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Such formulations may be used for continuous administrations which may be intravenous or subcutaneous with and/or without pump systems. Amino acids may be charged amino acids, preferably lysine, lysine acetate, arginine, glutamate and/or histidine. Surfactants may be detergents, preferably with a molecular weight of >1.2 KD and/or a polyether, preferably with a molecular weight of >3 KD. Non-limiting examples for preferred detergents are Tween 20, Tween 40, Tween 60, Tween 80 or Tween 85. Non-limiting examples for preferred polyethers are PEG 3000, PEG 3350, PEG 4000 or PEG 5000. Buffer systems used in the present invention can have a preferred pH of 5-9 and may comprise citrate, succinate, phosphate, histidine and acetate.

The compositions of the present invention can be administered to the subject at a suitable dose which can be determined e.g. by dose escalating studies by administration of increasing doses of the polypeptide of the invention exhibiting cross-species specificity described herein to non-chimpanzee primates, for instance macaques. As set forth above, the antibody or antigen binding fragment thereof or antibody construct of the invention exhibiting cross-species specificity described herein can be advantageously used in identical form in preclinical testing in non-chimpanzee primates and as drug in humans. These compositions can also be administered in combination with other proteinaceous and non-proteinaceous drugs. These drugs may be administered simultaneously with the composition comprising the polypeptide of the invention as defined herein or separately before or after administration of said polypeptide in timely defined intervals and doses. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the composition of the present invention might comprise proteinaceous carriers, like, e.g., serum albumin or immunoglobulin, preferably of human origin. It is envisaged that the composition of the invention might comprise, in addition to the polypeptide of the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art. It is also envisaged that the antibody or antigen binding fragment thereof or antibody construct of the present invention is applied in a co-therapy, i.e., in combination with another anti-cancer medicament.

The biological activity of the pharmaceutical composition defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Horning S J, Coiffier B, Shipp M A, Fisher R I, Connors J M, Lister T A, Vose J, Grillo-Lopez A, Hagenbeek A, Cabanillas F, Klippensten D, Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 April;17(4):1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a particular drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” means the time where 50% of an administered drug are eliminated through biological processes, e.g. metabolism, excretion, etc.

By “hepatic first-pass metabolism” is meant the propensity of a drug to be metabolized upon first contact with the liver, i.e. during its first pass through the liver.

“Volume of distribution” means the degree of retention of a drug throughout the various compartments of the body, like e.g. intracellular and extracellular spaces, tissues and organs, etc. and the distribution of the drug within these compartments.

“Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates, more onset and/or Cmax for a given amount of drug administered. “Bioavailability” means the amount of a drug in the blood compartment. “Lag time” means the time delay between the administration of the drug and its detection and measurability in blood or plasma.

“Tmax” is the time after which maximal blood concentration of the drug is reached, and “Cmax” is the blood concentration maximally obtained with a given drug. The time to reach a blood or tissue concentration of the drug which is required for its biological effect is influenced by all parameters. Pharmacokinetic parameters of bispecific single chain antibodies exhibiting cross-species specificity, which may be determined in preclinical animal testing in non-chimpanzee primates as outlined above, are also set forth e.g. in the publication by Schlereth et al. (Cancer Immunol. Immunother. 20 (2005), 1-12).

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the subject's own immune system. The term “patient” includes human and other mammalian subjects that receive either prophylactic or therapeutic treatment.

The term “effective and non-toxic dose” as used herein refers to a tolerable dose of an inventive antibody or antigen binding fragment thereof or antibody construct which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

The appropriate dosage, or therapeutically effective amount, of the antibody or antigen binding fragment thereof or antibody construct of the invention will depend on the condition to be treated, the severity of the condition, prior therapy, and the patient's clinical history and response to the therapeutic agent. The proper dose can be adjusted according to the judgment of the attending physician such that it can be administered to the patient one time or over a series of administrations. The pharmaceutical composition can be administered as a sole therapeutic or in combination with additional therapies such as anti-cancer therapies as needed.

The pharmaceutical compositions of this invention are particularly useful for parenteral administration, i.e., subcutaneously, intramuscularly, intravenously, intra-articular and/or intra-synovial. Parenteral administration can be by bolus injection or continuous infusion.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

In an internal analysis of proprietary mRNA expression data it has been surprisingly found that CDH19 expression is elevated in both primary and metastatic melanoma tumors compared to normal, untransformed tissues. Internal analysis also confirmed that expression of CDH19 in normal tissues is limited to neural crest derived peripheral nerve ganglia and nerve fibers. The differential CDH19 expression in normal and tumor tissues makes this protein attractive for cell-surface targeting therapeutics. Although CDH 19 was discussed as one marker as part of long lists of markers associated with some cancer types (see e.g. WO2009/055937) or Parkinson's disease (see e.g. WO2005/067391) CDH19 was never discussed as a prognostic marker or a drug target in connection with melanoma tumors.

As stated above, the present invention provides an isolated human antibody or antigen binding fragment thereof capable of binding to human CDH19 on the surface of a target cell. In a preferred embodiment the antibody or antigen binding fragment thereof comprises a monoclonal antibody or a fragment thereof.

The “CDH19 extracellular domain” or “CDH19 ECD” refers to a form of CDH19 which is essentially free of transmembrane and cytoplasmic domains of CDH19. It will be understood by the skilled artisan that the transmembrane domain identified for the CDH19 polypeptide of the present invention is identified pursuant to criteria routinely employed in the art for identifying that type of hydrophobic domain. The exact boundaries of a transmembrane domain may vary but most likely by no more than about 5 amino acids at either end of the domain specifically mentioned herein. A preferred human CDH19 ECD is shown in SEQ ID NO: 948 (aa residues 44-596). In this context it is understood that the CDH19 ECD represents the part of CDH19 on the surface of a target cell.

The affinity of the antibody or fragment thereof for human CDH19 is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM. The affinity of the first binding domain for macaque CDH19 is preferably ≤15 nM, more preferably ≤10 nM, even more preferably ≤5 nM, even more preferably ≤1 nM, even more preferably ≤0.5 nM, even more preferably ≤0.1 nM, and most preferably ≤0.05 nM or even ≤0.01 nM. The affinity can be measured for example in a Biacore assay or in a Scatchard assay, e.g. as described in the Examples. The affinity gap for binding to macaque CDH19 versus human CDH19 is preferably [1:10-1:5] or [5:1-10:1], more preferably [1:5-5:1], and most preferably [1:2-3:1] or even [1:1-3:1]. Other methods of determining the affinity are well-known to the skilled person.

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, human or fully human antibodies can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies are expected to minimize the immunogenic and allergic responses intrinsic to mouse or mouse-derivatized mAbs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with our generation of the first XenoMouse mouse strains, as published in 1994. (See Green et al. Nature Genetics 7:13-21 (1994)) The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620, filed Dec. 3, 1996, the disclosures of which are hereby incorporated by reference.

The production of the XenoMouse mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000, WO 03/47336. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V.sub.H genes, one or more D.sub.H genes, one or more J.sub.H genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B 1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference.Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996, 5,698,767, and 5,958,765.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against EGFRvIII in order to vitiate concerns and/or effects of HAMA or HACA response.

According to one embodiment the antibody of the present invention is a dimer comprising two fusion proteins created by fusing a CDH19 binding fragment of a CDH19 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

Alternatively, the antibody of the invention is a fusion protein comprising multiple CDH19 antibody polypeptides, with or without peptide linkers (spacer peptides). Among the suitable peptide linkers are those described in U.S. Pat. Nos. 4,751,180 and 4,935,233 or WO 88/09344.

Another method for preparing oligomeric CDH19 antibody derivatives involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising CDH19 antibody fragment or derivative fused to a leucine zipper peptide are expressed in suitable host cells, and the soluble oligomeric CDH19 antibody fragments or derivatives that form are recovered from the culture supernatant.

Covalent modifications of antigen binding proteins are included within the scope of this invention, and are generally, but not always, done post-translationally. For example, several types of covalent modifications of the antigen binding protein are introduced into the molecule by reacting specific amino acid residues of the antigen binding protein with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antigen binding proteins to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antigen binding protein included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antigen binding protein is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antigen binding protein is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, 1981, CRC Crit. Rev. Biochem., pp. 259-306.

Removal of carbohydrate moieties present on the starting antigen binding protein may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Another type of covalent modification of the antigen binding protein comprises linking the antigen binding protein to various non-proteinaceous polymers, including, but not limited to, various polyols such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antigen binding protein to facilitate the addition of polymers such as PEG.

In some embodiments, the covalent modification of the antigen binding proteins of the invention comprises the addition of one or more labels.

The term “labelling group” means any detectable label. Examples of suitable labelling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹i), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the antigen binding protein via spacer arms of various lengths to reduce potential steric hindrance. Various methods for labelling proteins are known in the art and may be used in performing the present invention.

In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antigen binding protein via spacer arms of various lengths to reduce potential steric hindrance. Various methods for labelling proteins are known in the art and may be used in performing the present invention.

Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al, 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al, 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

As described in appended example 2 a broad number of CDH19 specific binder has been characterized with respect to identified binding characteristics and those binders were grouped into five different bins, which refers to five different subgroups of CDH19 specific binding domains. Accordingly, in one embodiment the human antibody or antigen binding fragment thereof of the invention comprises a human binding domain or antigen binding fragment thereof comprising a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 52, CDR-H2 as depicted in SEQ ID NO: 53, CDR-H3 as depicted in SEQ ID NO: 54, CDR-L1 as depicted in SEQ ID NO: 220, CDR-L2 as depicted in SEQ ID NO: 221 and CDR-L3 as depicted in SEQ ID NO: 222, CDR-H1 as depicted in SEQ ID NO: 82, CDR-H2 as depicted in SEQ ID NO: 83, CDR-H3 as depicted in SEQ ID NO: 84, CDR-L1 as depicted in SEQ ID NO: 250, CDR-L2 as depicted in SEQ ID NO: 251 and CDR-L3 as depicted in SEQ ID NO: 252, CDR-H1 as depicted in SEQ ID NO: 82, CDR-H2 as depicted in SEQ ID NO: 83, CDR-H3 as depicted in SEQ ID NO: 84, CDR-L1 as depicted in SEQ ID NO: 250, CDR-L2 as depicted in SEQ ID NO: 251 and CDR-L3 as depicted in SEQ ID NO: 927, CDR-H1 as depicted in SEQ ID NO: 82, CDR-H2 as depicted in SEQ ID NO: 83, CDR-H3 as depicted in SEQ ID NO: 909, CDR-L1 as depicted in SEQ ID NO: 250, CDR-L2 as depicted in SEQ ID NO: 251 and CDR-L3 as depicted in SEQ ID NO: 927, CDR-H1 as depicted in SEQ ID NO: 52, CDR-H2 as depicted in SEQ ID NO: 53, CDR-H3 as depicted in SEQ ID NO: 54, CDR-L1 as depicted in SEQ ID NO: 220, CDR-L2 as depicted in SEQ ID NO: 221 and CDR-L3 as depicted in SEQ ID NO: 926, and CDR-H1 as depicted in SEQ ID NO: 52, CDR-H2 as depicted in SEQ ID NO: 53, CDR-H3 as depicted in SEQ ID NO: 904, CDR-L1 as depicted in SEQ ID NO: 220, CDR-L2 as depicted in SEQ ID NO: 221 and CDR-L3 as depicted in SEQ ID NO: 926; which all characterize binding domains for CDH19 grouped into bin 1;
(b) CDR-H1 as depicted in SEQ ID NO: 124, CDR-H2 as depicted in SEQ ID NO: 125, CDR-H3 as depicted in SEQ ID NO: 126, CDR-L1 as depicted in SEQ ID NO: 292, CDR-L2 as depicted in SEQ ID NO: 293 and CDR-L3 as depicted in SEQ ID NO: 294, CDR-H1 as depicted in SEQ ID NO: 130, CDR-H2 as depicted in SEQ ID NO: 131, CDR-H3 as depicted in SEQ ID NO: 132, CDR-L1 as depicted in SEQ ID NO: 298, CDR-L2 as depicted in SEQ ID NO: 299 and CDR-L3 as depicted in SEQ ID NO: 300, CDR-H1 as depicted in SEQ ID NO: 136, CDR-H2 as depicted in SEQ ID NO: 137, CDR-H3 as depicted in SEQ ID NO: 138, CDR-L1 as depicted in SEQ ID NO: 304, CDR-L2 as depicted in SEQ ID NO: 305 and CDR-L3 as depicted in SEQ ID NO: 306, CDR-H1 as depicted in SEQ ID NO: 142, CDR-H2 as depicted in SEQ ID NO: 143, CDR-H3 as depicted in SEQ ID NO: 144, CDR-L1 as depicted in SEQ ID NO: 310, CDR-L2 as depicted in SEQ ID NO: 311 and CDR-L3 as depicted in SEQ ID NO: 312, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 318, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 336, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 294, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 928, CDR-H1 as depicted in SEQ ID NO: 124, CDR-H2 as depicted in SEQ ID NO: 125, CDR-H3 as depicted in SEQ ID NO: 915, CDR-L1 as depicted in SEQ ID NO: 292, CDR-L2 as depicted in SEQ ID NO: 293 and CDR-L3 as depicted in SEQ ID NO: 929, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 336, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 942, CDR-H1 as depicted in SEQ ID NO: 166, CDR-H2 as depicted in SEQ ID NO: 167, CDR-H3 as depicted in SEQ ID NO: 168, CDR-L1 as depicted in SEQ ID NO: 334, CDR-L2 as depicted in SEQ ID NO: 335 and CDR-L3 as depicted in SEQ ID NO: 943, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 318, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 937, CDR-H1 as depicted in SEQ ID NO: 148, CDR-H2 as depicted in SEQ ID NO: 149, CDR-H3 as depicted in SEQ ID NO: 150, CDR-L1 as depicted in SEQ ID NO: 316, CDR-L2 as depicted in SEQ ID NO: 317 and CDR-L3 as depicted in SEQ ID NO: 938, CDR-H1 as depicted in SEQ ID NO: 148, CDR-H2 as depicted in SEQ ID NO: 149, CDR-H3 as depicted in SEQ ID NO: 919, CDR-L1 as depicted in SEQ ID NO: 316, CDR-L2 as depicted in SEQ ID NO: 317 and CDR-L3 as depicted in SEQ ID NO: 938, CDR-H1 as depicted in SEQ ID NO: 142, CDR-H2 as depicted in SEQ ID NO: 143, CDR-H3 as depicted in SEQ ID NO: 144, CDR-L1 as depicted in SEQ ID NO: 310, CDR-L2 as depicted in SEQ ID NO: 311 and CDR-L3 as depicted in SEQ ID NO: 935, CDR-H1 as depicted in SEQ ID NO: 142, CDR-H2 as depicted in SEQ ID NO: 143, CDR-H3 as depicted in SEQ ID NO: 918, CDR-L1 as depicted in SEQ ID NO: 310, CDR-L2 as depicted in SEQ ID NO: 311 and CDR-L3 as depicted in SEQ ID NO: 935, CDR-H1 as depicted in SEQ ID NO: 142, CDR-H2 as depicted in SEQ ID NO: 143, CDR-H3 as depicted in SEQ ID NO: 918, CDR-L1 as depicted in SEQ ID NO: 310, CDR-L2 as depicted in SEQ ID NO: 311 and CDR-L3 as depicted in SEQ ID NO: 936, CDR-H1 as depicted in SEQ ID NO: 136, CDR-H2 as depicted in SEQ ID NO: 137, CDR-H3 as depicted in SEQ ID NO: 138, CDR-L1 as depicted in SEQ ID NO: 304, CDR-L2 as depicted in SEQ ID NO: 305 and CDR-L3 as depicted in SEQ ID NO: 933, CDR-H1 as depicted in SEQ ID NO: 136, CDR-H2 as depicted in SEQ ID NO: 137, CDR-H3 as depicted in SEQ ID NO: 917, CDR-L1 as depicted in SEQ ID NO: 304, CDR-L2 as depicted in SEQ ID NO: 305 and CDR-L3 as depicted in SEQ ID NO: 934, CDR-H1 as depicted in SEQ ID NO: 130, CDR-H2 as depicted in SEQ ID NO: 131, CDR-H3 as depicted in SEQ ID NO: 132, CDR-L1 as depicted in SEQ ID NO: 298, CDR-L2 as depicted in SEQ ID NO: 299 and CDR-L3 as depicted in SEQ ID NO: 930, CDR-H1 as depicted in SEQ ID NO: 130, CDR-H2 as depicted in SEQ ID NO: 131, CDR-H3 as depicted in SEQ ID NO: 916, CDR-L1 as depicted in SEQ ID NO: 298, CDR-L2 as depicted in SEQ ID NO: 299 and CDR-L3 as depicted in SEQ ID NO: 931, and CDR-H1 as depicted in SEQ ID NO: 130, CDR-H2 as depicted in SEQ ID NO: 131, CDR-H3 as depicted in SEQ ID NO: 916, CDR-L1 as depicted in SEQ ID NO: 298, CDR-L2 as depicted in SEQ ID NO: 299 and CDR-L3 as depicted in SEQ ID NO: 932; which all characterize binding domains for CDH19 grouped into bin 2;
(c) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 100, CDR-H2 as depicted in SEQ ID NO: 101, CDR-H3 as depicted in SEQ ID NO: 102, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 118, CDR-H2 as depicted in SEQ ID NO: 119, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, CDR-H1 as depicted in SEQ ID NO: 154, CDR-H2 as depicted in SEQ ID NO: 155, CDR-H3 as depicted in SEQ ID NO: 156, CDR-L1 as depicted in SEQ ID NO: 322, CDR-L2 as depicted in SEQ ID NO: 323 and CDR-L3 as depicted in SEQ ID NO: 324, CDR-H1 as depicted in SEQ ID NO: 100, CDR-H2 as depicted in SEQ ID NO: 101, CDR-H3 as depicted in SEQ ID NO: 912, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 100, CDR-H2 as depicted in SEQ ID NO: 101, CDR-H3 as depicted in SEQ ID NO: 913, CDR-L1 as depicted in SEQ ID NO: 268, CDR-L2 as depicted in SEQ ID NO: 269 and CDR-L3 as depicted in SEQ ID NO: 270, CDR-H1 as depicted in SEQ ID NO: 94, CDR-H2 as depicted in SEQ ID NO: 95, CDR-H3 as depicted in SEQ ID NO: 910, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 94, CDR-H2 as depicted in SEQ ID NO: 95, CDR-H3 as depicted in SEQ ID NO: 911, CDR-L1 as depicted in SEQ ID NO: 262, CDR-L2 as depicted in SEQ ID NO: 263 and CDR-L3 as depicted in SEQ ID NO: 264, CDR-H1 as depicted in SEQ ID NO: 118, CDR-H2 as depicted in SEQ ID NO: 119, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, CDR-H1 as depicted in SEQ ID NO: 118, CDR-H2 as depicted in SEQ ID NO: 914, CDR-H3 as depicted in SEQ ID NO: 120, CDR-L1 as depicted in SEQ ID NO: 286, CDR-L2 as depicted in SEQ ID NO: 287 and CDR-L3 as depicted in SEQ ID NO: 288, and CDR-H1 as depicted in SEQ ID NO: 154, CDR-H2 as depicted in SEQ ID NO: 155, CDR-H3 as depicted in SEQ ID NO: 920, CDR-L1 as depicted in SEQ ID NO: 322, CDR-L2 as depicted in SEQ ID NO: 323 and CDR-L3 as depicted in SEQ ID NO: 324; which all characterize binding domains for CDH19 grouped into bin 3;
(d) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 172, CDR-L2 as depicted in SEQ ID NO: 173 and CDR-L3 as depicted in SEQ ID NO: 174, CDR-H1 as depicted in SEQ ID NO: 10, CDR-H2 as depicted in SEQ ID NO: 11, CDR-H3 as depicted in SEQ ID NO: 12, CDR-L1 as depicted in SEQ ID NO: 178, CDR-L2 as depicted in SEQ ID NO: 179 and CDR-L3 as depicted in SEQ ID NO: 180, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 196, CDR-L2 as depicted in SEQ ID NO: 197 and CDR-L3 as depicted in SEQ ID NO: 198, CDR-H1 as depicted in SEQ ID NO: 34, CDR-H2 as depicted in SEQ ID NO: 35, CDR-H3 as depicted in SEQ ID NO: 36, CDR-L1 as depicted in SEQ ID NO: 202, CDR-L2 as depicted in SEQ ID NO: 203 and CDR-L3 as depicted in SEQ ID NO: 204, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 214, CDR-L2 as depicted in SEQ ID NO: 215 and CDR-L3 as depicted in SEQ ID NO: 216, CDR-H1 as depicted in SEQ ID NO: 58, CDR-H2 as depicted in SEQ ID NO: 59, CDR-H3 as depicted in SEQ ID NO: 60, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 64, CDR-H2 as depicted in SEQ ID NO: 65, CDR-H3 as depicted in SEQ ID NO: 66, CDR-L1 as depicted in SEQ ID NO: 232, CDR-L2 as depicted in SEQ ID NO: 233 and CDR-L3 as depicted in SEQ ID NO: 234, CDR-H1 as depicted in SEQ ID NO: 70, CDR-H2 as depicted in SEQ ID NO: 71, CDR-H3 as depicted in SEQ ID NO: 72, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 328, CDR-L2 as depicted in SEQ ID NO: 329 and CDR-L3 as depicted in SEQ ID NO: 330, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 902, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 903, CDR-L1 as depicted in SEQ ID NO: 924, CDR-L2 as depicted in SEQ ID NO: 215 and CDR-L3 as depicted in SEQ ID NO: 216, CDR-H1 as depicted in SEQ ID NO: 46, CDR-H2 as depicted in SEQ ID NO: 47, CDR-H3 as depicted in SEQ ID NO: 48, CDR-L1 as depicted in SEQ ID NO: 925, CDR-L2 as depicted in SEQ ID NO: 215 and CDR-L3 as depicted in SEQ ID NO: 216, CDR-H1 as depicted in SEQ ID NO: 70, CDR-H2 as depicted in SEQ ID NO: 907, CDR-H3 as depicted in SEQ ID NO: 72, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 70, CDR-H2 as depicted in SEQ ID NO: 907, CDR-H3 as depicted in SEQ ID NO: 908, CDR-L1 as depicted in SEQ ID NO: 238, CDR-L2 as depicted in SEQ ID NO: 239 and CDR-L3 as depicted in SEQ ID NO: 240, CDR-H1 as depicted in SEQ ID NO: 28, CDR-H2 as depicted in SEQ ID NO: 901, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, CDR-H1 as depicted in SEQ ID NO: 58, CDR-H2 as depicted in SEQ ID NO: 905, CDR-H3 as depicted in SEQ ID NO: 906, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 58, CDR-H2 as depicted in SEQ ID NO: 905, CDR-H3 as depicted in SEQ ID NO: 60, CDR-L1 as depicted in SEQ ID NO: 226, CDR-L2 as depicted in SEQ ID NO: 227 and CDR-L3 as depicted in SEQ ID NO: 228, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 939, CDR-L2 as depicted in SEQ ID NO: 329 and CDR-L3 as depicted in SEQ ID NO: 330, CDR-H1 as depicted in SEQ ID NO: 160, CDR-H2 as depicted in SEQ ID NO: 921, CDR-H3 as depicted in SEQ ID NO: 162, CDR-L1 as depicted in SEQ ID NO: 939, CDR-L2 as depicted in SEQ ID NO: 329 and CDR-L3 as depicted in SEQ ID NO: 940, CDR-H1 as depicted in SEQ ID NO: 160, CDR-H2 as depicted in SEQ ID NO: 161, CDR-H3 as depicted in SEQ ID NO: 162, CDR-L1 as depicted in SEQ ID NO: 941, CDR-L2 as depicted in SEQ ID NO: 329 and CDR-L3 as depicted in SEQ ID NO: 330, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 196, CDR-L2 as depicted in SEQ ID NO: 197 and CDR-L3 as depicted in SEQ ID NO: 923, CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, CDR-H1 as depicted in SEQ ID NO: 28, CDR-H2 as depicted in SEQ ID NO: 901, CDR-H3 as depicted in SEQ ID NO: 30, CDR-L1 as depicted in SEQ ID NO: 922, CDR-L2 as depicted in SEQ ID NO: 197 and CDR-L3 as depicted in SEQ ID NO: 923, and CDR-H1 as depicted in SEQ ID NO: 28, CDR-H2 as depicted in SEQ ID NO: 29, CDR-H3 as depicted in SEQ ID NO: 30, CDR-L1 as depicted in SEQ ID NO: 939, CDR-L2 as depicted in SEQ ID NO: 329 and CDR-L3 as depicted in SEQ ID NO: 330; which all characterize binding domains for CDH19 grouped into bin 4; and
(e) CDR-H1 as depicted in SEQ ID NO: 76, CDR-H2 as depicted in SEQ ID NO: 77, CDR-H3 as depicted in SEQ ID NO: 78, CDR-L1 as depicted in SEQ ID NO: 244, CDR-L2 as depicted in SEQ ID NO: 245 and CDR-L3 as depicted in SEQ ID NO: 246, CDR-H1 as depicted in SEQ ID NO: 88, CDR-H2 as depicted in SEQ ID NO: 89, CDR-H3 as depicted in SEQ ID NO: 90, CDR-L1 as depicted in SEQ ID NO: 256, CDR-L2 as depicted in SEQ ID NO: 257 and CDR-L3 as depicted in SEQ ID NO: 258, CDR-H1 as depicted in SEQ ID NO: 106, CDR-H2 as depicted in SEQ ID NO: 107, CDR-H3 as depicted in SEQ ID NO: 108, CDR-L1 as depicted in SEQ ID NO: 274, CDR-L2 as depicted in SEQ ID NO: 275 and CDR-L3 as depicted in SEQ ID NO: 276, CDR-H1 as depicted in SEQ ID NO: 112, CDR-H2 as depicted in SEQ ID NO: 113, CDR-H3 as depicted in SEQ ID NO: 114, CDR-L1 as depicted in SEQ ID NO: 280, CDR-L2 as depicted in SEQ ID NO: 281 and CDR-L3 as depicted in SEQ ID NO: 282, and CDR-H1 as depicted in SEQ ID NO: 106, CDR-H2 as depicted in SEQ ID NO: 107, CDR-H3 as depicted in SEQ ID NO: 108, CDR-L1 as depicted in SEQ ID NO: 274, CDR-L2 as depicted in SEQ ID NO: 275 and CDR-L3 as depicted in SEQ ID NO: 276 which all characterize binding domains for CDH19 grouped into bin 5;

In a further embodiment of the human antibody or antigen binding fragment thereof of the invention the human binding domain or antigen binding fragment thereof comprises a VH region selected from the group consisting of VH regions
(a) as depicted in SEQ ID NO: 362, SEQ ID NO: 364, SEQ ID NO: 485, SEQ ID NO: 486, SEQ ID NO: 487, SEQ ID NO: 492, SEQ ID NO: 493, SEQ ID NO: 494, and SEQ ID NO: 495;
which all characterize binding domains for CDH19 grouped into bin 1;
(b) as depicted in SEQ ID NO: 342, SEQ ID NO: 366, SEQ ID NO: 370, SEQ ID NO: 344, SEQ ID NO: 372, SEQ ID NO: 368, SEQ ID NO: 496, SEQ ID NO: 497, SEQ ID NO: 498, SEQ ID NO: 499, SEQ ID NO: 500, SEQ ID NO: 508, SEQ ID NO: 509, SEQ ID NO: 510, SEQ ID NO: 511, SEQ ID NO: 512, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, and SEQ ID NO: 538;
which all characterize binding domains for CDH19 grouped into bin 2;
(c) as depicted in SEQ ID NO: 338, SEQ ID NO: 354, SEQ ID NO: 378, SEQ ID NO: 356, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 501, SEQ ID NO: 502, SEQ ID NO: 503, SEQ ID NO: 504, SEQ ID NO: 505, SEQ ID NO: 506, SEQ ID NO: 517, and SEQ ID NO: 518;
which all characterize binding domains for CDH19 grouped into bin 3;
(d) as depicted in SEQ ID NO: 352, SEQ ID NO: 360, SEQ ID NO: 388, SEQ ID NO: 386, SEQ ID NO: 340, SEQ ID NO: 346, SEQ ID NO: 374, SEQ ID NO: 348, SEQ ID NO: 390, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 488, SEQ ID NO: 489, SEQ ID NO: 490, SEQ ID NO: 491, SEQ ID NO: 513, SEQ ID NO: 514, SEQ ID NO: 515, SEQ ID NO: 516, SEQ ID NO: 540, SEQ ID NO: 541, SEQ ID NO: 542, and SEQ ID NO: 543;
which all characterize binding domains for CDH19 grouped into bin 4; and
(e) as depicted in SEQ ID NO: 376, SEQ ID NO: 392, SEQ ID NO: 358, SEQ ID NO: 350, and SEQ ID NO: 507;
which all characterize binding domains for CDH19 grouped into bin 5.

In another embodiment the human antibody or antigen binding fragment thereof of the invention comprises the human binding domain or antigen binding fragment thereof comprising a VL region selected from the group consisting of VL regions
(a) as depicted in SEQ ID NO: 418, SEQ ID NO: 420, SEQ ID NO: 580, SEQ ID NO: 581, SEQ ID NO: 582, SEQ ID NO: 587, SEQ ID NO: 588, SEQ ID NO: 589, and SEQ ID NO: 590;
which all characterize binding domains for CDH19 grouped into bin 1;
(b) as depicted in SEQ ID NO: 398, SEQ ID NO: 422, SEQ ID NO: 426, SEQ ID NO: 400, SEQ ID NO: 428, SEQ ID NO: 424, SEQ ID NO: 591, SEQ ID NO: 592, SEQ ID NO: 593, SEQ ID NO: 594, SEQ ID NO: 595, SEQ ID NO: 603, SEQ ID NO: 604, SEQ ID NO: 605, SEQ ID NO: 606, SEQ ID NO: 607, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, and SEQ ID NO: 633;
which all characterize binding domains for CDH19 grouped into bin 2;
(c) as depicted in SEQ ID NO: 394, SEQ ID NO: 410, SEQ ID NO: 434, SEQ ID NO: 412, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 596, SEQ ID NO: 597, SEQ ID NO: 598, SEQ ID NO: 599, SEQ ID NO: 600, SEQ ID NO: 601, SEQ ID NO: 612, and SEQ ID NO: 613;
which all characterize binding domains for CDH19 grouped into bin 3;
(d) as depicted in SEQ ID NO: 408, SEQ ID NO: 416, SEQ ID NO: 444, SEQ ID NO: 442, SEQ ID NO: 396, SEQ ID NO: 402, SEQ ID NO: 430, SEQ ID NO: 404, SEQ ID NO: 446, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 583, SEQ ID NO: 584, SEQ ID NO: 585, SEQ ID NO: 586, SEQ ID NO: 608, SEQ ID NO: 609, SEQ ID NO: 610, SEQ ID NO: 611, SEQ ID NO: 635, SEQ ID NO: 636, SEQ ID NO: 637, and SEQ ID NO: 638;
which all characterize binding domains for CDH19 grouped into bin 4; and
(e) as depicted in SEQ ID NO: 432, SEQ ID NO: 448, SEQ ID NO: 414, SEQ ID NO: 406, and SEQ ID NO: 602;
which all characterize binding domains for CDH19 grouped into bin 5.

The invention further provides an embodiment of the human antibody or antigen binding fragment thereof of the invention, wherein the human binding domain or antigen binding fragment thereof comprises a VH region and a VL region selected from the group consisting of:
(1) pairs of a VH region and a VL region as depicted in SEQ ID NOs: 362+418, SEQ ID NOs: 364+420, SEQ ID NOs: 485+580, SEQ ID NOs: 486+581, SEQ ID NOs: 487+582, SEQ ID NOs: 492+587, SEQ ID NOs: 493+588, SEQ ID NOs: 494+589, and SEQ ID NOs: 495+590;
all pairs grouped into bin 1;
(2) pairs of a VH region and a VL region as depicted in SEQ ID NOs: 342+398, SEQ ID NOs: 366+422, SEQ ID NOs: 370+426, SEQ ID NOs: 344+400, SEQ ID NOs: 372+428, SEQ ID NOs: 368+424, SEQ ID NOs: 496+591, SEQ ID NOs: 497+592, SEQ ID NOs: 498+593, SEQ ID NOs: 499+594, SEQ ID NOs: 500+595, SEQ ID NOs: 508+603, SEQ ID NOs: 509+604, SEQ ID NOs: 510+605, SEQ ID NOs: 511+606, SEQ ID NOs: 512+607, SEQ ID NOs: 519+614, SEQ ID NOs: 520+615, SEQ ID NOs: 521+616, SEQ ID NOs: 522+617, SEQ ID NOs: 523+618, SEQ ID NOs: 524+619, SEQ ID NOs: 525+620, SEQ ID NOs: 526+621, SEQ ID NOs: 527+622, SEQ ID NOs: 528+623, SEQ ID NOs: 529+624, SEQ ID NOs: 530+625, SEQ ID NOs: 531+626, SEQ ID NOs: 532+627, SEQ ID NOs: 533+628, SEQ ID NOs: 534+629, SEQ ID NOs: 535+630, SEQ ID NOs: 536+631, SEQ ID NOs: 537+632, and SEQ ID NOs: 538+633;
all pairs grouped into bin 2;
(3) pairs of a VH region and a VL region as depicted in SEQ ID NOs: 338+394, SEQ ID NOs: 354+410, SEQ ID NOs: 378+434, SEQ ID NOs: 356+412, SEQ ID NOs: 476+571, SEQ ID NOs: 477+572, SEQ ID NOs: 478+573, SEQ ID NOs: 479+574, SEQ ID NOs: 480+575, SEQ ID NOs: 481+576, SEQ ID NOs: 482+577, SEQ ID NOs: 483+578, SEQ ID NOs: 484+579, SEQ ID NOs: 501+596, SEQ ID NOs: 502+597, SEQ ID NOs: 503+598, SEQ ID NOs: 504+599, SEQ ID NOs: 505+600, SEQ ID NOs: 506+601, SEQ ID NOs: 517+612, and SEQ ID NOs: 518+613;
all pairs grouped into bin 3;
(4) pairs of a VH region and a VL region as depicted in SEQ ID NOs: 352+408, SEQ ID NOs: 360+416, SEQ ID NOs: 388+444, SEQ ID NOs: 386+442, SEQ ID NOs: 340+396, SEQ ID NOs: 346+402, SEQ ID NOs: 374+430, SEQ ID NOs: 348+404, SEQ ID NOs: 390+446, SEQ ID NOs: 463+558, SEQ ID NOs: 464+559, SEQ ID NOs: 465+560, SEQ ID NOs: 466+561, SEQ ID NOs: 467+562, SEQ ID NOs: 468+563, SEQ ID NOs: 469+564, SEQ ID NOs: 470+565, SEQ ID NOs: 471+566, SEQ ID NOs: 472+567, SEQ ID NOs: 473+568, SEQ ID NOs: 474+569, SEQ ID NOs: 475+570, SEQ ID NOs: 488+583, SEQ ID NOs: 489+584, SEQ ID NOs: 490+585, SEQ ID NOs: 491+586, SEQ ID NOs: 513+608, SEQ ID NOs: 514+609, SEQ ID NOs: 515+610, SEQ ID NOs: 516+611, SEQ ID NOs: 540+635, SEQ ID NOs: 541+636, SEQ ID NOs: 542+637, and SEQ ID NOs: 543+638;
all pairs grouped into bin 4; and
(5) pairs of a VH region and a VL region as depicted in SEQ ID NOs: 376+432, SEQ ID NOs: 392+448, SEQ ID NOs: 358+414, SEQ ID NOs: 350+406, and SEQ ID NOs: 507+602;
all pairs grouped into bin 5.

In a further embodiment the human binding domain or antigen binding fragment thereof comprises the groups of heavy and light chains having an amino acid sequence selected from the group consisting of
(1) a heavy and light chain as depicted in SEQ ID NOs: 644+680, SEQ ID NOs: 650+686, SEQ ID NOs: 747+842, SEQ ID NOs: 748+843, SEQ ID NOs: 749+844, SEQ ID NOs: 754+849, SEQ ID NOs: 755+850, SEQ ID NOs: 756+851, and SEQ ID NOs: 757+852;
all pairs grouped into bin 1;
(2) a heavy and light chain as depicted in SEQ ID NOs: 660+696, SEQ ID NOs: 662+698, SEQ ID NOs: 668+704, SEQ ID NOs: 674+710, SEQ ID NOs: 672+708, SEQ ID NOs: 658+694, SEQ ID NOs: 758+853, SEQ ID NOs: 759+854, SEQ ID NOs: 760+855, SEQ ID NOs: 761+856, SEQ ID NOs: 762+857, SEQ ID NOs: 770+865, SEQ ID NOs: 771+866, SEQ ID NOs: 772+867, SEQ ID NOs: 773+868, SEQ ID NOs: 774+869, SEQ ID NOs: 781+876, SEQ ID NOs: 782+877, SEQ ID NOs: 783+878, SEQ ID NOs: 784+879, SEQ ID NOs: 785+880, SEQ ID NOs: 786+881, SEQ ID NOs: 787+882, SEQ ID NOs: 788+883, SEQ ID NOs: 789+884, SEQ ID NOs: 790+885, SEQ ID NOs: 791+886, SEQ ID NOs: 792+887, SEQ ID NOs: 793+888, SEQ ID NOs: 794+889, SEQ ID NOs: 795+890, SEQ ID NOs: 796+891, SEQ ID NOs: 797+892, SEQ ID NOs: 798+893, SEQ ID NOs: 799+894, and SEQ ID NOs: 800+895;
all pairs grouped into bin 2;
(3) a heavy and light chain as depicted in SEQ ID NOs: 656+692, SEQ ID NOs: 654+690, SEQ ID NOs: 664+700, SEQ ID NOs: 670+706, SEQ ID NOs: 738+833, SEQ ID NOs: 739+834, SEQ ID NOs: 740+835, SEQ ID NOs: 741+836, SEQ ID NOs: 742+837, SEQ ID NOs: 743+838, SEQ ID NOs: 744+839, SEQ ID NOs: 745+840, SEQ ID NOs: 746+841, SEQ ID NOs: 763+858, SEQ ID NOs: 764+859, SEQ ID NOs: 765+860, SEQ ID NOs: 766+861, SEQ ID NOs: 767+862, SEQ ID NOs: 768+863, SEQ ID NOs: 779+874, and SEQ ID NOs: 780+875;
all pairs grouped into bin 3;
(4) a heavy and light chain as depicted in SEQ ID NOs: 640+676, SEQ ID NOs: 642+678, SEQ ID NOs: 646+682, SEQ ID NOs: 648+684, SEQ ID NOs: 666+702, SEQ ID NOs: 725+820, SEQ ID NOs: 726+821, SEQ ID NOs: 727+822, SEQ ID NOs: 728+823, SEQ ID NOs: 729+824, SEQ ID NOs: 730+825, SEQ ID NOs: 731+826, SEQ ID NOs: 732+827, SEQ ID NOs: 733+828, SEQ ID NOs: 734+829, SEQ ID NOs: 735+830, SEQ ID NOs: 736+831, SEQ ID NOs: 737+832, SEQ ID NOs: 750+845, SEQ ID NOs: 751+846, SEQ ID NOs: 752+847, SEQ ID NOs: 753+848, SEQ ID NOs: 775+870, SEQ ID NOs: 776+871, SEQ ID NOs: 777+872, SEQ ID NOs: 778+873, SEQ ID NOs: 802+897, SEQ ID NOs: 803+898, SEQ ID NOs: 804+899, and SEQ ID NOs: 805+900;
all pairs grouped into bin 4; and
(5) a heavy and light chain as depicted in SEQ ID NOs: 652+688, and SEQ ID NOs: 769+864
all pairs grouped into bin 5.

In another embodiment the invention is directed to an antibody construct comprising the human antibody or antigen binding fragment thereof capable of binding to human CDH19 on the surface of a target cell as described above that is conjugated to a chemotherapeutic agent.

In one embodiment of the antibody construct of the invention a linker conjugates the chemotherapeutic agent to the human antibody or antigen binding fragment thereof. Accordingly, embodiments of the antibody construct comprising of the invention include antibody drug conjugates (ADCs). Generally the antibody construct comprising of the invention comprises an antibody conjugated to a chemotherapeutic agent, e.g., a cytotoxic agent, a cytostatic agent, a toxin, or a radioactive agent. A linker molecule can be used to conjugate the drug to the antibody. A wide variety of linkers and drugs useful e.g. in ADC technology are known in the art and may be used in embodiments of the present invention. (See US20090028856; US2009/0274713; US2007/0031402; WO2005/084390; WO2009/099728; U.S. Pat. Nos. 5,208,020; 5,416,064; 5,475,092; 5,585,499; 6,436,931; 6,372,738; and 6,340,701, all incorporated herein by reference).

In certain embodiments, the antibody construct comprising of the invention comprises a linker made up of one or more linker components. Exemplary linker components include 6-maleimidocaproyl, maleimidopropanoyl, valine-citrulline, alanine-phenylalanine, p-aminobenzyloxycarbonyl, and those resulting from conjugation with linker reagents, including, but not limited to, N-succinimidyl 4-(2-pyridylthio) pentanoate (“SPP”), N-succinimidyl 4-(N-maleimidomethyl) cyclohexane-1 carboxylate (“SMCC,” also referred to herein also as “MCC”), and N-succinimidyl (4-iodo-acetyl) aminobenzoate (“SIAB”). Linkers may be a “cleavable” linker or a “non-cleavable” linker (Ducry and Stump, Bioconjugate Chem. 2010, 21, 5-13; incorporated herein by reference in its entirety) Cleavable linkers are designed to release the drug when subjected to certain environment factors, e.g., when internalized into the target cell. Cleavable linkers include acid labile linkers, protease sensitive linkers, photolabile linkers, dimethyl linker or disulfide-containing linkers. Non-cleavable linkers tend to remain covalently associated with at least one amino acid of the antibody and the drug upon internalization by and degradation within the target cell. An exemplary non-cleavable linker is MCC.

In a preferred embodiment of the antibody construct of the invention the linker is a non-cleavable linker.

It is also preferred that the linker in the antibody construct of the invention comprises MCC.

In a further embodiment of the antibody construct of the invention the chemotherapeutic agent is conjugated to one or more lysines contained in the human antibody or antigen binding fragment thereof.

In certain embodiments, the antibody of the invention is conjugated to a chemotherapeutic agent. Examples of chemotherapeutic agents include alkylating agents, such as thiotepa and cyclophosphamide (CYTOXAN.TM.); alkyl sulfonates such as busulfan, improsulfan and piposulfan; aziridines, such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics, such as the enediyne antibiotics (e.g. calicheamicin, especially calicheamicin.gammal and calicheamicin theta I, see, e.g., Angew Chem. Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, caminomycin, carzinophilin; chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil (5-FU); folic acid analogues, such as denopterin, methotrexate, pteropterin, trimetrexate; purine analogs, such as fludarabine, 6-mercaptopurine, thiamiprine, thioguanine; pyrimidine analogs such as, ancitabine, azacitidine, 6-azauridine, carmofur, cytarabine, dideoxyuridine, doxifluridine, enocitabine, floxuridine, 5-FU; androgens, such as calusterone, dromostanolone propionate, epitiostanol, mepitiostane, testolactone; anti-adrenals, such as aminoglutethimide, mitotane, trilostane; folic acid replenisher, such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidamine; maytansinoids, such as maytansine and ansamitocins; mitoguazone; mitoxantrone; mopidamol; nitracrine; pentostatin; phenamet; pirarubicin; podophyllinic acid; 2-ethylhydrazide; procarbazine; PSK.RTM.; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb Oncology, Princeton, N.J.) and doxetaxel (TAXOTERE.RTM., Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; 65 daunomycin; aminopterin; xeloda; ibandronate; CPT-11; topoisomerase inhibitor RFS 2000; difluoromethylomithine (DMFO); retinoic acid; capecitabine; and pharmaceutically acceptable salts, acids or derivatives of any of the above. Also included in this definition are anti-hormonal agents that act to regulate or inhibit hormone action on tumors, such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens, such as flutamide, nilutamide, bicalutamide, leuprolide, and goserelin; siRNA and pharmaceutically acceptable salts, acids or derivatives of any of the above. Other chemotherapeutic agents that can be used with the present invention are disclosed in US Publication No. 20080171040 or US Publication No. 20080305044 and are incorporated in their entirety by reference.

It is contemplated that an antibody may be conjugated to two or more different chemotherapeutic agents or a pharmaceutical composition may comprise a mixture of antibodies wherein the antibody component is identical except for being conjugated to a different chemotherapeutic agent. Such embodiments may be useful for targeting multiple biological pathways with a target cell.

In preferred embodiments, the antibody construct comprising of the invention comprises an antibody conjugated to one or more maytansinoid molecules, which are mitotic inhibitors that act by inhibiting tubulin polymerization. Maytansinoids, including various modifications, are described in U.S. Pat. Nos. 3,896,111; 4,151,042; 4,137,230; 4,248,870; 4,256,746; 4,260,608; 4,265,814; 4,294,757; 4,307,016; 4,308,268; 4,309,428; 4,313,946; 4,315,929; 4,317,821; 4,322,348; 4,331,598; 4,361,650; 4,364,866; 4,424,219; 4,450,254; 4,362,663; 4,371,533; and WO 2009/099728. Maytansinoid drug moieties may be isolated from natural sources, produced using recombinant technology, or prepared synthetically. Exemplary maytansinoids include C-19-dechloro (U.S. Pat. No. 4,256,746), C-20-hydroxy (or C-20-demethyl)+/−C-19-dechloro (U.S. Pat. Nos. 4,307,016 and 4,361,650), C-20-demethoxy (or C-20-acyloxy (—OCOR), +/− dechrolo (U.S. Pat. No. 4,294,757), C-9-SH (U.S. Pat. No. 4,424,219), C-14-alkoxymethyl (demethoxy/CH2OR) (U.S. Pat. No. 4,331,598), C-14-hydroxymethyl or acyloxymethyl (CH2OH or CH2OAc) (U.S. Pat. No. 4,450,254), C-15-hydroxy/acyloxy (U.S. Pat. No. 4,364,866), C-15-methoxy (U.S. Pat. Nos. 4,313,946 and 4,315,929), C-18-N-demethyl (U.S. Pat. Nos. 4,362,663 and 4,322,348), and 4,5-deoxy (U.S. Pat. No. 4,371,533).

Various positions on maytansinoid compounds may be used as the linkage position, depending upon the type of link desired. For example, for forming an ester linkage, the C-3 position having a hydroxyl group, the C-14 position modified with hydrozymethyl, the C-15 position modified with a hydroxyl a group, and the C-20 position having a hydroxyl group are all suitable (U.S. Pat. Nos. 5,208,020, RE39151, and 6913748; US Patent Appl. Pub. Nos. 20060167245 and 20070037972, and WO 2009099728).

Preferred maytansinoids include those known in the art as DM1, DM3, and DM4 (US Pat. Appl. Pub. Nos. 2009030924 and 20050276812, incorporated herein by reference).

In one embodiment of the antibody construct of the invention the chemotherapeutic agent is DM1. Accordingly, in a preferred embodiment the antibody construct of the invention is an the human antibody or antigen binding fragment thereof conjugated to one or more DM1 molecules.

ADCs containing maytansinoids, methods of making such ADCs, and their therapeutic use are disclosed in U.S. Pat. Nos. 5,208,020 and 5,416,064, US Pat. Appl. Pub. No. 20050276812, and WO 2009099728 (all incorporated by reference herein). Linkers that are useful for making maytansinoid ADCs are know in the art (U.S. Pat. No. 5,208,020 and US Pat. Appl. Pub. Nos. 2005016993 and 20090274713; all incorporated herein by reference). Maytansinoid ADCs comprising an SMCC linker may be prepared as disclosed in U.S. Pat. No. 2005/0276812.

In certain embodiments, the antibody construct comprising of the invention comprises an antibody conjugated to DM1 with an SMCC linker.

An antibody construct comprising of the invention may have 1 to 20 chemotherapeutic agents per antibody. Compositions of ADCs may be characterized by the average number of drug moieties per antibody molecule in the composition. The average number of drug moieties may be determined by conventional means such as mass spectrometry, immunoassay, and HPLC. In some instances, a homogeneous ADC population may be separated and purified by means of reverse phase HPLC or electrophoresis. Thus, pharmaceutical ADC compositions may contain a heterogeneous or homogeneous population of antibodies linked to 1, 2, 3, 4, 5, 6, 7 or more drug moieties.

Thus, in a preferred embodiment of the antibody construct of the invention the average number of DM1 molecules per antibody construct is between 1 and 10.

It is also preferred for the antibody construct of the invention that the average number of DM1 molecules per antibody construct is between 3 and 7.

Moreover, it is preferred for the antibody construct of the invention that the average number of DM1 molecules per antibody construct is between 4 and 6.

Embodiments of the invention include antibody constructs comprising an average of about 1, about 2, about 3, about 4, about 5, about 6, about 7, about 8, about 9, about 10, about 11, about 12, about 13, about 14, about 15, about 16, about 17, about 18, about 19, or about 20 DM1 molecules per antibody.

In a further alternative embodiment of the antibody construct of the invention the average number of DM1 molecules per antibody construct is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0.

In one embodiment the antibody respectively the antibody construct of the invention comprises an effector function-enhanced antibody. One of the functions of the Fc portion of an antibody is to communicate to the immune system when the antibody binds its target. This is considered “effector function”. Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc to Fc receptors on the surface of cells of the immune system. CDC is mediated through the binding of the Fc with proteins of the complement system, e.g., C1q.

The IgG subclasses vary in their ability to mediate effector functions. For example IgG1 is much superior to IgG2 and IgG4 at mediating ADCC and CDC. Thus, in embodiments wherein a cell expressing CDH19 is targeted for destruction, an anti-CDH19 IgG1 antibody would be preferred.

The effector function of an antibody can be increased, or decreased, by introducing one or more mutations into the Fc. Embodiments of the invention include antigen binding proteins, e.g., antibodies, having an Fc engineered to increase effector function (U.S. Pat. No. 7,317,091 and Strohl, Curr. Opin. Biotech., 20:685-691, 2009; both incorporated herein by reference in its entirety). Exemplary IgG1 Fc molecules having increased effector function include (based on the Kabat numbering scheme) those have the following substitutions:
S239D/1332E
S239D/A330S/1332E
S239D/A330L/1332E
S298A/D333A/K334A
P2471/A339D
P2471/A339Q
D280H/K290S
D280H/K290S/S298D
D280H/K290S/S298V
F243L/R292P/Y300L
F243L/R292P/Y300L/P396L
F243L/R292P/Y300L/V305l/P396L
G236A/S239D/1332E
K326A/E333A
K326W/E333S
K290E/S298G/T299A
K290N/S298G/T299A
K290E/S298G/T299A/K326E
K290N/S298G/T299A/K326E

Further embodiments of the invention include antibodies, having an Fc engineered to decrease effector function. Exemplary Fc molecules having decreased effector function include (based on the Kabat numbering scheme) those have the following substitutions:
N297A (IgG1)
L234A/L235A (IgG1)
V234A/G237A (IgG2)
L235A/G237A/E318A (IgG4)
H268Q/V309L/A330S/A331S (IgG2)
C220S/C226S/C229S/P238S (IgG1)
C226S/C229S/E233P/L234V/L235A (IgG1)
L234F/L235E/P331S (IgG1)
S267E/L328F (IgG1)

Another method of increasing effector function of IgG Fc-containing proteins is by reducing the fucosylation of the Fc. Removal of the core fucose from the biantennary complex-type oligosachharides attached to the Fc greatly increased ADCC effector function without altering antigen binding or CDC effector function. Several ways are known for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing B-1,4-N-acetylglucosaminyltransferase Ill and Golgi α-mannosidase II. Alternatively, the Fc-containing molecule may be expressed in a non-mammalian cell such as a plant cell, yeast, or prokaryotic cell, e.g., E. coli. Thus, in certain embodiments of the invention, a composition comprises an antibody, e.g., Ab1, Ab2, Ab3, Ab4, Ab5, Ab6, Ab7, or Ab8, having reduced fucosylation or lacking fucosylation altogether.

The invention further provides an isolated nucleic acid molecule or sequence encoding a human antibody or antigen binding fragment thereof of the invention.

Furthermore, the invention provides a vector comprising a nucleic acid sequence of the invention. Moreover, the invention provides a host cell transformed or transfected with the nucleic acid sequence of the invention or with a vector comprising the nucleic acid molecule.

In a further embodiment the invention provides a process for the production of a human antibody or an antigen binding fragment thereof of the invention, said process comprising culturing a host cell of the invention under conditions allowing the expression of the human antibody or antigen binding fragment thereof of the invention and recovering the produced antibody or antigen binding fragment thereof from the culture.

In a further embodiment the invention provides a process for the production of an antibody construct comprising a human antibody or an antigen binding fragment thereof of the invention, said process comprising culturing a host cell of the invention under conditions allowing the expression of the human antibody or antigen binding fragment thereof of the invention and recovering the produced antibody or antigen binding fragment thereof from the culture, and conjugating a chemotherapeutic agent to the recovered antibody or antigen binding fragment thereof to produce the antibody conjugate.

Moreover, the invention provides a pharmaceutical composition comprising a human antibody or antigen binding fragment thereof of the invention or an antibody construct of the invention or produced according to the process of the invention in admixture with a pharmaceutically acceptable carrier thereof.

The formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

Those “in need of treatment” include those already with the disorder, as well as those in which the disorder is to be prevented. The term “disease” is any condition that would benefit from treatment with the protein formulation described herein. This includes chronic and acute disorders or diseases including those pathological conditions that predispose the mammal to the disease in question. Non-limiting examples of diseases/disorders to be treated herein include proliferative disease, a tumorous disease, or an immunological disorder.

In some embodiments, the invention provides a pharmaceutical composition comprising a therapeutically effective amount of one or a plurality of the a human antibody or antigen binding fragment thereof of the invention or an antibody construct of the invention together with a pharmaceutically effective diluents, carrier, solubilizer, emulsifier, preservative, and/or adjuvant. In certain embodiments, the antigen binding protein is an antibody, including a drug-conjugated antibody or a bispecific antibody. Pharmaceutical compositions of the invention include, but are not limited to, liquid, frozen, and lyophilized compositions.

Preferably, formulation materials are nontoxic to recipients at the dosages and concentrations employed. In specific embodiments, pharmaceutical compositions comprising a therapeutically effective amount of a human antibody or antigen binding fragment thereof of the invention or an antibody construct of the invention.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antigen binding proteins of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefore. In certain embodiments of the invention, human antibody or antigen binding fragment thereof of the invention or the antibody construct of the invention compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the human antibody or antigen binding fragment thereof of the invention or the antibody construct of the invention may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired human antibody or antigen binding fragment thereof of the invention or the antibody construct of the invention in a pharmaceutically acceptable vehicle. A particularly suitable vehicle for parenteral injection is sterile distilled water in which the human antibody or antigen binding fragment thereof of the invention or the antibody construct of the invention is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Additional pharmaceutical compositions will be evident to those skilled in the art, including formulations involving human antibody or antigen binding fragment thereof of the invention or the antibody construct of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Aspects of the invention includes self-buffering human antibody or antigen binding fragment thereof of the invention or the antibody construct of the invention formulations, which can be used as pharmaceutical compositions, as described in international patent application WO 06138181A2 (PCT/US2006/022599), which is incorporated by reference in its entirety herein.

As discussed above, certain embodiments provide human antibody or antigen binding fragment thereof of the invention or the antibody construct of the invention protein compositions, particularly pharmaceutical compositions of the invention, that comprise, in addition to the human antibody or antigen binding fragment thereof of the invention or the antibody construct of the invention, one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution,” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions,” Pharm Biotechnol. 13: 159-75 (2002), each of which is herein incorporated by reference in its entirety, particularly in parts pertinent to excipients and processes of the same for self-buffering protein formulations in accordance with the current invention, especially as to protein pharmaceutical products and processes for veterinary and/or human medical uses.

Salts may be used in accordance with certain embodiments of the invention to, for example, adjust the ionic strength and/or the isotonicity of a formulation and/or to improve the solubility and/or physical stability of a protein or other ingredient of a composition in accordance with the invention.

As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic.” Destabilizing solutes are referred to as “chaotropic.” Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in human antibody or antigen binding fragment thereof of the invention or the antibody construct of the invention formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Among polyols useful in select embodiments of the invention is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use in accordance with the invention. In addition, sugars that form such reactive species, such as sucrose, which is hydrolyzed to fructose and glucose under acidic conditions, and consequently engenders glycation, also is not among preferred polyols of the invention in this regard. PEG is useful to stabilize proteins and as a cryoprotectant and can be used in the invention in this regard.

Embodiments of the human antibody or antigen binding fragment thereof of the invention or the antibody construct of the invention formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product.

Surfactants routinely are used to prevent, minimize, or reduce surface adsorption. Useful surfactants in the invention in this regard include polysorbate 20, polysorbate 80, other fatty acid esters of sorbitan polyethoxylates, and poloxamer 188.

Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of human antibody or antigen binding fragment thereof of the invention or the antibody construct of the invention formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard.

Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins.

Magnesium ions (10-120 mM) can be used to inhibit isomerization of aspartic acid to isoaspartic acid. Ca+2 ions (up to 100 mM) can increase the stability of human deoxyribonuclease. Mg+2, Mn+2, and Zn+2, however, can destabilize rhDNase. Similarly, Ca+2 and Sr+2 can stabilize Factor VIII, it can be destabilized by Mg+2, Mn+2 and Zn+2, Cu+2 and Fe+2, and its aggregation can be increased by Al+3 ions.

Embodiments of the human antibody or antigen binding fragment thereof of the invention or the antibody construct of the invention formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized--dual chamber cartridge, Pharmacia & Upjohn) contain phenol while Somatrope (Eli Lilly) is formulated with m-cresol. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (about 18 to 24 months). An important point to note is that preservative effectiveness should be demonstrated in the final formulation containing the active drug and all excipient components.

Human antibody or antigen binding fragment thereof of the invention or the antibody construct of the invention generally will be designed for specific routes and methods of administration, for specific administration dosages and frequencies of administration, for specific treatments of specific diseases, with ranges of bio-availability and persistence, among other things. Formulations thus may be designed in accordance with the invention for delivery by any suitable route, including but not limited to orally, aurally, ophthalmically, rectally, and vaginally, and by parenteral routes, including intravenous and intraarterial injection, intramuscular injection, and subcutaneous injection.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided. The therapeutically effective amount of a human antibody or antigen binding fragment thereof of the invention or the antibody construct of the invention protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the human antibody or antigen binding fragment thereof of the invention or the antibody construct of the invention is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In specific embodiments, the dosage may range from 1.0 μg/kg up to about 20 mg/kg, optionally from 10 μg/kg up to about 10 mg/kg or from 100 μg/kg up to about 5 mg/kg.

A therapeutic effective amount of a human antibody or antigen binding fragment thereof of the invention or the antibody construct of the invention preferably results in a decrease in severity of disease symptoms, in increase in frequency or duration of disease symptom-free periods or a prevention of impairment or disability due to the disease affliction. For treating CDH19-expressing tumors, a therapeutically effective amount of human antibody or antigen binding fragment thereof of the invention or the antibody construct of the invention, e.g. an anti-CDH19 antibody construct (ADC construct), preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy in human tumors.

Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163, all incorporated by reference herein.

In one embodiment the invention provides the human antibody or antigen binding fragment thereof of the invention, the antibody construct of the invention, or produced according to the process of the invention for use in the prevention, treatment or amelioration of a melanoma disease or metastatic melanoma disease. Preferably, the melanoma disease or metastatic melanoma disease is selected from the group consisting of superficial spreading melanoma, lentigo maligna, lentigo maligna melanoma, acral lentiginous melanoma and nodular melanoma.

The invention also provides a method for the treatment or amelioration of a melanoma disease or metastatic melanoma disease, comprising the step of administering to a subject in need thereof the antibody or antigen binding fragment thereof of the invention, the antibody construct of the invention, an antibody or antigen binding fragment thereof of the invention or the antibody construct of the invention produced according to the process of the invention or a pharmaceutical composition of the invention.

In a preferred embodiment method the invention the melanoma disease or metastatic melanoma disease is selected from the group consisting of superficial spreading melanoma, lentigo maligna, lentigo maligna melanoma, acral lentiginous melanoma and nodular melanoma.

In a further embodiment, the invention provides a kit comprising an antibody or antigen binding fragment thereof of the invention, an antibody construct of the invention, an antibody or antigen binding fragment thereof of the invention or the antibody construct produced according to the process of the invention, a vector of the invention, and/or a host cell of the invention.

It should be understood that the inventions herein are not limited to particular methodology, protocols, or reagents, as such can vary. The discussion and examples provided herein are presented for the purpose of describing particular embodiments only and are not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

The following examples illustrate the invention. These examples should not be construed as to limit the scope of this invention. The examples are included for purposes of illustration, and the present invention is limited only by the claims.

### Example 1—Fully Human Monoclonal Antibodies Against CDH19

### 1.1 Immunization:

Fully human antibodies to Cadherin-19 (CDH19) were generated using XENOMOUSE® technology, transgenic mice engineered to express diverse repertoires of fully human IgGk and IgGλ antibodies of the corresponding isotype. (U.S. Pat. Nos. 6,114,598; 6,162,963; 6,833,268; 7,049,426; 7,064,244, which are incorporated herein by reference in their entirety; Green et al., 1994, Nature Genetics 7:13-21; Mendez et al., 1997, Nature Genetics 15:146-156; Green and Jakobovitis, 1998, J. Ex. Med. 188:483-495; Kellermann and Green, Current Opinion in Biotechnology 13, 593-597, 2002).

Mice were immunized with multiple forms of Cadherin-19 immunogen, including: (1) full length human and cynomologous (“cyno”) monkey cadherin-19, (2) secreted Cadherin-19 ecto-domain (amino acids 1-596), and (3) a truncated membrane bound form of human cadherin-19 (amino acids 1-624). Mice were immunized over a period of 8 to 10 weeks with a range of 16-18 boosts.

Sera were collected at approximately 5 and 9 weeks after the first injection and specific titers were determined by FACs staining of recombinant Cadherin-19 receptor transiently expressed on CHO—S cells. A total of 37 animals were identified with specific immune responses, these animals were pooled into 3 groups and advanced to antibody generation.

### 1.2 Preparation of Monoclonal Antibodies

Animals exhibiting suitable titers were identified, and lymphocytes were obtained from draining lymph nodes and, if necessary, pooled for each cohort. Lymphocytes were dissociated from lymphoid tissue by grinding in a suitable medium (for example, Dulbecco's Modified Eagle Medium (DMEM); obtainable from Invitrogen, Carlsbad, CA) to release the cells from the tissues, and suspended in DMEM. B cells were selected and/or expanded using standard methods, and fused with suitable fusion partner using techniques that were known in the art.

After several days of culture, the hybridoma supernatants were collected and subjected to screening assays as detailed in the examples below, including confirmation of binding to human and cynomologous monkey as well as the ability to kill cell lines in secondary antibody-drug conjugate Bioassays. Hybridoma lines that were identified to have the binding and functional properties of interest were then further selected and subjected to standard cloning and subcloning techniques. Clonal lines were expanded in vitro, and the secreted human antibodies obtained for analysis and V gene sequencing was performed.

### 1.3 Selection of Cadherin-19 Receptor Specific Binding Antibodies by FMAT

After 14 days of culture, hybridoma supernatants were screened for CDH19-specific monoclonal antibodies by Fluorometric Microvolume Assay Technology (FMAT) (Applied Biosystems, Foster City, CA). The supernatants were screened against adherent CHO cells transiently transfected with human Cadherin-19 and counter screened against CHO cells transiently transfected with the same expression plasmid that did not contain the Cadherin-19 gene.

After multiple screening campaigns, a panel of 1570 anti-Cadherin-19 binding hybridoma lines were identified and advanced to further characterization assays.

### Example 2—Assessment of Fully Human Monoclonal Antibodies Against CDH19

### 2.1 Additional Binding Characterization by Flow Cytometry (FACs)

FACS binding assays were performed to evaluate the binding of the anti-Cadherin-19 receptor specific antibodies to endogenous Cadherin-19 receptor expressed on the CHL-1 tumor cell lines. In addition, cross-reactive binding to murine and cynomologous monkey Cadherin-19 orthologues was also evaluated by FACs using recombinant forms of the various receptors transiently expressed on 293T cells.

FACs assays were performed by incubating hybridoma supernatants with 10,000 to 25,000 cells in PBS/2% Fetal bovine serum/2 mM Calcium Chloride at 4° C. for one hour followed by two washes with PBS/2% Fetal bovine serum/2 mM Calcium Chloride. Cells were then treated with florochrome-labeled secondary antibodies at 4° C. followed by one wash. The cells were resuspended in 50 μl of PBS/2% FBS and antibody binding was analyzed using a FACSCalibur™ instrument.

2.2 Antibody Drug Conjugate Screening of Fully Human Antibodies Derived from XenoMouse@Hybridomas

Cell killing through antibody drug conjugates requires the delivery of the conjugate into a cell through internalization and the catabolism of the drug-conjugate into a form that it is toxic to the cell. To identify antibodies with these properties, CDH19-positive cell lines (Colo-699 or CHL-1) were seeded at low cell densities and allowed to adhere overnight in a 384 well plate. XENOMOUSE® hybridoma samples containing fully human anti-CDH19 antibodies were then added to these cells in the presence of a high concentration of a goat anti-human Fc monovalent Fab conjugated with DM1 (DM1-Fab) at a relatively low drug-antibody ratio (DAR) (˜1.3). The cells were incubated for 96 hours at 37° C. and 5% CO₂ in the presence of the antibody samples and the DM1-Fab. At the end of this time, the cell viability was assessed using the CellTiter-Glo® Luminescent Cell Viability reagent (Promega) according to manufacturer's recommendations.

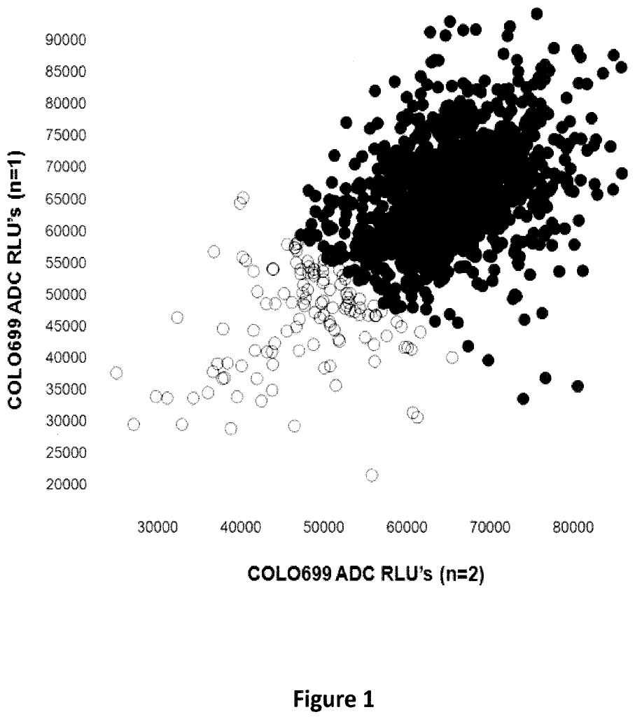

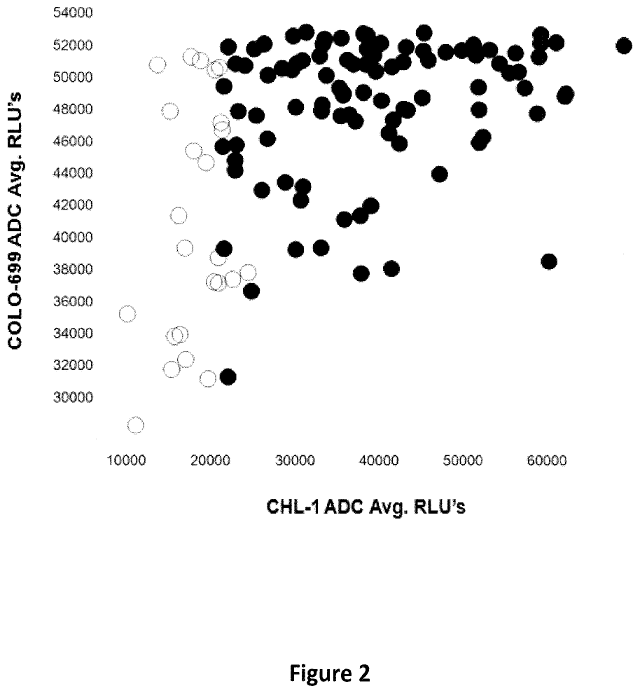

An example of the cell viability data with the Colo-699 cells is shown in FIG. 1 and FIG. 2. The antibodies capable of delivering the DM1-Fab to the cells and inhibiting the cell growth read out with a lower luminescent signal (RLU). The top antibodies of interest from this screen are observed in the lower left corner of FIG. 1 and are denoted as open circles. These antibodies were taken forward into a cell viability assay on CHL-1 cells. The average cell viability data from the CHL-1 assay is plotted against the average cell viability data from the Colo-699 assay (FIG. 2). The antibodies that had activity on both the Colo-699 and the CHL-1 cells are denoted as open circles on the left-hand side of the FIG. 2.

This assay was run concurrently with the FACs antibody binding assay above (2.2), and the results from these two studies were used to select the antibodies for further characterization. In total, 1570 antibodies were run through these cell based viability assays and approximately 44 antibodies were selected on the bases of in vitro cell killing and/or antibody binding for sub-cloning,V gene sequencing and expressed in recombinant form for further characterization assays as described below.

These 44 antibodies were again assayed as in Example 2 and 19 antibodies were selected that contained unique sequences. Of these 19 antibodies, 18 antibodies were analyzed and their properties characterized in Table 2 below. The data in this table was generated using FACs binding on recombinant human and cynomologous CDH-19, +/− Calcium (Ca⁺²) binding data on 293/CDH-19 transfectants, binding to endogenous CDH-19 on CHL-1 and Colo699 tumor cells and competition with the antibody designated as 4A9 in the table. These experiments provided the further characterizations for the grouping of these antibodies into 5 groups or bins.

TABLE 2
Binning of Lead panel using Antibody Binding Information

| | LMR | | |
|---|---|---|---|
| Bin | Sequence/ | Clone | |
| ID | Ab ID | ID | Bin Characteristics |
| 1 | 13589 | 4A9 | High Endogenous binding, Calcium |
| | 13591 | 4F7 | insensitive, sequence clustered, moderate |
| | | | cyno complete 4A9 competitor |
| 2 | 13885 | 19B5 | High Endogenous binding, Calcium |
| | 13880 | 25F8 | insensitive, sequence |
| | 13882 | 26D1 | clustered, Good cyno, |
| | 13881 | 26F12 = | partial 4A9 competitor |
| | | 27B3 | |
| | 13878 | 16H2 = | |
| | | 20D3 = | |
| | | 23E7 | |
| | 13879 | 22D1 | |
| 3 | 13877 | 22G10 | High Endogenous binding, moderate |
| | 13874 | 17H8 = | 293 binding, Calcium insensitive, |
| | | 23B6 = | 2 sequence clusters, moderate |
| | | 28D10 | cyno, partial 4A9 competitor, |
| | 13883 | 25G10 | 22G10 best binder in bin. |
| | 13875 | 16C1 | |
| 4 | 13590 | 4610 | Low Endogenous and |
| | 13586 | 4F3 | recombinant binding, |
| | 13592 | 4A2 | Calcium sensitive, sequence |
| | 13884 | 23A10 | diverse group, comparable |
| | 13588 | 2G6 | cyno, No 4A9 competition |
| 5 | 13876 | 16A4 | Best endogenous binder, moderate |
| | | | recombinant binder, calcium insensitive, |
| | | | very weak cyno, No 4A9 competition. |

Of these 18 antibodies. 8 antibodies were selected for further analysis of their epitope binding as described below. At least one representative antibody from each bin was selected for further analysis.

### Example 3—Epitope Prediction

### Epitope Prediction by 4A9 Antibody Competition and by Human/Mouse Cadherin-19 Chimeras

A 4A9 binding competition method was developed to identify antibodies that compete with 4A9 binding. In 96-well V-bottom plates (Sarstedt #82.1583.001), 50,000 transiently transfected 293T cells were incubated with 5 μg/ml of purified anti-CDH19 antibodies for 1 hr at 4° C. followed by one wash with PBS/2% FBS. 25 μl of 5 μg/ml Alexa647-labelled 4A9 was then added to each well and the plates incubated for 1 hour at 4° C. Cells were then washed two times and the amount of cell associated Alexa647-labelled 4A9 was quantitated by flow cytometry.

The experiments included negative controls consisting of PBS/2% FBS only. The average signal observed in these negative control experiments was adopted as the maximum possible signal for the assay. Antibodies were compared to this maximum signal and a percent inhibition was calculated for each well (% Inhibition=(1-(FL4 Geomean with the anti-CDH19 antibodies/Maximum FL4 Geomean signal)).

Domain binding was determined by flow cytometry as above on 293T cells transiently transfected with plasmids consisting of single or dual human CDH19 cadherin repeat domain replacements into the mouse Cadherin19 backbone cloned into the pTT5 expression vector immediately preceded by native human or murine CDH19 leader sequences and a Flag tag (SEQ ID NO: 968). The experiment included assaying the anti-CDH19 antibodies against mouse Cadherin19 to determine suitability for binning on these human/mouse chimeras. The data from these experiments are presented in the Table below entitled as follows:

TABLE 3
Calcium Sensitive Binding and Epitope Prediction Summary

| | | | | Com- | | | | | | | | | | |
|---|---|---|---|---|---|---|---|---|---|---|---|---|---|---|
| | | | | petes | Hu | | Hu | | Hu | | Hu | | Mu | |
| | | | Ca2+ | with | EC1- | Hu | EC1- | Hu | EC2- | Hu | EC4- | Hu | EC1- | Predicted |
| Clone | Ab | | Sensitive | 4A9 | 5 | EC1 | 2 | EC2 | 3 | EC3 | 5 | EC5 | 5 | Epitope |
| ID | ID | Bin | Binding | (13589) | A | B | C | D | E | F | G | H | I | Region |
| 4A9 | 13589 | 1 | No | Yes | + | + | + | − | − | − | − | − | − | 44-141 |
| | 14056 | 1 | No | Yes | + | + | + | − | − | − | − | − | − | |
| | 14057 | 1 | No | Yes | + | + | + | − | − | − | − | − | − | |
| 25F8 | 13880 | 2 | No | Yes | + | + | + | − | − | − | − | − | − | |
| | 14094 | 2 | No | Yes | + | + | + | − | − | − | − | − | − | |
| | 14096 | 2 | No | Yes | + | + | + | − | − | − | − | − | − | |
| 26D1 | 13882 | 2 | No | Yes | + | + | + | − | − | − | − | − | − | |
| | 14088 | 2 | No | Yes | + | + | + | − | − | − | − | − | − | |
| 17H8 | 13874 | 3 | No | Yes | + | + | + | − | − | − | − | − | − | |
| | 14045 | 3 | No | Yes | + | + | + | − | − | − | − | − | − | |
| | 14048 | 3 | No | Yes | + | + | + | − | − | − | − | − | − | |
| 4A2 | 13592 | 4 | Yes | No | + | − | − | − | + | + | − | − | − | 250-364 |
| | 14026 | 4 | Yes | No | + | − | − | − | + | + | − | − | − | |
| 4610 | 13590 | 4 | Yes | No | + | − | − | − | + | + | − | − | − | |
| | 14055 | 4 | Yes | No | + | − | − | − | + | + | − | − | − | |
| | 14054 | 4 | Yes | No | + | − | − | − | + | + | − | − | − | |
| 2G6 | 13588 | 4 | Yes | No | + | + | + | + | + | + | + | + | + | un- |
| | 14304 | 4 | Yes | No | + | + | + | + | + | + | + | + | + | assignable |
| | 14039 | 4 | Yes | No | + | + | + | + | + | + | + | + | + | |
| 16A4 | 13876 | 5 | No | No | + | + | + | − | − | − | − | − | − | Unassigned |
| | 14071 | 5 | No | No | + | + | + | − | − | − | − | − | − | complex |
| | | | | | | | | | | | | | | epitope |

| | | | | | | | | | |
|---|---|---|---|---|---|---|---|---|---|
| Rat anti-FLAG | + | + | + | + | + | + | + | + | + |

Legend Table 3
Human and/or murine chimera constructs
A = huCDH19(44-772) (see SEQ ID NO: 944)
B = huCDH19(44-141)::muCDH19(140-770) (see SEQ ID NO: 952)
C = huCDH19(44-249)::muCDH19(248-770) (see SEQ ID NO: 954)
D = muCDH19(44-139)::huCDH19(142-249)::muCDH19(248-770) (see SEQ ID NO: 956)
E = muCDH19(44-139)::huCDH19(142-364)::muCDH19(363-770) (see SEQ ID NO: 958)
F = muCDH19(44-247)::huCDH19(250-364)::muCDH19(363-770) (see SEQ ID NO: 960)
G = muCDH19(44-362)::huCDH19(365-772) (see SEQ ID NO: 962)
H = muCDH19(44-461)::huCDH19(464-772) (see SEQ ID NO: 964)
I = muCDH19(44-770) (see SEQ ID NO: 966)

### Epitope Prediction by Human/Chicken Cadherin-19 Chimeras

Domain binding was determined by flow cytometry on 293T cells transiently transfected with plasmids consisting of single human CDH19 cadherin repeat domain replacements into the chicken Cadherinl9 backbone cloned into the pTT5 expression vector immediately preceded by native human or chicken CDH19 leader sequences and a Flag tag. The experiment included assaying a subset of anti-CDH19 antibodies against chicken Cadherin19 to determine suitability for binning on these human/chicken chimeras.

The following binding assay was completed in presence of 2 mM CaCl2. In 96-well V-bottom plates (Costar 3897), 50,000 transiently transfected 293T cells were incubated with 5 μg/ml of purified anti-CDH19 antibodies for 1 hr at 4° C. followed by two washes with PBS/2% FBS. 50 μl of 5 μg/ml Alexa647-labelled anti-human IgG secondary antibody (Jackson Immuno 109-605-098) and 2 μg/ml 7AAD (Sigma A9400) was then added to each well and the plates incubated for 15 minutes at 4° C. Cells were then washed one time and the amount of cell associated Alexa647-labelled Ab was quantitated by flow cytometry. The experiments included mock transfected controls. The data from these experiments are presented in the Table below, n.d.=not determined.

TABLE 4
Antibody Bin C Epitope Prediction Summary

| | | | Hu | Ck | | | | | Pre- |
|---|---|---|---|---|---|---|---|---|---|
| | | | EC1- | Ed1- | Hu | Hu | Hu | Hu | dicted |
| Clone | Ab. | | 5 | 5 | EC1 | EC2 | EC3 | EC5 | Epitope |
| ID | ID | Bin | A | J | K | L | M | O | Region |
| 4A9 | 13589 | 1 | + | − | + | − | − | − | 44-141 |
| 26F12 | 13881 | 2 | + | − | + | − | − | − | Bin A |
| 25F8 | 14096 | 2 | + | − | + | − | − | − | |
| 26D1 | 13882 | 2 | + | − | + | − | − | − | |
| 17H8 | 13874 | 3 | + | − | + | − | − | − | |
| 16A4 | 14071 | 5 | + | − | + | − | − | − | |
| 4A2 | 13592 | 4 | + | − | − | − | + | − | 250- |
| 4B10 | 13590 | 4 | + | − | − | − | + | − | 364 |
| 2G6 | 13588 | 4 | + | − | − | − | + | − | Bin B |
| 23A10 | 14077 | 4 | + | − | − | − | + | − | |

| | | | | | | | |
|---|---|---|---|---|---|---|---|
| Rat anti-FLAG | + | + | + | + | + | + | control |

Positive Binding (+)
Negative Binding (−)
Legend Table 4
Human and/or chicken chimera constructs
A = huCDH19(44-772) (see SEQ ID NO: 944)
J = ckCDH19(44-776) (see SEQ ID NO: 970)
K = huCDH19(44-141)::ckCDH19(142-776) (see SEQ ID NO: 971)
L = ckCDH19(44-141)::huCDH19(142-249)::ckCDH19(250-776) (see SEQ ID NO: 972)
M = ckCDH19(44-249)::huCDH19(250-364)::ckCDH19(365-776) (see SEQ ID NO: 973)
N = ckCDH19(44-364)::huCDH19(365-463)::ckCDH19(469-776) (see SEQ ID NO: 974)
O = ckCDH19(44-468)::huCDH19(464-772) (see SEQ ID NO: 975)

Epitope Prediction by macaque/dog or rat/macaque Cadherin-19 Chimeras

Domain binding was determined by flow cytometry on 293T cells transiently transfected with plasmids consisting of rhesus macaque CDH19 cadherin repeat domain 1 or segments domain 1 (designated EC1a, EC1b, EC1c) replacements into the dog Cadherinl9 backbone, or rat CDH19 cadherin repeat domain 2 replacement into the rhesus Cadherin19 backbone cloned into the pTT5 expression vector immediately preceded by native rhesus or canine CDH19 leader sequences and a Flag tag. The experiment included assaying a subset of anti-CDH19 antibodies against dog, rat and macaque Cadherinl9 to determine suitability for binning on these macaque/dog and rat/rhesus chimeras.

The following binding assay was completed in presence of 2 mM CaCl2. In 96-well V-bottom plates (Costar 3897), 50,000 transiently transfected 293T cells were incubated with 5 μg/ml of purified anti-CDH19 antibodies for 1 hr at 4° C. followed by two washes with PBS/2% FBS. 50 μl of 5 μg/ml Alexa647-labelled anti-human IgG secondary antibody (Jackson Immuno 109-605-098) and 2 μg/ml 7AAD (Sigma A9400) was then added to each well and the plates incubated for 15 minutes at 4° C. Cells were then washed one time and the amount of cell associated Alexa647-labelled Ab was quantitated by flow cytometry. The experiments included mock transfected controls. The data from these experiments are presented in the Table below, n.d.=not determined.

TABLE 5
Antibody BinA Epitope prediction Summary

| | | | Rh | Ca | rh | rh | rh | ra | Ra | Predicted |
|---|---|---|---|---|---|---|---|---|---|---|
| | | | EC1-5 | EC1-5 | EC1 | EC1a | EC1b | EC2 | EC1-5 | Epitope |
| Clone ID | Ab. ID | Bin | P | Q | R | S | T | V | W | Region |
| 4A9 | 13589 | 1 | + | − | + | − | − | − | − | 44-141 |
| | | | | | | | | | | Bin A.1 |
| 26F12 | 13881 | 2 | + | − | + | + | + | − | − | 44-141 |
| 25F8 | 14096 | 2 | + | − | + | + | + | − | − | Bin A.2 |
| 26D1 | 13882 | 2 | + | − | + | + | + | − | − | (44- |
| | | | | | | | | | | 114) |
| 17H8 | 13874 | 3 | + | − | + | + | − | − | − | 44-141 |
| | | | | | | | | | | Bin A.3 |
| 16A4 | 14071 | 5 | + | − | + | + | − | n.d. | + | (44-65) |
| 4A2 | 13592 | 4 | + | − | n.d. | n.d. | n.d. | n.d. | + | 250- |
| 4B10 | 13590 | 4 | + | + | n.d. | n.d. | n.d. | n.d. | + | 364 |
| 2G6 | 13588 | 4 | + | + | n.d. | n.d. | n.d. | n.d. | + | Bin B |
| 23A10 | 14077 | 4 | + | + | n.d. | n.d. | n.d. | n.d. | + | |

| | | | | | | | |
|---|---|---|---|---|---|---|---|
| Rat anti-FLAG | + | + | + | + | + | + | + |

Positive Binding (+)
Negative Binding (−)
Not Determined (n.d.)
Legend Table 5
Rhesus macaque, dog, and/or rat chimera constructs
P = rhCDH19(44-772) (see SEQ ID NO: 976)
Q = caCDH19(44-770) (see SEQ ID NO: 977)
R = rhCDH19(44-141)::caCDH19(141-770) (see SEQ ID NO: 978)
S = rhCDH19(44-65)::caCDH19(65-770) (see SEQ ID NO: 979)
T = caCDH19(44-87)::rhCDH19(89-114)::caCDH19(115-770) (see SEQ ID NO: 980)
U = caCDH19(44-120)::rhCDH19(122-137)::caCDH19(137-770) (see SEQ ID NO: 981)
V = rhCDH19(44-141)::raCDH19(140-247)::rhCDH19(250-772) (see SEQ ID NO: 982)
W = raCDH19(44-770) (see SEQ ID NO: 983)

The data summarized in table 5 allowed for segregating the binder of Bin A 44-141 into the following subgroups:
Bin A.1 44-141
Bin A.2 44-141 (44-114)
Bin A.3 44-141 (44-65)
   Epitope Prediction by rat/mouse or human/mouse Cadherin-19 Chimeras

Domain binding was determined by flow cytometry on 293T cells transiently transfected with plasmids consisting of rat CDH19 cadherin repeat domain 3 substitutions (designated EC3a, EC3b) or human CDH19 cadherin repeat domain 3 substitution (designated EC3c) into the mouse Cadherin19 backbone cloned into the pTT5 expression vector immediately preceded by native mouse CDH19 leader sequence and a Flag tag. The experiment included assaying a subset of anti-CDH19 antibodies against human, rat and mouse Cadherin19 to determine suitability for binning on these rat/mouse and human/mouse chimeras.

The following binding assay was completed in presence of 2 mM CaCl2. In 96-well V-bottom plates (Costar 3897), 50,000 transiently transfected 293T cells were incubated with 5 μg/ml of purified anti-CDH19 antibodies for 1 hr at 4° C. followed by two washes with PBS/2% FBS. 50 μl of 5 μg/ml Alexa647-labelled anti-human IgG secondary antibody (Jackson Immuno 109-605-098) and 2 μg/ml 7AAD (Sigma A9400) was then added to each well and the plates incubated for 15 minutes at 4° C. Cells were then washed one time and the amount of cell associated Alexa647-labelled Ab was quantitated by flow cytometry. The experiments included mock transfected controls. The data from these experiments are presented in the Table below, n.d.=not determined.

TABLE 6
Antibody Bin B Epitope Prediction Summary

| | | | Hu | Mo | Ra | | | | Pre- |
|---|---|---|---|---|---|---|---|---|---|
| | | | EC1- | EC1- | EC1- | Ra | Ra | Hu | dicted |
| Clone | Ab. | | 5 | 5 | 5 | EC3c | EC3b | EC3a | Epitope |
| ID | ID | Bin | A | I | W | X | Y | Z | Region |
| 4A9 | 13589 | 1 | + | − | − | n.d. | n.d. | n.d. | 44-141 |
| 26F12 | 13881 | 2 | + | − | − | n.d. | n.d. | n.d. | Bin A |
| 25F8 | 14096 | 2 | + | − | − | n.d. | n.d. | n.d. | |
| 26D1 | 13882 | 2 | + | − | − | n.d. | n.d. | n.d. | |
| 17H8 | 13874 | 3 | + | − | − | n.d. | n.d. | n.d. | |
| 16A4 | 14071 | 5 | + | − | + | n.d. | n.d. | n.d. | |
| 4A2 | 13592 | 4 | + | − | + | + | − | − | 250- |
| 4B10 | 13590 | 4 | + | − | + | + | − | − | 364 |
| | | | | | | | | | (324- |
| | | | | | | | | | 327) |
| | | | | | | | | | Bin B.2 |
| 2G6 | 13588 | 4 | + | + | + | + | + | + | 250-364 |
| 23A10 | 14077 | 4 | + | + | + | n.d. | n.d. | n.d. | Bin B.1 |

| | | | | | | | |
|---|---|---|---|---|---|---|---|
| Rat anti- | + | + | + | + | + | + | control |
| FLAG | | | | | | | |

Positive Binding (+)
Negative Binding (−)
Not Determined (n.d.)
Legend Table 6
Rat/mouse or human/mouse chimera constructs
A = huCDH19(44-772) (see SEQ ID NO: 944)
I = muCDH19(44-770) (see SEQ ID NO: 966)
W = raCDH19(44-770) (see SEQ ID NO: 983)
X = muCDH19(44-323)::raCDH19(324-327)::muCDH19(328-770) (see SEQ ID NO: 984)
Y = muCDH19(44-770)::raCDH19(290, 299, 308) (see SEQ ID NO: 985)
Z = muCDH19(44-770)::huCDH19(271) (see SEQ ID NO: 986)

The data summarized in table 4 allowed for segregating the binder of Bin B 250-364 into the following subgroups:
Bin B.1 250-364
Bin B.2 250-364 (324-327) by rodent numeration as referenced in table 6, corresponding to residues (326-329) within human and macaque CDH19.

### Example 4—Hotspot/Covariant Mutants

A total of 18 antibodies were analyzed for potential hotspots and covariance violations. The designed variants (shown below) outline amino acid substitutions capable of reducing and/or avoiding isomerization, deamidation, oxidation, covariance violations, and the like. The 80 engineered variants together with the 15 parental antibodies, thus totaling 95 sequences, were taken forward to the cloning, expression, and purification processes. Site-directed mutagenesis was performed on the engineered variants in a 96-well format. The parental antibodies and engineered variants were expressed by high throughput transient transfection in HEK 293-6E cells, purified using a modified AKTA auto-sampler and assayed for activity and biophysical characteristics. The 3 parental antibodies that had either free (unpaired) Cys or N-glycosylation site were not taken forward in this process. Those were replaced with the engineered version of the parental antibodies. The designed variants outline amino acid substitutions capable of reducing and/or avoiding isomerization, deamidation, oxidation, covariance violations, immunogenicity and the like. It will be appreciated that these variant sequences are examples of engineered antibodies within the meaning of the present application but single point and/or multiple point mutations can be combined in any combinatorial manner in order to arrive at a final desired antigen binding molecule or antibody.

### Example 5—CDH19 mRNA Expression Pattern

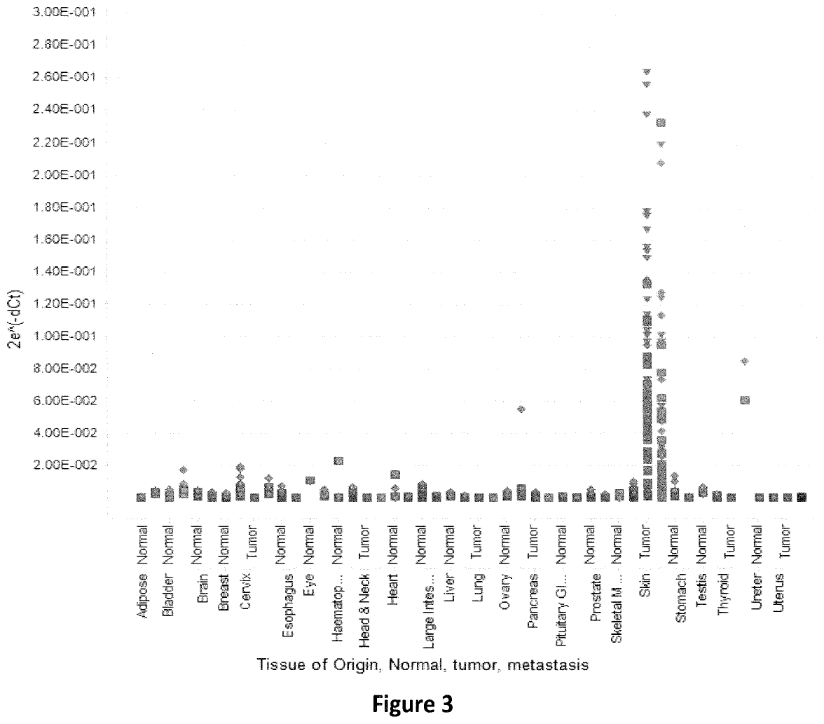

RNA was extracted from individual patient tissues representing tumor (>70% tumor content by cell count) or normal (0% tumor content by cell count). Individual tissues were homogenized using TisssueLyzer (Qiagen, Valencia, CA) and total RNA extracted and purified by the mirVana total RNA extraction kit (Life Technologies, Foster City, CA). RNA quality and quantity checked by NanoDrop (NanoDrop, Wilmington, DE) spectrophotometer readings and Bioanalyzer RNA profiling (Agilient Technologies, Santa Clara, CA). RNA was DNAse treated with DNA-free kit (Life Technologies, Foster City, CA) and reverse transcribed according to manufacturer's specifications using random hexamers in the High Capacity cDNA Reverse Transcription Kit (Life Technologies, Foster City, CA). Quantitative Real Time Polymerase Chain Reaction (qRT-PCR) was performed on cDNA using primers to CDH19, probeset Hs00253534_ml, (Life Technologies, Foster City, CA) or the housekeeping gene human ACTB (primers CCT GGC ACC CAG CAC AA; GCC GAT CCA CAC GGA GTA CT; probe ATC AAG ATC ATT GCT CCT CCT GAG CG). 10 μL qRT-PCR reaction components; 1.0 ng/μL cDNA, 2xUniversal PCR Master Mix (Life Technologies, Foster City, CA), gene expression assay (ACTB; 75 nM primers, 150 nM probe. EPOR; 300 nM primers, 250 nM probe) Following the qRT-PCR amplification program: (1) activation at 50° C. for 2 min; (2) denaturation at 95° C. for 10 min; (3) amplification 40 cycles at 95° C. for 15 s and 60° C. for 1 min with fluorescence capture at each step (ABI PRISM 7900HT Sequence Detection Systems, Applied Biosystems). Threshold cycle values (C_{T}) were determined, using Sequence Detector software version 2.3 (Applied Biosystems) and transformed to 2^{-ACT} for relative expression of CDH19 specific transcript to ACTB. The results are shown in FIG. 3. Of 54 unique metastatic and primary melanoma samples, the majority can be seen to overexpress CDH19 mRNA relative to the expression in samples from normal tissue.

### Example 6—CDH19 Protein Expression

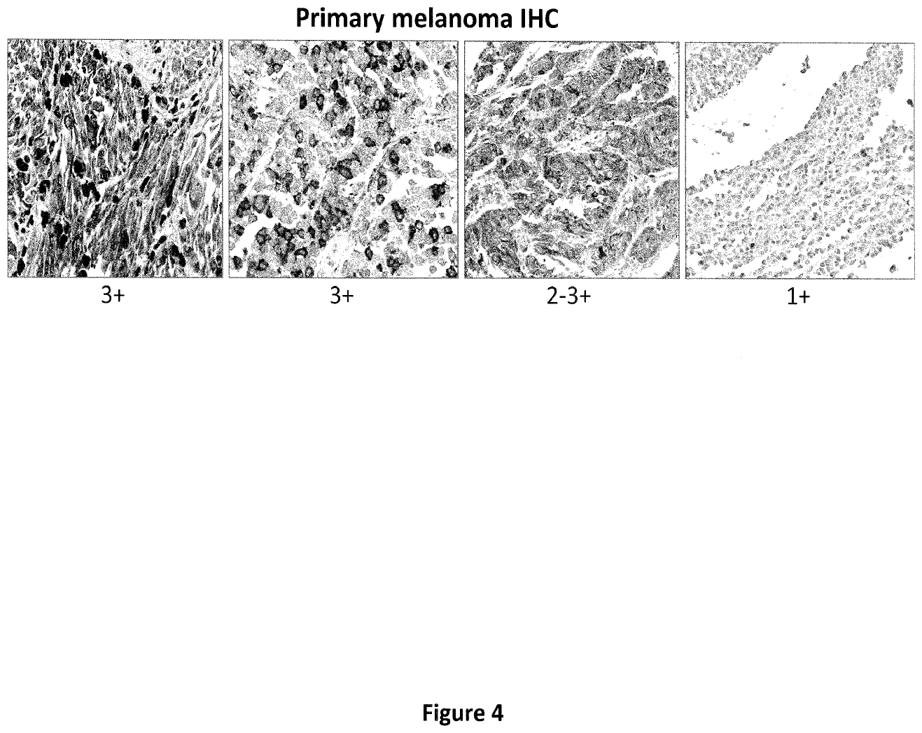

Expression of CDH19 protein was analyzed in human tumor samples by IHC and the results are shown in FIG. 4. Samples were fixed in 10% neutral buffered formalin for 24 hours, dehydrated and paraffin embedded. 4 μm sections were cut. Sections were deparaffinized first and then heated in DIVA Decloaker solution (Biocare) for 40 minutes for antigen retrieval. Remaining IHC steps were performed at room temperature in a DAKO Autostainer. Sections were incubated for 10 minutes with Peroxidazed 1 (Biocare) to block endogenous peroxidase, followed by incubation for 10 minutes with background sniper (Biocare) to reduce nonspecific background. Section were incubated for 60 minutes with CDH19 antibody (Novo Biologicals, Catalog #H00028513-B01P) at 5 μg/ml, then incubated for 30 minutes with Envision+ HRP anti-mouse polymer (DAKO), followed by DAB+(DAKO) for 5 minutes. Sections were counterstained with hematoxylin (DAKO) approximately for 1 minute. CDH19 expression could be detected in 62% of tumors examined (staining intensity ≥1+ in 101 of 162 samples). 51% of the tumor samples demonstrated medium to high expression (staining intensity of 2+ to 3+ in 83 of 162 samples). CDH19 showed dense and distinct membrane staining in many samples, although in some tumors heterogeneity was noted.

### Example 7—Selection of Model Cell Lines

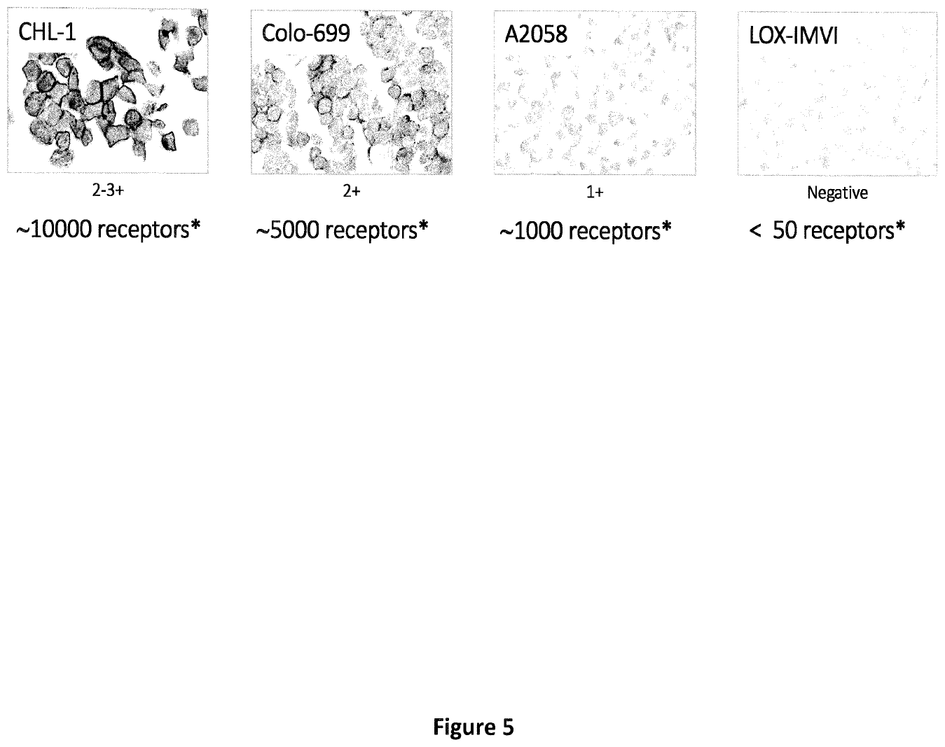

Tumor cell lines were analyzed by flow cytometry and IHC to identify model systems with CDH19 expression similar to human tumors. Human anti-huCDH19 IgG4 antibody 4A2 was purified directly from hybridoma conditioned media. For flow cytometry, 2×10⁵ cells were incubated with 200 nM of the CDH19 4A2 antibody that was conjugated to PE at a 1:1 ratio. The incubation and subsequent wash steps were performed in the presence of 1.2 mM calcium. A tube of QuantiBRITE PE lyophilized beads with four levels of PE (BD, cat #340495) was simultaneously prepared according to the manufacturer's instructions. The beads were analyzed by flow cytometry to generate a standard curve. The PE median values obtained from the melanoma lines after FACS analysis were then calibrated against the standard curve to calculate the antibodies bound per cell (ABC), which provides an estimate of the number of receptors on each cell. IHC was performed as described in Example 6 and the results are provided in FIG. 5. The melanoma cell line CHL-1 expresses about 10,000 CDH19 molecules on the cell surface, while Colo699 cells express about 5,000 receptors. Both cell lines represent tumors with medium to high expression levels based on IHC. Expression in A2058 is very low, while LOX cells do not express any detectable CDH19 protein.

### Example 8—Preparation of Antibody Drug Conjugates

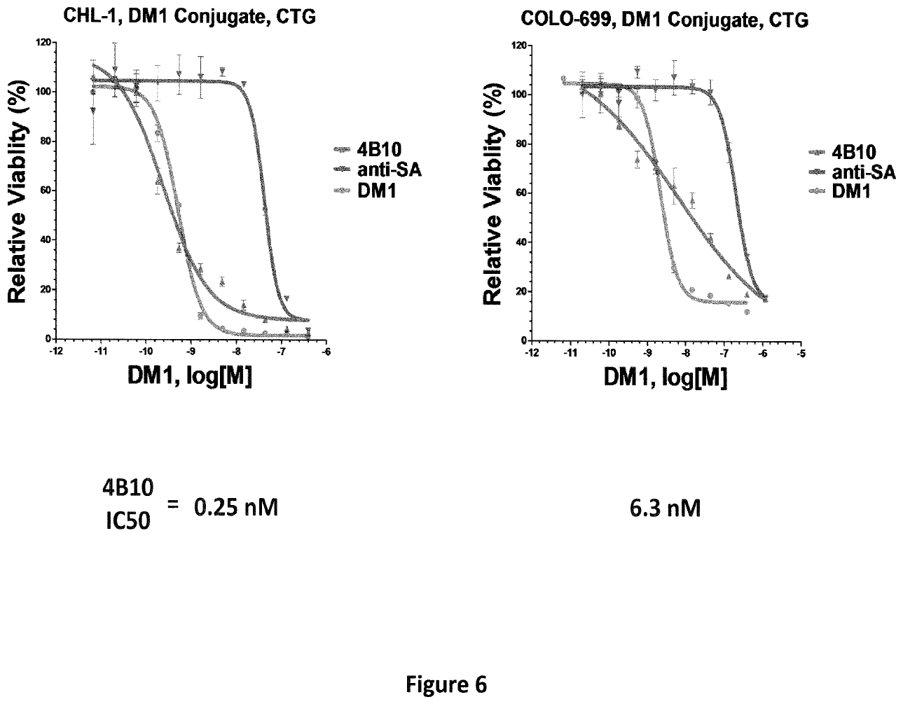

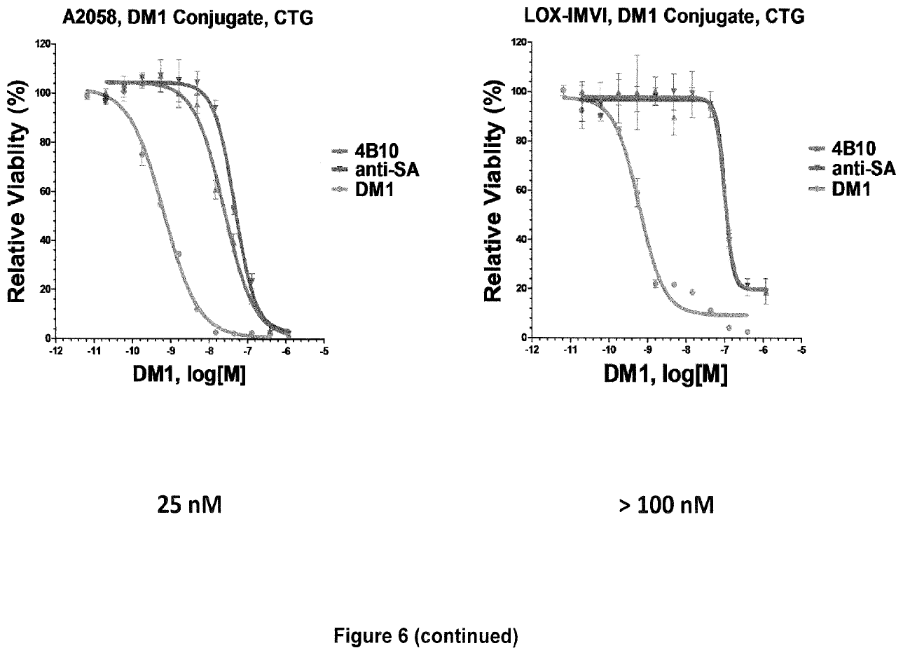

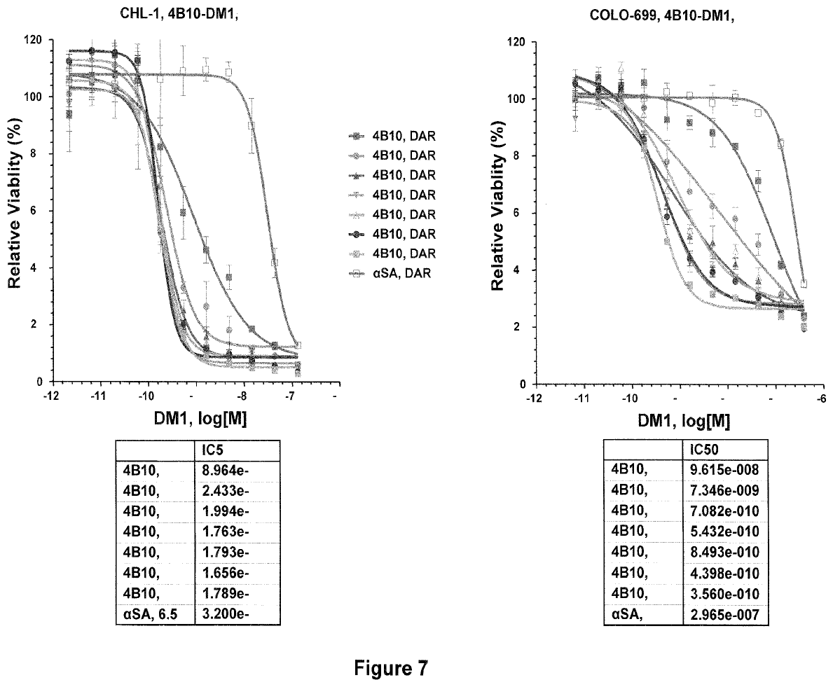

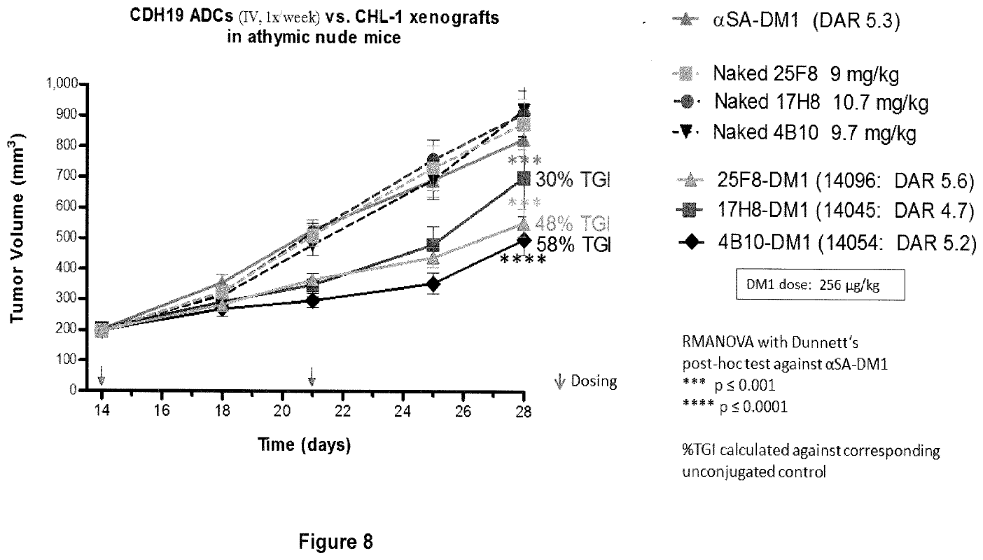

DNA sequences encoding the heavy chain and light chain components of anti-CDH19 antibodies were subcloned into mammalian expression vector pTT5 and transiently co-transfected into 293-6E cells, as described in published US2005/0170450 which is incorporated in its entirety by reference. Antibodies were purified from conditioned media by protein A affinity and ion exchange chromatography. Antibodies were incubated at 3 to 5 mg/ml with 4 to 13 equivalents of SMCC-DM1 in neutral to slightly basic buffered solutions containing 50 mM sodium chloride, 2 mM EDTA, and from 5 to 15% dimethylacetamide at room temperature for up to 5 hours or at 4° C. for up to 18 hours. Conjugation to DM1 and DAR determination for conjugates, is described in U.S. Pat. No. 7,368,565 and related U.S. Pat. No. 7,851,432, which are herein incorporated in their entirety by reference. Resultant antibody drug conjugates (ADCs) were purified from solutes and unconjugated drug by gel permeation or ion exchange chromatography. UV spectrophotometric measurements at 252 nm and 280 nm combined with respective molar extinction coefficients of SMCC-DM1 and antibody as defined by amino acid composition were used to algebraically determine the concentration of drug (CD) and antibody (CAb) components of ADC preparations which could be used to calculate a drug to antibody ratio (DAR) as described in U.S. Pat. No. 7,368,565. DAR determinations of ADCs were more accurately made by similar algebraic calculations based on integrated peaks measured at 252 nm and 280 nm in analytical size exclusion chromatography. Orthogonal LC/MS methods were also used to qualitatively assess random drug distribution profiles by mass. The table below describes ADCs used in the experiments for which the results are provided in FIG. 6 (lots 1,2), FIG. 7 (lots 3-10), and FIG. 8 (lots 11-14), which are representative of typical ADC preparations.

| Exam- | ADC | | | |
|---|---|---|---|---|
| ple | lot | ID | hu anti-huCDH19 IgG1 antibody | DAR |
| FIG. 6 | 1 | 13590 | 4B10 | 3.6 |
| FIG. 6 | 2 | 1462 | anti-SA (anti-streptavidin control) | 4.5 |
| FIG. 7 | 3 | 13590 | 4B10 | 2.5 |
| FIG. 7 | 4 | 13590 | 4B10 | 4.1 |
| FIG. 7 | 5 | 13590 | 4B10 | 5.1 |
| FIG. 7 | 6 | 13590 | 4B10 | 5.8 |
| FIG. 7 | 7 | 13590 | 4B10 | 5 |
| FIG. 7 | 8 | 13590 | 4B10 | 6.3 |
| FIG. 7 | 9 | 13590 | 4B10 | 7.4 |
| FIG. 7 | 10 | 1462 | anti-SA (anti-streptavidin control) | 6.5 |
| FIG. 8 | 11 | 14096 | 25F8.1 (K45Q,S102A,D111E) | 5.6 |
| | | | VL + (F90Y) VH | |
| FIG. 8 | 12 | 14045 | 17H8.2 (G149R) VL | 4.7 |
| FIG. 8 | 13 | 14054 | 4B10 (H450,A90T) VL + | 5.2 |
| | | | (R17G) VH | |
| FIG. 8 | 14 | 1462 | anti-SA (anti-streptavidin control) | 5.3 |

### Example 9—Activity of CDH19 Targeting ADCs in Model Cell Lines

The CDH19 recognizing parental antibody 4B10 (Ab ID 13590) was covalently coupled to the toxin DM1 as described in Example 8. The tumor cells were plated in 384-well microtiter plates on Day 1, and on Day 2, the ADC was titrated on the cells and incubated for additional 72 h. Cell viability was determined at the end of the experiment with CellTiterGlo reagent (Promega) according to the manufacturer's instructions. Unconjugated, free DM1 served as a positive control, and a streptavidin recognizing antibody/DM1 conjugate served as a negative control to detect non specific binding. IC50s were determined with a non-linear, 4 parameter curve fit and are shown in FIG. 6.

### Example 10—Effect of Drug to Antibody Ratio (DAR) on ADC Potency

In order to assess the effect of the drug antibody ratio on the potency of the ADC molecule, the CDH19 recognizing parental antibody 4B10 (Ab ID 13590) was coupled with different amounts of DM1 as indicated in FIG. 7. The effect of DARs on ADC potency was determined in cell viability assays as described in Example 9. An increased DAR leads to increases in potency for a given DM1 concentration. This effect is more pronounced on tumor cells with lower CDH19 expression.

### Example 11—Efficacy of CDH19 Targeting ADCs In Vivo

Three CDH19 recognizing engineered variant antibodies (Ab IDs 14096, 14045, 14054) were coupled to DM1 and tested in xenograft experiments. CHL-1 cells were suspended in a solution of 50% serum free medium and 50% Matrigel, and implanted subcutaneously in the flank of female athymic nude mice. Each mouse received five million cells in a volume of 200 μl. When tumors reached approximately 200 mm³, mice were sorted into seven groups of 10 mice each with equivalent mean and SD tumor size per group, and dosed with test agents or controls. All treatments were administered IV in a volume of 200 μl. Tumors were measured two times per week using calipers. Length, width and height measurements were taken A repeated measures ANOVA with Dunnett's post-hoc test was used to compare the difference in tumor volume between each CDH19 targeting ADC and a non-specific control ADC (anti-streptavidin coupled to DM1). The percentage of tumor growth inhibition was calculated for each CDH19 targeting ADC compared to the corresponding unconjugated antibody. All three reagents demonstrate significant inhibition of tumor growth in mice as shown in FIG. 8.

### Example 12—Internalization of CDH19 Following ADC Binding

Human anti-huCDH19 IgG4 antibody 4A2 was purified directly from hybridoma conditioned media and conjugated with SMCC-DM1 as described in example 8. Because the exact sequence of parental 4A2 was unknown at the time, the DAR of this IgG4 ADC was estimated to be 4.4 using a molecular weight of 150,000 Da and an extinction coefficient of 225,000 at 280 nm. CHL-1 melanoma cells were incubated with either unconjugated or DM1 conjugated CDH19 recognizing parental antibody 4A2 in complete medium at 4° C. or for 2 h at 37° C. After a brief wash in PBS, cells were fixed in 3% formaldehyde/PBS for 20 min. Fixed cells were washed, blocked and permeabilized in TBST/1% BSA/5% normal donkey serum/0.3% TX-100 and incubated with rabbit anti-EEA1 (CST #3288). Following another wash step, the samples were incubated with donkey anti mouse Alexa 488 and donkey anti rabbit Alexa 554. Images were taken with a 63x oil lens on a Zeiss LSM 510 confocal microscope. A review of the images demonstrate that both the parental and DM1 conjugated antibody detect the membrane bound CDH19 at 4° C. but get quickly internalized and co-localize with endosome markers at 37° C. Thus, both the unconjugated and DM1 conjugated CDH19 antibodies are internalized by melanoma cells, and the conjugation of the drug does not appear to interfere with the internalization of the CDH19 antibody.

### Example 13—Efficacy of CDH19 Targeting ADCs In Vivo

13.1: 4B10-DM1 Moderately Inhibited Tumor Growth at 182 μg/kg DM1 in CHL-1 Xenografts

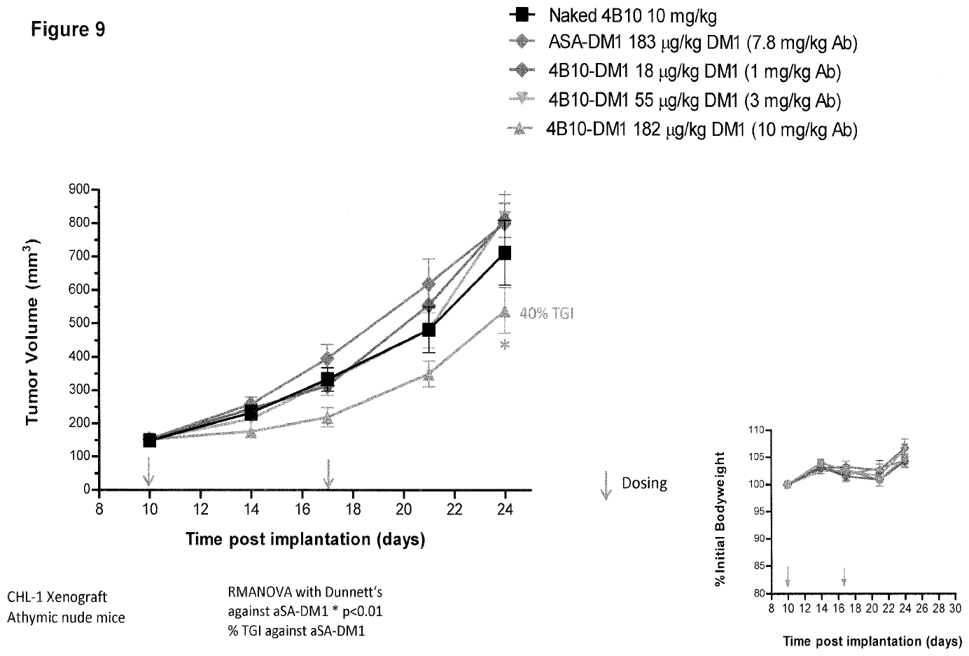

A study was conducted to examine the effect of the anti-CDH19 ADC 4B10-DM1 administered once per week for two weeks in CHL-1 xenografts. CHL-1 cells were suspended in a solution of 50% serum free medium and 50% Matrigel, and implanted subcutaneously in the flank of female athymic nude mice. Each mouse received five million cells in a volume of 200 μl. When tumors reached approximately 150 mm3, mice were sorted into groups of 10 mice each with equivalent mean and SD tumor size per group and dosed with test agents or controls. All treatments were administered IV in a volume of 200 μl. Tumors were measured two times per week using calipers (length, width and height measurement). Body weights were recorded at each measurement. A repeated measures ANOVA with Dunnett's post-hoc test was used to compare the difference in tumor volume between mice treated with 4B10-DM1 and the ADC control. The percentage of tumor growth inhibition was calculated against the ADC control. The results are shown in FIG. 9.

### 13.2: Increasing the DAR Did Not Increase Tumor Growth Inhibition in CHL-1 Xenografts

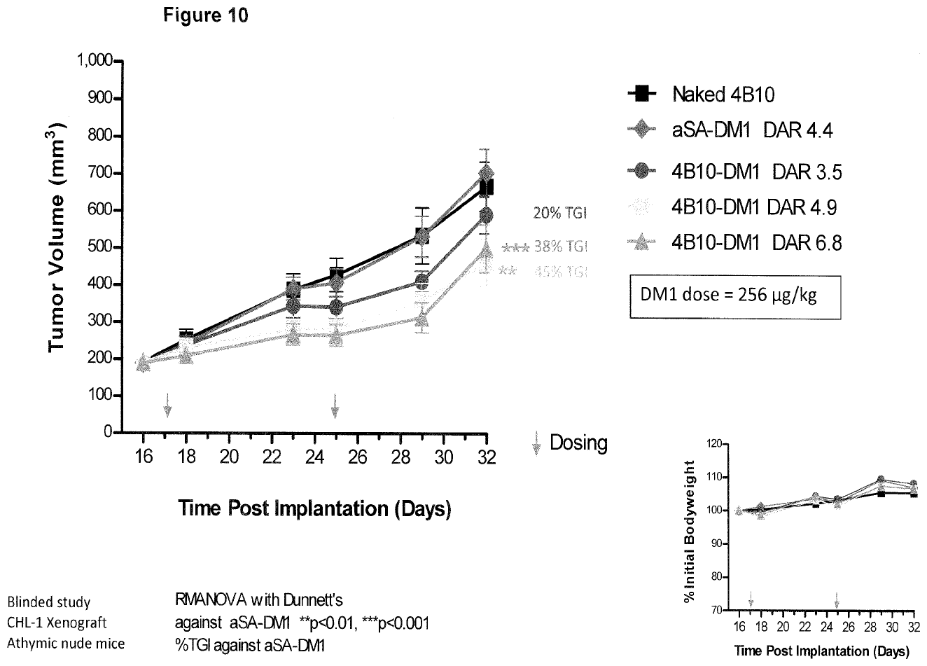

A study was conducted to examine the effect of drug:antibody ratio (DAR) on efficacy of the anti-CDH19 ADC 4B10-DM1 administered once per week for two weeks in CHL-1 xenografts. CHL-1 cells were suspended in a solution of 50% serum free medium and 50% Matrigel, and implanted subcutaneously in the flank of female athymic nude mice. Each mouse received five million cells in a volume of 200 μl. When tumors reached approximately 200 mm3, mice were sorted into groups of 10 mice each with equivalent mean and SD tumor size per group and dosed with test agents or controls. All treatments were administered IV in a volume of 200 μl. Tumors were measured two times per week using calipers (length, width and height measurement). Body weights were recorded at each measurement. A repeated measures ANOVA with Dunnett's post-hoc test was used to compare the difference in tumor volume between mice treated with 4B10-DM1 and the ADC control. The percentage of tumor growth inhibition was calculated against the ADC control. The results are shown in FIG. 10.

### 13.3: Anti-CDH19 ADCs Moderately Inhibited Tumor Growth in COL0699 Xenografts

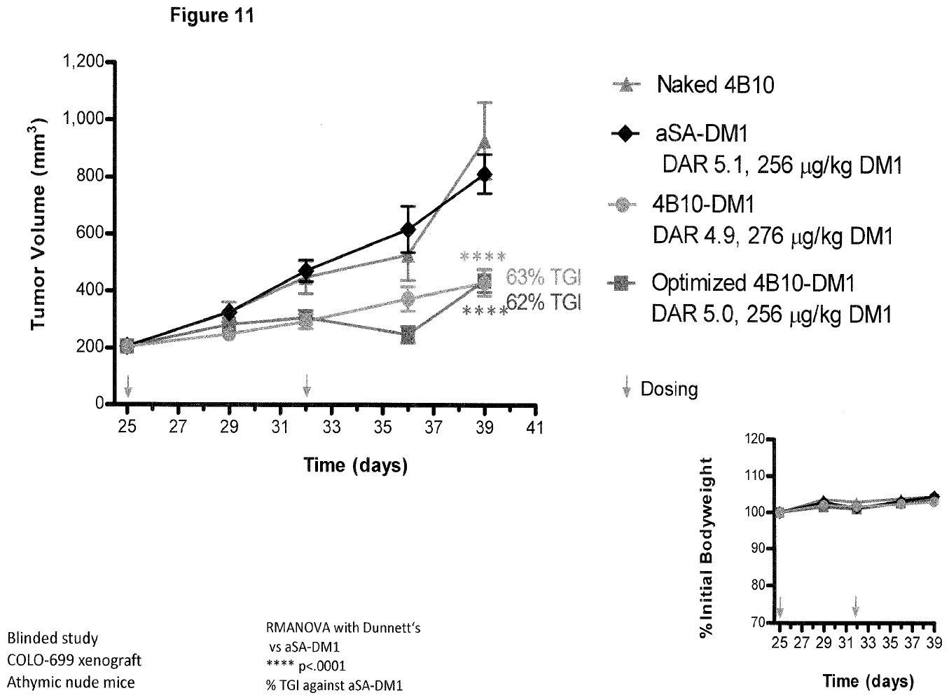

A study was conducted to examine the effects of anti-CDH19 ADC 4B10-DM1 and an optimized variant administered once per week for two weeks on COL0699 xenografts. COL0699 cells were suspended in a solution of 50% serum free medium and 50% Matrigel, and implanted subcutaneously in the flank of female athymic nude mice. Each mouse received five million cells in a volume of 200 μl. When tumors reached approximately 200 mm3, mice were sorted into groups of 10 mice each with equivalent mean and SD tumor size per group, and dosed with test agents or controls. All treatments were administered IV in a volume of 200 μl. Tumors were measured two times per week using calipers (length, width and height measurement). Body weights were recorded at each measurement. A repeated measures ANOVA with Dunnett's post-hoc test was used to compare the difference in tumor volume between mice treated with 4B10-DM1 and the ADC control. The percentage of tumor growth inhibition was calculated against the ADC control. A similar study was conducted as described above (data not shown) that resulted in the same trends for tumor growth inhibition, however, that study did not reach statistical significance. The results are shown in FIG. 11.

### Sequence Table:

TABLE Ia
HEAVY CHAIN CDRs

| Ab | Type | CDR 1 | CDR 2 | CDR 3 |
|---|---|---|---|---|
| 1D10 | NA | AGCTATGGCATGCAC | GTTATATGGTATGATGGAAGT | AGGGCCGGTATAATAGGAAC |
| 2C12 | | | AATAAATACTATGCAGACTCC | TACAGGCTACTACTACGGTA |
| | | | GTGAAGGGC | TGGACGTC |
| | | SEQ ID NO: 1 | SEQ ID NO: 2 | SEQ ID NO: 3 |
| | AA | SYGMH | VIWYDGSNKYYADSVKG | RAGIIGTTGYYYGMDV |
| | | SEQ ID NO: 4 | SEQ ID NO: 5 | SEQ ID NO: 6 |
| 1F10 | NA | AGTGGTGGTTACTACT | TACATCTATTACAGTGGGAGC | GATGGAAGCAGTGGCTGGTA |
| | | GGAGC | ACCTACTACAACCCGTCCCTC | CTTCCAGCAC |
| | | | ACGAGT | |
| | | SEQ ID NO: 7 | SEQ ID NO: 8 | SEQ ID NO: 9 |
| | AA | SGGYYWS | YIYYSGSTYYNPSLTS | DGSSGWYFQH |
| | | SEQ ID NO: 10 | SEQ ID NO: 11 | SEQ ID NO: 12 |
| 2C12_LC#1 | NA | AGCTATGGCATGCAC | GTTATATGGTATGATGGAAGT | AGGGCCGGTATAATAGGAAC |
| | | | AATAAATACTATGCAGACTCC | TACAGGCTACTACTACGGTA |
| | | | GTGAAGGGC | TGGACGTC |
| | | SEQ ID NO: 13 | SEQ ID NO: 14 | SEQ ID NO: 15 |
| | AA | SYGMH | VIWYDGSNKYYADSVKG | RAGIIGTTGYYYGMDV |
| | | SEQ ID NO: 16 | SEQ ID NO: 17 | SEQ ID NO: 18 |
| 2G6_LC#1 | NA | AGCTATGGCATGCAC | TTTATATGGTATGATGGAAGT | AGGGCCGGTATAATAGGAAC |
| | | | AATAAATACTATGCAGACTCC | TATAGGCTACTACTACGGTA |
| | | | GTGAAGGAC | TGGACGTC |
| | | SEQ ID NO: 19 | SEQ ID NO: 20 | SEQ ID NO: 21 |
| | AA | SYGMH | FIWYDGSNKYYADSVKD | RAGIIGTIGYYYGMDV |
| | | SEQ ID NO: 22 | SEQ ID NO: 23 | SEQ ID NO: 24 |
| 2G6 | NA | AGCTATGGCATGCAC | TTTATATGGTATGATGGAAGT | AGGGCCGGTATAATAGGAAC |
| | | | AATAAATACTATGCAGACTCC | TATAGGCTACTACTACGGTA |
| | | | GTGAAGGAC | TGGACGTC |
| | | SEQ ID NO: 25 | SEQ ID NO: 26 | SEQ ID NO: 27 |
| | AA | SYGMH | FIWYDGSNKYYADSVKD | RAGIIGTIGYYYGMDV |
| | | SEQ ID NO: 28 | SEQ ID NO: 29 | SEQ ID NO: 30 |
| 2H12 | NA | AGCTATGGCATGCAC | GTTATATGGTATGATGGAAGT | AGGGCCGGTATAATAGGAAC |
| | | | AATAAATACTATACAGACTCC | TACAGGCTACTACTACGGTA |
| | | | GTGAAGGGC | TGGACGTC |
| | | SEQ ID NO: 31 | SEQ ID NO: 32 | SEQ ID NO: 33 |
| | AA | SYGMH | VIWYDGSNKYYTDSVKG | RAGIIGTTGYYYGMDV |
| | | SEQ ID NO: 34 | SEQ ID NO: 35 | SEQ ID NO: 36 |
| 2H12_LC#2 | NA | AGCTATGGCATGCAC | GTTATATGGTATGATGGAAGT | AGGGCCGGTATAATAGGAAC |
| | | | AATAAATACTATACAGACTCC | TACAGGCTACTACTACGGTA |
| | | | GTGAAGGGC | TGGACGTC |
| | | SEQ ID NO: 37 | SEQ ID NO: 38 | SEQ ID NO: 39 |
| | AA | SYGMH | VIWYDGSNKYYTDSVKG | RAGIIGTTGYYYGMDV |
| | | SEQ ID NO: 40 | SEQ ID NO: 41 | SEQ ID NO: 42 |
| 4A2 | NA | AGTAGTGGTTACTACT | TACATCTATTACACTGGGAGC | GATGGAAGCAGTGGCTGGTA |
| 5B4 | | GGAGC | GCCTACTACAACCCGTCCCTC | CTTCCAGTAT |
| 5C5 | | | AAGAGT | |
| | | SEQ ID NO: 43 | SEQ ID NO: 44 | SEQ ID NO: 45 |
| | AA | SSGYYWS | YIYYTGSAYYNPSLKS | DGSSGWYFQY |
| | | SEQ ID NO: 46 | SEQ ID NO: 47 | SEQ ID NO: 48 |
| 4A9 | NA | GGTTACTACTGGAGC | TATTTCTCTTACAGTGGGAGC | AACTGGGCCTTCCACTTTGA |
| | | | ACCAACTACAACCCCTCCCTC | CTTC |
| | | | AAGAGT | |
| | | SEQ ID NO: 49 | SEQ ID NO: 50 | SEQ ID NO: 51 |
| | AA | GYYWS | YFSYSGSTNYNPSLKS | NWAFHFDF |
| | | SEQ ID NO: 52 | SEQ ID NO: 53 | SEQ ID NO: 54 |
| 4B10 | NA | AGCTATGACATGCAC | GTTATATCATATGATGGAACT | GAACGATATTTTGACTGGTC |
| 4C2 | | | AATGAATACTATGCAGACTCC | TTTTGACTAC |
| | | | GTGAAGGGC | |
| | | SEQ ID NO: 55 | SEQ ID NO: 56 | SEQ ID NO: 57 |
| | AA | SYDMH | VISYDGTNEYYADSVKG | ERYFDWSFDY |
| | | SEQ ID NO: 58 | SEQ ID NO: 59 | SEQ ID NO: 60 |
| 4D2 | NA | AGTTATGACATGCAC | GTTATATCATATGATGGAACT | GAACGATATTTTGACTGGTC |
| | | | AATGAATACTATGCAGACTCC | TTTTGACTAC |
| | | | GTGAAGGGC | |
| | | SEQ ID NO: 61 | SEQ ID NO: 62 | SEQ ID NO: 63 |
| | AA | SYDMH | VISYDGTNEYYADSVKG | ERYFDWSFDY |
| | | SEQ ID NO: 64 | SEQ ID NO: 65 | SEQ ID NO: 66 |
| 4D3 | NA | AGCTATGACATGGAC | GTTATATGGTATGATGGAAGT | GAAACTGGGGAGGgCTGGTA |
| 4F3 | | | AATAAAtacTATGCAGACTCC | CTTCGAtctc |
| | | | GTGAGGGGC | |
| | | SEQ ID NO: 67 | SEQ ID NO: 68 | SEQ ID NO: 69 |
| | AA | SYDMD | VIWYDGSNKYYADSVRG | ETGEGWYFDL |
| | | SEQ ID NO: 70 | SEQ ID NO: 71 | SEQ ID NO: 72 |
| 4E10 | NA | AGCTATGACATGCAC | GTTATATGGTATGATGGAAGT | GAGTATAGGTACAGCTGGTA |
| | | | AATAAATACTATGCAGACTCC | CTTTGACTAC |
| | | | GTGAAGGGC | |
| | | SEQ ID NO: 73 | SEQ ID NO: 74 | SEQ ID NO: 75 |
| | AA | SYDMH | VIWYDGSNKYYADSVKG | EYRYSWYFDY |
| | | SEQ ID NO: 76 | SEQ ID NO: 77 | SEQ ID NO: 78 |
| 4F7 | NA | AGTTACTCCTGGAGC | TATATCTATTACAGTGGGAGC | AACTGGGCCTTCCACTTTGA |
| | | | ACCAACTACAACCCCTCCCTC | CTAC |
| | | | AAGAGT | |
| | | SEQ ID NO: 79 | SEQ ID NO: 80 | SEQ ID NO: 81 |
| | AA | SYSWS | YIYYSGSTNYNPSLKS | NWAFHFDY |
| | | SEQ ID NO: 82 | SEQ ID NO: 83 | SEQ ID NO: 84 |
| 5E3 | NA | AGCTATAGCATGCAC | TCCATTAGTAGTAGTAGTAGT | GGGGAAACTGGAACTAACTA |
| | | | TACATATACTACGCAGACTCA | CTACTACTACGGTATGGACG |
| | | | GTGAAGGGC | TC |
| | | SEQ ID NO: 85 | SEQ ID NO: 86 | SEQ ID NO: 87 |
| | AA | SYSMH | SISSSSSYIYYADSVKG | GETGTNYYYYGMDV |
| | | SEQ ID NO: 88 | SEQ ID NO: 89 | SEQ ID NO: 90 |
| 17H8 | NA | AGTTACTACTGGAGC | TATATCTATTACATTGGGAGC | GATTCCCGGTATAGAAGTGG |
| 23B6 | | | ACCAACTACAACCCCTCCCTC | CTGGTACGATGCTTTTGATA |
| 28D10 | | | AAGAGT | TC |
| | | SEQ ID NO: 91 | SEQ ID NO: 92 | SEQ ID NO: 93 |
| | AA | SYYWS | YIYYIGSTNYNPSLKS | DSRYRSGWYDAFDI |
| | | SEQ ID NO: 94 | SEQ ID NO: 95 | SEQ ID NO: 96 |
| 16C1 | NA | GGTTACTACTGGAGC | TATATCTATTACATTGGGAGC | GATGGGAGCAGTGGCTGGTA |
| | | | ACCAACTACAACCCCTCCCTC | CCGGTGGTTCGACCCC |
| | | | AAGAGT | |
| | | SEQ ID NO: 97 | SEQ ID NO: 98 | SEQ ID NO: 99 |
| | AA | GYYWS | YIYYIGSTNYNPSLKS | DGSSGWYRWFDP |
| | | SEQ ID NO: 100 | SEQ ID NO: 101 | SEQ ID NO: 102 |
| 16A4 | NA | AGTTACTACTGGAGC | TATATCTATTACAGTGGGAGC | GATCAAAGGCGGATAGCAGC |
| | | | ACCAATTACAACCCCTCCCTC | AGCTGGTACCCACTTCTACG |
| | | | AAGAGT | GTATGGACGTC |
| | | SEQ ID NO: 103 | SEQ ID NO: 104 | SEQ ID NO: 105 |
| | AA | SYYWS | YIYYSGSTNYNPSLKS | DQRRIAAAGTHFYGMDV |
| | | SEQ ID NO: 106 | SEQ ID NO: 107 | SEQ ID NO: 108 |
| 16E2 | NA | AGCTATGGCATGCAC | GTGATATGGTATGATGGAAGT | GACGGGTGGGAGCTGTCCTT |
| 17E10 | | | AATAAATACTATGCAGACTCC | TGACTAC |
| 20B12 | | | GTGAAGGGC | |
| | | SEQ ID NO: 109 | SEQ ID NO: 110 | SEQ ID NO: 111 |
| | AA | SYGMH | VIWYDGSNKYYADSVKG | DGWELSFDY |
| | | SEQ ID NO: 112 | SEQ ID NO: 113 | SEQ ID NO: 114 |
| 22G10 | NA | AGTTATGCCATGAAC | ACTATTAGTGGTGGTGGTGCT | GGGGGAATGGGGGGATACTA |
| | | | AACACATACTACGCAGACTCC | CTACGGTATGGACGTC |
| | | | GTGAAGGGC | |
| | | SEQ ID NO: 115 | SEQ ID NO: 116 | SEQ ID NO: 117 |
| | AA | SYAMN | TISGGGANTYYADSVKG | GGMGGYYYGMDV |
| | | SEQ ID NO: 118 | SEQ ID NO: 119 | SEQ ID NO: 120 |
| 16H2 | NA | AGCTACTTTATTCAC | ATAATCAACCCTATTAGTGTT | GGGGGGATACAGCTATGGTT |
| 20D3 | | | AGCACAAGCTACGCACAGAAG | ACATTTTGACTAC |
| 23E7 | | | TTCCAGGGC | |
| | | SEQ ID NO: 121 | SEQ ID NO: 122 | SEQ ID NO: 123 |
| | AA | SYFIH | IINPISVSTSYAQKFQG | GGIQLWLHFDY |
| | | SEQ ID NO: 124 | SEQ ID NO: 125 | SEQ ID NO: 126 |
| 22D1 | NA | AGCTACTTTATTCAC | ATAATCAACCCTATTAGTGTT | GGGGGGATACAGCTATGGTT |
| | | | AGCACAAGCTACGCACAGAAG | ACATTTGGACTAC |
| | | | TTCCAGGGC | |
| | | SEQ ID NO: 127 | SEQ ID NO: 128 | SEQ ID NO: 129 |
| | AA | SYFIH | IINPISVSTSYAQKFQG | GGIQLWLHLDY |
| | | SEQ ID NO: 130 | SEQ ID NO: 131 | SEQ ID NO: 132 |
| 25F8 | NA | AGCTACTATATTCAC | ATAATCAACCCCAGTGGTGGT | GGGGGAATACAGCTATGGTT |
| | | | AGCACAAGGTACGCACAGAAG | ACATTttGACTAC |
| | | | TTCCAGGGC | |
| | | SEQ ID NO: 133 | SEQ ID NO: 134 | SEQ ID NO: 135 |
| | AA | SYYIH | IINPSGGSTRYAQKFQG | GGIQLWLHFDY |
| | | SEQ ID NO: 136 | SEQ ID NO: 137 | SEQ ID NO: 138 |
| 26F12 | NA | AACTACTATATGTCC | ATAATCAACCCTAGTGGTGGT | GGGGGGATACAACTATGGTT |
| 27B3 | | | GACTCAACCTACGCACAGAAG | ACATTTTGACTAC |
| | | | TTCCAGGGC | |
| | | SEQ ID NO: 139 | SEQ ID NO: 140 | SEQ ID NO: 141 |
| | AA | NYYMS | IINPSGGDSTYAQKFQG | GGIQLWLHFDY |
| | | SEQ ID NO: 142 | SEQ ID NO: 143 | SEQ ID NO: 144 |
| 26D1 | NA | AGCTACTATATGTCC | ATAATCCACCCTAGTGGTGGT | GGGGGGATAAAACTATGGTT |
| | | | GACACAACCTACGCACAGAAG | ACATTTTGACTAT |
| | | | TTCCAGGGC | |
| | | SEQ ID NO: 145 | SEQ ID NO: 146 | SEQ ID NO: 147 |
| | AA | SYYMS | IIHPSGGDTTYAQKFQG | GGIKLWLHFDY |
| | | SEQ ID NO: 148 | SEQ ID NO: 149 | SEQ ID NO: 150 |
| 25G10 | NA | GGTTACTACTGGAGC | TATATCTATTACATTGGGAGC | GATGGGAGCAGTGGCTGGTA |
| | | | ACCAACTACAACCCCTCCCTC | CCGGTGGTTCGACCCC |
| | | | AAGAGT | |
| | | SEQ ID NO: 151 | SEQ ID NO: 152 | SEQ ID NO: 153 |
| | AA | GYYWS | YIYYIGSTNYNPSLKS | DGSSGWYRWFDP |
| | | SEQ ID NO: 154 | SEQ ID NO: 155 | SEQ ID NO: 156 |
| 23A10 | NA | CGCTATGGCATACAC | GTTATATGGTATGATGGAAGT | AGGGCCGGTATACCTGGAAC |
| | | | AATAAATACTATGCAGACTCC | TACGGGCTACTACTATGGTA |
| | | | GTGAAGGGC | TGGACGTC |
| | | SEQ ID NO: 157 | SEQ ID NO: 158 | SEQ ID NO: 159 |
| | AA | RYGIH | VIWYDGSNKYYADSVKG | RAGIPGTTGYYYGMDV |
| | | SEQ ID NO: 160 | SEQ ID NO: 161 | SEQ ID NO: 162 |
| 19B5 | NA | AGCTACTTTATTCAC | ATTATCAACCCTATTAGTGTT | GGGGGGATACAGCTATGGTT |
| | | | AGCACAAGCTACGCACAGAAG | ACATTTGGACTAC |
| | | | TTCCAGGGC | |
| | | SEQ ID NO: 163 | SEQ ID NO: 164 | SEQ ID NO: 165 |
| | AA | SYFIH | IINPISVSTSYAQKFQG | GGIQLWLHLDY |
| | | SEQ ID NO: 166 | SEQ ID NO: 167 | SEQ ID NO: 168 |

TABLE Ib
LIGHT CHAIN CDRs

| Ab | Type | CDR 1 | CDR 2 | CDR 3 |
|---|---|---|---|---|
| 1D10 | NA | TCTGGAGATAGATTGG | CAAGATACCAAGCGGCCCTCA | CAGGCGTGGGACAGCAGCAC |
| 2C12 | | GGGAAAAATATACTTG | | TGTGGTA |
| | | C | | |
| | | SEQ ID NO: 169 | SEQ ID NO: 170 | SEQ ID NO: 171 |
| | AA | SGDRLGEKYTC | QDTKRPS | QAWDSSTVV |
| | | SEQ ID NO: 172 | SEQ ID NO: 173 | SEQ ID NO: 174 |
| 1F10 | NA | AGGGCCAGTCGGAGTA | GGTCCATCCAGCAGGGCCACT | CAGCAGTATGGTAGCTCATT |
| | | TTAGCAGCAGCTACTT | | CACT |
| | | AGCC | | |
| | | SEQ ID NO: 175 | SEQ ID NO: 176 | SEQ ID NO: 177 |
| | AA | RASRSISSSYLA | GPSSRAT | QQYGSSFT |
| | | SEQ ID NO: 178 | SEQ ID NO: 179 | SEQ ID NO: 180 |
| 2C12_LC#1 | NA | AGGtCTAGTCAAAGcc | AAGGTTTCTAACTGGGactct | ATGCAAGGTATAGTGTGGCC |
| | | tcgtaTACAGTGATGG | | GTGCAGT |
| | | AAACAcctACTTGAAT | | |
| | | SEQ ID NO: 181 | SEQ ID NO: 182 | SEQ ID NO: 183 |
| | AA | RSSQSLVYSDGNTYLN | KVSNWDS | MQGIVWPCS |
| | | SEQ ID NO: 184 | SEQ ID NO: 185 | SEQ ID NO: 186 |
| 2G6_LC#1 | NA | AGGTCTAGTCAAAGCC | CAGGTTTCTAACTGGGACTCT | ATGCAAGATACACTGTGGCC |
| | | TCGTATACAGTGATGG | | GTGCAGT |
| | | AAACACCTACTTGAAT | | |
| | | SEQ ID NO: 187 | SEQ ID NO: 188 | SEQ ID NO: 189 |
| | AA | RSSQSLVYSDGNTYLN | QVSNWDS | MQDTLWPCS |
| | | SEQ ID NO: 190 | SEQ ID NO: 191 | SEQ ID NO: 192 |
| 2G6 | NA | TCTGGAGATAGGTTGG | CAAGATACCAAGCGGCCCTCA | CAGGCGTGGGACAGCAGCAC |
| | | GGGAAAAATATACTTG | | TGTGGTA |
| | | C | | |
| | | SEQ ID NO: 193 | SEQ ID NO: 194 | SEQ ID NO: 195 |
| | AA | SGDRLGEKYTC | QDTKRPS | QAWDSSTVV |
| | | SEQ ID NO: 196 | SEQ ID NO: 197 | SEQ ID NO: 198 |
| 2H12 | NA | TCTGGAGATAGATTGG | CAAGATACCAAGCGGCCCTCA | CAGGCGTGGGACAGCAGCAC |
| | | GGGAAAAATATACTTG | | TGTGGTA |
| | | C | | |
| | | SEQ ID NO: 199 | SEQ ID NO: 200 | SEQ ID NO: 201 |
| | AA | SGDRLGEKYTC | QDTKRPS | QAWDSSTVV |
| | | SEQ ID NO: 202 | SEQ ID NO: 203 | SEQ ID NO: 204 |
| 2H12_LC#2 | NA | AGGTCTAGTCAAAGCC | AAGGTTTCTAACTGGGACTCT | ATGCAAGATACACTGTGGCC |
| | | TCGTATACAGTGATGG | | GTGCAGT |
| | | AAACACCTACTTGAAT | | |
| | | SEQ ID NO: 205 | SEQ ID NO: 206 | SEQ ID NO: 207 |
| | AA | RSSQSLVYSDGNTYLN | KVSNWDS | MQDTLWPCS |
| | | SEQ ID NO: 208 | SEQ ID NO: 209 | SEQ ID NO: 210 |
| 4A2 | NA | AGGgcCAGTCGGAATA | GGTCCATCCAGCAGGGccaCT | CAGCAGTATGGtagctCATT |
| 5B4 | | TTAGCAGCAGCTACtt | | CACT |
| 5C5 | | aGCC | | |
| | | SEQ ID NO: 211 | SEQ ID NO: 212 | SEQ ID NO: 213 |
| | AA | RASRNISSSYLA | GPSSRAT | QQYGSSFT |
| | | SEQ ID NO: 214 | SEQ ID NO: 215 | SEQ ID NO: 216 |
| 4A9 | NA | ACTGGGAGCAGCTCCA | GGTAACAACAATCGGCCCTCA | CAGTCCTATGACAGCagACT |
| | | ACATCGGGACAGGTTA | | GAGTGGTTGGGTG |
| | | TGCTGTACAC | | |
| | | SEQ ID NO: 217 | SEQ ID NO: 218 | SEQ ID NO: 219 |
| | AA | TGSSSNIGTGYAVH | GNNNRPS | QSYDSRLSGWV |
| | | SEQ ID NO: 220 | SEQ ID NO: 221 | SEQ ID NO: 222 |
| 4B10 | NA | AGGGCCAGTCAGAGTG | GGTGCATCCAGCAGGGCCACT | CAGCAGTACAGTAACTCgtg |
| 4C2 | | TTAGCAACACCTACTT | | GACG |
| | | AGCC | | |
| | | SEQ ID NO: 223 | SEQ ID NO: 224 | SEQ ID NO: 225 |
| | AA | RASQSVSNTYLA | GASSRAT | QQYSNSWT |
| | | SEQ ID NO: 226 | SEQ ID NO: 227 | SEQ ID NO: 228 |
| 4D2 | NA | AGGGCCAGTCAGAGTG | GGTGCATCCAGCAGGGCCGCT | CagcagTATAGTAacTcgtg |
| | | TTAGCAACACCTACTT | GACG | |
| | AGCC | | | |
| | | SEQ ID NO: 229 | SEQ ID NO: 230 | SEQ ID NO: 231 |
| | AA | RASQSVSNTYLA | GASSRAA | QQYSNSWT |
| | | SEQ ID NO: 232 | SEQ ID NO: 233 | SEQ ID NO: 234 |
| 4D3 | NA | AGGGCCAGTCAGAGTG | GGTGCATCCAGCAGGGCCACT | CAGCAGTATGGTAGCTCGTG |
| 4F3 | | TTAGCAGCAGCTACTT | | GACG |
| | | AGCC | | |
| | | SEQ ID NO: 235 | SEQ ID NO: 236 | SEQ ID NO: 237 |
| | AA | RASQSVSSSYLA | GASSRAT | QQYGSSWT |
| | | SEQ ID NO: 238 | SEQ ID NO: 239 | SEQ ID NO: 240 |
| 4E10 | NA | AGGGCCAGTCAGAGTG | GGTGCATCCAGCAGGGTCACT | CAGCAATATAGTAACTCGTG |
| | | TTGGCAGCAGCTACTT | | GACG |
| | | AGCC | | |
| | | SEQ ID NO: 241 | SEQ ID NO: 242 | SEQ ID NO: 243 |
| | AA | RASQSVGSSYLA | GASSRVT | QQYSNSWT |
| | | SEQ ID NO: 244 | SEQ ID NO: 245 | SEQ ID NO: 246 |
| 4F7 | NA | ACTGGGAGCAGCTCCA | GGTAACAGCAATCGGCCCTCA | CAGTCCTATGACAGCAGTCT |
| | | ATATCGGGACAGGTTA | | GAGTGGTTGGGTG |
| | | TGATGTACAC | | |
| | | SEQ ID NO: 247 | SEQ ID NO: 248 | SEQ ID NO: 249 |
| | AA | TGSSSNIGTGYDVH | GNSNRPS | QSYDSSLSGWV |
| | | SEQ ID NO: 250 | SEQ ID NO: 251 | SEQ ID NO: 252 |
| 5E3 | NA | TCTGGAGATAAATTGG | CAAGATAGCAAGCGGCCCTCA | CAGGCGTGGGACAGCAGCAC |
| | | GGGATGAATATGCTTG | | TGTGGTA |
| | | C | | |
| | | SEQ ID NO: 253 | SEQ ID NO: 254 | SEQ ID NO: 255 |
| | AA | SGDKLGDEYAC | QDSKRPS | QAWDSSTVV |
| | | SEQ ID NO: 256 | SEQ ID NO: 257 | SEQ ID NO: 258 |
| 17H8 | NA | AGGGCCAGTCAGAGTG | GGTGCATCCAGCAGGGCCACT | CAGCAGTATGGTAAATCACC |
| 23B6 | | TTGCCGGCAGCTACCT | | GATCACC |
| 28D10 | | AGCC | | |
| | | SEQ ID NO: 259 | SEQ ID NO: 260 | SEQ ID NO: 261 |
| | AA | RASQSVAGSYLA | GASSRAT | QQYGKSPIT |
| | | SEQ ID NO: 262 | SEQ ID NO: 263 | SEQ ID NO: 264 |
| 16C1 | NA | AGGGCCAGCCAGAGTG | GGTGCATCCAGCAGGGCCACT | CAGCAGTATGGTAACTCACC |
| | | TTAGCAGCAGCTACTT | | GCTCACT |
| | | AGCC | | |
| | | SEQ ID NO: 265 | SEQ ID NO: 266 | SEQ ID NO: 267 |
| | AA | RASQSVSSSYLA | GASSRAT | QQYGNSPLT |
| | | SEQ ID NO: 268 | SEQ ID NO: 269 | SEQ ID NO: 270 |
| 16A4 | NA | AGGGCCAGTCAGAGTG | GGTACATCCAGCAGGGCCACT | CAGCAGTACGGTAGCTCACC |
| | | TTAGCAGCAGTTATTT | | TTTCACT |
| | | AGCC | | |
| | | SEQ ID NO: 271 | SEQ ID NO: 272 | SEQ ID NO: 273 |
| | AA | RASQSVSSSYLA | GTSSRAT | QQYGSSPFT |
| | | SEQ ID NO: 274 | SEQ ID NO: 275 | SEQ ID NO: ***276 |
| 16E2 | NA | CGGGCGAGTCAGGGCA | GCTGCATCCAGTTTGCAAAGT | CAACACTATTTTACTTACCC |
| 17E10 | | TTAGCAATTATTTAGC | | TCGGACG |
| 20B12 | | C | | |
| | | SEQ ID NO: 277 | SEQ ID NO: 278 | SEQ ID NO: 279 |
| | AA | RASQGISNYLA | AASSLQS | QHYFTYPRT |
| | | SEQ ID NO: 280 | SEQ ID NO: 281 | SEQ ID NO: 282 |
| 22G10 | NA | AGGGCCAGTCAGAGTA | GGTGCATTTACCAGGGCCACT | CAGCAGTATAATTACTGGCC |
| | | TTAGCAGCAACTTAGC | | GCTCACT |
| | | C | | |
| | | SEQ ID NO: 283 | SEQ ID NO: 284 | SEQ ID NO: 285 |
| | AA | RASQSISSNLA | GAFTRAT | QQYNYWPLT |
| | | SEQ ID NO: 286 | SEQ ID NO: 287 | SEQ ID NO: 288 |
| 16H2 | NA | TCTGGAAGCAGCTCCA | ACTAATAATCAGCGGCCCTCA | GCAACATGGGATGACAGCCT |
| 20D3 | | ACATCGGAAGTAATTT | | GAATGGTTGGGTG |
| 23E7 | | TGTAAAC | | |
| | | SEQ ID NO: 289 | SEQ ID NO: 290 | SEQ ID NO: 291 |
| | AA | SGSSSNIGSNFVN | TNNQRPS | ATWDDSLNGWV |
| | | SEQ ID NO: 292 | SEQ ID NO: 293 | SEQ ID NO: 294 |
| 22D1 | NA | TCTGGAAGCAGCTCCA | ACTAATAATCAGCGGCCCTCA | GCAACATGGGATGACAGTAT |
| | | ACATCGGAAGCAATTT | | GAATGGTTGGGTG |
| | | TGTAAAC | | |
| | | SEQ ID NO: 295 | SEQ ID NO: 296 | SEQ ID NO: 297 |
| | AA | SGSSSNIGSNFVN | TNNQRPS | ATWDDSMNGWV |
| | | SEQ ID NO: 298 | SEQ ID NO: 299 | SEQ ID NO: 300 |
| 25F8 | NA | TCTGGAAGCAGCTCCA | ACTAATAATCAGCGGCCCTCA | GCAGCATGGGATGACAGCCT |
| | | ACATCGGAAGGAATTT | | GAATGGTTGGGTG |
| | | TGTAAAC | | |
| | | SEQ ID NO: 301 | SEQ ID NO: 302 | SEQ ID NO: 303 |
| | AA | SGSSSNIGRNFVN | TNNQRPS | AAWDDSLNGWV |
| | | SEQ ID NO: 304 | SEQ ID NO: 305 | SEQ ID NO: 306 |
| 26F12 | NA | TCTGGAAGCCGCTCCA | ACTAATTATCAGCGGCCCTCA | GCAGTATGGGATGACAGCCT |
| 27B3 | | ACATCGGAAGTAATTT | | GAATGGTTGGGTG |
| | | TGTAAAC | | |
| | | SEQ ID NO: 307 | SEQ ID NO: 308 | SEQ ID NO: 309 |
| | AA | SGSRSNIGSNFVN | TNYQRPS | AVWDDSLNGWV |
| | | SEQ ID NO: 310 | SEQ ID NO: 311 | SEQ ID NO: 312 |
| 26D1 | NA | TCTGGAAGCCGCTCCA | ACTAATAATCAGCGGCCCTCA | GCAGTATGGGATGACAGCCT |
| | | ACATCGGAAGTAATTT | | GAATGGTTGGGTG |
| | | TGTAAAC | | |
| | | SEQ ID NO: 313 | SEQ ID NO: 314 | SEQ ID NO: 315 |
| | AA | SGSRSNIGSNFVN | TNNQRPS | AVWDDSLNGWV |
| | | SEQ ID NO: 316 | SEQ ID NO: 317 | SEQ ID NO: 318 |
| 25G10 | NA | AGGGCCAGTCAGAGTG | GGTGCATCCAGCAGGGCCACT | CAGCAGTATGGTAACTCACC |
| | | TTAGCAGCAGCTACTT | | GCTCACT |
| | | AGCC | | |
| | | SEQ ID NO: 319 | SEQ ID NO: 320 | SEQ ID NO: 321 |
| | AA | RASQSVSSSYLA | GASSRAT | QQYGNSPLT |
| | | SEQ ID NO: 322 | SEQ ID NO: 323 | SEQ ID NO: 324 |
| 23A10 | NA | TCTGGAGATAGATTGG | CAAGATAATAAGTGGCCCTCA | CAGGCGTGGGACAGCAGcac |
| | | GGGAGAAATATGTTTG | | TGTGGTA |
| | | C | | |
| | | SEQ ID NO: 325 | SEQ ID NO: 326 | SEQ ID NO: 327 |
| | AA | SGDRLGEKYVC | QDNKWPS | QAWDSSTVV |
| | | SEQ ID NO: 328 | SEQ ID NO: 329 | SEQ ID NO: 330 |
| 19B5 | NA | TCTGGAAGCAGGTCCA | ACTAATAATCAGCGGCCCTCA | GCAACATGGGATGACAGTAT |
| | | ACATCGGAAGCAATTT | | GAATGGTTGGGTG |
| | | TGTAAAC | | |
| | | SEQ ID NO: 331 | SEQ ID NO: 332 | SEQ ID NO: 333 |
| | AA | SGSRSNIGSNFVN | TNNQRPS | ATWDDSMNGWV |
| | | SEQ ID NO: 334 | SEQ ID NO: 335 | SEQ ID NO: 336 |

### Anti-CDH19 Variable Region Amino Acid Sequences and Polynucleotide Sequences

TABLE IIa
Heavy Chain Variable Region Polynucleotide and Amino acid Sequences

| SEQ | | | | |
|---|---|---|---|---|
| ID | | | | |
| NO. | DESIGNATION | SOURCE | TYPE | SEQUENCE |
| 337 | 17H8 | artificial | nt | CAGGTGCAGCTGCAGGAGTCGGGCCCAGGACTGGTGAAGCCTT |
| | 23B6 | | | CGGAGACCCTGTCCCTCACGTGCACTGTCTCTGGTGGCTCCAT |
| | 28D10 | | | CAATAGTTACTACTGGAGCTGGATCCGGCAGCCCCCAGGGAAG |
| | | | | GGACTGGAGTGGATTGGGTATATCTATTACATTGGGAGCACCA |
| | | | | ACTACAACCCCTCCCTCAAGAGTCGCGTCACCATATCAGTAGA |
| | | | | CACGTCCAAGAACCAGTTCTCCCTGAAGCTGAGCTCTGTGACC |
| | | | | GCTGCGGACACGGCCCTGTATTACTGTGCGAGAGATTCCCGGT |
| | | | | ATAGAAGTGGCTGGTACGATGCTTTTGATATCTGGGGCCAAGG |
| | | | | GACAATGGTCACCGTCTCTTCA |
| 338 | 17H8 | artificial | aa | QVQLQESGPGLVKPSETLSLTCTVSGGSINSYYWSWIRQPPGK |
| | 23B6 | | | GLEWIGYIYYIGSTNYNPSLKSRVTISVDTSKNQFSLKLSSVT |
| | 28D10 | | | AADTALYYCARDSRYRSGWYDAFDIWGQGTMVTVSS |
| 339 | 4A2 | artificial | nt | CAGGTGCAGCTGCAGGAGTCGGGCCCAGGACTGGTGAAGCCTT |
| | 5B4 | | | CACAGACCCTGTCCCTCACCTGCACTGTCTCTGGTGGCTCCAT |
| | 5C5 | | | CAGCAGTAGTGGTTACTACTGGAGCTGGATCCGCCAGCACCCA |
| | | | | GGGAAGGGCCTGGAGTGGATTGGGTACATCTATTACACTGGGA |
| | | | | GCGCCTACTACAACCCGTCCCTCAAGAGTCGAGTTACCATATC |
| | | | | AGTAGACACGTCTAAGAACCAGTTCTCCCTGAAGCTGAGCTCT |
| | | | | GTGACTGCCGCGGACACGGCCGTGTATTACTGTGCGAGAGATG |
| | | | | GAAGCAGTGGCTGGTACTTCCAGTATTGGGGCCAGGGCACCCT |
| | | | | GGTCACCGTCTCCTCA |
| 340 | 4A2 | artificial | aa | QVQLQESGPGLVKPSQTLSLTCTVSGGSISSSGYYWSWIRQHP |
| | 5B4 | | | GKGLEWIGYIYYTGSAYYNPSLKSRVTISVDTSKNQFSLKLSS |
| | 5C5 | | | VTAADTAVYYCARDGSSGWYFQYWGQGTLVTVSS |
| 341 | 16H2 | artificial | nt | CAGGTGCAGCTGGTGCAGTCTGGGGCTGAGGTGAAGAAGCCTG |
| | 20D3 | | | GGGCCTCAGTGAAGGTTTCCTGCAAGGTTTCTGGATACACCTT |
| | 23E7 | | | CACCAGCTACTTTATTCACTGGGTGCGCCAGGCCCCTGGACAA |
| | | | | GGGCTTGAGTGGATGGGAATAATCAACCCTATTAGTGTTAGCA |
| | | | | CAAGCTACGCACAGAAGTTCCAGGGCAGAGTCACCATGACCAG |
| | | | | GGACACGTCCACGAGCACAGTCTTCATGGAGCTGAGCAGCCTG |
| | | | | AGATCTGAGGACACGGCCGTGTATTACTGTGCGCGAGGGGGGA |
| | | | | TACAGCTATGGTTACATTTTGACTACTGGGGCCAGGGAACCCT |
| | | | | GGTCACCGTCTCCTCA |
| 342 | 16H2 | artificial | aa | QVQLVQSGAEVKKPGASVKVSCKVSGYTFTSYFIHWVRQAPGQ |
| | 20D3 | | | GLEWMGIINPISVSTSYAQKFQGRVTMTRDTSTSTVFMELSSL |
| | 23E7 | | | RSEDTAVYYCARGGIQLWLHFDYWGQGTLVTVSS |
| 343 | 26F12 | artificial | nt | CAGGTGCAGTTGGTGCAGTCTGGGGCTGAGGTGAAGAAGCCTG |
| | 27B3 | | | GGGCCTCAGTGAAGGTTTCCTGCAAGGCATCTAGATACACCTT |
| | | | | CACCAACTACTATATGTCCTGGGTGCGACAGGCCCCTGGACAA |
| | | | | GGGCTTGAGTGGATGGGAATAATCAACCCTAGTGGTGGTGACT |
| | | | | CAACCTACGCACAGAAGTTCCAGGGCAGACTCACCATGACCGG |
| | | | | GGACACGTCCACGAGCACAGTCTACATGGAGCTGAGCAGCCTG |
| | | | | AGATCTGAGGACACGGCCGTGTATTACTGTGCGAGAGGGGGGA |
| | | | | TACAACTATGGTTACATTTTGACTACTGGGGCCAGGGAACCCT |
| | | | | GGTCACCGTCTCCTCA |
| 344 | 26F12 | artificial | aa | QVQLVQSGAEVKKPGASVKVSCKASRYTFTNYYMSWVRQAPGQ |
| | 27B3 | | | GLEWMGIINPSGGDSTYAQKFQGRLTMTGDTSTSTVYMELSSL |
| | | | | RSEDTAVYYCARGGIQLWLHFDYWGQGTLVTVSS |
| 345 | 4B10 | artificial | nt | CAGGTGCAGTTGGTGGAGTCTGGGGGAGGCGTGGTCCAGCCTG |
| | 4C2 | | | GGAGGTCCCTGAGACTCTCCTGTGCAGCCTCTGGATTCACCTT |
| | | | | CAGTAGCTATGACATGCACTGGGTCCGCCAGGCTCCAGGCAAG |
| | | | | GGGCTGGAGTGGGTGGCAGTTATATCATATGATGGAACTAATG |
| | | | | AATACTATGCAGACTCCGTGAAGGGCCGATTCACCATCTCCAG |
| | | | | AGACACTTCCAAGAACACGCTGTATTTGCAAATGAACAGCCTG |
| | | | | AGAGCTGAGGACACGGCTGTATATTACTGTGCGAGAGAACGAT |
| | | | | ATTTTGACTGGTCTTTTGACTACTGGGGCCAGGGAACCCTGGT |
| | | | | CAGTGTCTCCTCA |
| 346 | 4B10 | artificial | aa | QVQLVESGGGVVQPGRSLRLSCAASGFTFSSYDMHWVRQAPGK |
| | 4C2 | | | GLEWVAVISYDGTNEYYADSVKGRFTISRDTSKNTLYLQMNSL |
| | | | | RAEDTAVYYCARERYFDWSFDYWGQGTLVSVSS |
| 347 | 4D3 | artificial | nt | CAGGTGCAGCTGGTGGAGTCTGGGGGAGGCGTGGTCCAGCCTG |
| | 4F3 | | | GGAGGTCCCTGAGACTCTCCTGTGCAGCGTCTGGATTCTCCTT |
| | | | | CAGTAGCTATGACATGGACTGGGTCCGCCAGACTCCAGGCAAG |
| | | | | GGGCTGGAGTGGGTGGCAGTTATATGGTATGATGGAAGTAATA |
| | | | | AATACTATGCAGACTCCGTGAGGGGCCGATTCACCATCTCCAG |
| | | | | AGACAATTCCAAGAACACGCTGTTTCTGCAAATGAACAGCCTG |
| | | | | AGAGTCGAGGACACGGCTGTGTATTACTGTGCGAGAGAAACTG |
| | | | | GGGAGGGCTGGTACTTCGATCTCTGGGGCCGTGGCACCCTGGT |
| | | | | CACTGTCTCCTCA |
| 348 | 4D3 | artificial | aa | QVQLVESGGGVVQPGRSLRLSCAASGFSFSSYDMDWVRQTPGK |
| | 4F3 | | | GLEWVAVIWYDGSNKYYADSVRGRFTISRDNSKNTLFLQMNSL |
| | | | | RVEDTAVYYCARETGEGWYFDLWGRGTLVTVSS |
| 349 | 16E2 | artificial | nt | CAGGTGCAGCTGGTGGAGTCTGGGGGAGGCGTGGTCCAGCCTG |
| | 17E10 | | | GGAGGTCCCTGAGACTCTCCTGTGCAGCGTCTGGATTCATCTT |
| | 20B12 | | | CAGTAGCTATGGCATGCACTGGGTCCGCCAGACTCCAGGCAAG |
| | | | | GGGCTGGAGTGGGTGGCAGTGATATGGTATGATGGAAGTAATA |
| | | | | AATACTATGCAGACTCCGTGAAGGGCCGATTCACCATCTCCAG |
| | | | | AGACATTTCCAAGAACACGCTGTATCTGCAAATGAACAGCCTG |
| | | | | AGAGTCGAGGACACGGCTGTGTATTACTGTGCGAGAGACGGGT |
| | | | | GGGAGCTGTCCTTTGACTACTGGGGCCAGGGAACCCTGGTCAC |
| | | | | CGTCTCCTCA |
| 350 | 16E2 | artificial | aa | QVQLVESGGGVVQPGRSLRLSCAASGFIFSSYGMHWVRQTPGK |
| | 17E10 | | | GLEWVAVIWYDGSNKYYADSVKGRFTISRDISKNTLYLQMNSL |
| | 20B12 | | | RVEDTAVYYCARDGWELSFDYWGQGTLVTVSS |
| 351 | 1D10 | artificial | nt | CAGGTGCAGCTGGTGGAGTCTGGGGGAGGCGTGGTCCAGCCTG |
| | 2C12 | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGTCAGTTATATGGTATGATGGAAGTAATA |
| | | | | AATACTATGCAGACTCCGTGAAGGGCCGATTCACCATCTCCAG |
| | | | | AGACAATTCCAAGAACACGCTGTATCTGCAAATGAATAGCCTG |
| | | | | AGAGCTGAGGACACGGCTGTGTATTACTGCGCGAGAAGGGCCG |
| | | | | GTATAATAGGAACTACAGGCTACTACTACGGTATGGACGTCTG |
| | | | | GGGCCAAGGGACCACGGTCACCGTCTCCTCA |
| 352 | 1D10 | artificial | aa | QVQLVESGGGVVQPGRSLRLSCAASGFTFSSYGMHWVRQAPGK |
| | 2C12 | | | GLEWVSVIWYDGSNKYYADSVKGRFTISRDNSKNTLYLQMNSL |
| | | | | RAEDTAVYYCARRAGIIGTTGYYYGMDVWGQGTTVTVSS |
| 353 | 16C1 | artificial | nt | CAGGTGCAGCTGCAGGAGTCGGGCCCAGGACTGGTGAAGCCTT |
| | | | | CGGAGACCCTGTCCCTCACTTGTACTGTCTCTGGTGGCTCCAT |
| | | | | CAGTGGTTACTACTGGAGCTGGATCCGGCAGCCCCCAGGGAAG |
| | | | | GGACTGGAGTGGATTGGGTATATCTATTACATTGGGAGCACCA |
| | | | | ACTACAACCCCTCCCTCAAGAGTCGAGTCACCATGTCAATAGA |
| | | | | CACGTCCAAGAACCAGTTCTCCCTGACGCTGAGCTCTTTGACC |
| | | | | GCTGCGGACACGGCCGTGTATTTCTGTGCGAGAGATGGGAGCA |
| | | | | GTGGCTGGTACCGGTGGTTCGACCCCTGGGGCCAGGGAACCCT |
| | | | | GGTCACCGTCTCCTCA |
| 354 | 16C1 | artificial | aa | QVQLQESGPGLVKPSETLSLTCTVSGGSISGYYWSWIRQPPGK |
| | | | | GLEWIGYIYYIGSTNYNPSLKSRVTMSIDTSKNQFSLTLSSLT |
| | | | | AADTAVYFCARDGSSGWYRWFDPWGQGTLVTVSS |
| 355 | 25G10 | artificial | nt | CAGGTGCAGCTGCAGGAGTCGGGCCCAGGACTGGTGAAGCCTT |
| | | | | CGGAGACCCTGTCCCTCACCTGCACTGTCTCTGGTGGCTCCAT |
| | | | | CAGTGGTTACTACTGGAGCTGGATCCGGCAGCCCCCAGGGAAG |
| | | | | GGACTGGAGTGGATTGGGTATATCTATTACATTGGGAGCACCA |
| | | | | ACTACAACCCCTCCCTCAAGAGTCGAGTCACCATGTCAGTAGA |
| | | | | CACGTCCAAGAACCAGTTCTCCCTGAAGCTGAGCTCTGTGACC |
| | | | | GCTGCGGACACGGCCGTGTATTACTGTGCGAGAGATGGGAGCA |
| | | | | GTGGCTGGTACCGGTGGTTCGACCCCTGGGGCCAGGGAACCCT |
| | | | | GGTCACCGTCTCCTCA |
| 356 | 25G10 | artificial | aa | QVQLQESGPGLVKPSETLSLTCTVSGGSISGYYWSWIRQPPGK |
| | | | | GLEWIGYIYYIGSTNYNPSLKSRVTMSVDTSKNQFSLKLSSVT |
| | | | | AADTAVYYCARDGSSGWYRWFDPWGQGTLVTVSS |
| 357 | 16A4 | artificial | nt | CAGGTGCAGCTGCAGGAGTCgGGCCCAGGACTGGCGAAgcctt |
| | | | | cGGAGACcctgtccctcacctgCACTGTCTCTGGTGACTCCAT |
| | | | | CACTAGTTACTACTGGAGCTGGATCCGGCAGCCCCCAGGGAAG |
| | | | | GGACTGGAGTGGATTGGGTATATCTATTACAGTGGGAGCACCA |
| | | | | ATTACAACCCCTCCCTCAAGAGTCGAGTCACCATATCAGTAGA |
| | | | | CACGTCCAAGAACCAGTTCTCCCTGAAGCTGAGTTCTGTGACC |
| | | | | GCTGCGGACACGGCCGTGTATTACTGTGCGAGAGATCAAAGGC |
| | | | | GGATAGCAGCAGCTGGTACCCACTTCTACGGTATGGACGTCTG |
| | | | | GGGCCAAGGGACCACGGTCACCGTCTCCTCA |
| 358 | 16A4 | artificial | aa | QVQLQESGPGLAKPSETLSLTCTVSGDSITSYYWSWIRQPPGK |
| | | | | GLEWIGYIYYSGSTNYNPSLKSRVTISVDTSKNQFSLKLSSVT |
| | | | | AADTAVYYCARDQRRIAAAGTHFYGMDVWGQGTTVTVSS |
| 359 | 1F10 | artificial | nt | CAGGTGCAGCTGCAGGAGTCGGGCCCAGGACTGGTGAAGCCTT |
| | | | | CACAGACCCTGTCCCTCACCTGCACTGTCTCTGGTGGCTCCAT |
| | | | | CAGCAGTGGTGGTTACTACTGGAGCTGGATCCGCCAGCACCCA |
| | | | | GGGAAGGGCCTGGAGTGGATTGGGTACATCTATTACAGTGGGA |
| | | | | GCACCTACTACAACCCGTCCCTCACGAGTCGAGTTACCATATC |
| | | | | AGTAGACACGTCTAAGAACCAGTTCTCCCTGAAGCTGAGCTCT |
| | | | | GTGACTGCCGCGGACACGGCCGTGTATTACTGTGCGAGAGATG |
| | | | | GAAGCAGTGGCTGGTACTTCCAGCACTGGGGCCAGGGCACCCT |
| | | | | GGTCACCGTCTCCTCA |
| 360 | 1F10 | artificial | aa | QVQLQESGPGLVKPSQTLSLTCTVSGGSISSGGYYWSWIRQHP |
| | | | | GKGLEWIGYIYYSGSTYYNPSLTSRVTISVDTSKNQFSLKLSS |
| | | | | VTAADTAVYYCARDGSSGWYFQHWGQGTLVTVSS |
| 361 | 4A9 | artificial | nt | CAGGTGCAGCTGCAGGAGTCGGGCCCAGGACTGGTGAAGCCTT |
| | | | | CGGAGACCCTGTCCCTCACCTGCACTGTCTCTGGTGGCTCCAT |
| | | | | CAGTGGTTACTACTGGAGCTGGATCCGGCAGCCCCCAGGAAAG |
| | | | | GGACTGGAGTGGTTTGCATATTTCTCTTACAGTGGGAGCACCA |
| | | | | ACTACAACCCCTCCCTCAAGAGTCGAGTCACCTTATCAGTAGA |
| | | | | CACGTCCAAGAACCAGTTCTCCCTGAAGCTGAGCTCTGTGACC |
| | | | | GCTGCGGACACGGCCGTGTATTACTGTGCGAGGAACTGGGCCT |
| | | | | TCCACTTTGACTTCTGGGGCCAGGGAACCCTGGTCACCGTCTC |
| | | | | CCA |
| 362 | 4A9 | artificial | aa | QVQLQESGPGLVKPSETLSLTCTVSGGSISGYYWSWIRQPPGK |
| | | | | GLEWFAYFSYSGSTNYNPSLKSRVTLSVDTSKNQFSLKLSSVT |
| | | | | AADTAVYYCARNWAFHFDFWGQGTLVTVSS |
| 363 | 4F7 | artificial | nt | CAGGTGCAGCTGCAGGAGTCGGGCCCAGGACTGGTGAAGCCTT |
| | | | | CGGAGACCCTGTCCCTCACCTGCACTGTCTCTGGTGGCTCCAT |
| | | | | CAGTAGTTACTCCTGGAGCTGGATCCGGCAGCCCCCAGGGAAG |
| | | | | GGACTGGAGTGGATTGGGTATATCTATTACAGTGGGAGCACCA |
| | | | | ACTACAACCCCTCCCTCAAGAGTCGAGTCACCATATCATTAGA |
| | | | | CACGTCCAAGAACCAGTTCTCCCTGAAGCTGAGCTCTGTGACC |
| | | | | GCTGCGGACACGGCCGTGTATTACTGTGCGAGGAACTGGGCCT |
| | | | | TCCACTTTGACTACTGGGGCCAGGGAACCCTGGTCACCGTCTC |
| | | | | CTCA |
| 364 | 4F7 | artificial | aa | QVQLQESGPGLVKPSETLSLTCTVSGGSISSYSWSWIRQPPGK |
| | | | | GLEWIGYIYYSGSTNYNPSLKSRVTISLDTSKNQFSLKLSSVT |
| | | | | AADTAVYYCARNWAFHFDYWGQGTLVTVSS |
| 365 | 22D1 | artificial | nt | CAGGTGCAGCTGGTGCAGTCTGGGGCTGAGGTGAAGAAGCCTG |
| | | | | GGGCCTCAGTGAGGGTTTCCTGCAAGGTTTCTGGATACACCTT |
| | | | | CACCAGCTACTTTATTCACTGGGTACGCCAGGCCCCTGGACAA |
| | | | | GGGCTTGAGTGGATGGGAATAATCAACCCTATTAGTGTTAGCA |
| | | | | CAAGCTACGCACAGAAGTTCCAGGGCAGAGTCACCATGACCAG |
| | | | | GGACACGTCCACGAGCACAGTCTTCATGGAGCTGAGCAGCCTG |
| | | | | AGATCTGAGGACACGGCCGTGTATTACTGTGCGCGAGGGGGGA |
| | | | | TACAGCTATGGTTACATTTGGACTACTGGGGCCAGGGAACCCT |
| | | | | GGTCACCGTCTCCTCA |
| 366 | 22D1 | artificial | aa | QVQLVQSGAEVKKPGASVRVSCKVSGYTFTSYFIHWVRQAPGQ |
| | | | | GLEWMGIINPISVSTSYAQKFQGRVTMTRDTSTSTVFMELSSL |
| | | | | RSEDTAVYYCARGGIQLWLHLDYWGQGTLVTVSS |
| 367 | 19B5 | artificial | nt | CAGGTGCAGTTGGTGCAGTCTGGGGCTGAGGTGAAGAAGCCTG |
| | | | | GGGCCTCAGTGAAGGTTTCCTGCAAGGTTTCTGGATACACCTT |
| | | | | CACCAGCTACTTTATTCACTGGGTGCGCCAGGCCCCTGGACAA |
| | | | | GGGCTTGAATGGATGGGAATTATCAACCCTATTAGTGTTAGCA |
| | | | | CAAGCTACGCACAGAAGTTCCAGGGCAGAGTCACCATGACCAG |
| | | | | GGACACGTCCACGAGCACAGTCTTCATGGAGCTGAGCAGcCTG |
| | | | | AGATCTGAGGACACGGCCGTGTATTACTGTGCGCGAGGGGGGA |
| | | | | TACAGCTATGGTTACATTTGGACTACTGGGGCCAGGGAACCCT |
| | | | | GGTCACCGTCTCCTCA |
| 368 | 19B5 | artificial | aa | QVQLVQSGAEVKKPGASVKVSCKVSGYTFTSYFIHWVRQAPGQ |
| | | | | GLEWMGIINPISVSTSYAQKFQGRVTMTRDTSTSTVFMELSSL |
| | | | | RSEDTAVYYCARGGIQLWLHLDYWGQGTLVTVSS |
| 369 | 25F8 | artificial | nt | CAGGTGCAGCTGGTGCAGTCTGGGGCTGAGGTGAAGAAGCCTG |
| | | | | GGGCCTCAGTGAAGGTTTCCTGCAAGGCATCTGGATACACCTT |
| | | | | CACCAGCTACTATATTCACTGGGTGCGCCAGGCCCCTGGACAA |
| | | | | GGACTTGAGTGGATGGGAATAATCAACCCCAGTGGTGGTAGCA |
| | | | | CAAGGTACGCACAGAAGTTCCAGGGCAGAGTCACCATGACCAG |
| | | | | GGACACGTCCACGAGCACAGTCTTCATGGAGCTGAGCagcctG |
| | | | | AGATCTGAGGACACGGCCGTGTATTACTGTGCGCGAGGGGGAA |
| | | | | TACAGCTATGGTTACATTttGACTACTGGGGCCAGGGAACCCT |
| | | | | GGTCACCGTCTCCTCA |
| 370 | 25F8 | artificial | aa | QVQLVQSGAEVKKPGASVKVSCKASGYTFTSYYIHWVRQAPGQ |
| | | | | GLEWMGIINPSGGSTRYAQKFQGRVTMTRDTSTSTVFMELSSL |
| | | | | RSEDTAVYYCARGGIQLWLHFDYWGQGTLVTVSS |
| 371 | 26D1 | artificial | nt | CAGGTGCAGTTGGTGCAGTCTGGGGCTGAGGTGAAGAAGCCTG |
| | | | | GGGCCTCAGTGAAGGTTTCCTGTAAGGCATCTAGATACACCTT |
| | | | | CACCAGCTACTATATGTCCTGGGTGCGACAGGCCCCTGGACAA |
| | | | | GGGCTTGAGTGGATGGGAATAATCCACCCTAGTGGTGGTGACA |
| | | | | CAACCTACGCACAGAAGTTCCAGGGCAGAGTCACCATGACCGG |
| | | | | GGACACGTCCACGAGCACAGTCTACATGGAGCTGAGCAGCCTG |
| | | | | AGATCTGAGGACACGGCCGTGTATTACTGTGCGAGAGGGGGGA |
| | | | | TAAAACTATGGTTACATTTTGACTATTGGGGCCAGGGAACCCT |
| | | | | GGTCACCGTCTCCTCA |
| 372 | 26D1 | artificial | aa | QVQLVQSGAEVKKPGASVKVSCKASRYTFTSYYMSWVRQAPGQ |
| | | | | GLEWMGIIHPSGGDTTYAQKFQGRVTMTGDTSTSTVYMELSSL |
| | | | | RSEDTAVYYCARGGIKLWLHFDYWGQGTLVTVSS |
| 373 | 4D2 | artificial | nt | CAGGTGCAGCTGGTGGAGTCTGGGGGAGGCGTGGTCCAGCCTG |
| | | | | GGAGGTCCCTGAGACTCTCCTGTGCAGCCTCTGGATTCACCTT |
| | | | | CAGTAGTTATGACATGCACTGGGTCCGCCAGGCTCCAGGCAAG |
| | | | | GGGCTGGAGTGGGTGGCAGTTATATCATATGATGGAACTAATG |
| | | | | AATACTATGCAGACTCCGTGAAGGGCCGATTCACCATCTCCAG |
| | | | | AGACACTTCCAAGAACACGCTGTATTTGCAAATGAACAGCCTG |
| | | | | AGAGCTGAGGACACGGCTGTATATTACTGTGCGAGAGAACGAT |
| | | | | ATTTTGACTGGTCTTTTGACTACTGGGGCCAGGGAACCCTGGT |
| | | | | CAGTGTCTCCTCA |
| 374 | 4D2 | artificial | aa | QVQLVESGGGVVQPGRSLRLSCAASGFTFSSYDMHWVRQAPGK |
| | | | | GLEWVAVISYDGTNEYYADSVKGRFTISRDTSKNTLYLQMNSL |
| | | | | RAEDTAVYYCARERYFDWSFDYWGQGTLVSVSS |
| 375 | 4E10 | artificial | nt | CAGGTGCAGCTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGACATGCACTGGGTCCGCCAGGCTCCAGGCAAG |
| | | | | GGGCTGGAGTGGGTGGCAGTTATATGGTATGATGGAAGTAATA |
| | | | | AATACTATGCAGACTCCGTGAAGGGCCGATTCACCATCTCCAG |
| | | | | AGACAATTCCACGAACACGCTGCATCTGCAAATGAACAGCCCG |
| | | | | AGAGCCGAGGACACGGCTGTGTACTACTGTGCGAGAGAGTATA |
| | | | | GGTACAGCTGGTACTTTGACTACTGGGGCCAGGGAACCCTGGT |
| | | | | CACCGTCTCCTCA |
| 376 | 4E10 | artificial | aa | QVQLVESGGGVVQPGRSLRLSCAASGFTFSSYDMHWVRQAPGK |
| | | | | GLEWVAVIWYDGSNKYYADSVKGRFTISRDNSTNTLHLQMNSP |
| | | | | RAEDTAVYYCAREYRYSWYFDYWGQGTLVTVSS |
| 377 | 22G10 | artificial | nt | GAGGTGCAACTGTTGGAGTCTGGGGGAGGCTTGGTACAGCCTG |
| | | | | GGGGGTCCCTGAGACTCTCCTGTGCAGCCTCTGGATTCACCTT |
| | | | | TAGCAGTTATGCCATGAACTGGGTCCGCCAGGCTCCAGGGAAG |
| | | | | GGGCTGGAGTGGGTCTCAACTATTAGTGGTGGTGGTGCTAACA |
| | | | | CATACTACGCAGACTCCGTGAAGGGCCGGTTCACCATCTCCAG |
| | | | | TGACAATTCCAAGAGCACGCTGTATCTGCAAATGAACAGCCTG |
| | | | | AGAGCCGCGGACACGGCCGTATATCACTGTGCGAAAGGGGGAA |
| | | | | TGGGGGGATACTACTACGGTATGGACGTCTGGGGCCAAGGGAC |
| | | | | CACGGTCACCGTCTCCTCA |
| 378 | 22G10 | artificial | aa | EVQLLESGGGLVQPGGSLRLSCAASGFTFSSYAMNWVRQAPGK |
| | | | | GLEWVSTISGGGANTYYADSVKGRFTISSDNSKSTLYLQMNSL |
| | | | | RAADTAVYHCAKGGMGGYYYGMDVWGQGTTVTVSS |
| 379 | 2C12_LC#1 | artificial | nt | CAGGTGCAGCTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGTCAGTTATATGGTATGATGGAAGTAATA |
| | | | | AATACTATGCAGACTCCGTGAAGGGCCGATTCACCATCTCCAG |
| | | | | AGACAATTCCAAGAACACGCTGTATCTGCAAATGAATAGCCTG |
| | | | | AGAGCTGAGGACACGGCTGTGTATTACTGCGCGAGAAGGGCCG |
| | | | | GTATAATAGGAACTACAGGCTACTACTACGGTATGGACGTCTG |
| | | | | GGGCCAAGGGACCACGGTCACCGTCTCCTCA |
| 380 | 2C12_LC#1 | artificial | aa | QVQLVESGGGVVQPGRSLRLSCAASGFTFSSYGMHWVRQAPGK |
| | | | | GLEWVSVIWYDGSNKYYADSVKGRFTISRDNSKNTLYLQMNSL |
| | | | | RAEDTAVYYCARRAGIIGTTGYYYGMDVWGQGTTVTVSS |
| 381 | 2H12_LC#2 | artificial | nt | CAGGTGCAGCTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGGCAGTTATATGGTATGATGGAAGTAATA |
| | | | | AATACTATACAGACTCCGTGAAGGGCCGATTCACCATCTCCAG |
| | | | | AGACAATTCCAAGAACACGCTGTATCTGCAAATGAATAGCCTG |
| | | | | AGAGCTGAGGACACGGCTGTGTATTACTGTGCGAGAAGGGCCG |
| | | | | GTATAATAGGAACTACAGGCTACTACTACGGTATGGACGTCTG |
| | | | | GGGCCAAGGGACCACGGTCACCGTCTCCTCA |
| 382 | 2H12_LC#2 | artificial | aa | QVQLVESGGGVVQPGRSLRLSCAASGFTFSSYGMHWVRQAPGK |
| | | | | GLEWVAVIWYDGSNKYYTDSVKGRFTISRDNSKNTLYLQMNSL |
| | | | | RAEDTAVYYCARRAGIIGTTGYYYGMDVWGQGTTVTVSS |
| 383 | 2G6_LC#1 | artificial | nt | CAGGTGCAGTTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGGCATTTATATGGTATGATGGAAGTAATA |
| | | | | AATACTATGCAGACTCCGTGAAGGACCGATTCACCATCTCCAG |
| | | | | AGACAATTCCAAGAACACGCTGTATCTGCAAATGAAAAGCCTG |
| | | | | AGAGCTGAGGACACGGCTGTGTATTACTGTGCGAGAAGGGCCG |
| | | | | GTATAATAGGAACTATAGGCTACTACTACGGTATGGACGTCTG |
| | | | | GGGCCAAGGGACCACGGTCACCGTCTCCTCA |
| 384 | 2G6_LC#1 | artificial | aa | QVQLVESGGGVVQPGRSLRLSCAASGFTFSSYGMHWVRQAPGK |
| | | | | GLEWVAFIWYDGSNKYYADSVKDRFTISRDNSKNTLYLQMKSL |
| | | | | RAEDTAVYYCARRAGIIGTIGYYYGMDVWGQGTTVTVSS |
| 385 | 2H12 | artificial | nt | CAGGTGCAGCTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGGCAGTTATATGGTATGATGGAAGTAATA |
| | | | | AATACTATACAGACTCCGTGAAGGGCCGATTCACCATCTCCAG |
| | | | | AGACAATTCCAAGAACACGCTGTATCTGCAAATGAATAGCCTG |
| | | | | AGAGCTGAGGACACGGCTGTGTATTACTGTGCGAGAAGGGCCG |
| | | | | GTATAATAGGAACTACAGGCTACTACTACGGTATGGACGTCTG |
| | | | | GGGCCAAGGGACCACGGTCACCGTCTCCTCA |
| 386 | 2H12 | artificial | aa | QVQLVESGGGVVQPGRSLRLSCAASGFTFSSYGMHWVRQAPGK |
| | | | | GLEWVAVIWYDGSNKYYTDSVKGRFTISRDNSKNTLYLQMNSL |
| | | | | RAEDTAVYYCARRAGIIGTTGYYYGMDVWGQGTTVTVSS |
| 387 | 2G6 | artificial | nt | CAGGTGCAGTTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTAGCTATGGCATGCACTGGGTCCGCCAGGCTCCAGGCAAG |
| | | | | GGGCTGGAGTGGGTGGCATTTATATGGTATGATGGAAGTAATA |
| | | | | AATACTATGCAGACTCCGTGAAGGACCGATTCACCATCTCCAG |
| | | | | AGACAATTCCAAGAACACGCTGTATCTGCAAATGAAAAGCCTG |
| | | | | AGAGCTGAGGACACGGCTGTGTATTACTGTGCGAGAAGGGCCG |
| | | | | GTATAATAGGAACTATAGGCTACTACTACGGTATGGACGTCTG |
| | | | | GGGCCAAGGGACCACGGTCACCGTCTCCTCA |
| 388 | 2G6 | artificial | aa | QVQLVESGGGVVQPGRSLRLSCAASGFTFSSYGMHWVRQAPGK |
| | | | | GLEWVAFIWYDGSNKYYADSVKDRFTISRDNSKNTLYLQMKSL |
| | | | | RAEDTAVYYCARRAGIIGTIGYYYGMDVWGQGTTVTVSS |
| 389 | 23A10 | artificial | nt | CAGGTGCAGCTGGTGGAGTCTGGGGGAGGCGTGGTCCAGCCTG |
| | | | | GGAGGTCCCTGAGACTCTCCTGTGCAGCGTCTGGATTCACCTT |
| | | | | CAGTCGCTATGGCATACACTGGGTCCGCCAGGCTCCAGGCAAG |
| | | | | GGGCTGGAGTGGGTGGCAGTTATATGGTATGATGGAAGTAATA |
| | | | | AATACTATGCAGACTCCGTGAAGGGCCGATTCACCATCTCCAG |
| | | | | AGACAATTCCAAGAACACGCTGTATCTGCTAATGAACAGCCTG |
| | | | | AGAGCCGAGGACTCGGCTGTGTATTACTGTGCGAGAAGGGCCG |
| | | | | GTATACCTGGAACTACGGGCTACTACTATGGTATGGACGTCTG |
| | | | | GGGCCAAGGGACCACGGTCACCGTCTCCTCA |
| 390 | 23A10 | artificial | aa | QVQLVESGGGVVQPGRSLRLSCAASGFTFSRYGIHWVRQAPGK |
| | | | | GLEWVAVIWYDGSNKYYADSVKGRFTISRDNSKNTLYLLMNSL |
| | | | | RAEDSAVYYCARRAGIPGTTGYYYGMDVWGQGTTVTVSS |
| 391 | 5E3 | artificial | nt | GAGGTGCAGTTGGTGGAGTCTGGGGGAGGCCTGGTCAAGCCTG |
| | | | | GGGGGTCCCTGAGACTCTCCTGTGCAGCCTCTGGATTCACCTT |
| | | | | CAGTAGCTATAGCATGCACTGGGTCCGCCAGGCTCCAGGGAAG |
| | | | | GGGCTGGAGTGGGTCTCATCCATTAGTAGTAGTAGTAGTTACA |
| | | | | TATACTACGCAGACTCAGTGAAGGGCCGATTCACCATCTCCAG |
| | | | | AGACAACGCCAAGAACTCACTGTATCTGCAAATGAACAGCCTG |
| | | | | AGAGCCGAGGACACGGCTGTGTATTACTGTGCGAGAGGGGAAA |
| | | | | CTGGAACTAACTACTACTACTACGGTATGGACGTCTGGGGCCA |
| | | | | AGGGACCACGGTCACCGTCTCCTCA |
| 392 | 5E3 | artificial | aa | EVQLVESGGGLVKPGGSLRLSCAASGFTFSSYSMHWVRQAPGK |
| | | | | GLEWVSSISSSSSYIYYADSVKGRFTISRDNAKNSLYLQMNSL |
| | | | | RAEDTAVYYCARGETGTNYYYYGMDVWGQGTTVTVSS |

TABLE IIB
Light Chain Variable Region Polynucleotide and Amino acid Sequences

| SEQ | | | | |
|---|---|---|---|---|
| ID | | | | |
| NO. | DESIGNATION | SOURCE | TYPE | SEQUENCE |
| 393 | 17H8 | artificial | nt | GACATTGTATTGACGCAGtctCCAGGCACCCTGTCTTTGTCTC |
| | 23B6 | | | CAGGGGAAAGAGCCACCCTCTCCTGCAGGGCCAGTCAGAGTGT |
| | 28D10 | | | TGCCGGCAGCTACCTAGCCTGGTACCAGCAGAAACCTGGCCAG |
| | | | | GCTCCCAGGCTCCTCATCTCTGGTGCATCCAGCAGGGCCACTG |
| | | | | GCATCCCAGACAGGTTCAGTGGCAGTGGGTCTGGGACAGACTT |
| | | | | CACTCTCACCATCAGCAGACTGGAGCCTGAAGATTTTGCAGTG |
| | | | | TATTACTGTCAGCAGTATGGTAAATCACCGATCACCTTCGGCC |
| | | | | AAGGGACACGACTGGAGATGAAAGGA |
| 394 | 17H8 | artificial | aa | DIVLTQSPGTLSLSPGERATLSCRASQSVAGSYLAWYQQKPGQ |
| | 23B6 | | | APRLLISGASSRATGIPDRFSGSGSGTDFTLTISRLEPEDFAV |
| | 28D10 | | | YYCQQYGKSPITFGQGTRLEMKG |
| 395 | 4A2 | artificial | nt | GAAATTGTGTTGACGCAGTCTCCAGGCACCCTGTCTTTGTCTC |
| | 5B4 | | | CAGGGGAAAGAGCCACCCTCTCTTGCAGGGCCAGTCGGAATAT |
| | 5C5 | | | TAGCAGCAGCTACTTAGCCTGGTACCAGCAGAAACCTGGCCAG |
| | | | | GCTCCCAGGCTCCTCATCTATGGTCCATCCAGCAGGGCCACTG |
| | | | | GCATCCCAGACAGGTTCAGTGGCAGTGGGTCTGGGACAGACTT |
| | | | | CACTCTCACCATCAGCAGACTGGAGCCTGAAGATTTTACAGTG |
| | | | | TATTACTGTCAGCAGTATGGTAGCTCATTCACTTTCGGCCCTG |
| | | | | GGACCAAAGTGGATATCAAACGA |
| 396 | 4A2 | artificial | aa | EIVLTQSPGTLSLSPGERATLSCRASRNISSSYLAWYQQKPGQ |
| | 5B4 | | | APRLLIYGPSSRATGIPDRFSGSGSGTDFTLTISRLEPEDFTV |
| | 5C5 | | | YYCQQYGSSFTFGPGTKVDIKR |
| 397 | 16H2 | artificial | nt | CAGTCTGCGCTGACTCAGCCACCCTCAGCGACTGGGACCCCCG |
| | 20D3 | | | GGCAGAGGGTCACCATCTCTTGTTCTGGAAGCAGCTCCAACAT |
| | 23E7 | | | CGGAAGTAATTTTGTAAACTGGTACAAACAACTCCCAGGAACG |
| | | | | GCCCCCAAAGTCCTCATCTATACTAATAATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGTCTGAT |
| | | | | TATTACTGTGCAACATGGGATGACAGCCTGAATGGTTGGGTGT |
| | | | | TCGGCGGAGGGACCAAGCTGACCGTCCTAGGT |
| 398 | 16H2 | artificial | aa | QSALTQPPSATGTPGQRVTISCSGSSSNIGSNFVNWYKQLPGT |
| | 20D3 | | | APKVLIYTNNQRPSGVPDRFSGSKSGTSASLAISGLQSEDESD |
| | 23E7 | | | YYCATWDDSLNGWVFGGGTKLTVLG |
| 399 | 26F12 | artificial | nt | CAGTCTGTGCTGACTCAGTCACCCTCAGCGTCTGGGACCCCCG |
| | 27B3 | | | GGCAGAAGGTCACCATCTCTTGTTCTGGAAGCCGCTCCAACAT |
| | | | | CGGAAGTAATTTTGTAAACTGGTACCAGCAGCTCCCAGGAACG |
| | | | | GCCCCCAAACTCCTCATCTATACTAATTATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGGCTGAT |
| | | | | TATTACTGTGCAGTATGGGATGACAGCCTGAATGGTTGGGTGT |
| | | | | TCGGCGGAGGGACCAAGCTGACCGTCCTAGGT |
| 400 | 26F12 | artificial | aa | QSVLTQSPSASGTPGQKVTISCSGSRSNIGSNFVNWYQQLPGT |
| | 27B3 | | | APKLLIYTNYQRPSGVPDRFSGSKSGTSASLAISGLQSEDEAD |
| | | | | YYCAVWDDSLNGWVFGGGTKLTVLG |
| 401 | 4B10 | artificial | nt | GAAATTGTATTGACGCAGTCTCCAGGCACCCTGTCTTTGTCTC |
| | 4C2 | | | CAGGGGAAAGAGCCACCCTCTCCTGCAGGGCCAGTCAGAGTGT |
| | | | | TAGCAACACCTACTTAGCCTGGTACCATCAGAGACCTGGCCAG |
| | | | | GCTCCCAGGCTCCTCATCTATGGTGCATCCAGCAGGGCCACTG |
| | | | | GCATCCCAGACAGATTCAGTGGCAGTGGGTCTGGGACAGACTT |
| | | | | CGCTCTCACCATCAGCAGTCTGGAGCCTGAAGATTTTGCAGTG |
| | | | | TATTACTGTCAGCAGTACAGTAACTCgtgGACGTTCGGCCAAG |
| | | | | GGACCAAGGTGGAAATCAaacGA |
| 402 | 4B10 | artificial | aa | EIVLTQSPGTLSLSPGERATLSCRASQSVSNTYLAWYHQRPGQ |
| | 4C2 | | | APRLLIYGASSRATGIPDRFSGSGSGTDFALTISSLEPEDFAV |
| | | | | YYCQQYSNSWTFGQGTKVEIKR |
| 403 | 4D3 | artificial | nt | GAAATTGTGTTGACGCAGTCTCCAGGCACCCTGTCTTTGTCTC |
| | 4F3 | | | CAGGGGAAAGAGCCACCCTCTCCTGCAGGGCCAGTCAGAGTGT |
| | | | | TAGCAGCAGCTACTTAGCCTGGTACCAGCAGAAACCTGGCCAG |
| | | | | GCTCCCAGGCTCCTCATCTATGGTGCATCCAGCAGGGCCACTG |
| | | | | GCATCCCAGACAGGTTCAGTGGCAGTGGGTCTGGGACAGACTT |
| | | | | CACTCTCACCATCAGCAGACTGGAACCTGAGGATTTTGCAGTG |
| | | | | TATTACTGTCAGCAGTATGGTAGCTCGTGGACGTTCGGCCAAG |
| | | | | GGACCAAGGTGGAAATCAAACGA |
| 404 | 4D3 | artificial | aa | EIVLTQSPGTLSLSPGERATLSCRASQSVSSSYLAWYQQKPGQ |
| | 4F3 | | | APRLLIYGASSRATGIPDRFSGSGSGTDFTLTISRLEPEDFAV |
| | | | | YYCQQYGSSWTFGQGTKVEIKR |
| 405 | 16E2 | artificial | nt | GACATCCAGATGACCCAGTCTCCATCCTCACTGTCTGCATCTG |
| | 17E10 | | | TAGGAGACAGAGTCACCATCACTTGTCGGGCGAGTCAGGGCAT |
| | 20B12 | | | TAGCAATTATTTAGCCTGGTTACAGCAGAAACCAGGGAAAGCC |
| | | | | CCTAAGTCCCTGATCTATGCTGCATCCAGTTTGCAAAGTGGGG |
| | | | | TCCCATCAAAGTTCAGCGGCAGTGGATCTGGGACAGATTTCAC |
| | | | | TCTCACCATCAGCAGCCTGCAGCCTGAAGATTTTGCAACTTAT |
| | | | | TACTGCCAACACTATTTTACTTACCCTCGGACGTTCGGCCAAG |
| | | | | GGACCAAGGTGGAAATCAAACGA |
| 406 | 16E2 | artificial | aa | DIQMTQSPSSLSASVGDRVTITCRASQGISNYLAWLQQKPGKA |
| | 17E10 | | | PKSLIYAASSLQSGVPSKFSGSGSGTDFTLTISSLQPEDFATY |
| | 20B12 | | | YCQHYFTYPRTFGQGTKVEIKR |
| 407 | 1D10 | artificial | nt | TCCTATGCGCTGACTCAGCCACCCTCAGTGTCCGTGTCCCCAG |
| | 2C12 | | | GACAGACAGCCAGCCTCACCTGCTCTGGAGATAGATTGGGGGA |
| | | | | AAAATATACTTGCTGGTATCAGCAGAGGCCAGGCCAGTCCCCT |
| | | | | TTGCTGGTCATCTATCAAGATACCAAGCGGCCCTCAGGGATCC |
| | | | | CTGAGCGATTCTCTGGCTCCACCTCTGGTAACACAGCCACTCT |
| | | | | GACCATCAGCGGGACCCAGGCTATGGATGAGGCTGACTATTAC |
| | | | | TGTCAGGCGTGGGACAGCAGCACTGTGGTATTCGGCGGAGGGA |
| | | | | CCAAGCTGACCGTCCTAGGT |
| 408 | 1D10 | artificial | aa | SYALTQPPSVSVSPGQTASLTCSGDRLGEKYTCWYQQRPGQSP |
| | 2C12 | | | LLVIYQDTKRPSGIPERESGSTSGNTATLTISGTQAMDEADYY |
| | | | | CQAWDSSTVVFGGGTKLTVLG |
| 409 | 16C1 | artificial | nt | GAAATTGTGTTGACGCAGTCTCCAGGCACCCTGTCTTTGTCTC |
| | | | | CAGGGGAAAGAGCCACCCTCTCCTGCAGGGCCAGCCAGAGTGT |
| | | | | TAGCAGCAGCTACTTAGCCTGGTACCAGCAGAAACCTGGCCAG |
| | | | | GCTCCCAGGCTCCTCATCTTTGGTGCATCCAGCAGGGCCACTG |
| | | | | GCATCCCAGACAGGTTCAGTGGCAGTGGGTCTGGGACAGACTT |
| | | | | CACTCTCACCATCAGCGGACTGGAGCCTGAAGATTTTGCAGTG |
| | | | | TATCACTGTCAGCAGTATGGTAACTCACCGCTCACTTTCGGCG |
| | | | | GAGGGACCAAGGTGGAGATCAAACGA |
| 410 | 16C1 | artificial | aa | EIVLTQSPGTLSLSPGERATLSCRASQSVSSSYLAWYQQKPGQ |
| | | | | APRLLIFGASSRATGIPDRFSGSGSGTDFTLTISGLEPEDFAV |
| | | | | YHCQQYGNSPLTFGGGTKVEIKR |
| 411 | 25G10 | artificial | nt | GAAATTGTGTTGACGCAGTCTCCAGGCACCCTGTCTTTGTCTC |
| | | | | CAGGGGAAAGAGCCACCCTCTCCTGCAGGGCCAGTCAGAGTGT |
| | | | | TAGCAGCAGCTACTTAGCCTGGTACCAGCAGAAACCTGGCCAG |
| | | | | GCTCCCAGGCTCCTCATCTTTGGTGCATCCAGCAGGGCCACTG |
| | | | | GCATCCCAGACAGGTTCAGTGGCAGTGGGTCTGGGACAGactT |
| | | | | CACTCTCACCATCAGCAGACTGGAGCCTGAAGATTTTGCAGTG |
| | | | | TATCACTGTCAGCAGTATGGTAACTCACCGCTCACTTTCGGCG |
| | | | | GAGGGACCAAGGTGGAGATCAAACGA |
| 412 | 25G10 | artificial | aa | EIVLTQSPGTLSLSPGERATLSCRASQSVSSSYLAWYQQKPGQ |
| | | | | APRLLIFGASSRATGIPDRFSGSGSGTDFTLTISRLEPEDFAV |
| | | | | YHCQQYGNSPLTFGGGTKVEIKR |
| 413 | 16A4 | artificial | nt | GAAATTGTGTTGACGCAGTCTCCAGGCACCCTGTCTTTGTCTC |
| | | | | CAGGGGAAAGAGCCACCCtCTCCTGCAGGGCCAGTCAGAGTGT |
| | | | | TAGCAGCAGTTATTTAGCCTGGTACCAGCAGAAACCTGGCCAG |
| | | | | GCTCCCAGGCTCCTCATCTATGGTACATCCAGCAGGGCCACTG |
| | | | | GCATCCCAGACAGGTTCAGTGGCAGTGGGTCTGGGACAGACTT |
| | | | | CACTCTCACCATCAGCAGACTGGAGCCTGAAGATTTTGCAGTG |
| | | | | TATTATTGTCAGCAGTACGGTAGCTCACCTTTCACTTTCGGCG |
| | | | | GAGGGACCAAGGTGGAGATCAAACGA |
| 414 | 16A4 | artificial | aa | EIVLTQSPGTLSLSPGERATLSCRASQSVSSSYLAWYQQKPGQ |
| | | | | APRLLIYGTSSRATGIPDRFSGSGSGTDFTLTISRLEPEDFAV |
| | | | | YYCQQYGSSPFTFGGGTKVEIKR |
| 415 | 1F10 | artificial | nt | GAAATTGTGTTGACGCAGTCTCCAGGCACCCTGTCTTTGTCTC |
| | | | | CAGGGGAAAGAGCCACCCTCTCCTGCAGGGCCAGTCGGAGTAT |
| | | | | TAGCAGCAGCTACTTAGCCTGGTACCAGCAGAAACCTGGCCAG |
| | | | | GCTCCCAGGCTCCTCATCTATGGTCCATCCAGCAGGGCCACTG |
| | | | | GCATCCCAGACAGGTTCAGTGGCAGTGGGTCTGGGACAGACTT |
| | | | | CACTCTCACCATCAGCAGACTGGAGCCTGAAGATTTTGCAGTG |
| | | | | TATTACTGTCAGCAGTATGGTAGCTCATTCACTTTCGGCCCTG |
| | | | | GGACCAAAGTGGATATCAAACGA |
| 416 | 1F10 | artificial | aa | EIVLTQSPGTLSLSPGERATLSCRASRSISSSYLAWYQQKPGQ |
| | | | | APRLLIYGPSSRATGIPDRFSGSGSGTDFTLTISRLEPEDFAV |
| | | | | YYCQQYGSSETFGPGTKVDIKR |
| 417 | 4A9 | artificial | nt | CAGTCTGTGCTGACGCAGCCGCCCTCAGTGTCTGGGGCCCCAG |
| | | | | GACAGAGGGTCACCATCTCCTGCACTGGGAGCAGCTCCAACAT |
| | | | | CGGGACAGGTTATGCTGTACACTGGTACCAGCAGTTTCCAGGA |
| | | | | ACAGCCCCCAAACTCCTCATCTATGGTAACAACAATCGGCCCT |
| | | | | CAGGGGTTCCTGACCGATTCTCTGGCTCCAAGTCTGGCACCTC |
| | | | | AGCCTCCCTGGCCATCACTGGGCTCCAGGCTGAGGATGAGGCT |
| | | | | GATTATTACTGCCAGTCCTATGACAGCAGACTGAGTGGTTGGG |
| | | | | TGTTCGGCGGAGGGACCAAGCTGACCGTCCTAGGT |
| 418 | 4A9 | artificial | aa | QSVLTQPPSVSGAPGQRVTISCTGSSSNIGTGYAVHWYQQFPG |
| | | | | TAPKLLIYGNNNRPSGVPDRFSGSKSGTSASLAITGLQAEDEA |
| | | | | DYYCQSYDSRLSGWVFGGGTKLTVLG |
| 419 | 4F7 | artificial | nt | CAGTCTGTgcTGACGCAGCCGCCCTCAGTGTCTGGGGCCCCAG |
| | | | | GGCAGAGGGTCACCATCTCCTGCACTGGGAGCAGCTCCAATAT |
| | | | | CGGGACAGGTTATGATGTACACTGGTATCAGCAGcttcCAGGA |
| | | | | ACAGCCCCCAAACTCCTCATCCATGGTAACAGCAATCGGCCCT |
| | | | | CAGGGGTCCCTGACCGATTCTCTGGCTCCAAGTCTGGCACCTC |
| | | | | AGCCTCCCTGGCCATCACTGGGCTCCAGGCTGAGGATGAGGCT |
| | | | | GATTATTACTGCCAGTCCTATGACAGCAGTCTGAGTGGTTGGG |
| | | | | TGTTCGGCGGAGGGACCAGGTTGACCGTCCTAGGT |
| 420 | 4F7 | artificial | aa | QSVLTQPPSVSGAPGQRVTISCTGSSSNIGTGYDVHWYQQLPG |
| | | | | TAPKLLIHGNSNRPSGVPDRFSGSKSGTSASLAITGLQAEDEA |
| | | | | DYYCQSYDSSLSGWVFGGGTRLTVLG |
| 421 | 22D1 | artificial | nt | CAGTCTGCGCTGACTCAGCCACCCTCAGCGACTGGGACCCCCG |
| | | | | GGCAGAGGGTCACCATCTCTTGTTCTGGAAGCAGCTCCAACAT |
| | | | | CGGAAGCAATTTTGTAAACTGGTACAAGCAGCTCCCAGGAACG |
| | | | | GCCCCCAAAGTCCTCATCTATACTAATAATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGTCTGAT |
| | | | | TATTACTGTGCAACATGGGATGACAGTATGAATGGTTGGGTGT |
| | | | | TCGGCGGAGGGACCAAGCTGACCGTCCTAGGT |
| 422 | 22D1 | artificial | aa | QSALTQPPSATGTPGQRVTISCSGSSSNIGSNFVNWYKQLPGT |
| | | | | APKVLIYTNNQRPSGVPDRFSGSKSGTSASLAISGLQSEDESD |
| | | | | YYCATWDDSMNGWVFGGGTKLTVLG |
| 423 | 19B5 | artificial | nt | CAGTCTGCGCTGACTCAGCCACCCTCAACGACTGGGACCCCCG |
| | | | | GGCAGAGGGTCACCATCTCTTGTTCTGGAAGCAGGTCCAACAT |
| | | | | CGGAAGCAATTTTGTAAACTGGTACAAGCAGCTCCCAGGAACG |
| | | | | GCCCCCAAAGTCCTCATCTATACTAATAATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGTCTGAT |
| | | | | TATTACTGCGCAACATGGGATGACAGTATGAATGGTTGGGTGT |
| | | | | TCGGCGGAGGGACCAAACTGACCGTCCTAGGT |
| 424 | 19B5 | artificial | aa | QSALTQPPSTTGTPGQRVTISCSGSRSNIGSNFVNWYKQLPGT |
| | | | | APKVLIYTNNQRPSGVPDRFSGSKSGTSASLAISGLQSEDESD |
| | | | | YYCATWDDSMNGWVFGGGTKLTVLG |
| 425 | 25F8 | artificial | nt | CAGTCTGCGCTGactCAGCCACCCTCAGCGACTGGGACCCCCG |
| | | | | GGCAGAGGGTCACCATCTCTTGTTCTGGAAGCAGCTCCAACAT |
| | | | | CGGAAGGAATTTTGTAAACTGGTATAAGCAGCTCCCAGGAACG |
| | | | | GCCCCCAAAGTCCTCATTTATACTAATAATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGTCTGAT |
| | | | | TATTACTGTGCAGCATGGGATGACAGCCTGAATGGTTGGGTGT |
| | | | | TCGGCGGAGGGACCAAGCTGACCGTCCTAGGT |
| 426 | 25F8 | artificial | aa | QSALTQPPSATGTPGQRVTISCSGSSSNIGRNFVNWYKQLPGT |
| | | | | APKVLIYTNNQRPSGVPDRFSGSKSGTSASLAISGLQSEDESD |
| | | | | YYCAAWDDSLNGWVFGGGTKLTVLG |
| 427 | 26D1 | artificial | nt | CACTCTGTGCTGACTCAGTCACCCTCAGCGTCTGGGACCCCCG |
| | | | | GACAGAGGGTCACCATCTCTTGTTCTGGAAGCCGCTCCAACAT |
| | | | | CGGAAGTAATTTTGTAAACTGGTACCAGCAGCTCCCAGGAACG |
| | | | | GCCCCCAAACTCCTCATCTATACTAATAATCAGCGGCCCTCAG |
| | | | | GGGTCCCTGACCGATTCTCTGGCTCCAAGTCTGGCACCTCAGC |
| | | | | CTCCCTGGCCATCAGTGGGCTCCAGTCTGAGGATGAGGCTGAT |
| | | | | TATTACTGTGCAGTATGGGATGACAGCCTGAATGGTTGGGTGT |
| | | | | TCGGCGGAGGGACCAAGCTGACCGTCCTAGGT |
| 428 | 26D1 | artificial | aa | HSVLTQSPSASGTPGQRVTISCSGSRSNIGSNFVNWYQQLPGT |
| | | | | APKLLIYTNNQRPSGVPDRFSGSKSGTSASLAISGLQSEDEAD |
| | | | | YYCAVWDDSLNGWVFGGGTKLTVLG |
| 429 | 4D2 | artificial | nt | GAAATTGTATTGACGCAGTCTCCAGGCACCCTGTCTTTGTCTC |
| | | | | CAGGGGAAAGAGCCACCCTCTCCTGCAGGGCCAGTCAGAGTGT |
| | | | | TAGCAACACCTACTTAGCCTGGTACCATCAGAGACCTGGCCAG |
| | | | | GCTCCCAGGCTCCTCATCTATGGTGCATCCAGCAGGGCCGCTG |
| | | | | GCATCCCAGACAGGTTCAGTGGCAGTGGGTCTGGGACAGACTT |
| | | | | CACTCTCACCATCAGCAGACTGGAGCCTGAAGATTTTGCAGTG |
| | | | | TATTACTGTCAGCAGTATAGTAACTCGTGGACGTTCGGCCAAG |
| | | | | GGACCAAGGTGGAAATCAAACGA |
| 430 | 4D2 | artificial | aa | EIVLTQSPGTLSLSPGERATLSCRASQSVSNTYLAWYHQRPGQ |
| | | | | APRLLIYGASSRAAGIPDRFSGSGSGTDFTLTISRLEPEDFAV |
| | | | | YYCQQYSNSWTFGQGTKVEIKR |
| 431 | 4E10 | artificial | nt | GAAATTGTGTTGACGCAGTCTCCAGGCACCCTGTCTTTGTCTC |
| | | | | CAGGGGAAAGAGCCACCCTCTCCTGCAGGGCCAGTCAGAGTGT |
| | | | | TGGCAGCAGCTACTTAGCCTGGTACCAGCAGAAACCTGGCCAG |
| | | | | GCTCCCAGGCTCCTCATCTATGGTGCATCCAGCAGGGTCACTG |
| | | | | GCATCCCAGACAGGTTCAGTGGCAGTGGGTCTGGGACAGATTT |
| | | | | CACTCTCACCATCAGCAGACTGGAGCCTGAAGATTTTGCAGTG |
| | | | | TATTACTGTCAGCAATATAGTAACTCGTGGACGTTCGGCCAAG |
| | | | | GGACCAAGGGGAAATCAAACGA |
| 432 | 4E10 | artificial | aa | EIVLTQSPGTLSLSPGERATLSCRASQSVGSSYLAWYQQKPGQ |
| | | | | APRLLIYGASSRVTGIPDRFSGSGSGTDFTLTISRLEPEDFAV |
| | | | | YYCQQYSNSWTFGQGTKVEIKR |
| 433 | 22G10 | artificial | nt | GAAATAGTGATGACGCAGTCTCCAGTCACCCTGTCTCTGTCTC |
| | | | | TAGGGGAAAGAGCCACCCTCTCCTGCAGGGCCAGTCAGAGTAT |
| | | | | TAGCAGCAACTTAGCCTGGTTCCAGCAGAAACCTGGCCAGGCT |
| | | | | CCCAGACTCCTCATCTATGGTGCATTTACCAGGGCCACTGGTA |
| | | | | TCCCAGCCAGGGTCAGTGGCAGTGGGTCTGGGACAGAGTTCAC |
| | | | | TCTCACCATCAGCAGCCTGCAGTCTGAAGATTTTGCAGTTTAT |
| | | | | TACTGTCAGCAGTATAATTACTGGCCGCTCACTTTCGGCGGAG |
| | | | | GGACCAAGGTGGAGATCAAGCGA |
| 434 | 22G10 | artificial | aa | EIVMTQSPVTLSLSLGERATLSCRASQSISSNLAWFQQKPGQA |
| | | | | PRLLIYGAFTRATGIPARVSGSGSGTEFTLTISSLQSEDFAVY |
| | | | | YCQQYNYWPLTFGGGTKVEIKR |
| 435 | 2C12_LC#1 | artificial | nt | GATGTTGTGATGactCAGtCTccActctccctgcCCGTCACCC |
| | | | | TTGGACAGCCGGcctCCAtctcctgCAGGtCTAGTCAAAGcct |
| | | | | cgtaTACAGTGATGGAAACAcctACTTGAATTGGTTTCAGCAG |
| | | | | AGGCCAGGCCAATCTCCAAGGcgcctaATTTATAAGGTTTCTA |
| | | | | ACTGGGactctGGGGtCCCAGACAGATTCAGCgGCAGTGGGTC |
| | | | | AGGCActGATTTCACactGAAAAtCAGCAGGGTGGaggctgaG |
| | | | | GATGTTGGGGTTTATTactgCATGCAAGGTATAGTGTGGCCGT |
| | | | | GCAGTTTTGGCCAGGGGACCAAGCTGGAGATCAAaCgA |
| 436 | 2C12_LC#1 | artificial | aa | DVVMTQSPLSLPVTLGQPASISCRSSQSLVYSDGNTYLNWFQQ |
| | | | | RPGQSPRRLIYKVSNWDSGVPDRFSGSGSGTDFTLKISRVEAE |
| | | | | DVGVYYCMQGIVWPCSFGQGTKLEIKR |
| 437 | 2H12_LC#2 | artificial | nt | GATGTTGTGATGACTCAGTCTCCACTCTCCCTGCCCGTCACCC |
| | | | | TTGGACAGCCGGCCTCCATCTCCTGCAGGTCTAGTCAAAGCCT |
| | | | | CGTATACAGTGATGGAAACACCTACTTGAATTGGTTTCAGCAG |
| | | | | AGGCCAGGCCAATCTCCAAGGCGCCTAATTTATAAGGTTTCTA |
| | | | | ACTGGGACTCTGGGGTCCCAGACAGAATCAGCGGCAGTGGGTC |
| | | | | AGGCACCGATTTCACACTGAAAATCAGCAGGGTGGAGGCTGAG |
| | | | | GATGTTGGGGTTTATTACTGCATGCAAGATACACTGTGGCCGT |
| | | | | GCAGTTTTGGCCAGGGGACCAAGCTGGAGATCAAACGA |
| 438 | 2H12_LC#2 | artificial | aa | DVVMTQSPLSLPVTLGQPASISCRSSQSLVYSDGNTYLNWFQQ |
| | | | | RPGQSPRRLIYKVSNWDSGVPDRISGSGSGTDFTLKISRVEAE |
| | | | | DVGVYYCMQDTLWPCSFGQGTKLEIKR |
| 439 | 2G6_LC#1 | artificial | nt | GaTGTTGTGATGACTCagtctccACTCTCCCTGCCCGTCACCC |
| | | | | ttggacaGCCGGCCTccaTCTCCTGCAGGTCTAGTCAAAGCCT |
| | | | | CGTATACAGTGATGGAAACACCTACTTGAATTGGTTTCAGCAG |
| | | | | AGGCCAGGCCAATCTCCACGGCGCCTAATTTATCAGGTTTCTA |
| | | | | ACTGGGACTCTGGGGTCCCAGACAGATTCAGCGGCAGTGGGTC |
| | | | | AGGCACTGATTTCACACTGAAAATCAGCAGGGTGGAGGCTGAG |
| | | | | GATGTTGGGATTTATTACTGCATGCAAGATACACTGTGGCCGT |
| | | | | GCAGTTTTGGCCAGGGGACCAAGCTGGAGATCAAACGA |
| 440 | 2G6_LC#1 | artificial | aa | DVVMTQSPLSLPVTLGQPASISCRSSQSLVYSDGNTYLNWFQQ |
| | | | | RPGQSPRRLIYQVSNWDSGVPDRFSGSGSGTDFTLKISRVEAE |
| | | | | DVGIYYCMQDTLWPCSFGQGTKLEIKR |
| 441 | 2H12 | artificial | nt | TCCTATGAGCTGACTCAGCCACCCTCAGTGTCCGTGTCCCCAG |
| | | | | GACAGACAGCCAGCATCACCTGCTCTGGAGATAGATTGGGGGA |
| | | | | AAAATATACTTGCTGGTATCAGCAGAGGCCAGGCCAGTCCCCT |
| | | | | TTGCTGGTCATCTATCAAGATACCAAGCGGCCCTCAGGGATCC |
| | | | | CTGAGCGATTCTCTGGCTCCAACTCTGGTAACACAGCCACTCT |
| | | | | GACCATCAGCGGGACCCAGCCTATGGATGAGGCTGACTATTAC |
| | | | | TGTCAGGCGTGGGACAGCAGCACTGTGGTATTCGGCGGAGGGA |
| | | | | CCAAGCTGACCGTCCtAGGT |
| 442 | 2H12 | artificial | aa | SYELTQPPSVSVSPGQTASITCSGDRLGEKYTCWYQQRPGQSP |
| | | | | LLVIYQDTKRPSGIPERFSGSNSGNTATLTISGTQPMDEADYY |
| | | | | CQAWDSSTVVFGGGTKLTVLG |
| 443 | 2G6 | artificial | nt | TCCTATGAACTGACTCAGCCACCCTCAGTGTCCGTGTCCCCAG |
| | | | | GACAGACAGCCAGCATCACCTGCTCTGGAGATAGGTTGGGGGA |
| | | | | AAAATATACTTGCTGGTATCAGCAGAGGCCAGGCCAGTCCCCT |
| | | | | TTGCTGGTCATCTATCAAGATACCAAGCGGCCCTCAGGGATCC |
| | | | | CTGAGCGATTCTCTGGCTCCAACTCTGGTAACACAGCCACTCT |
| | | | | GACCATCAGCGGGACCCAGGCTATGGATGAGGCTGACTATTAC |
| | | | | TGTCAGGCGTGGGACAGCAGCACTGTGGTATTCGGCGGAGGGA |
| | | | | CCAAGCTGACCGTCCTAGGT |
| 444 | 2G6 | artificial | aa | SYELTQPPSVSVSPGQTASITCSGDRLGEKYTCWYQQRPGQSP |
| | | | | LLVIYQDTKRPSGIPERFSGSNSGNTATLTISGTQAMDEADYY |
| | | | | CQAWDSSTVVFGGGTKLTVLG |
| 445 | 23A10 | artificial | nt | TCCTATGAGCTGACTCAGCCACCCTCAGTGTCCGTGTCCCCAG |
| | | | | GACAGACAGCCAGCATCACCTGCTCTGGAGATAGATTGGGGGA |
| | | | | GAAATATGTTTGCTGGTATCAGCAGAAGCCAGGCCAGTCCCCT |
| | | | | ATACTGGTCATCTATCAAGATAATAAGTGGCCCTCAGGGATCC |
| | | | | CTGAGCGATTCTCTGGCTCCAACTCTGGGAACACAGCCACTCT |
| | | | | GACCATCAGCGGGACCCAGGCTATGGATGAGGCTGACTATTAC |
| | | | | TGTCAGGCGTGGGACAGCAGCACTGTGGTATTCGGCGGGGGGA |
| | | | | CCAAGCTGACCGTCCTAGGT |
| 446 | 23A10 | artificial | aa | SYELTQPPSVSVSPGQTASITCSGDRLGEKYVCWYQQKPGQSP |
| | | | | ILVIYQDNKWPSGIPERFSGSNSGNTATLTISGTQAMDEADYY |
| | | | | CQAWDSSTVVFGGGTKLTVLG |
| 447 | 5E3 | artificial | nt | TCCTATGAGCTGACTCAGCCACCCTCAGTGTCCGTGTCCCCAG |
| | | | | GACAGACAGCCAGCATCACCTGCTCTGGAGATAAATTGGGGGA |
| | | | | TGAATATGCTTGCTGGTATCAGCAGAAGCCAGGCCAGTCCCCT |
| | | | | GTGCTGGTCATCTATCAAGATAGCAAGCGGCCCTCAGGGATCC |
| | | | | CTGAGCGATTCTCTGGCTCCAACTCTGGGAACACAGCCACTCT |
| | | | | GACCATCAGCGGGACCCAGGCTATGGATGAGGCTGACTATTAC |
| | | | | TGTCAGGCGTGGGACAGCAGCACTGTGGTATTCGGCGGAGGGA |
| | | | | CCAAGCTGACCGTCCTAGGT |
| 448 | 5E3 | artificial | aa | SYELTQPPSVSVSPGQTASITCSGDKLGDEYACWYQQKPGQSP |
| | | | | VIVIYQDSKRPSGIPERFSGSNSGNTATLTISGTQAMDEADYY |
| | | | | CQAWDSSTVVFGGGTKLTVLG |

TABLE IIc
Heavy Chain Variable Region Polynucleotide and Amino acid Sequences

13586 HC [hu anti-<huCDH19> 4F3 VH]
QVQLVESGGGVVQPGRSLRLSCAASGFSFSSYDMDWVRQTPGKGLEWVAVIWYDGSNKYYADSVRG
RFTISRDNSICNTLFLQMNSLRVEDTAVYYCARETGEGWYFDLWGRGTLVTVSS
SEQ ID NO: 449
13589 HC [hu anti-<huCDH19> 4A9 VH]
QVQLQESGPGLVKPSETLSLTCTVSGGSISGYYWSWIRQPPGKGLEWFAYFSYSGSTNYNPSLKSRVTLS
VDTSKNQFSLKLSSVTAADTAVYYCARNWAFHFDFWGQGTLVTVSS
SEQ ID NO: 450
13590 HC [hu anti-<huCDH19> 4B10 VH]
QVQLVESGGGVVQPGRSLRLSCAASGFTFSSYDMHWVRQAPGKGLEWVAVISYDGTNEYYADSVKGR
FTISRDTSKNTLYLQMNSLRAEDTAVYYCARERYFDWSFDYWGQGTLVSVSS
SEQ ID NO: 451
13874 HC [hu anti-<huCDH19> 17118.2 VH]
QVQLQESGPGLVKPSETLSLTCTVSGGSINSYYWSWIRQPPGKGLEWIGYIYYIGSTNYNPSLKSRVTISV
DTSKNQFSLKLSSVTAADTALYYCARDSRYRSGWYDAFDIWGQGTMVTVSS
SEQ ID NO: 452
13875 HC [hu anti-<huCDH19> 16C1.1 VH]
QVQLQESGPGLVKPSETLSLTCTVSGGSISGYYWSWIRQPPGKGLEWIGYIYYIGSTNYNPSLKSRVTMS
IDTSKNQFSLTLSSLTAADTAVYFCARDGSSGWYRWFDPWGQGTLVTVSS
SEQ ID NO: 453
13876 HC [hu anti-<huCDH19> 16A4.1 VH]
QVQLQESGPGLAKPSETLSLTCTVSGDSITSYYWSWIRQPPGKGLEWIGYIYYSGSTNYNPSLKSRVTISV
DTSKNQFSLKLSSVTAADTAVYYCARDQRRIAAAGTHFYGMDVWGQGTTVTVSS
SEQ ID NO: 454
13877 HC [hu anti-<huCDH19> 22G10.1 VH]
EVQLLESGGGLVQPGGSLRLSCAASGFTFSSYAMNWVRQAPGKGLEWVSTISGGGANTYYADSVKGR
FTISSDNSKSTLYLQMNSLRAADTAVYHCAKGGMGGYYYGMDVWGQGTTVTVSS
SEQ ID NO: 455
13878 HC [hu anti-<huCDH19> 20D3.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHFDYWGQGTLVTVSS
SEQ ID NO: 456
13879 HC [hu anti-<huCDH19> 22D1.1 VH]
QVQLVQSGAEVKKPGASVRVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 457
13880 HC [hu anti-<huCDH19> 25F8.1 VH]
QVQLVQSGAEVKKPGASVKVSCKASGYTFTSYYIHWVRQAPGQGLEWMGIINPSGGSTRYAQKFQGR
VTMTRDTSTSTVFMELSSLRSEDTAVYYCARGGIQLWLHFDYWGQGTLVTVSS
SEQ ID NO: 458
13881 HC [hu anti-<huCDH19> 26F12.1 VH]
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSS
SEQ ID NO: 459
13882 HC [hu anti-<huCDH19> 26D1.1 VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLWLHFDYWGQGTLVTVSS
SEQ ID NO: 460
13883 HC [hu anti-<huCDH19> 25G10.1 VH]
QVQLQESGPGLVKPSETLSLTCTVSGGSISGYYWSWIRQPPGKGLEWIGYIYYIGSTNYNPSLKSRVTMS
VDTSKNQFSLKLSSVTAADTAVYYCARDGSSGWYRWFDPWGQGTLVTVSS
SEQ ID NO: 461
13885 HC [hu anti-<huCDH19> 19B5.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 462
14022 HC [hu anti-<huCDH19> 4A2 VH]
QVQLQESGPGLVKPSQTLSLTCTVSGGSISSSGYYWSWIRQHPGKGLEWICIYIYYTGSAYYNPSLKSRV
TISVDTSKNQFSLKLSSVTAADTAVYYCARDGSSGWYFQYWGQGTLVTVSS
SEQ ID NO: 463
14024 HC [hu anti-<huCDH19> 4A2 (1-472)(Q7E, H47P) VH]
QVQLQESGPGLVKPSETLSLTCTVSGGSISSSGYYWSWIRQPPGKGLEWIGYIYYTGSAYYNPSLKSRVT
ISVDTSKNQFSLKLSSVTAADTAVYYCARDGSSGWYFQYWGQGTLVTVSS
SEQ ID NO: 464
14025 HC [hu anti-<huCDH19> 4A2 VH]
QVQLQESGPGLVKPSQTLSLTCTVSGGSISSSGYYWSWIRQHPGKGLEWIGYIYYTGSAYYNPSLKSRV
TISVDTSKNQFSLKLSSVTAADTAVYYCARDGSSGWYFQYWGQGTLVTVSS
SEQ ID NO: 465
14026 HC [hu anti-<huCDH19> 4A2 (1-472)(Q17E, H47P) VH]
QVQLQESGPGLVKPSETLSLTCTVSGGSISSSGYYWSWIRQPPGKGLEWIGYIYYTGSAYYNPSLKSRVT
ISVDTSKNQFSLKLSSVTAADTAVYYCARDGSSGWYFQYWGQGTLVTVSS
SEQ ID NO: 466
14027 HC [hu anti-<huCDH19> 4A2 (1-472)(Q17E, H47P, D111E) VH]
QVQLQESGPGLVKPSETLSLTCTVSGGSISSSGYYWSWIRQPPGKGLEWIGYIYYTGSAYYNPSLKSRVT
ISVDTSKNQFSLKLSSVTAADTAVYYCAREGSSGWYFQYWGQGTLVTVSS
SEQ ID NO: 467
14028 HC [hu anti-<huCDH19> 4A2 (1-472)(Q17E, H47P, D111E, W134Y) VH]
QVQLQESGPGLVKPSETLSLTCTVSGGSISSSGYYWSWIRQPPGKGLEWIGYIYYTGSAYYNPSLKSRVT
ISVDTSKNQFSLKLSSVTAADTAVYYCAREGSSGYYFQYWGQGTLVTVSS
SEQ ID NO: 468
14029 HC [hu anti-<huCDH19> 4A2 VH]
QVQLQESGPGLVKPSQTLSLTCTVSGGSISSSGYYWSWIRQHPGKGLEWIGYIYYTGSAYYNPSLKSRV
TISVDTSKNQFSLKLSSVTAADTAVYYCARDGSSGWYFQYWGQGTLVTVSS
SEQ ID NO: 469
14030 HC [hu anti-<huCDH19> 4F3 (1-471)(R17G) VH]
QVQLVESGGGVVQPGGSLRLSCAASGFSFSSYDMDWVRQTPGKGLEWVAVIWYDGSNKYYADSVRG
RFTISRDNSKNTLFLQMNSLRVEDTAVYYCARETGEGWYFDLWGRGTLVTVSS
SEQ ID NO: 470
14031 HC [hu anti-<huCDH19> 4F3 (1-471)(R17G, T47A) VH]
QVQLVESGGGVVQPGGSLRLSCAASGFSFSSYDMDWVRQAPGKGLEWVAVIWYDGSNKYYADSVRG
RFTISRDNSICNTLFLQMNSLRVEDTAVYYCARETGEGWYFDLWGRGTLVTVSS
SEQ ID NO: 471
14032 HC [hu anti-<huCDH19> 4F3 (1-471)(R17G, T47A, R141Q) VH]
QVQLVESGGGVVQPGGSLRLSCAASGFSFSSYDMDWVRQAPGKGLEWVAVIWYDGSNKYYADSVRG
RFTISRDNSKNTLFLQMNSLRVEDTAVYYCARETGEGWYFDLWGQGTLVTVSS
SEQ ID NO: 472
14033 HC [hu Anti-<huCDH19> 4F3 (1-471)(R17G, T47A, D61E, D72E, R141Q)
VH]
QVQLVESGGGVVQPGGSLRLSCAASGFSFSSYDMDWVRQAPGKGLEWVAVIWYEGSNKYYAESVRG
RFTISRDNSICNTLFLQMNSLRVEDTAVYYCARETGEGWYFDLWGQGTLVTVSS
SEQ ID NO: 473
14034 HC [hu anti-<huCDH19> 4F3 (1-471)(R17G, T47A, D61E, D72E, W134Y,
R141Q) VH]
QVQLVESGGGVVQPGGSLRLSCAASGFSFSSYDMDWVRQAPGKGLEWVAVIWYEGSNKYYAESVRG
RFTISRDNSKNTLFLQMNSLRVEDTAVYYCARETGEGYYFDLWGQGTLVTVSS
SEQ ID NO: 474
14039 HC [hu anti-<huCDH19> 2G6 (1-477)(R17G, D61E, D72E, K94N) VH]
QVQLVESGGGVVQPGGSLRLSCAASGFTFSSYGMHWVRQAPGKGLEWVAFIWYEGSNKYYAESVKD
RFTISRDNSKNTLYLQMNSLRAEDTAVYYCARRAGIIGTIGYYYGMDVWGQGTTVTVSS
SEQ ID NO: 475
14040 HC [hu anti-<huCDH19> 16C1.1 VH]
QVQLQESGPGLVKPSETLSLTCTVSGGSISGYYWSWIRQPPGKGLEWIGYIYYIGSTNYNPSLKSRVTMS
IDTSKNQFSLTLSSLTAADTAVYFCARDGSSGWYRWFDPWGQGTLVTVSS
SEQ ID NO: 476
14041 HC [hu anti-<huCDH19> 16C1.1 (1-469)(T92K) VH]
QVQLQESGPGLVKPSETLSLTCTVSGGSISCiYYWSWIRQPPGKGLEWIGYIYYIGSTNYNPSLKSRVTMS
IDTSKNQFSLKLSSLTAADTAVYFCARDGSSGWYRWFDPWGQGTLVTVSS
SEQ ID NO: 477
14042 HC [hu anti-<huCDH19> 16C1.1 (1-469)(T92K, D109E) VH]
QVQLQESGPGLVKPSETLSLTCTVSGGSISCiYYWSWIRQPPGKGLEWIGYIYYIGSTNYNPSLKSRVTMS
IDTSKNQFSLKLSSLTAADTAVYFCAREGSSGWYRWFDPWGQGTLVTVSS
SEQ ID NO: 478
14043 HC [hu anti-<huCDH19> 16C1.1 (1-469)(T92K, W132Y, W135Y) VH]
QVQLQESGPGLVKPSETLSLTCTVSGGSISCiYYWSWIRQPPGKGLEWIGYIYYIGSTNYNPSLKSRVTMS
IDTSKNQFSLKLSSLTAADTAVYFCARDGSSGYYRYFDPWGQGTLVTVSS
SEQ ID NO: 479
14044 HC [hu anti-<huCDH19> 16C1.1 (1-469)(T92K) VH]
QVQLQESGPGLVKPSETLSLTCTVSGGSISCiYYWSWIRQPPGKGLEWIGYIYYIGSTNYNPSLKSRVTMS
IDTSKNQFSLKLSSLTAADTAVYFCARDGSSGWYRWFDPWGQGTLVTVSS
SEQ ID NO: 480
14045 HC [hu anti-<huCDH19> 17118.2 VH]
QVQLQESGPGLVKPSETLSLTCTVSGGSINSYYWSWIRQPPGKGLEWIGYIYYIGSTNYNPSLKSRVTISV
DTSKNQFSLKLSSVTAADTALYYCARDSRYRSGWYDAFDIWGQGTMVTVSS
SEQ ID NO: 481
14046 HC [hu anti-<huCDH19> 17118.2 (1-471)(D109E) VH]
QVQLQESGPGLVKPSETLSLTCTVSGGSINSYYWSWIRQPPGKGLEWIGYIYYIGSTNYNPSLKSRVTISV
DTSKNQFSLKLSSVTAADTALYYCARESRYRSGWYDAFDIWGQGTMVTVSS
SEQ ID NO: 482
14047 HC [hu anti-<huCDH19> 17118.2 (1-471)(D109E, W132Y) VH]
QVQLQESGPGLVKPSETLSLTCTVSGGSINSYYWSWIRQPPGKGLEWIGYIYYIGSTNYNPSLKSRVTISV
DTSKNQFSLKLSSVTAADTALYYCARESRYRSGYYDAFDIWGQGTMVTVSS
SEQ ID NO: 483
14048 HC [hu anti-<huCDH19> 17118.2 (1-471)(D109E) VH]
QVQLQESGPGLVKPSETLSLTCTVSGGSINSYYWSWIRQPPGKGLEWIGYIYYIGSTNYNPSLKSRVTISV
DTSKNQFSLKLSSVTAADTALYYCARESRYRSGWYDAFDIWGQGTMVTVSS
SEQ ID NO: 484
14049 HC [hu anti-<huCDH19> 4F7 VH]
QVQLQESGPGLVKPSETLSLTCTVSGGSISSYSWSWIRQPPGKGLEWIGYIYYSGSTNYNPSLKSRVTISL
DTSKNQFSLKLSSVTAADTAVYYCARNWAFHFDYWGQGTLVTVSS
SEQ ID NO: 485
14050 HC [hu anti-<huCDH19> 4F7 VVH]
QVQLQESGPGLVKPSETLSLTCTVSGGSISSYSWSWIRQPPGKGLEWIGYIYYSGSTNYNPSLKSRVTISL
DTSKNQFSLKLSSVTAADTAVYYCARNWAFHFDYWGQGTLVTVSS
SEQ ID NO: 486
14051 HC [hu anti-<huCDH19> 4F7 (1-468)(W113Y) VH]
QVQLQESGPGLVKPSETLSLTCTVSGGSISSYSWSWIRQPPGKGLEWIGYIYYSGSTNYNPSLKSRVTISL
DTSKNQFSLKLSSVTAADTAVYYCARNYAFHFDYWGQGTLVTVSS
SEQ ID NO: 487
14052 HC [hu anti-<huCDH19> 4B10 (1-471)(R17G, D61E, D72E, W134Y) VH]
QVQLVESGGGVVQPGGSLRLSCAASGFTFSSYDMHWVRQAPGKGLEWVAVISYEGTNEYYAESVKGR
FTISRDTSKNTLYLQMNSLRAEDTAVYYCARERYFDYSFDYWCiQGTLVSVSS
SEQ ID NO: 488
14053 HC [hu anti-<huCDH19> 4B10 VH]
QVQLVESGGGVVQPGRSLRLSCAASGFTFSSYDMHWVRQAPGKGLEWVAVISYDGTNEYYADSVKGR
FTISRDTSKNTLYLQMNSLRAEDTAVYYCARERYFDWSFDYWGQGTLVSVSS
SEQ ID NO: 489
14054 HC [hu anti-<huCDH19> 4B10 (1-471)(R17G) VH]
QVQLVESGGGVVQPGGSLRLSCAASGFTFSSYDMHWVRQAPGKGLEWVAVISYDGTNEYYADSVKG
RFTISRDTSKNTLYLQMNSLRAEDTAVYYCARERYFDWSFDYWGQGTLVSVSS
SEQ ID NO: 490
14055 HC [hu anti-<huCDH19> 4B10 (1-471)(R17G, D61E, D72E) VH]
QVQLVESGGGVVQPGGSLRLSCAASGFTFSSYDMHWVRQAPGKGLEWVAVISYEGTNEYYAESVKGR
FTISRDTSKNTLYLQMNSLRAEDTAVYYCARERYFDWSFDYWGQGTLVSVSS
SEQ ID NO: 491
14056 HC [hu anti-<huCDH19> 4A9 VH]
QVQLQESGPGLVKPSETLSLTCTVSGGSISGYYWSWIRQPPGKGLEWFAYFSYSGSTNYNPSLKSRVTLS
VDTSKNQFSLKLSSVTAADTAVYYCARNWAFHFDFWGQGTLVTVSS
SEQ ID NO: 492
14057 HC [hu anti-<huCDH19> 4A9 (1-468)(F55I, A56G) VH]
QVQLQESGPGLVKPSETLSLTCTVSGGSISGYYWSWIRQPPGKGLEWIGYFSYSGSTNYNPSLKSRVTLS
VDTSKNQFSLKLSSVTAADTAVYYCARNWAFHFDFWGQGTLVTVSS
SEQ ID NO: 493
14058 HC [hu anti-<huCDH19> 4A9 (1-468)(F55I, A56G) VH]
QVQLQESGPGLVKPSETLSLTCTVSGGSISGYYWSWIRQPPGKGLEWIGYFSYSGSTNYNPSLKSRVTLS
VDTSKNQFSLKLSSVTAADTAVYYCARNWAFHFDFWGQGTLVTVSS
SEQ ID NO: 494
14059 HC [hu anti-<huCDH19> 4A9 (1-468)(F55I, A56G, W113Y) VH]
QVQLQESGPGLVKPSETLSLTCTVSGGSISGYYWSWIRQPPGKGLEWIGYFSYSGSTNYNPSLKSRVTLS
VDTSKNQFSLKLSSVTAADTAVYYCARNYAFHFDFWGQGTLVTVSS
SEQ ID NO: 495
14060 HC [hu anti-<huCDH19> 20D3.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHFDYWGQGTLVTVSS
SEQ ID NO: 496
14061 HC [hu anti-<huCDH19> 20D3.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHFDYWGQGTLVTVSS
SEQ ID NO: 497
14062 HC [hu anti-<huCDH19> 20D3.1 (1-469)(W133Y) VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLYLHFDYWGQGTLVTVSS
SEQ ID NO: 498
14063 HC [hu anti-<huCDH19> 20D3.1 (1-469)(W133Y) VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLYLHFDYWGQGTLVTVSS
SEQ ID NO: 499
14064 HC [hu anti-<huCDH19> 20D3.1 (1-469)(W133Y) VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLYLHFDYWGQGTLVTVSS
SEQ ID NO: 500
14065 HC [hu anti-<huCDH19> 22G10.1 (1-470)(S82R, A99E) VH]
EVQLLESGGGLVQPGGSLRLSCAASGFTFSSYAMNWVRQAPGKGLEWVSTISGGGANTYYADSVKGR
FTISRDNSKSTLYLQMNSLRAEDTAVYHCAKGGMGGYYYGMDVWGQGTTVTVSS
SEQ ID NO: 501
14066 HC [hu anti-<huCDH19> 22G10.1 (1-470)(A99E, H105Y) VH]
EVQLLESGGGLVQPGGSLRLSCAASGFTFSSYAMNWVRQAPGKGLEWVSTISGGGANTYYADSVKGR
FTISSDNSKSTLYLQMNSLRAEDTAVYYCAKGGMGGYYYCiMDVWGQGTTVTVSS
SEQ ID NO: 502
14067 HC [hu anti-<huCDH19> 22G10.1 (1-470)(A99E) VH]
EVQLLESGGGLVQPGGSLRLSCAASGFTFSSYAMNWVRQAPGKGLEWVSTISGGGANTYYADSVKGR
FTISSDNSKSTLYLQMNSLRAEDTAVYHCAKGGMGGYYYGMDVWGQGTTVTVSS
SEQ ID NO: 503
14068 HC [hu anti-<huCDH19> 22G10.1 (1-470)(A99E) VH]
EVQLLESGGGLVQPGGSLRLSCAASGFTFSSYAMNWVRQAPGKGLEWVSTISGGGANTYYADSVKGR
FTISSDNSKSTLYLQMNSLRAEDTAVYHCAKGGMGGYYYGMDVWGQGTTVTVSS
SEQ ID NO: 504
14069 HC [hu anti-<huCDH19> 22G10.1 (1-470)(D72E, A99E) VH]
EVQLLESGGGLVQPGGSLRLSCAASGFTFSYAMNWVRQAPGKGLEWVSTISGGGANTYYAESVKGRF
TISSDNSKSTLYLQMNSLRAEDTAVYHCAKGGMGGYYYGMDVWGQGTTVTVSS
SEQ ID NO: 505
14070 HC [hu anti-<huCDH19> 22G10.1 (1-470)(H105Y) VH]
EVQLLESGGGLVQPGGSLRLSCAASGFTFSSYAMNWVRQAPGKGLEWVSTISGGGANTYYADSVKGR
FTISSDNSKSTLYLQMNSLRAADTAVYYCAKGGMGGYYYGMDVWGQGTTVTVSS
SEQ ID NO: 506
14071 HC [hu anti-<huCDH19> 16A4.1 (1-474)(T144L) VH]
QVQLQESGPGLAKPSETLSLTCTVSGDSITSYYWSWIRQPPGKGLEWIGYIYYSGSTNYNPSLKSRVTISV
DTSKNQFSLKLSSVTAADTAVYYCARDQRRIAAAGTHFYGMDVWGQGTLVTVSS
SEQ ID NO: 507
14072 HC [hu anti-<huCDH19> 19B5.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 508
14073 HC [hu anti-<huCDH19> 19B5.1 (1-469)(W133Y) VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLYLHLDYWGQGTLVTVSS
SEQ ID NO: 509
14074 HC [hu anti-<huCDH19> 19B5.1 VH]
QVQLVQSGAEVKKPGASVKVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 510
14075 HC [hu anti-<huCDH19> 19B5.1 VH]
QVQLVQSGAEVKKPGASVKVSCKVSGYTFTSYFIHWVRQAPGQGLEWMGIINPISVSTSYAQKFQGRV
TMTRDTSTSTVFMELS SLRSEDTAVYYCARGGIQLWLHLDYWGQGTLVTVSS
SEQ ID NO: 511
14076 HC [hu anti-<huCDH19> 19B5.1 (1-469)(W133Y) VH]
QVQLVQSGAEVKKPGASVKVSCKVSGYTFTSYFIHWVRQAPGQGLEWMGIINPISVSTSYAQKFQGRV
TMTRDTSTSTVFMELS SLRSEDTAVYYCARGGIQLYLHLDYWGQGTLVTVSS
SEQ ID NO: 512
14077 HC [hu anti-<huCDH19> 23A10.3 (1-474)(L92Q) VH]
QVQLVESGGGVVQPGRSLRLSCAASGFTFSRYGIHWVRQAPGKGLEWVAVIWYDGSNKYYADSVKGR
FTISRDNSKNTLYLQMNSLRAEDSAVYYCARRAGIPGTTGYYYGMDVWGQGTTVTVSS
SEQ ID NO: 513
14078 HC [hu anti-<huCDH19> 23A10.3 (1-474)(R17G, L92Q) VH]
QVQLVESGGGVVQPGGSLRLSCAASGFTFSRYGIHWVRQAPGKGLEWVAVIWYDGSNKYYADSVKG
RFTISRDNSKNTLYLQMNSLRAEDSAVYYCARRAGIPGTTGYYYGMDVWGQGTTVTVSS
SEQ ID NO: 514
14079 HC [hu anti-<huCDH19> 23A10.3 (1-474)(R17G, D61E, D72E,
L92Q) VH]
QVQLVESGGGVVQPGGSLRLSCAASGFTFSRYGIHWVRQAPGKGLEWVAVIWYEGSNKYYAESVKGR
FTISRDNSKNTLYLQMNSLRAEDSAVYYCARRAGIPGTTGYYYGMDVWGQGTTVTVSS
SEQ ID NO: 515
14080 HC [hu anti-<huCDH19> 23A10.3 VH]
QVQLVESGGGVVQPGRSLRLSCAASGFTFSRYGIHWVRQAPGKGLEWVAVIWYDGSNKYYADSVKGR
FTISRDNSKNTLYLLMNSLRAEDSAVYYCARRAGIPGTTGYYYGMDVWGQGTTVTVSS
SEQ ID NO: 516
14081 HC [hu anti-<huCDH19> 25G10.1 VH]
QVQLQESGPGLVKPSETLSLTCTVSGGSISGYYWSWIRQPPGKGLEWIGYIYYIGSTNYNPSLKSRVTMS
VDTSKNQFSLKLSSVTAADTAVYYCARDGSSGWYRWFDPWGQGTLVTVSS
SEQ ID NO: 517
14082 HC [hu anti-<huCDH19> 25G10.1 (1-469)(D109E, W132Y, W135Y) VH]
QVQLQESGPGLVKPSETLSLTCTVSGGSISGYYWSWIRQPPGKGLEWIGYIYYIGSTNYNPSLKSRVTMS
VDTSKNQFSLKLSSVTAADTAVYYCAREGSSGYYRYFDPWGQGTLVTVSS
SEQ ID NO: 518
14083 HC [hu anti-<huCDH19> 26D1.1 VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLWLHFDYWGQGTLVTVSS
SEQ ID NO: 519
14084 HC [hu anti-<huCDH19> 26D1.1 VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLWLHFDYWGQGTLVTVSS
SEQ ID NO: 520
14085 HC [hu anti-<huCDH19> 26D1.1 VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLWLHFDYWGQGTLVTVSS
SEQ ID NO: 521
14086 HC [hu anti-<huCDH19> 26D1.1 VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLWLHFDYWGQGTLVTVSS
SEQ ID NO: 522
14087 HC [hu anti-<huCDH19> 26D1.1 (1-469)(W133Y) VH]
QVQLVQSGAEVKKPGASVKVSCKASRYTFTSYYMSWVRQAPGQGLEWMGIIHPSGGDTTYAQKFQGR
VTMTGDTSTSTVYMELSSLRSEDTAVYYCARGGIKLYLHFDYWGQGTLVTVSS
SEQ ID NO: 523
14088 HC [hu anti-<huCDH19> 26D1.1 (1-469)(R27G, G82R) VH]
QVQLVQSGAEVKKPGASVKVSCKASGYTFTSYYMSWVRQAPGQGLEWMGIIHPSGGDTTYAQKFQGR
VTMTRDTSTSTVYMELSSLRSEDTAVYYCARGGIKLWLHFDYWGQGTLVTVSS
SEQ ID NO: 524
14089 HC [hu anti-<huCDH19> 26F12.1 VH]
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSS
SEQ ID NO: 525
14090 HC [hu anti-<huCDH19> 26F12.1 VH]
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSS
SEQ ID NO: 526
14091 HC [hu anti-<huCDH19> 26F12.1 (1-469)(W133Y) VH]
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLYLHFDYWGQGTLVTVSS
SEQ ID NO: 527
14092 HC [hu anti-<huCDH19> 26F12.1 (1-469)(W133Y) VH]
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLYLHFDYWGQGTLVTVSS
SEQ ID NO: 528
14093 HC [hu anti-<huCDH19> 25F8.1 VH]
QVQLVQSGAEVKKPGASVKVSCKASGYTFTSYYIHWVRQAPGQGLEWMGIINPSGGSTRYAQKFQGR
VTMTRDTSTSTVFMELSSLRSEDTAVYYCARGGIQLWLHFDYWGQGTLVTVSS
SEQ ID NO: 529
14094 HC [hu anti-<huCDH19> 25F8.1 VH]
QVQLVQSGAEVKKPGASVKVSCKASGYTFTSYYIHWVRQAPGQGLEWMGIINPSGGSTRYAQKFQGR
VTMTRDTSTSTVFMELSSLRSEDTAVYYCARGGIQLWLHFDYWGQGTLVTVSS
SEQ ID NO: 530
14095 HC [hu anti-<huCDH19> 25F8.1 (1-469)(F90Y) VH]
QVQLVQSGAEVKKPGASVKVSCKASGYTFTSYYIHWVRQAPGQGLEWMGIINPSGGSTRYAQKFQGR
VTMTRDTSTSTVYMELSSLRSEDTAVYYCARGGIQLWLHFDYWGQGTLVTVSS
SEQ ID NO: 531
14096 HC [hu anti-<huCDH19> 25F8.1 (1-469)(F90Y) VH]
QVQLVQSGAEVKKPGASVKVSCKASGYTFTSYYIHWVRQAPGQGLEWMGIINPSGGSTRYAQKFQGR
VTMTRDTSTSTVYMELSSLRSEDTAVYYCARGGIQLWLHFDYWGQGTLVTVSS
SEQ ID NO: 532
14097 HC [hu anti-<huCDH19> 25F8.1 (1-469)(F90Y, W133Y) VH]
QVQLVQSGAEVKKPGASVKVSCKASGYTFTSYYIHWVRQAPGQGLEWMGIINPSGGSTRYAQKFQGR
VTMTRDTSTSTVYMELSSLRSEDTAVYYCARGGIQLYLHFDYWGQGTLVTVSS
SEQ ID NO: 533
14098 HC [hu anti-<huCDH19> 22D1.1 VH]
QVQLVQSGAEVKKPGASVRVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 534
14099 HC [hu anti-<huCDH19> 22D1.1 VH]
QVQLVQSGAEVKKPGASVRVSCKVSGYTFTSYFIHWVRQAPGQGLEWMGIINPISVSTSYAQKFQGRV
TMTRDTSTSTVFMELSSLRSEDTAVYYCARGGIQLWLHLDYWGQGTLVTVSS
SEQ ID NO: 535
14100 HC [hu anti-<huCDH19> 22D1.1 (1-469)(W133Y) VH]
QVQLVQSGAEVKKPGASVRVSCKVSGYTFTSYFIHWVRQAPGQGLEWMGIINPISVSTSYAQKFQGRV
TMTRDTSTSTVFMELSSLRSEDTAVYYCARGGIQLYLHLDYWGQGTLVTVSS
SEQ ID NO: 536
14101 HC [hu anti-<huCDH19> 22D1.1 (1-469)(W133Y) VH]
QVQLVQSGAEVKKPGASVRVSCKVSGYTFTSYFIHWVRQAPGQGLEWMGIINPISVSTSYAQKFQGRV
TMTRDTSTSTVFMELSSLRSEDTAVYYCARGGIQLYLHLDYWGQGTLVTVSS
SEQ ID NO: 537
14102 HC [hu anti-<huCDH19> 22D1.1 (1-469)(F90Y) VH]
QVQLVQSGAEVKKPGASVRVSCKVSGYTFTSYFIHWVRQAPGQGLEWMGIINPISVSTSYAQKFQGRV
TMTRDTSTSTVYMELSSLRSEDTAVYYCARGGIQLWLHLDYWGQGTLVTVSS
SEQ ID NO: 538
13591 HC [hu anti-<huCDH19> 4F7 VH]
QVQLQESGPGLVKPSETLSLTCTVSGGSISSYSWSWIRQPPGKOLEWIGYIYYSGSTNYNPSLKSRVTISL
DTSKNQFSLKLSSVTAADTAVYYCARNWAFHFDYWGQGTLVTVSS
SEQ ID NO: 539
14301 HC [hu anti-<huCDH19> 2G6 VH]
QVQLVESGGGVVQPGRSLRLSCAASGFTFSSYGMHWVRQAPGKGLEWVAFIWYDGSNKYYADSVKD
RFTISRDNSKNTLYLQMKSLRAEDTAVYYCARRAGIIGTIGYYYGMDVWGQGTTVTVSS
SEQ ID NO: 540
14302 HC [hu anti-<huCDH19> 2G6 (1-477)(R17G, K94N) VH]
QVQLVESGGGVVQPGGSLRLSCAASGFTFSSYGMHWVRQAPGKGLEWVAFIWYDGSNKYYADSVKD
RFTISRDNSKNTLYLQMNSLRAEDTAVYYCARRAGIIGTIGYYYGMDVWGQGTTVTVSS
SEQ ID NO: 541
14303 HC [hu anti-<huCDH19> 2G6 (1-477)(D61E, D72E) VH]
QVQLVESGGGVVQPGRSLRLSCAASGFTFSSYGMHWVRQAPGKGLEWVAFIWYEGSNKYYAESVKD
RFTISRDNSKNTLYLQMKSLRAEDTAVYYCARRAGIIGTIGYYYGMDVWGQGTTVTVSS
SEQ ID NO: 542
14304 HC [hu anti-<huCDH19> 2G6 (1-477)(R17G) VH]
QVQLVESGGGVVQPGGSLRLSCAASGFTFSSYGMHWVRQAPGKGLEWVAFIWYDGSNKYYADSVKD
RFTISRDNSKNTLYLQMKSLRAEDTAVYYCARRAGIIGTIGYYYGMDVWGQGTTVTVSS
SEQ ID NO: 543

TABLE IId
Light Chain Variable Region Amino acid Sequences

13586 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 544
13589 LC [hu anti-<huCDH19> 4A9 VL]
QSVLTQPPSVSGAPGQRVTISCTGSSSNIGTGYAVHWYQQFPGTAPKLLIYGNNNRPSGVPDRFSGSKSG
TSASLAITGLQAEDEADYYCQSYDSRLSGWVFGGGTKLTVLG
SEQ ID NO: 545
13590 LC [hu anti-<huCDH19> 4B10 VL]
EIVLTQSPGTLSLSPGERATLSCRASQSVSNTYLAWYHQRPGQAPRLLIYGASSRATGIPDRFSGSGSGTD
FALTISSLEPEDFAVYYCQQYSNSWTFGQGTKVEIKR
SEQ ID NO: 546
13874 LC [hu anti-<huCDH19> 17H8.2 VL]
DIVLTQSPGTLSLSPGERATLSCRASQSVAGSYLAWYQQKPGQAPRLLISGASSRATGIPDRFSGSGSGT
DFTLTISRLEPEDFAVYYCQQYGKSPITFGQGTRLEMKG
SEQ ID NO: 547
13875 LC [hu anti-<huCDH19> 16C1.1 VL]
EIVLTQSPGTLSLSPGERATLSCRASQSVSSSYLAWYQQKPGQAPRLLIFGASSRATGIPDRFSGSGSGTD
FTLTISGLEPEDFAVYHCQQYGNSPLTFGGGTKVEIKR
SEQ ID NO: 548
13876 LC [hu anti-<huCDH19> 16A4.1 VL]
EIVLTQSPGTLSLSPGERATLSCRASQSVSSSYLAWYQQKPGQAPRLLIYGTSSRATGIPDRFSGSGSGTD
FTLTISRLEPEDFAVYYCQQYGSSPFTFGGGTKVEIKR
SEQ ID NO: 549
13877 LC [hu anti-<huCDH19> 22G10.1 VL]
EIVMTQSPVTLSLSLGERATLSCRASQSISSNLAWFQQKPGQAPRLLIYGAFTRATGIPARVSGSGSGTEF
TLTISSLQSEDFAVYYCQQYNYWPLTFGGGTKVEIKR
SEQ ID NO: 552
13878 LC [hu anti-<huCDH19> 20D3.1 VL]
QSALTQPPSATGTPGQRVTISCSGSSSNIGSNFVNWYKQLPGTAPKVLIYTNNQRPSGVPDRFSGSKSGTS
ASLAISGLQSEDESDYYCATWDDSLNGWVFGGGTKLTVLG
SEQ ID NO: 554
13879 LC [hu anti-<huCDH19> 22D1.1 VL]
QSALTQPPSATGTPGQRVTISCSGSSSNIGSNFVNWYKQLPGTAPKVLIYTNNQRPSGVPDRFSGSKSGTS
ASLAISGLQSEDESDYYCATWDDSMNGWVFGGGTKLTVLG
SEQ ID NO: 555
13880 LC [hu anti-<huCDH19> 25F8.1 VL]
QSALTQPPSATGTPGQRVTISCSGSSSNIGRNFVNWYKQLPGTAPKVLIYTNNQRPSGVPDRFSGSKSGT
SASLAISGLQSEDESDYYCAAWDDSLNGWVFGGGTKLTVLG
SEQ ID NO: 556
13881 LC [hu anti-<huCDH19> 26F12.1 VL]
QSVLTQSPSASGTPGQKVTISCSGSRSNIGSNFVNWYQQLPGTAPKLLIYTNYQRPSGVPDRFSGSKSGTS
ASLAISGLQSEDEADYYCAVWDDSLNGWVFGGGTKLTVLG
SEQ ID NO: 557
13882 LC [hu anti-<huCDH19> 26D1.1 VL]
HSVLTQSPSASGTPGQRVTISCSGSRSNIGSNFVNWYQQLPGTAPKLLIYTNNQRPSGVPDRFSGSKSGTS
ASLAISGLQSEDEADYYCAVWDDSLNGWVFGGGTKLTVLG
SEQ ID NO: 555
13883 LC [hu anti-<huCDH19> 25G10.1 VL]
EIVLTQSPGTLSLSPGERATLSCRASQSVSSSYLAWYQQKPGQAPRLLIFGASSRATGIPDRFSGSGSGTD
FTLTISRLEPEDFAVYHCQQYGNSPLTFGGGTKVEIKR
SEQ ID NO: 556
13885 LC [hu anti-<huCDH19> 19B5.1 VL]
QSALTQPPSTTGTPGQRVTISCSGSRSNIGSNFVNWYKQLPGTAPKVLIYTNNQRPSGVPDRFSGSKSGTS
ASLAISGLQSEDESDYYCATWDDSMNGWVFGGGTKLTVLG
SEQ ID NO: 557
14022 LC [hu anti-<huCDH19> 4A2 (1-236)(N30Q) VL]
EIVLTQSPGTLSLSPGERATLSCRASRQISSSYLAWYQQKPGQAPRLLIYGPSSRATGIPDRFSGSGSGTDF
TLTISRLEPEDFTVYYCQQYGSSFTFGPGTKVDIKR
SEQ ID NO: 558
14024 LC [hu anti-<huCDH19> 4A2 (1-236)(N30Q, T102A, P141Q) VL]
EIVLTQSPGTLSLSPGERATLSCRASRQISSSYLAWYQQKPGQAPRLLIYGPSSRATGIPDRFSGSGSGTDF
TLTISRLEPEDFAVYYCQQYGSSFTFGQGTKVDIKR
SEQ ID NO: 559
14025 LC [hu anti-<huCDH19> 4A2 (1-236)(N30Q, T102A) VL]
EIVLTQSPGTLSLSPGERATLSCRASRQISSSYLAWYQQKPGQAPRLLIYGPSSRATGIPDRFSGSGSGTDF
TLTISRLEPEDFAVYYCQQYGSSFTFGPGTKVDIKR
SEQ ID NO: 560
14026 LC [hu anti-<huCDH19> 4A2 (1-236)(N30Q, T102A) VL]
EIVLTQSPGTLSLSPGERATLSCRASRQISSSYLAWYQQKPGQAPRLLIYGPSSRATGIPDRFSGSGSGTDF
TLTISRLEPEDFAVYYCQQYGSSFTFGPGTKVDIKR
SEQ ID NO: 561
14027 LC [hu anti-<huCDH19> 4A2 (1-236)(N30Q, T102A, P141Q) VL]
EIVLTQSPGTLSLSPGERATLSCRASRQISSSYLAWYQQKPGQAPRLLIYGPSSRATGIPDRFSGSGSGTDF
TLTISRLEPEDFAVYYCQQYGSSFTFGQGTKVDIKR
SEQ ID NO: 562
14028 LC [hu anti-<huCDH19> 4A2 (1-236)(N30Q, T102A, P141Q) VL]
EIVLTQSPGTLSLSPGERATLSCRASRQISSSYLAWYQQKPGQAPRLLIYGPSSRATGIPDRFSGSGSGTDF
TLTISRLEPEDFAVYYCQQYGSSFTFGQGTKVDIKR
SEQ ID NO: 563
14029 LC [hu anti-<huCDH19> 4A2 (1-236)(R29Q, N30S) VL]
EIVLTQSPGTLSLSPGERATLSCRASQSISSSYLAWYQQKPGQAPRLLIYGPSSRATGIPDRFSGSGSGTDF
TLTISRLEPEDFTVYYCQQYGSSFTFGPGTKVDIKR
SEQ ID NO: 564
14030 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 565
14031 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 566
14032 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 567
14033 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 568
14034 LC [hu anti-<huCDH19> 4F3 VL]
EIVLTQSPGTLSLSPGERATLSCRASQSVSSSYLAWYQQKPGQAPRLLIYGASSRATGIPDRFSGSGSGTD
FTLTISRLEPEDFAVYYCQQYGSSWTFGQGTKVEIKR
SEQ ID NO: 569
14039 LC [hu anti-<huCDH19> 2G6 (1-234)(C42S, D110E) VL]
SYELTQPPSVSVSPGQTASITCSGDRLGEKYTSWYQQRPGQSPLLVIYQDTKRPSGIPERFSGSNSGNTAT
LTISGTQAMDEADYYCQAWESSTVVFGGGTKLTVLG
SEQ ID NO: 570
14040 LC [hu anti-<huCDH19> 16C1.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISGLEPEDFAVYYCQQYGNSPLTFGGGTKVEIKR
SEQ ID NO: 571
14041 LC [hu anti-<huCDH19> 16C1.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISGLEPEDFAVYYCQQYGNSPLTFGGGTKVEIKR
SEQ ID NO: 572
14042 LC [hu anti-<huCDH19> 16C1.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISGLEPEDFAVYYCQQYGNSPLTFGGGTKVEIKR
SEQ ID NO: 573
14043 LC [hu anti-<huCDH19> 16C1.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISGLEPEDFAVYYCQQYGNSPLTFGGGTKVEIKR
SEQ ID NO: 574
14044 LC [hu anti-<huCDH19> 16C1.1 (1-235)(G95R, H105Y, G141Q) VL]
EIVLTQSPGTLSLSPGERATLSCRASQSVSSSYLAWYQQKPGQAPRLLIFGASSRATGIPDRFSGSGSGTD
FTLTISRLEPEDFAVYYCQQYGNSPLTFGQGTKVEIKR
SEQ ID NO: 575
14045 LC [hu anti-<huCDH19> 17H8.2 (1-235)(G149R) VL]
DIVLTQSPGTLSLSPGERATLSCRASQSVAGSYLAWYQQKPGQAPRLLISGASSRATGIPDRFSGSGSGT
DFTLTISRLEPEDFAVYYCQQYGKSPITFGQGTRLEMKR
SEQ ID NO: 576
14046 LC [hu anti-<huCDH19> 17H8.2 (1-235)(G149R) VL]
DIVLTQSPGTLSLSPGERATLSCRASQSVAGSYLAWYQQKPGQAPRLLISGASSRATGIPDRFSGSGSGT
DFTLTISRLEPEDFAVYYCQQYGKSPITFGQGTRLEMKR
SEQ ID NO: 577
14047 LC [hu anti-<huCDH19> 17H8.2 (1-235)(G149R) VL]
DIVLTQSPGTLSLSPGERATLSCRASQSVAGSYLAWYQQKPGQAPRLLISGASSRATGIPDRFSGSGSGT
DFTLTISRLEPEDFAVYYCQQYGKSPITFGQGTRLEMKR
SEQ ID NO: 578
14048 LC [hu anti-<huCDH19> 17H8.2 (1-235)(S57Y, G149R) VL]
DIVLTQSPOTLSLSPGERATLSCRASQSVAGSYLAWYQQKPGQAPRLLIYGASSRATGIPDRFSGSGSGT
DFTLTISRLEPEDFAVYYCQQYGKSPITFGQGTRLEMKR
SEQ ID NO: 579
14049 LC [hu anti-<huCDH19> 4F7 (1-239)(H57Y) VL]
QSVLTQPPSVSGAPGQRVTISCTGSSSNIGTGYDVHWYQQLPGTAPKLLIYGNSNRPSGVPDRFSGSKSG
TSASLAITGLQAEDEADYYCQSYDSSLSGWVFGGGTRLTVLG
SEQ ID NO: 580
14050 LC [hu anti-<huCDH19> 4F7 (1-239)(H57Y, D110E) VL]
QSVLTQPPSVSGAPGQRVTISCTGSSSNIGTGYDVHWYQQLPGTAPKLLIYGNSNRPSGVPDRFSGSKSG
TSASLAITGLQAEDEADYYCQSYESSLSGWVFGGGTRLTVLG
SEQ ID NO: 581
14051 LC [hu anti-<huCDH19> 4F7 (1-239)(D110E) VL]
QSVLTQPPSVSGAPGQRVTISCTGSSSNIGTGYDVHWYQQLPGTAPKLLIHGNSNRPSGVPDRFSGSKSG
TSASLAITGLQAEDEADYYCQSYESSLSGWVFGGGTRLTVLG
SEQ ID NO: 582
14052 LC [hu anti-<huCDH19> 4B10 (1-236)(H45Q, A90T) VL]
EIVLTQSPOTLSLSPGERATLSCRASQSVSNTYLAWYQQRPGQAPRLLIYGASSRATGIPDRFSGSGSGTD
FTLTISSLEPEDFAVYYCQQYSNSWTFGQGTKVEIKR
SEQ ID NO: 583
14053 LC [hu anti-<huCDH19> 4B10 (1-236)(H45Q, A90T) VL]
EIVLTQSPOTLSLSPGERATLSCRASQSVSNTYLAWYQQRPGQAPRLLIYGASSRATGIPDRFSGSGSGTD
FTLTISSLEPEDFAVYYCQQYSNSWTFGQGTKVEIKR
SEQ ID NO: 584
14054 LC [hu anti-<huCDH19> 4B10 (1-236)(H45Q, A90T) VL]
EIVLTQSPOTLSLSPGERATLSCRASQSVSNTYLAWYQQRPGQAPRLLIYGASSRATGIPDRFSGSGSGTD
FTLTISSLEPEDFAVYYCQQYSNSWTFGQGTKVEIKR
SEQ ID NO: 585
14055 LC [hu anti-<huCDH19> 4B10 (1-236)(H45Q, A90T) VL]
EIVLTQSPOTLSLSPGERATLSCRASQSVSNTYLAWYQQRPGQAPRLLIYGASSRATGIPDRFSGSGSGTD
FTLTISSLEPEDFAVYYCQQYSNSWTFGQGTKVEIKR
SEQ ID NO: 586
14056 LC [hu anti-<huCDH19> 4A9 (1-239)(F47L) VL]
QSVLTQPPSVSGAPGQRVTISCTGSSSNIGTGYAVHWYQQLPGTAPKLLIYGNNNRPSGVPDRFSGSKSG
TSASLAITGLQAEDEADYYCQSYDSRLSGWVFGGGTKLTVLG
SEQ ID NO: 587
14057 LC [hu anti-<huCDH19> 4A9 (1-239)(F47L) VL]
QSVLTQPPSVSGAPGQRVTISCTGSSSNIGTGYAVHWYQQLPGTAPKLLIYGNNNRPSGVPDRFSGSKSG
TSASLAITGLQAEDEADYYCQSYDSRLSGWVFGGGTKLTVLG
SEQ ID NO: 588
14058 LC [hu anti-<huCDH19> 4A9 (1-239)(F47L, D110E) VL]
QSVLTQPPSVSGAPGQRVTISCTGSSSNIGTGYAVHWYQQLPGTAPKLLIYGNNNRPSGVPDRFSGSKSG
TSASLAITGLQAEDEADYYCQSYESRLSGWVFGGGTKLTVLG
SEQ ID NO: 589
14059 LC [hu anti-<huCDH19> 4A9 (1-239)(F47L, D110E) VL]
QSVLTQPPSVSGAPGQRVTISCTGSSSNIGTGYAVHWYQQLPGTAPKLLIYGNNNRPSGVPDRFSGSKSG
TSASLAITGLQAEDEADYYCQSYESRLSGWVFGGGTKLTVLG
SEQ ID NO: 590
14060 LC [hu anti-<huCDH19> 20D3.1 (1-235)(S102A) VL]
QSALTQPPSATGTPGQRVTISCSGSSSNIGSNFVNWYKQLPGTAPKVLIYTNNQRPSGVPDRFSGSKSGTS
ASLAISGLQSEDEADYYCATWDDSLNGWVFGGGTKLTVLG
SEQ ID NO: 591
14061 LC [hu anti-<huCDH19> 20D3.1 (1-235)(K45Q, S102A) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDDSLNGWVFGGGTKLTVLG
SEQ ID NO: 592
14062 LC [hu anti-<huCDH19> 20D3.1 (1-235)(K45Q, S102A) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDDSLNGWVFGGGTKLTVLG
SEQ ID NO: 593
14063 LC [hu anti-<huCDH19> 20D3.1 (1-235)(K45Q, S102A, D111E, N135Q) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDESLQGWVFGGGTKLTVLG
SEQ ID NO: 594
14064 LC [hu anti-<huCDH19> 20D3.1 (1-235)(W109Y) VL]
QSALTQPPSATGTPGQRVTISCSGSSSNIGSNFVNWYKQLPGTAPKVLIYTNNQRPSGVPDRFSGSKSGTS
ASLAISGLQSEDESDYYCATYDDSLNGWVFGGGTKLTVLG
SEQ ID NO: 595
14065 LC [hu anti-<huCDH19> 22G10.1 VL]
EIVMTQSPVTLSLSLGERATLSCRASQSISSNLAWFQQKPGQAPRLLIYGAFTRATGIPARVSGSGSGTEF
TLTISSLQSEDFAVYYCQQYNYWPLTFGGGTKVEIKR
SEQ ID NO: 596
14066 LC [hu anti-<huCDH19> 22G10.1 VL]
EIVMTQSPVTLSLSLGERATLSCRASQSISSNLAWFQQKPGQAPRLLIYGAFTRATGIPARVSGSGSGTEF
TLTISSLQSEDFAVYYCQQYNYWPLTFGGGTKVEIKR
SEQ ID NO: 597
14067 LC [hu anti-<huCDH19> 22G10.1 (1-234)(Q97E, S98P) VL]
EIVMTQSPVTLSLSLGERATLSCRASQSISSNLAWFQQKPGQAPRLLIYGAFTRATGIPARVSGSGSGTEF
TLTISSLEPEDFAVYYCQQYNYWPLTFGGGTKVEIKR
SEQ ID NO: 598
14068 LC [hu anti-<huCDH19> 22G10.1 (1-234)(V78F, Q97E, S98P) VL]
EIVMTQSPVTLSLSLGERATLSCRASQSISSNLAWFQQKPGQAPRLLIYGAFTRATGIPARFSOSGSGTEF
TLTISSLEPEDFAVYYCQQYNYWPLTFGGGTKVEIKR
SEQ ID NO: 599
14069 LC [hu anti-<huCDH19> 22G10.1 (1-234)(V78F, Q97E, S98P) VL]
EIVMTQSPVTLSLSLGERATLSCRASQSISSNLAWFQQKPGQAPRLLIYGAFTRATGIPARFSGSGSGTEF
TLTISSLEPEDFAVYYCQQYNYWPLTFGGGTKVEIKR
SEQ ID NO: 600
14070 LC [hu anti-<huCDH19> 22G10.1 VL]
EIVMTQSPVTLSLSLGERATLSCRASQSISSNLAWFQQKPGQAPRLLIYGAFTRATGIPARVSGSGSGTEF
TLTISSLQSEDFAVYYCQQYNYWPLTFGGGTKVEIKR
SEQ ID NO: 601
14071 LC [hu anti-<huCDH19> 16A4.1 (1-235)(G141Q) VL]
EIVLTQSPGTLSLSPGERATLSCRASQSVSSSYLAWYQQKPGQAPRLLIYGTSSRATGIPDRFSGSGSGTD
FTLTISRLEPEDFAVYYCQQYGSSPFTFGQGTKVEIKR
SEQ ID NO: 602
14072 LC [hu anti-<huCDH19> 19B5.1 (1-235)(K45Q, S102A) VL]
QSALTQPPSTTGTPGQRVTISCSGSRSNIGSNFVNWYQQLPGTAPKVLIYTNNQRPSGVPDRFSGSKSGTS
ASLAISGLQSEDEADYYCATWDDSMNGWVFGGGTKLTVLG
SEQ ID NO: 603
14073 LC [hu anti-<huCDH19> 19B5.1 (1-235)(K45Q, S102A) VL]
QSALTQPPSTTGTPGQRVTISCSGSRSNIGSNFVNWYQQLPGTAPKVLIYTNNQRPSGVPDRFSGSKSGTS
ASLAISGLQSEDEADYYCATWDDSMNGWVFGGGTKLTVLG
SEQ ID NO: 604
14074 LC [hu anti-<huCDH19> 19B5.1 (1-235)(T11V, K45Q, S102A) VL]
QSALTQPPSVTGTPGQRVTISCSGSRSNIGSNFVNWYQQLPGTAPKVLIYTNNQRPSGVPDRFSGSKSGT
SASLAISGLQSEDEADYYCATWDDSMNGWVFGGGTKLTVLG
SEQ ID NO: 605
14075 LC [hu anti-<huCDH19> 19B5.1 (1-235)(T11V, K45Q, S102A, D111E,
N135Q) VL]
QSALTQPPSVTGTPGQRVTISCSGSRSNIGSNFVNWYQQLPGTAPKVLIYTNNQRPSGVPDRFSGSKSGT
SASLAISGLQSEDEADYYCATWDESMQGWVFGGGTKLTVLG
SEQ ID NO: 606
14076 LC [hu anti-<huCDH19> 19B5.1 (1-235)(T11V, K45Q, S102A, W109Y,
D111E, N135Q) VL]
QSALTQPPSVTGTPGQRVTISCSGSRSNIGSNFVNWYQQLPGTAPKVLIYTNNQRPSGVPDRFSGSKSGT
SASLAISGLQSEDEADYYCATYDESMQGWVFGGGTKLTVLG
SEQ ID NO: 607
14077 LC [hu anti-<huCDH19> 23A10.3 (1-231)(C42S) VL]
SYELTQPPSVSVSPGQTASITCSGDRLGEKYVSWYQQKPGQSPILVIYQDNKWPSGIPERFSGSNSGNTA
TLTISGTQAMDEADYYCQAWDSSTVVFGGGTKLTVLG
SEQ ID NO: 608
14078 LC [hu anti-<huCDH19> 23A10.3 (1-231)(C42S) VL]
SYELTQPPSVSVSPGQTASITCSGDRLGEKYVSWYQQKPGQSPILVIYQDNKWPSGIPERFSGSNSGNTA
TLTISGTQAMDEADYYCQAWDSSTVVFGGGTKLTVLG
SEQ ID NO: 609
14079 LC [hu anti-<huCDH19> 23A10.3 (1-231)(C42S, D110E) VL]
SYELTQPPSVSVSPGQTASITCSGDRLGEKYVSWYQQKPGQSPILVIYQDNKWPSGIPERFSGSNSGNTA
TLTISGTQAMDEADYYCQAWESSTVVFGGGTKLTVLG
SEQ ID NO: 610
14080 LC [hu anti-<huCDH19> 23A10.3 (1-231)(C42Y) VL]
SYELTQPPSVSVSPGQTASITCSGDRLGEKYVYWYQQKPGQSPILVIYQDNKWPSGIPERFSGSNSGNTA
TLTISGTQAMDEADYYCQAWDSSTVVFGGGTKLTVLG
SEQ ID NO: 611
14081 LC [hu anti-<huCDH19> 25G10.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISRLEPEDFAVYYCQQYGNSPLTFGGGTKVEIKR
SEQ ID NO: 612
14082 LC [hu anti-<huCDH19> 25G10.1 (1-235)(H105Y) VL]
EIVLTQSPGTLSLSPGERATLSCRASQSVSSSYLAWYQQKPGQAPRLLIFGASSRATGIPDRFSGSGSGTD
FTLTISRLEPEDFAVYYCQQYGNSPLTFGGGTKVEIKR
SEQ ID NO: 613
14083 LC [hu anti-<huCDH19> 26D1.1 (1-235)(S7P) VL]
HSVLTQPPSASGTPGQRVTISCSGSRSNIGSNFVNWYQQLPGTAPKLLIYTNNQRPSGVPDRFSGSKSGTS
ASLAISGLQSEDEADYYCAVWDDSLNGWVFGGGTKLTVLG
SEQ ID NO: 614
14084 LC [hu anti-<huCDH19> 26D1.1 (1-235)(H1Q, S7P) VL]
QSVLTQPPSASGTPGQRVTISCSGSRSNIGSNFVNWYQQLPGTAPKLLIYTNNQRPSGVPDRFSGSKSGTS
ASLAISGLQSEDEADYYCAVWDDSLNGWVFGGGTKLTVLG
SEQ ID NO: 615
14085 LC [hu anti-<huCDH19> 26D1.1 (1-235)(H1Q, S7P, W109Y) VL]
QSVLTQPPSASGTPGQRVTISCSGSRSNIGSNFVNWYQQLPGTAPKLLIYTNNQRPSGVPDRFSGSKSGTS
ASLAISGLQSEDEADYYCAVYDDSLNGWVFGGGTKLTVLG
SEQ ID NO: 616
14086 LC [hu anti-<huCDH19> 26D1.1 (1-235)(H1Q, S7P, W109Y, D111E, N135Q) VL]
QSVLTQPPSASGTPGQRVTISCSGSRSNIGSNFVNWYQQLPGTAPKLLIYTNNQRPSGVPDRFSGSKSGTS
ASLAISGLQSEDEADYYCAVYDESLQGWVFGGGTKLTVLG
SEQ ID NO: 617
14087 LC [hu anti-<huCDH19> 26D1.1 (1-235)(H1Q, S7P, W109Y, D111E, N135Q) VL]
QSVLTQPPSASGTPGQRVTISCSGSRSNIGSNFVNWYQQLPGTAPKLLIYTNNQRPSGVPDRFSGSKSGTS
ASLAISGLQSEDEADYYCAVYDESLQGWVFGGGTKLTVLG
SEQ ID NO: 618
14088 LC [hu anti-<huCDH19> 26D1.1 (1-235)(H1Q, S7P) VL]
QSVLTQPPSASGTPGQRVTISCSGSRSNIGSNFVNWYQQLPGTAPKLLIYTNNQRPSGVPDRFSGSKSGTS
ASLAISGLQSEDEADYYCAVWDDSLNGWVFGGGTKLTVLG
SEQ ID NO: 619
14089 LC [hu anti-<huCDH19> 26F12.1 (1-235)(S7P) VL]
QSVLTQPPSASGTPGQKVTISCSGSRSNIGSNFVNWYQQLPGTAPKLLIYTNYQRPSGVPDRFSGSKSGTS
ASLAISGLQSEDEADYYCAVWDDSLNGWVFGGGTKLTVLG
SEQ ID NO: 620
14090 LC [hu anti-<huCDH19> 26F12.1 (1-235)(S7P, D111E) VL]
QSVLTQPPSASGTPGQKVTISCSGSRSNIGSNFVNWYQQLPGTAPKLLIYTNYQRPSGVPDRFSGSKSGTS
ASLAISGLQSEDEADYYCAVWDESLNGWVFGGGTKLTVLG
SEQ ID NO: 621
14091 LC [hu anti-<huCDH19> 26F12.1 (1-235)(S7P, D111E) VL]
QSVLTQPPSASGTPGQKVTISCSGSRSNIGSNFVNWYQQLPGTAPKLLIYTNYQRPSGVPDRFSGSKSGTS
ASLAISGLQSEDEADYYCAVWDESLNGWVFGGGTKLTVLG
SEQ ID NO: 622
14092 LC [hu anti-<huCDH19> 26F12.1 (1-235)(S7P, W109Y, D111E, N135Q) VL]
QSVLTQPPSASGTPGQKVTISCSGSRSNIGSNFVNWYQQLPGTAPKLLIYTNYQRPSGVPDRFSGSKSGTS
ASLAISGLQSEDEADYYCAVYDESLQGWVFGGGTKLTVLG
SEQ ID NO: 623
14093 LC [hu anti-<huCDH19> 25F8.1 (1-235)(K45Q) VL]
QSALTQPPSATGTPGQRVTISCSGSSSNIGRNFVNWYQQLPGTAPKVLIYTNNQRPSGVPDRFSGSKSGT
SASLAISGLQSEDESDYYCAAWDDSLNGWVFGGGTKLTVLG
SEQ ID NO: 624
14094 LC [hu anti-<huCDH19> 25F8.1 (1-235)(K45Q, S102A) VL]
QSALTQPPSATGTPGQRVTISCSGSSSNIGRNFVNWYQQLPGTAPKVLIYTNNQRPSGVPDRFSGSKSGT
SASLAISGLQSEDEADYYCAAWDDSLNGWVFGGGTKLTVLG
SEQ ID NO: 625
14095 LC [hu anti-<huCDH19> 25F8.1 (1-235)(K45Q, S102A) VL]
QSALTQPPSATGTPGQRVTISCSGSSSNIGRNFVNWYQQLPGTAPKVLIYTNNQRPSGVPDRFSGSKSGT
SASLAISGLQSEDEADYYCAAWDDSLNGWVFGGGTKLTVLG
SEQ ID NO: 626
14096 LC [hu anti-<huCDH19> 25F8.1 (1-235)(K45Q, S102A, D111E) VL]
QSALTQPPSATGTPGQRVTISCSGSSSNIGRNFVNWYQQLPGTAPKVLIYTNNQRPSGVPDRFSGSKSGT
SASLAISGLQSEDEADYYCAAWDESLNGWVFGGGTKLTVLG
SEQ ID NO: 627
14097 LC [hu anti-<huCDH19> 25F8.1 (1-235)(K45Q, S102A, D111E, N135Q) VL]
QSALTQPPSATGTPGQRVTISCSGSSSNIGRNFVNWYQQLPGTAPKVLIYTNNQRPSGVPDRFSGSKSGT
SASLAISGLQSEDEADYYCAAWDESLQGWVFGGGTKLTVLG
SEQ ID NO: 628
14098 LC [hu anti-<huCDH19> 22D1.1 (1-235)(K45Q, S102A) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDDSMNGWVFGGGTKLTVLG
SEQ ID NO: 629
14099 LC [hu anti-<huCDH19> 22D1.1 (1-235)(K45Q, S102A, D111E, N135Q) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDESMQGWVFGGGTKLTVLG
SEQ ID NO: 630
14100 LC [hu anti-<huCDH19> 22D1.1 (1-235)(K45Q, S102A, W109Y, D111E,
N135Q) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATYDESMQGWVFGGGTKLTVLG
SEQ ID NO: 631
14101 LC [hu anti-<huCDH19> 22D1.1 (1-235)(K45Q, S102A, W109Y) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATYDDSMNGWVFGGGTKLTVLG
SEQ ID NO: 632
14102 LC [hu anti-<huCDH19> 22D1.1 (1-235)(K45Q, S102A) VL]
QSALTQPPSATGTPGQRVTISCSGSSSNIGSNFVNWYQQLPGTAPKVLIYTNNQRPSGVPDRFSGSKSGTS
ASLAISGLQSEDEADYYCATWDDSMNGWVFGGGTKLTVLG
SEQ ID NO: 633
13591 LC [hu anti-<huCDH19> 4F7 VL]
QSVLTQPPSVSGAPGQRVTISCTGSSSNIGTGYDVHWYQQLPGTAPKLLIHGNSNRPSGVPDRFSGSKSG
TSASLAITGLQAEDEADYYCQSYDSSLSGWVFGGGTRLTVLG
SEQ ID NO: 634
14301 LC [hu anti-<huCDH19> 2G6 (1-234)(D110E) VL]
SYELTQPPSVSVSPGQTASITCSGDRLGEKYTCWYQQRPGQSPLLVIYQDTKRPSGIPERFSGSNSGNTAT
LTISGTQAMDEADYYCQAWESSTVVFGGGTKLTVLG
SEQ ID NO: 635
14302 LC [hu anti-<huCDH19> 2G6 (1-234)(C42S, D110E) VL]
SYELTQPPSVSVSPGQTASITCSGDRLGEKYTSWYQQRPGQSPLLVIYQDTKRPSGIPERFSGSNSGNTAT
LTISGTQAMDEADYYCQAWESSTVVFGGGTKLTVLG
SEQ ID NO: 636
14303 LC [hu anti-<huCDH19> 2G6 (1-234)(C42S, D110E) VL]
SYELTQPPSVSVSPGQTASITCSGDRLGEKYTSWYQQRPGQSPLLVIYQDTKRPSGIPERFSGSNSGNTAT
LTISGTQAMDEADYYCQAWESSTVVFGGGTKLTVLG
SEQ ID NO: 637
14304 LC [hu anti-<huCDH19> 23A10.3 (1-231)(C42S) VL]
SYELTQPPSVSVSPGQTASITCSGDRLGEKYVSWYQQKPGQSPILVIYQDNKWPSGIPERFSGSNSGNTA
TLTISGTQAMDEADYYCQAWDSSTVVFGGGTKLTVLG
SEQ ID NO: 638

### Anti-CDH19 Variable and Constant Region Polynucleotide and Amino Acid Sequences

TABLE IIIa
Heavy Chain Variable and Contant
Region Polynucleotide and Amino acid Sequences

2G6
CAGGTGCAGTTGGTGGAGTCTGGGGGAGGCGTGGTCCAGCCTGGGAGGTCCCTGAGACTCTCCTGT
GCAGCGTCTGGATTCACCTTCAGTAGCTATGGCATGCACTGGGTCCGCCAGGCTCCAGGCAAGGGG
CTGGAGTGGGTGGCATTTATATGGTATGATGGAAGTAATAAATACTATGCAGACTCCGTGAAGGAC
CGATTCACCATCTCCAGAGACAATTCCAAGAACACGCTGTATCTGCAAATGAAAAGCCTGAGAGCT
GAGGACACGGCTGTGTATTACTGTGCGAGAAGGGCCGGTATAATAGGAACTATAGGCTACTACTAC
GGTATGGACGTCTGGGGCCAAGGGACCACGGTCACCGTCTCTAGTGCCTCCACCAAGGGCCCATCG
GTCTTCCCCCTGGCACCCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCTGACCAGCGGCGTGCAC
ACCTTCCCGGCTGTCCTACAGTCCTCAGGACTCTACTCCCTCAGCAGCGTGGTGACCGTGCCCTCC
AGCAGCTTGGGCACCCAGACCTACATCTGCAACGTGAATCACAAGCCCAGCAACACCAAGGTGGAC
AAGAAAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGCTC
ACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATGA
SEQ ID NO: 639
QVQLVESGGGVVQPGRSLRLSCAASGFTFSSYGMHWVRQAPGKGLEWVAFIWYDGSNKYYADSVKD
RFTISRDNSKNTLYLQMKSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 640
4A2
CAGGTGCAGCTGCAGGAGTCGGGCCCAGGACTGGTGAAGCCTTCACAGACCCTGTCCCTCACCTGC
ACTGTCTCTGGTGGCTCCATCAGCAGTAGTGGTTACTACTGGAGCTGGATCCGCCAGCACCCAGGG
AAGGGCCTGGAGTGGATTGGGTACATCTATTACACTGGGAGCGCCTACTACAACCCGTCCCTCAAG
AGTCGAGTTACCATATCAGTAGACACGTCTAAGAACCAGTTCTCCCTGAAGCTGAGCTCTGTGACT
CCCGCGGACACGGCCGTGTATTACTCTGCGAGAGATGGAAGCAGTGGCTGGTACTTCCAGTATTGG
GGCCAGGGCACCCTGGTCACCGTCTCTAGTGCCTCCACCAAGGGCCCATCGGTCTTCCCCCTGGCA
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATCCTCCGTGATCCATGAGGCTCTCCACAACCACTACACG
CAGAAGAGCCTCTCCCTGTCTCCGGGTAAATGA
SEQ ID NO: 641
QVQLQESGPGLVKPSQTLSLTCTVSGGSISSSGYYWSWIRQHPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 642
4A9
CAGGTGCAGCTGCAGGAGTCGGGCCCAGGACTGGTGAAGCCTTCGGAGACCCTGTCCCTCACCTGC
ACTGTCTCTGGTGGCTCCATCAGTGGTTACTACTGGAGCTGGATCCGGCAGCCCCCAGGAAAGGGA
CTGGAGTGGTTTGCATATTTCTCTTACAGTGGGAGCACCAACTACAACCCCTCCCTCAAGAGTCGA
GTCACCTTATCAGTAGACACGTCCAAGAACCAGTTCTCCCTGAAGCTGAGCTCTGTGACCGCTGCG
GACACGGCCGTGTATTACTGTGCGAGGAACTGGGCCTTCCACTTTGACTTCTGGGGCCAGGGAACC
CTGGTCACCGTCTCTAGTGCCTCCACCAAGGGCCCATCGGTCTTCCCCCTGGCACCCTCCTCCAAG
AGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAAAGTTGAGCCCAAATCTTGTGAC
AAAACTCACACATGCCCACCGTGCCCAGCACCTGAACTCCTGGGGGGACCGTCAGTCTTCCTCTTC
CCCCCAAAACCCAAGGACACCCTCATTATCTCCCGGACCCCTGAGGTCACATGCGTGGTGGTCGAC
GTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCA
TCCCGGGAGGAGATGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTC
TCCCTGTCTCCGGGTAAATGA
SEQ ID NO: 643
QVQLQESGPGLVKPSETLSLTCTVSGGSISGYYWSWIRQPPGKGLEWFAYFSYSGSTNYNPSLKSR
VTLSVDTSKNQFSLKLSSVTAADTAVYYCARNWAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 644
4B10
CAGGTGCAGCTGGTGGAGTCTGGGGGAGGCGTGGTCCAGCCTGGGAGGTCCCTGAGACTCTCCTGT
GCAGCCTCTGGATTCACCTTCAGTAGCTATGACATGCACTGGGTCCGCCAGGCTCCAGGCAAGGGG
CTGGAGTGGGTGGCAGTTATATCATATGATGGAACTAATGAATACTATGCAGACTCCGTGAAGGGC
CGATTCACCATCTCCAGAGACACTTCCAAGAACACGCTGTATTTGCAAATGAACAGCCTGAGAGCT
GAGGACACGGCTGTATATTACTGTGCGAGAGAACGATATTTTGACTGGTCTTTTGACTACTGGGGC
CAGGGAACCCTGGTCAGCGTCTCTAGTGCCTCCACCAAGGGCCCATCGGTCTTCCCCCTGGCACCC
TCCTCCAAGAGCACCTCTGGGGGCACAGCGGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACAGGTGTACACC
CTGCCCCCATCCCGGGAGGAGATGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTATAGCAAGCTCACCGTGGACAAGAGCAGG
TGGCAGCAGGGGAACGTCTTCTCATGCTCCGTGATGCATGAGGCTCTGCACAACCACTACACGCAG
AAGAGCCTCTCCCTGTCTCCGGGTAAATGA
SEQ ID NO: 645
QVQLVESGGGVVQPGRSLRLSCAASGFTFSSYDMHWVRQAPGKGLEWVAVISYDGTNEYYADSVKG
RFTISRDTSKNTLYLQMNSLRAEDTAVYYCARERYFDWSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 646
4F3
CAGGTGCAGCTGGTGGAGTCTGGGGGAGGCGTGGTCCAGCCTGGGAGGTCCCTGAGACTCTCCTGT
GCAGCGTCTGGATTCTCCTTCAGTAGCTATGACATGTACTCGGTCCGCCAGACTCCAGGCAAGGGG
CTGGAGTGGGTGGCAGTTATATGGTATGATGGAAGTAATAAATACTATGCAGACTCCGTGAGGGGC
CGATTCACCATCTCCAGAGACAATTCCAAGAACACGCTGTTTCTGCAAATGAACAGCCTGAGAGTC
GAGGACACGGCTGTGTATTACTGTGCGAGAGAAACTGGGGAGGGCTGGTACTTCGATCTCTGGGGC
CGTGGCACCCTGGTCACCGTCTCTAGTGCCTCCACCAAGGGCCCATCGGTCTTCCCCCTGGCACCC
TCCTCCAAGAGCACCTCTGCGGGCACAGCGGCCCTGGGCTCCCTGCTCAAGGACTACTTCCCCGAA
CCGGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGTTGAGCCCAAA
TCTTGTGACAAAACTCACACATGCCCACCGTGCCCAGCACCTGAACTCCTGGGGGGACCGTCAGTC
TTCCTCTTCCCCCCAAAACCCAAGTACACCCTCATGATCTCCCGGACCCCTGAGGTCACATGCGTG
GTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACAGGTGTACACC
CTCCCCCCATCCCCGTAGGAGATGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTATAGCAAGCTCACCGTGGACAAGAGCAGG
TGGCAGCAGGGGAACGTCTTCTCATGCTCCGTGATGCATGAGGCTCTGCACAACCACTACACGCAG
AAGAGCCTCTCCCTGTCTCCGGGTAAATGA
SEQ ID NO: 647
QVQLVESGGGVVQPGRSLRLSCAASGFSFSSYDMDWVRQTPGKGLEWVAVIWYDGSNKYYADSVRG
RFTISRDNSKNTLFLQMNSLRVEDTAVYYCARETGEGWYFDLWGRGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 648
4F7
CAGGTGCAGCTGCAGGAGTCGGGCCCAGGACTGGTGAAGCCTTCGGAGACCCTGTCCCTCACCTGC
ACTGTCTCTGGTGGCTCCATCAGTAGTTACTCCTGGAGCTGGATCCGGCAGCCCCCAGGGAAGGGA
CTGGAGTGGATTGGGTATATCTATTACAGTGGGAGCACCAACTACAACCCCTCCCTCAAGAGTCGA
GTCACCATATCATTAGACACGTCCAAGAACCAGTTCTCCCTGAAGCTGAGCTCTGTGACCGCTGCG
GACACGGCCGTGTATTACTGTGCGAGGAACTGGGCCTTCCACTTTGACTACTGGGGCCAGGGAACC
CTGGTCACCGTCTCTAGTGCCTCCACCAAGGGCCCATCGGTCTTCCCCCTGGCACCCTCCTCCAAG
AGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAAAGTTGAGCCCAAATCTTGTGAC
AAAACTCACACATGCCCACCGTGCCCAGCACCTGAACTCCTGGGGGGACCGTCAGTCTTCCTCTTC
CCCCCAAAACCCAAGGACACCCTCATGATCTCCCGGACCCCTGAGGTCACATGCGTGGTGGTGGAC
GTGAGCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCA
TCCCGGGAGGAGATGACCAAGAACCAGGTCAGCCTGACCTGCCTGGTCAAAGGCTTCTATCCCAGC
GACATCGCCGTGGAGTGGGAGAGCAATGGGCAGCCGGAGAACAACTACAAGACCACGCCTCCCGTG
CTGGACTCCGACGGCTCCTTCTTCCTCTATAGCAAGCTCACCGTGGACAAGAGCAGGTGGCAGCAG
GGGAACGTCTTCTCATGCTCCGTGATGCATGAGGCTCTGCACAACCACTACACGCAGAAGAGCCTC
TCCCTGTCTCCGGGTAAATGA
SEQ ID NO: 649
QVQLQESGPGLVKPSETLSLTCTVSGGSISSYSWSWIRQPPGKGLEWIGYIYYSGSTNYNPSLKSR
VTISLDTSKNQFSLKLSSVTAADTAVYYCARNWAFHFDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 650
16A4
CAGGTGCAGCTGCAGGAGTCGGGCCCAGGACTGGCGAAGCCTTCGGAGACCCTGTCCCTCACCTGC
ACTGTCTCTGGTGACTCCATCACTAGTTACTACTGGAGCTGGATCCGCCAGCCCCCAGGGAAGGGA
CTGGAGTGGATTGGGTATATCTATTACAGCGGGAGCACCAATTACAACCCCTCCCTCAAGAGTCGA
GTCACCATATCAGTAGACACGTCCAAGAACCAGTTCTCCCTGAAGCTGAGTTCTGTGACCGCTGCG
GACACGGCCGTGTATTACTGTGCGAGAGATCAAAGGCGGATAGCAGCAGCTGGTACCCACTTCTAC
GGTATGGACGTCTGGGGCCAAGGGACCACGGTCACTGTCTCCTCAGCTTCCACCAAGGGCCCATCC
GTCTTCCCCCTGGCGCCCTCCTCCAAGAGCACCTCTGGGGGCACAGCGCCCCTGGGCTGCCTGGTC
AAGGACTACTTCCCCGAACCGGTGACGGTGTCGTGGAACTCAGGGGCCCTGACCAGCGGCGTGCAC
ACCTTCCCGGCTGTCCTACAGTCCTCAGGACTCTACTCCCTCAGCAGCGTGGTGACCGTGCCCTCC
AGCAGCTTGGGCACCCAGACCTACATCTGCAACGTGAATCACAAGCCCAGCAACACCAAGGTGGAC
AAGAAAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGCAGCCCCGA
GAACCACAGGTCTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGCTC
ACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATGA
SEQ ID NO: 651
QVQLQESGPGLAKPSETLSLTCTVSGDSITSYYWSWIRQPPGKGLEWIGYIYYSGSTNYNPSLKSR
VTISVDTSKNQFSLKLSSVTAADTAVYYCARDQRRIAAAGTHFYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 652
16C1
CAGGTGCAGCTGCAGGAGTCGGGCCCAGGACTGGTGAAGCCTTCGGAGACCCTGTCCCTCACTTGT
ACTGTCTCTGGTGGCTCCATCAGTGGTTACTACTGGAGCTGGATCCGGCAGCCCCCAGGGAAGGGA
CTGGAGTGGATTGGGTATATCTATTACATTGGGAGCACCAACTACAACCCCTCCCTCAAGAGTCGA
GTCACCATGTCAATAGACACGTCCAAGAACCAGTTCTCCCTGACGCTGAGCTCTTTGACCGCTGCG
GACACGGCCGTGTATTTCTGTGCGAGAGATGGGAGCAGTGGCTGGTACCGGTGGTTCGACCCCTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 653
QVQLQESGPGLVKPSETLSLTCTVSGGSISGYYWSWIRQPPGKGLEWIGYIYYIGSTNYNPSLKSR
VTMSIDTSKNQFSLTLSSLTAADTAVYFCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 654
17H8
CAGGTGCAGCTGCAGGAGTCGGGCCCAGGACTGGTGAAGCCTTCGGAGACCCTGTCCCTCACGTGC
ACTGTCTCTGGTGGCTCCATCAATAGTTACTACTGGAGCTGGATCCGGCAGCCCCCAGGGAAGGGA
CTGGAGTGGATTGGGTATATCTATTACATTGGGAGCACCAACTACAACCCCTCCCTCAAGAGTCGC
GTCACCATATCAGTAGACACGTCCAAGAACCAGTTCTCCCTGAAGCTGAGCTCTGTGACCGCTGCG
GACACGGCCCTGTATTACTGTGCGAGAGATTCCCGGTATAGAAGTGGCTGGTACGATGCTTTTGAT
ATCTGGGGCCAAGGGACAATGGTCACCGTCTCTTCAGCTTCCACCAAGGGCCCATCCGTCTTCCCC
CTGGCGCCCTCCTCCAAGAGCACCTCTGGGGGCACAGCGGCCCTGGGCTGCCTGGTCAAGGACTAC
TTCCCCGAACCGGTGACGGTGTCGTGGAACTCAGGGGCCCTGACCAGCGGCGTGCACACCTTCCCG
GCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAAAGTT
GAGCCCAAATCTTGTGACAAAACTCACACATGCCCACCGTGCCCAGCACCTGAACTCCTGGGGGGA
CCGTCAGTCTTCCTCTTCCCCCCAAAACCCAAGGACACCCTCATGATCTCCCGGACCCCTGAGGTC
ACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCAAGGTCTCCAAC
AAAGCCCTCCCAGCCCCCATCGAGAAAACCATCTCCAAAGCCAAAGGGCAGCCCCGAGAACCACAG
GTGTACACCCTGCCCCCATCCCGGGAGGAGATGACCAAGAACCAGGTCAGCCTGACCTGCCTGGTC
AAAGGCTTCTATCCCAGCGACATCGCCGTGGAGTGGGAGAGCAATGGGCAGCCGGAGAACAACTAC
AAGACCACGCCTCCCGTGCTGGACTCCGACGGCTCCTTCTTCCTCTATAGCAAGCTCACCGTGGAC
AAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGTAAATGA
SEQ ID NO: 655
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARDSRYRSGWYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 656
19B5
CAGGTGCAGTTGGTGCAGTCTGGGGCTGAGGTGAAGAAGCCTGGGGCCTCAGTGAAGGTTTCCTGC
AAGGTTTCTGGATACACCTTCACCAGCTACTTTATTCACTGGGTGCGCCAGGCCCCTGGACAAGGG
CTTGAATGGATGGGAATTATCAACCCTATTAGTGTTAGCACAAGCTACGCACAGAAGTTCCAGGGC
AGAGTCACCATGACCAGGGACACGTCCACGAGCACAGTCTTCATGGAGCTGAGCAGCCTGAGATCT
GAGGACACGGCCGTCTATTACTCTGCGCGAGGGGGGATACAGCTATGGTTACATTTGGACTACTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 657
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 658
20D3
CAGGTGCAGCTGGTGCAGTCTGGGGCTGAGGTGAAGAAGCCTGGGGCCTCAGTGAAGGTTTCCTGC
AAGGTTTCTGGATACACCTTCACCAGCTACTTTATTCACTGGGTGCGCCAGGCCCCTGGACAAGGG
CTTGAGTGGATGGGAATAATCAACCCTATTAGTGTTAGCACAAGCTACGCACAGAAGTTCCAGGGC
AGAGTCACCATGACCAGGGACACGTCCACGAGCACAGTCTTCATGGAGCTGAGCAGCCTGAGATCT
GAGGACACGGCCGTGTATTACTGTGCGCGAGGGGGGATACAGCTATGGTTACATTTTGACTACTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 659
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 660
22D1
CAGGTGCAGCTGGTGCAGTCTGGGGCTGAGGTGAAGAAGCCTGGGGCCTCAGTGAGGGTTTCCTGC
AAGGTTTCTGGATACACCTTCACCAGCTACTTTATTCACTGGGTACGCCAGGCCCCTGGACAAGGG
CTTGAGTGGATGGGAATAATCAACCCTATTAGTGTTAGCACAAGCTACGCACAGAAGTTCCAGGGC
AGAGTCACCATGACCAGGGACACGTCCACGAGCACAGTCTTCATGGAGCTGAGCAGCCTGAGATCT
GAGGACACGGCCGTCTATTACTCTGCGCGACTGGGGGATACAGCTATGGTTACATTTTGACTACTG
GGGCCAGGGAACCCTGGTCACCGTCTCCTCAGCTTCCACCAAGGGCCCATCCGTCTTCCCCCTGGC
GCCCTCCTCCAAGAGCACCTCTGGGGGCACAGCGGCCCTGGGCTGCCTGGTCAAGGACTACTTCCC
CGAACCGGTGACGGTGTCGTGGAACTCAGGGGCCCTGACCAGCGGCGTGCACACCTTCCCGGCTGT
CCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAAAGTTGAGCC
CAAATCTTGTGACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAGGTGTA
CACCCTGCCCCCATCCCGGGAGGAGATGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTATAGCAAGCTCACCGTGGACAAGAG
CAGGTGGCAGCAGGGGAACGTCTTCTCATGCTCCGTGATGCATGAGGCTCTGCACAACCACTACAC
GCAGAAGAGCCTCTCCCTGTCTCCGGGTAAATGA
SEQ ID NO: 661
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDCTVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 662
22G10
GAGGTGCAACTGTTGGAGTCTGGGGGAGGCTTGGTACAGCCTGGGGGGTCCCTGAGACTCTCCTGT
GCAGCCTCTGGATTCACCTTTAGCAGTTATGCCATGAACTGGGTCCGCCAGGCTCCAGGGAAGGGG
CTGGAGTGGGTCTCAACTATTAGTGGTGGTGGTGCTAACACATACTACGCAGACTCCGTGAAGGGC
CGGTTCACCATCTCCAGTGACAATTCCAAGAGCACGCTGTATCTGCAAATGAACAGCCTGAGAGCC
GCGGACACGGCCGTATATCACTGTGCGAAAGGGGGAATGGGGGGATACTACTACGGTATGGACGTC
TGGGGCCAAGGGACCACGGTCACCGTCTCCTCAGCTTCCACCAAGGGCCCATCCGTCTTCCCCCTG
GCGCCCTCCTCCAAGAGCACCTCTGGGGGCACAGCGGCCCTGGGCTGCCTGGTCAAGGACTACTTC
CCCGAACCGGTGACGGTGTCGTGGAACTCAGGGGCCCTGACCAGCGGCGTGCACACCTTCCCGGCT
GTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAAAGTTGAG
CCCAAATCTTGTGACAAAACTCACACATGCCCACCGTGCCCAGCACCTGAACTCCTGGGGGGACCG
TCAGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCGTG
GAGGTGCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGAACCACAGGTG
TACACCCTGCCCCCATCCCGGGAGGAGATGACCAAGAACCAGGTCAGCCTGACCTGCCTGGTCAAA
GGCTTCTATCCCAGCGACATCGCCGTGGAGTGGGAGAGCAATGGGCAGCCGGAGAACAACTACAAG
ACCACGCCTCCCGTGCTGGACTCCGACGGCTCCTTCTTCCTCTATAGCAAGCTCACCGTGGACAAG
AGCAGGTGGCAGCAGGGGAACGTCTTCTCATGCTCCGTGATGCATGAGGCTCTGCACAACCACTAC
ACGCAGAAGAGCCTCTCCCTGTCTCCGGGTAAATGA
SEQ ID NO: 663
EVQLLESGGGLVQPGGSLRLSCAASGFTFSSYAMNWVRQAPGKGLEWVSTISGGGANTYYADSVKG
RFTISSDNSKSTLYLQMNSLRAADTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 664
23A10
CAGGTGCAGCTGGTGGAGTCTGGGGGAGGCGTGGTCCAGCCTGGGAGGTCCCTGAGACTCTCCTGT
GCAGCGTCTGGATTCACCTTCAGTCGCTATGGCATACACTGGGTCCGCCAGGCTCCAGGCAAGGGG
CTGGAGTGGGTGGCAGTTATATGGTATGATGGAAGTAATAAATACTATGCAGACTCCGTGAAGGGC
CGATTCACCATCTCCAGAGACAATTCCAAGAACACGCTGTATCTGCTAATGAACAGCCTGAGAGCC
GAGGACTCGGCTGTGTATTACTGTCCGAGAAGGGCCGCTATACCTCGAACTACGGGCTACTACTAT
GGTATGGACGTCTGGGGCCAAGGGACCACGGTCACCGTCTCCTCAGCTTCCACCAAGGGCCCATCC
GTCTTCCCCCTGGCGCCCTCCTCCAAGAGCACCTCTGGGGGCACAGCGGCCCTGGGCTGCCTGGTC
AAGGACTACTTCCCCGAACCGGTGACGGTGTCGTGGAACTCAGGGGCCCTGACCAGCGGCGTGCAC
ACCTTCCCGGCTGTCCTACAGTCCTCAGGACTCTACTCCCTCAGCAGCGTGGTGACCGTGCCCTCC
AGCAGCTTGGGCACCCAGACCTACATCTGCAACGTGAATCACAAGCCCAGCAACACCAAGGTGGAC
AAGAAAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGACC
CCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGCAGCCCCGA
GAACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAG
AACAACTACAAGACCACGCCTCCCGTGCTGGACTCCGACGGCTCCTTCTTCCTCTATAGCAAGCTC
ACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATGA
SEQ ID NO: 665
QVQLVESGGGVVQPGRSLRLSCAASGFTFSRYGIHWVRQAPGKGLEWVAVIWYDGSNKYYADSVKG
RFTISRDNSKNTLYLLMNSLRAEDSAVYYCARRAGIPGTTGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 666
25F8
CAGGTGCAGCTGGTGCAGTCTGGGGCTGAGGTGAAGAAGCCTGGGGCCTCAGTGAAGGTTTCCTGC
AAGGCATCTGGATACACCTTCACCAGCTACTATATTCACTGGGTGCGCCAGGCCCCTGGACAAGGA
CTTGAGTGGATGGGAATAATCAACCCCAGTGGTGGTAGCACAAGGTACGCACAGAAGTTCCAGGGC
AGAGTCACCATGACCAGGGACACGTCCACGAGCACAGTCTTCATGGAGCTGAGCAGCCTGAGATCT
GAGGACACGGCCGTGTATTACTGTGCGCGAGGGGGAATACAGCTATGGTTACATTTTGACTACTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATCGGCAGCCCGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 667
QVQLVQSGAEVKKPGASVKVSCKASGYTFTSYYIHWVRQAPGQGLEWMGIINPSGGSTRYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 668
25G10
CAGGTGCAGCTGCAGGAGTCGGGCCCAGGACTGGTGAAGCCTTCGGAGACCCTGTCCCTCACCTGC
ACTGTCTCTGGTGGCTCCATCAGTGGTTACTACTGGAGCTGGATCCGGCAGCCCCCAGGGAAGGGA
CTGGAGTGGATTGGGTATATCTATTACATTGGGAGCACCAACTACAACCCCTCCCTCAAGAGTCGA
GTCACCATGTCAGTAGACACGTCCAAGAACCAGTTCTCCCTGAAGCTGAGCTCTGTGACCGCTGCG
GACACGCCCGTGTATTACTGTGCGAGAGATGGGAGCAGTGGCTGGTACCGGTGGTTCGACCCCTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 669
QVQLQESGPGLVKPSETLSLTCTVSGGSISGYYWSWIRQPPGKGLEWIGYIYYIGSTNYNPSLKSR
VTMSVDTSKNQFSLKLSSVTAADTAVYYCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPG
SEQ ID NO: 670
26D1
CAGGTGCAGTTGGTGCAGTCTGGGGCTGAGGTGAAGAAGCCTGGGGCCTCAGTGAAGGTTTCCTGT
AAGGCATCTAGATACACCTTCACCAGCTACTATATGTCCTGGGTGCGACAGGCCCCTGGACAAGGG
CTTGAGTGGATGGGAATAATCCACCCTAGTGGTGGTGACACAACCTACGCACAGAAGTTCCAGGGC
AGAGTCACCATGACCGGGGACACGTCCACGAGCACAGTCTACATGGAGCTGAGCAGCCTGAGATCT
GAGGACACGGCCGTGTATTACTGTGCGAGAGGGGGGATAAAACTATGGTTACATTTTGACTATTGG
GGCCAGGGAACCCTGGTCACCGTCTCCTCAGCTTCCACCAAGGGCCCATCCGTCTTCCCCCTGGCG
CCCTCCTCCAAGAGCACCTCTGGGGGCACAGCGGCCCTGGGCTGCCTGGTCAAGGACTACTTCCCC
GAACCGGTGACGGTGTCGTGGAACTCAGGGGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACC
CAGACCTACATCTGCAACGTGAATCACAAGCCCAGCAACACCAAGGTGGACAAGAAAGTTGAGCCC
AAATCTTGTGACAAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTGAGGTCACATGC
GTGGTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGCGTC
CTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTAC
ACCCTGCCCCCATCCCGGGAGGAGATGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGACC
ACGCCTCCCGTGCTGGACTCCGACGGCTCCTTCTTCCTCTATAGCAAGCTCACCGTGGACAAGAGC
AGGTGGCAGCAGGGGAACGTCTTCTCATGCTCCGTGATGCATGAGGCTCTGCACAACCACTACACG
CAGAAGAGCCTCTCCCTGTCTCCGGGTAAATGA
SEQ ID NO: 671
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 672
26F12
CAGGTGCAGTTGGTGCAGTCTGGGGCTGAGGTGAAGAAGCCTGGGGCCTCAGTGAAGGTTTCCTGC
AAGGCATCTAGATACACCTTCACCAACTACTATATGTCCTGGGTGCGACAGGCCCCTGGACAAGGG
CTTGAGTGGATGGGAATAATCAACCCTAGTGGTGGTGACTCAACCTACGCACAGAAGTTCCAGGGC
AGACTCACCATGACCGGGGACACGTCCACGAGCACAGTCTACATGGAGCTGAGCAGCCTGAGATCT
GAGGACACCTGCCGTCTATTACTCTGCGAGAGGTTGGATACAACTATGGTTACATTTTGACTACTG
GGGCCAGGGAACCCTGGTCACCGTCTCCTCAGCTTCCACCAAGGGCCCATCCGTCTTCCCCCTGGC
GCCCTCCTCCAAGAGCACCTCTGGGGGCACAGCGGCCCTGGGCTGCCTGGTCAAGGACTACTTCCC
CGAACCGGTGACGGTGTCGTGGAACTCAGGGGCCCTGACCAGCGGCGTGCACACCTTCCCGGCTGT
CCTACAGTCCTCAGGACTCTACTCCCTCAGCAGCGTGGTGACCGTGCCCTCCAGCAGCTTGGGCAC
CCAGACCTACATCTGCAACGTGAATCACAAGCCCAGCAACACCAAGGTGGACAAGAAAGTTGAGCC
CAAATCTTGTGACAAAACTCACACATGCCCACCGTGCCCAGCACCTGAACTCCTGGGGGGACCGTC
AGTCTTCCTCTTCCCCCCAAAACCCAAGGACACCCTCATGATCTCCCGGACCCCTGAGGTCACATG
CGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCAAGGTCTCCAACAAAGC
CCTCCCAGCCCCCATCGAGAAAACCATCTCCAAAGCCAAAGGGCAGCCCCGAGAACCACAGGTGTA
CACCCTGCCCCCATCCCGGGAGGAGATGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTATAGCAAGCTCACCGTGGACAAGAG
CAGGTGGCAGCAGGGGAACGTCTTCTCATGCTCCGTGATGCATGAGGCTCTGCACAACCACTACAC
GCAGAAGAGCCTCTCCCTGTCTCCGGGTAAATGA
SEQ ID NO: 673
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 674

TABLE IIIb
Light Chain Variable and Contant
Region Polynucleotide and Amino acid Sequences

2G6
TCCTATGAACTGACTCAGCCACCCTCAGTGTCCGTGTCCCCAGGACAGACAGCCAGCATCACCTGC
TCTGGAGATAGGTTGGGGGAAAAATATACTTGCTGGTATCAGCAGAGGCCAGGCCAGTCCCCTTTG
CTGGTCATCTATCAAGATACCAAGCGGCCCTCAGGGATCCCTGAGCGATTCTCTGGCTCCAACTCT
GGTAACACAGCCACTCTGACCATCAGCGGGACCCAGGCTATGGATGAGGCTGACTATTACTGTCAG
GCGTGGGACAGCAGCACTGTGGTATTCGGCGGAGGGACCAAGCTGACCGTCCTAGGTCAGCCCAAG
GCCAACCCCACTGTCACTCTGTTCCCGCCCTCCTCTGAGGAGCTCCAAGCCAACAAGGCCACACTA
GTGTGTCTGATCAGTGACTTCTACCCGGGAGCTGTGACAGTGGCCTGGAAGGCAGATGGCAGCCCC
GTCAAGGCGGGAGTGGAGACCACCAAACCCTCCAAACAGAGCAACAACAAGTACGCGGCCAGCAGC
TACCTGAGCCTGACGCCCGAGCAGTGGAAGTCCCACAGAAGCTACAGCTGCCAGGTCACGCATGAA
GGGAGCACCGTGGAGAAGACAGTGGCCCCTACAGAATGTTCATGA
SEQ ID NO: 675
SYELTQPPSVSVSPGQTASITCSGDRLGEKYTCWYQQRPGQSPLLVIYQDTKRPSGIPERFSGSNS
GNTATLTISGTQAMDEADYYCQAWDSSTVVFGGGTKLTVLGQPKANPTVTLFPPSSEELQANKATL
VCLISDFYPGAVTVAWKADGSPVKAGVETTKPSKQSNNKYAASSYLSLTPEQWKSHRSYSCQVTHE
GSTVEKTVAPTECS
SEQ ID NO: 676
4A2
GAAATTGTGTTGACGCAGTCTCCAGGCACCCTGTCTTTGTCTCCAGGGGAAAGAGCCACCCTCTCC
TGCAGGGCCAGTCGGAATATTAGCAGCAGCTACTTAGCCTGGTACCAGCAGAAACCTGGCCAGGCT
CCCAGGCTCCTCATCTATGGTCCATCCAGCAGGGCCACTGGCATCCCAGACAGGTTCAGTGGCAGT
GGGTCTGGGACAGACTTCACTCTCACCATCAGCAGACTGGAGCCTGAAGATTTTACAGTGTATTAC
TGTCAGCAGTATGGTAGCTCATTCACTTTCGGCCCTGGGACCAAAGTGGATATCAAACGTACGGTG
GCTGCACCATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTGA
SEQ ID NO: 677
EIVLTQSPGTLSLSPGERATLSCRASRNISSSYLAWYQQKPGQAPRLLIYGPSSRATGIPDRFSGS
GSGTDFTLTISRLEPEDFTVYYCQQYGSSFTFGPGTKVDIKRTVAAPSVFIFPPSDEQLKSGTASV
VCLLNNFYPREAKVQWKVDNALQSGNSQESVTEQDSKDSTYSLSSTLTLSKADYEKHKVYACEVTH
QGLSSPVTKSFNRGEC
SEQ ID NO: 678
4A9
CAGTCTGTGCTGACGCAGCCGCCCTCAGTGTCTGGGGCCCCAGGACAGAGGGTCACCATCTCCTGC
ACTGGGAGCAGCTCCAACATCGGGACAGGTTATGCTGTACACTGGTACCAGCAGTTTCCAGGAACA
GCCCCCAAACTCCTCATCTATGGTAACAACAATCGGCCCTCAGGGGTTCCTGACCGATTCTCTGGC
TCCAAGTCTGGCACCTCAGCCTCCCTGGCCATCACTGGGCTCCAGGCTGAGGATGAGGCTGATTAT
TACTGCCAGTCCTATGACAGCAGACTGAGTGGTTGGGTGTTCGGCGGAGGGACCAAGCTGACCGTC
CTAGGTCAGCCCAAGGCCAACCCCACTGTCACTCTGTTCCCGCCCTCCTCTGAGGAGCTCCAAGCC
AACAAGGCCACACTAGTGTGTCTGATCAGTGACTTCTACCCGGGAGCTGTGACAGTGGCCTGGAAG
GCAGATGGCAGCCCCGTCAAGGCGGGAGTGGAGACCACCAAACCCTCCAAACAGAGCAACAACAAG
TACGCGGCCAGCAGCTACCTGAGCCTGACGCCCGAGCAGTGGAAGTCCCACAGAAGCTACAGCTGC
CAGGTCACGCATGAAGGGAGCACCGTGGAGAAGACAGTGGCCCCTACAGAATGTTCATGA
SEQ ID NO: 679
QSVLTQPPSVSGAPGQRVTISCTGSSSNIGTGYAVHWYQQFPGTAPKLLIYGNNNRPSGVPDRFSG
SKSGTSASLAITGLQAEDEADYYCQSYDSRLSGWVFGGGTKLTVLGQPKANPTVTLFPPSSEELQA
NKATLVCLISDFYPGAVTVAWKADGSPVKAGVETTKPSKQSNNKYAASSYLSLTPEQWKSHRSYSC
QVTHEGSTVEKTVAPTECS
SEQ ID NO: 680
4B10
GAAATTGTATTGACGCAGTCTCCAGGCACCCTGTCTTTGTCTCCAGGGGAAAGAGCCACCCTCTCC
TGCAGGGCCAGTCAGAGTGTTAGCAACACCTACTTAGCCTGGTACCATCAGAGACCTGGCCAGGCT
CCCAGGCTCCTCATCTATGGTGCATCCAGCAGGGCCACTGGCATCCCAGACAGATTCAGTGGCAGT
GGGTCTGGGACAGACTTCGCTCTCACCATCAGCAGTCTGGAGCCTGAAGATTTTGCAGTGTATTAC
TGTCAGCAGTACAGTAACTCGTGGACGTTCGGCCAAGGGACCAAGGTGGAAATCAAACGAACTGTG
GCTGCACCATCTGTCTTCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTGA
SEQ ID NO: 681
EIVLTQSPGTLSLSPGERATLSCRASQSVSNTYLAWYHQRPGQAPRLLIYGASSRATGIPDRFSGS
GSGTDFALTISSLEPEDFAVYYCQQYSNSWTFGQGTKVEIKRTVAAPSVFIFPPSDEQLKSGTASV
VCLLNNFYPREAKVQWKVDNALQSGNSQESVTEQDSKDSTYSLSSTLTLSKADYEKHKVYACEVTH
QGLSSPVTKSFNRGEC
SEQ ID NO: 682
4F3
GAAATTGTGTTGACGCAGTCTCCAGGCACCCTGTCTTTGTCTCCAGGGGAAAGAGCCACCCTCTCC
TGCAGGGCCAGTCAGAGTGTTAGCAGCAGCTACTTAGCCTGGTACCAGCAGAAACCTGGCCAGGCT
CCCAGGCTCCTCATCTATGGTGCATCCAGCAGGGCCACTGGCATCCCAGACAGGTTCAGTGGCAGT
GGGTCTGGGACAGACTTCACTCTCACCATCAGCAGACTGGAACCTGAGGATTTTGCAGTGTATTAC
TGTCAGCAGTATGGTAGCTCGTGGACGTTCGGCCAAGGGACCAAGGTGGAAATCAAACGTACGGTG
GCTGCACCATCTGTCTTCATCTTCCCGCCATCTGATGAGCAGTTGAAATCTGGAACTGCCTCTGTT
GTGTGCCTCCTGAATAACTTCTATCCCAGAGAGGCCAAAGTACAGTCGAAGGTGGATAACGCCCTC
CAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTGA
SEQ ID NO: 683
EIVLTQSPGTLSLSPGERATLSCRASQSVSSSYLAWYQQKPGQAPRLLIYGASSRATGIPDRFSGS
GSGTDFTLTISRLEPEDFAVYYCQQYGSSWTFGQGTKVEIKRTVAAPSVFIFPPSDEQLKSGTASV
VCLLNNFYPREAKVQWKVDNALQSGNSQESVTEQDSKDSTYSLSSTLTLSKADYEKHKVYACEVTH
QGLSSPVTKSFNRGEC
SEQ ID NO: 684
4F7
CAGTCTGTGCTGACGCAGCCGCCCTCAGTGTCTGGGGCCCCAGGGCAGAGGGTCACCATCTCCTGC
ACTGGGAGCAGCTCCAATATCGGGACAGGTTATGATGTACACTGGTATCAGCAGCTTCCAGGAACA
GCCCCCAAACTCCTCATCCATGGTAACAGCAATCGGCCCTCAGGGGTCCCTGACCGATTCTCTGGC
TCCAAGTCTGGCACCTCAGCCTCCCTGGCCATCACTGGGCTCCAGGCTGAGGATGAGGCTGATTAT
TACTGCCAGTCCTATGACAGCAGTCTGAGTGGTTGGGTGTTCGGCGGAGGGACCAGGTTGACCGTC
CTAGGTCAGCCCAAGGCCAACCCCACTGTCACTCTGTTCCCGCCCTCCTCTGAGGAGCTCCAAGCC
AACAAGGCCACACTAGTGTGTCTGATCAGTGACTTCTACCCGGGAGCTGTGACAGTGGCCTGGAAG
GCAGATGGCAGCCCCGTCAAGGCGGGAGTGGAGACCACCAAACCCTCCAAACAGAGCAACAACAAG
TACGCGGCCAGCAGCTACCTGAGCCTGACGCCCGAGCAGTGGAAGTCCCACAGAAGCTACAGCTGC
CAGGTCACGCATGAAGGGAGCACCGTGGAGAAGACAGTGGCCCCTACAGAATGTTCATGA
SEQ ID NO: 685
QSVLTQPPSVSGAPGQRVTISCTGSSSNIGTGYDVHWYQQLPGTAPKLLIHGNSNRPSGVPDRFSG
SKSGTSASLAITGLQAEDEADYYCQSYDSSLSGWVFGGGTRLTVLGQPKANPTVTLFPPSSEELQA
NKATLVCLISDFYPGAVTVAWKADGSPVKAGVETTKPSKQSNNKYAASSYLSLTPEQWKSHRSYSC
QVTHEGSTVEKTVAPTECS
SEQ ID NO: 686
16A4
GAAATTGTGTTGACGCAGTCTCCAGGCACCCTGTCTTTGTCTCCAGGGGAAAGAGCCACCCTCTCC
TGCAGGGCCAGTCAGAGTGTTAGCAGCAGTTATTTAGCCTGGTACCAGCAGAAACCTGGCCAGGCT
CCCAGGCTCCTCATCTATGGTACATCCAGCAGGGCCACTGGCATCCCAGACAGGTTCAGTGGCAGT
GGGTCTGGGACAGACTTCACTCTCACCATCAGCAGACTGGAGCCTGAAGATTTTGCAGTGTATTAT
TGTCAGCAGTACGGTAGCTCACCTTTCACTTTCGGCGGAGGGACCAAGGTGGAGATCAAACGAACT
GTGGCTGCACCATCTGTCTTCATCTTCCCGCCATCTGATGAGCAGTTGAAATCTGGTACCGCCTCT
GTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTGA
SEQ ID NO: 687
EIVLTQSPGTLSLSPGERATLSCRASQSVSSSYLAWYQQKPGQAPRLLIYGTSSRATGIPDRFSGS
GSGTDFTLTISRLEPEDFAVYYCQQYGSSPFTFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVT
HQGLSSPVTKSFNRGEC
SEQ ID NO: 688
16C1
GAAATTGTGTTGACGCAGTCTCCAGGCACCCTGTCTTTGTCTCCAGGGGAAAGAGCCACCCTCTCC
TGCAGGGCCATCCAGAGTGTTAGCAGCAGCTACTTAGCCTGGTACCAGCAGAAACCTGGCCAGGCT
CCCAGGCTCCTCATCTTTGGTGCATCCAGCAGGGCCACTGGCATCCCAGACAGGTTCAGTGGCAGT
GGGTCTGGGACAGACTTCACTCTCACCATCAGCGGACTGGAGCCTGAAGATTTTGCAGTGTATCAC
TGTCAGCAGTATGGTAACTCACCGCTCACTTTCGGCGGAGGGACCAAGGTGGAGATCAAACGAACT
GTGGCTGCACCATCTGTCTTCATCTTCCCGCCATCTGATGAGCAGTTGAAATCTGGTACCGCCTCT
GTTGTGTGCCTCCTGAATAACTTCTATCCCAGAGAGGCCAAAGTACAGTCGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTCTTGA
SEQ ID NO: 689
EIVLTQSPGTLSLSPGERATLSCRASQSVSSSYLAWYQQKPGQAPRLLIFGASSRATGIPDRFSGS
GSGTDFTLTISGLEPEDFAVYHCQQYGNSPLTFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVT
HQGLSSPVTKSFNRGEC
SEQ ID NO: 690
17H8
GACATTGTATTGACGCAGTCTCCAGGCACCCTGTCTTTGTCTCCAGGGGAAAGAGCCACCCTCTCC
TGCAGGGCCAGTCAGAGTGTTGCCGGCAGCTACCTAGCCTGGTACCAGCAGAAACCTGGCCAGGCT
CCCAGGCTCCTCATCTCTCGTCCATCCAGCAGGGCCACTGGCATCCCAGACAGGTTCAGTGGCAGT
GGGTCTGGGACAGACTTCACTCTCACCATCAGCAGACTGGAGCCTGAAGATTTTGCAGTGTATTAC
TGTCAGCAGTATGGTAAATCACCGATCACCTTCGGCCAAGGGACACGACTGGAGATGAAAGGAACT
GTGGCTGCACCATCTGTCTTCATCTTCCCGCCATCTGATGAGCAGTTGAAATCTGGTACCGCCTCT
GTTGTGTGCCTGCTGAATAACTTCTATCCCAGAGAGGCCAAAGTACAGTGGAAGGTGGATAACGCC
CTCCAATCGGGTAACTCCCAGGAGAGTCTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGGCCTGAGCTCGCCCGTCACAAAGAGCTTCAACAGGGGAGAGTGTTGA
SEQ ID NO: 691
DIVLTQSPGTLSLSPGERATLSCRASQSVAGSYLAWYQQKPGQAPRLLISGASSRATGIPDRFSGS
GSGTDFTLTISRLEPEDFAVYYCQQYGKSPITFGQGTRLEMKGTVAAPSVFIFPPSDEQLKSGTAS
VVCLLNNFYPREAKVQWKVDNALQSGNSQESVTEQDSKDSTYSLSSTLTLSKADYEKHKVYACEVT
HQGLSSPVTKSFNRGEC
SEQ ID NO: 692
19B5
CAGTCTGCGCTGACTCAGCCACCCTCAACGACTGGGACCCCCGGGCAGAGGGTCACCATCTCTTGT
TCTGGAAGCAGGTCCAACATCGGAAGCAATTTTGTAAACTGGTACAAGCAGCTCCCAGGAACGGCC
CCCAAAGTCCTCATCTATACTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGTCTGATTATTAC
TGCGCAACATGGGATGACAGTATGAATGGTTGGGTGTTCGGCGGAGGGACCAAACTGACCGTCCTA
GGTCAGCCCAAGGCTGCCCCCTCGGTCACTCTGTTCCCACCCTCCTCTGAGGAGCTTCAAGCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGGCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 693
QSALTQPPSTTGTPGQRVTISCSGSRSNIGSNFVNWYKQLPGTAPKVLIYTNNQRPSGVPDRFSGS
KSGTSASLAISGLQSEDESDYYCATWDDSMNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 694
20D3
CAGTCTCCGCTGACTCAGCCACCCTCAGCGACTGGGACCCCCGGGCAGAGGGTCACCATCTCTTGT
TCTGGAAGCAGCTCCAACATCGGAAGCAATTTTGTAAACTGGTACAAGCAGCTCCCAGGAACGGCC
CCCAAAGTCCTCATCTATACTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGTCTGATTATTAC
TGTGCAACATGGGATGACAGCCTGAATGGTTGGGTGTTCGGCGGAGGGACCAAGCTGACCGTCCTA
GGTCAGCCCAAGGCTTCCCCCTCGCTCACTCTCTTCCCACCCTCCTCTGAGGAGCTTCAATCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGGCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 695
QSALTQPPSATGTPGQRVTISCSGSSSNIGSNFVNWYKQLPGTAPKVLIYTNNQRPSGVPDRFSGS
KSGTSASLAISGLQSEDESDYYCATWDDSLNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 696
22D1
CAGTCTGCGCTGACTCAGCCACCCTCAGCGACTGGGACCCCCGGGCAGAGGGTCACCATCTCTTGT
TCTGGAAGCAGCTCCAACATCGGAAGCAATTTTGTAAACTGGTACAAGCAGCTCCCAGGAACCGCC
CCCAAAGTCCTCATCTATACTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGTCTGATTATTAC
TGTGCAACATGGGATGACAGTATGAATGGTTGGGTGTTCGGCGGAGGGACCAAGCTGACCGTCCTA
GGTCAGCCCAAGGCTGCCCCCTCGGTCACTCTGTTCCCACCCTCCTCTGAGGAGCTTCAAGCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGGCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 697
QSALTQPPSATGTPGQRVTISCSGSSSNIGSNFVNWYKQLPGTAPKVLIYTNNQRPSGVPDRFSGS
KSGTSASLAISGLQSEDESDYYCATWDDSMNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 698
22G10
GAAATAGTGATGACGCAGTCTCCAGTCACCCTGTCTCTGTCTCTAGGGGAAAGAGCCACCCTCTCC
TGCAGGGCCAGTCAGAGTATTAGCAGCAACTTAGCCTGGTTCCAGCAGAAACCTGGCCAGGCTCCC
AGACTCCTCATCTATGGTGCATTTACCAGGGCCACTGGTATCCCAGCCAGGGTCAGTGGCAGTGGG
TCTGGGACAGAGTTCACTCTCACCATCAGCAGCCTGCAGTCTGAAGATTTTGCAGTTTATTACTGT
CAGCAGTATAATTACTGGCCGCTCACTTTCGGCGGAGGGACCAAGGTGGAGATCAAGCGAACTGTG
GCTGCACCATCTGTCTTCATCTTCCCGCCATCTGATGAGCAGTTGAAATCTGGTACCGCCTCTGTT
GTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTTGA
SEQ ID NO: 699
EIVMTQSPVTLSLSLGERATLSCRASQSISSNLAWFQQKPGQAPRLLIYGAFTRATGIPARVSGSG
SGTEFTLTISSLQSEDFAVYYCQQYNYWPLTFGGGTKVEIKRTVAAPSVFIFPPSDEQLKSGTASV
VCLLNNFYPREAKVQWKVDNALQSGNSQESVTEQDSKDSTYSLSSTLTLSKADYEKHKVYACEVTH
QGLSSPVTKSFNRGEC
SEQ ID NO: 700
23A10
TCCTATGAGCTGACTCAGCCACCCTCAGTGTCCGTGTCCCCAGGACAGACAGCCAGCATCACCTGC
TCTGGAGATAGATTGGGGGAGAAATATGTTTGCTGGTATCAGCAGAAGCCAGGCCAGTCCCCTATA
CTGGTCATCTATCAAGATAATAAGTGGCCCTCAGGGATCCCTGAGCGATTCTCTGGCTCCAACTCT
GGGAACACAGCCACTCTGACCATCAGCGGGACCCAGGCTATGGATGAGGCTGACTATTACTGTCAG
GCGTGGGACAGCAGCACTGTGGTATTCGGCGGGGGGACCAAGCTGACCGTCCTAGGTCAGCCCAAG
GCTGCCCCCTCGGTCACTCTGTTCCCACCCTCCTCTGAGGAGCTTCAAGCCAACAAGGCCACACTG
GTGTGTCTCATAAGTGACTTCTACCCGGGAGCCGTGACAGTGGCCTGGAAGGCAGATAGCAGCCCC
GTCAAGGCGGGAGTGGAGACCACCACACCCTCCAAACAAAGCAACAACAAGTACGCGGCCAGCAGC
TATCTGAGCCTGACGCCTGAGCAGTGGAAGTCCCACAGAAGCTACAGCTGCCAGGTCACGCATGAA
GGGAGCACCGTGGAGAAGACAGTGGCCCCTACAGAATGTTCATGA
SEQ ID NO: 701
SYELTQPPSVSVSPGQTASITCSGDRLGEKYVCWYQQKPGQSPILVIYQDNKWPSGIPERFSGSNS
GNTATLTISGTQAMDEADYYCQAWDSSTVVFGGGTKLTVLGQPKAAPSVTLFPPSSEELQANKATL
VCLISDFYPGAVTVAWKADSSPVKAGVETTTPSKQSNNKYAASSYLSLTPEQWKSHRSYSCQVTHE
GSTVEKTVAPTECS
SEQ ID NO: 702
25F8
CAGTCTGCGCTGACTCAGCCACCCTCAGCGACTGGGACCCCCGGGCAGAGGGTCACCATCTCTTGT
TCTGGAAGCAGCTCCAACATCGGAAGGAATTTTGTAAACTGGTATAAGCAGCTCCCAGGAACGGCC
CCCAAAGTCCTCATTTATACTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGTCTGATTATTAC
TGTGCAGCATGGGATGACAGCCTGAATGGTTGGGTGTTCGGCGGAGGGACCAAGCTGACCGTCCTA
GGTCAGCCCAAGGCTGCCCCCTCGGTCACTCTGTTCCCACCCTCCTCTGAGGAGCTTCAAGCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGGCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 703
QSALTQPPSATGTPGQRVTISCSGSSSNIGRNFVNWYKQLPGTAPKVLIYTNNQRPSGVPDRFSGS
KSGTSASLAISGLQSEDESDYYCAAWDDSLNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 704
25G10
GAAATTGTGTTGACGCAGTCTCCAGGCACCCTGTCTTTGTCTCCAGGGGAAAGAGCCACCCTCTCC
TGCAGGGCCAGTCAGAGTGTTAGCAGCAGCTACTTAGCCTGGTACCAGCAGAAACCTGGCCAGGCT
CCCAGGCTCCTCATCTTTGGTGCATCCAGCAGGGCCACTGGCATCCCAGACAGGTTCAGTGGCAGT
GGGTCTGGGACAGACTTCACTCTCACCATCAGCAGACTGGAGCCTGAAGATTTTGCAGTGTATCAC
TGTCAGCAGTATGGTAACTCACCGCTCACTTTCGGCGGAGGGACCAAGGTGGAGATCAAACGAACT
GTGGCTGCACCATCTGTCTTCATCTTCCCGCCATCTGATGAGCAGTTGAAATCTGGTACCGCCTCT
GTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACC
CATCAGGCTCCTGAGCTCGCCCGTCACAAACTAGCTTCAACAGGGCTAGAGTCTTCTA
SEQ ID NO: 705
EIVLTQSPGTLSLSPGERATLSCRASQSVSSSYLAWYQQKPGQAPRLLIFGASSRATGIPDRFSGS
GSGTDFTLTISRLEPEDFAVYHCQQYGNSPLTFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVT
HQGLSSPVTKSFNRGEC
SEQ ID NO: 706
26D1
CACTCTGTGCTGACTCAGTCACCCTCAGCGTCTGGGACCCCCGGACAGAGGGTCACCATCTCTTGT
TCTGGAAGCCGCTCCAACATCGGAAGTAATTTTGTAAACTGGTACCAGCAGCTCCCAGGAACGGCC
CCCAAACTCCTCATCTATACTAATAATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGTATGGGATGACAGCCTGAATGGTTGGGTGTTCGGCGGAGGGACCAAGCTGACCGTCCTA
GGTCAGCCCAAGGCTGCCCCCTCGGTCACTCTGTTCCCACCCTCCTCTGAGGAGCTTCAAGCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGCCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 707
HSVLTQSPSASGTPGQRVTISCSGSRSNIGSNFVNWYQQLPGTAPKLLIYTNNQRPSGVPDRFSGS
KSGTSASLAISGLQSEDEADYYCAVWDDSLNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 708
26F12
CAGTCTGTGCTGACTCAGTCACCCTCAGCGTCTGGGACCCCCGGGCAGAAGGTCACCATCTCTTGT
TCTGGAAGCCGCTCCAACATCGGAAGTAATTTTGTAAACTGGTACCAGCAGCTCCCAGGAACGGCC
CCCAAACTCCTCATCTATACTAATTATCAGCGGCCCTCAGGGGTCCCTGACCGATTCTCTGGCTCC
AAGTCTGGCACCTCAGCCTCCCTGGCCATCAGTGGGCTCCAGTCTGAGGATGAGGCTGATTATTAC
TGTGCAGTATGGGATGACAGCCTGAATGGTTGGGTGTTCGGCGGAGGGACCAAGCTGACCGTCCTA
GGTCAGCCCAAGGCTGCCCCCTCGGTCACTCTGTTCCCACCCTCCTCTGAGGAGCTTCAAGCCAAC
AAGGCCACACTGGTGTGTCTCATAAGTGACTTCTACCCGGGAGCCGTGACAGTGGCCTGGAAGGCA
GATAGCAGCCCCGTCAAGGCGGGAGTGGAGACCACCACACCCTCCAAACAAAGCAACAACAAGTAC
GCGGCCAGCAGCTATCTGAGCCTGACGCCTGAGCAGTGGAAGTCCCACAGAAGCTACAGCTGCCAG
GTCACGCATGAAGGGAGCACCGTGGAGAAGACAGTGGCCCCTACAGAATGTTCATGA
SEQ ID NO: 709
QSVLTQSPSASGTPGQKVTISCSGSRSNIGSNFVNWYQQLPGTAPKLLIYTNYQRPSGVPDRFSGS
KSGTSASLAISGLQSEDEADYYCAVWDDSLNGWVFGGGTKLTVLGQPKAAPSVTLFPPSSEELQAN
KATLVCLISDFYPGAVTVAWKADSSPVKAGVETTTPSKQSNNKYAASSYLSLTPEQWKSHRSYSCQ
VTHEGSTVEKTVAPTECS
SEQ ID NO: 710

TABLE IIIc
Heavy Chain Variable and Contant
Region Polynucleotide and Amino acid Sequences

13586_HC [hu anti-<huCDH19> 4F3 VH]::huIgG1z
QVQLVESGGGVVQPGRSLRLSCAASGFSFSSYDMDWVRQTPGKGLEWVAVIWYDGSNKYYADSVRG
RFTISRDNSKNTLFLQMNSLRVEDTAVYYCARETGEGWYFDLWGRGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 711
13589_HC [hu anti-<huCDH19> 4A9 VH]::huIgG1z
QVQLQESGPGLVKPSETLSLTCTVSGGSISGYYWSWIRQPPGKGLEWFAYFSYSGSTNYNPSLKSR
VTLSVDTSKNQFSLKLSSVTAADTAVYYCARNWAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 712
13590_HC [hu anti-<huCDH19> 4B10 VH]::huIgG1z
QVQLVESGGGVVQPGRSLRLSCAASGFTFSSYDMHWVRQAPGKGLEWVAVISYDGTNEYYADSVKG
RFTISRDTSKNTLYLQMNSLRAEDTAVYYCARERYFDWSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 713
13874_HC [hu anti-<huCDH19> 17H8.2 VH]::huIgG1z
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARDSRYRSGWYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 714
13875_HC [hu anti-<huCDH19> 16C1.1 VH]::huIgG1z
QVQLQESGPGLVKPSETLSLTCTVSGGSISGYYWSWIRQPPGKGLEWIGYIYYIGSTNYNPSLKSR
VTMSIDTSKNQFSLTLSSLTAADTAVYFCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 715
13876_HC [hu anti-<huCDH19> 16A4.1 VH]::huIgG1z
QVQLQESGPGLAKPSETLSLTCTVSGDSITSYYWSWIRQPPGKGLEWIGYIYYSGSTNYNPSLKSR
VTISVDTSKNQFSLKLSSVTAADTAVYYCARDQRRIAAAGTHFYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 716
13877_HC [hu anti-<huCDH19> 22G10.1 VH]::huIgG1z
EVQLLESGGGLVQPGGSLRLSCAASGFTFSSYAMNWVRQAPGKGLEWVSTISGGGANTYYADSVKG
RFTISSDNSKSTLYLQMNSLRAADTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 717
13878_HC [hu anti-<huCDH19> 20D3.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 718
13879_HC [hu anti-<huCDH19> 22D1.1 VH]::huIgG1z
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 719
13880_HC [hu anti-huCDH19> 25F8.1 VH]::huIgG1z
QVQLVQSGAEVKKPGASVKVSCKASGYTFTSYYIHWVRQAPGQGLEWMGIINPSGGSTRYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 720
13881_HC [hu anti-<huCDH19> 26F12.1 VH]::huIgG1z
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 721
13882_HC [hu anti-<huCDH19> 26D1.1 VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 722
13883_HC [hu anti-<huCDH19> 25G10.1 VH]::huIgG1z
QVQLQESGPGLVKPSETLSLTCTVSGGSISGYYWSWIRQPPGKGLEWIGYIYYIGSTNYNPSLKSR
VTMSVDTSKNQFSLKLSSVTAADTAVYYCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 723
13885_HC [hu anti-<huCDH19> 19B5.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARCiGIQLWLHLDYWGQGTLVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 724
14022_HC [hu anti-<huCDH19> 4A2 VH]::huIgG1z
QVQLQESGPGLVKPSQTLSLTCTVSGGSISSSGYYWSWIRQHPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 725
14024_HC [hu anti-<huCDH19> 4A2 (1-472)(Q17E,H47P) VH]::huIgG1z
QVQLQESGPGLVKPSETLSLTCTVSGGSISSSGYYWSWIRQPPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 726
14025_HC [hu anti-<huCDH19> 4A2 VH]::huIgG1z
QVQLQESGPGLVKPSQTLSLTCTVSGGSISSSGYYWSWIRQHPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 727
14026_HC [hu anti-<huCDH19> 4A2 (1-472)(Q17E,H47P) VH]::huIgG1z
QVQLQESGPGLVKPSETLSLTCTVSGGSISSSGYYWSWIRQPPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 728
14027_HC [hu anti-<huCDH19> 4A2 (1-472)
(Q17E,H47P,D111E) VH]::huIgG1z
QVQLQESGPGLVKPSETLSLTCTVSGGSISSSGYYWSWIRQPPGKGLEWIGYIYYTGSAYYNPSLK
SRVTISVDTSKNQFSLKLSSVTAADTAVYYCAREGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 729
14028_HC [hu anti-<huCDH19> 4A2 (1-472)
(Q17E,H47P,D111E,W134Y) VH]::huIgG1z
QVQLQESGPGLVKPSETLSLTCTVSGGSISSSGYYWSWIRQPPGKGLEWIGYIYYTGSAYYNPSLK
SRVTISVDTSKNQFSLKLSSVTAADTAVYYCAREGSSGYYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 730
14029_HC [hu anti-<huCDH19> 4A2 VH]::huIgG1z
QVQLQESGPGLVKPSQTLSLTCTVSGGSISSSGYYWSWIRQHPGKGLEWIGYIYYTGSAYYNPSLK
SRVTISVDTSKNQFSLKLSSVTAADTAVYYCARDGSSGWYFQYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 731
14030_HC [hu anti-<huCDH19> 4F3 (1-471)(R17G) VH]::huIgG1z
QVQLVESGGGVVQPGGSLRLSCAASGFSFSSYDMDWVRQTPGKGLEWVAVIWYDGSNKYYADSVRG
RFTISRDNSKNTLFLQMNSLRVEDTAVYYCARETGEGWYFDLWGRGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 732
14031_HC [hu anti-<huCDH19> 4F3 (1-471)(R17G,T47A) VH]::huIgG1z
QVQLVESGGGVVQPGGSLRLSCAASGFSFSSYDMDWVRQAPGKGLEWVAVIWYDGSNKYYADSVRG
RFTISRDNSKNTLFLQMNSLRVEDTAVYYCARETGEGWYFDLWGRGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 733
14032_HC [hu anti-<huCDH19> 4F3 (1-471)
(R17G,T47A,R141Q) VH]::huIgG1z
QVQLVESGGGVVQPGGSLRLSCAASGFSFSSYDMDWVRQAPGKGLEWVAVIWYDGSNKYYADSVRG
RFTISRDNSKNTLFLQMNSLRVEDTAVYYCARETGEGWYFDLWGQGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 734
14033_HC [hu anti-<huCDH19> 4F3 (1-471)
(R17G,T47A,D61E,D72E,R141Q) VH]::huIgG1z
QVQLVESGGGVVQPGGSLRLSCAASGFSFSSYDMDWVRQAPGKGLEWVAVIWYEGSNKYYAESVRG
RFTISRDNSKNTLFLQMNSLRVEDTAVYYCARETGEGWYFDLWGQGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 735
14034_HC [hu anti-<huCDH19> 4F3 (1-471)
(R17G,T47A,D61E,D72E,W134Y,R141Q) VH]::huIgG1z
QVQLVESGGGVVQPGGSLRLSCAASGFSFSSYDMDWVRQAPGKGLEWVAVIWYEGSNKYYAESVRG
RFTISRDNSKNTLFLQMNSLRVEDTAVYYCARETGEGYYFDLWGQGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 736
14039_HC [hu anti-<huCDH19> 2G6 (1-477)
(R17G,D61E,D72E,K94N) VH]::huIgG1z
QVQLVESGGGVVQPGGSLRLSCAASGFTFSSYGMHWVRQAPGKGLEWVAFIWYEGSNKYYAESVKD
RFTISRDNSKNTLYLQMNSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 737
14040_HC [hu anti-<huCDH19> 16C1.1 VH]::huIgG1z
QVQLQESGPGLVKPSETLSLTCTVSGGSISGYYWSWIRQPPGKGLEWIGYIYYIGSTNYNPSLKSR
VTMSIDTSKNQFSLTLSSLTAADTAVYFCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 738
14041_HC [hu anti-<huCDH19> 16C1.1 (1-469)(T92K) VH]::huIgG1z
QVQLQESGPGLVKPSETLSLTCTVSGGSISGYYWSWIRQPPGKGLEWIGYIYYIGSTNYNPSLKSR
VTMSIDTSKNQFSLKLSSLTAADTAVYFCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 739
14042_HC [hu anti-<huCDH19> 16C1.1 (1-469)
(T92K,D109E) VH]::huIgG1z
QVQLQESGPGLVKPSETLSLTCTVSGGSISGYYWSWIRQPPGKGLEWIGYIYYIGSTNYNPSLKSR
VTMSIDTSKNQFSLKLSSLTAADTAVYFCAREGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 740
14043_HC [hu anti-<huCDH19> 16C1.1 (1-469)
(T92K,W132Y,W135Y) VH]::huIgG1z
QVQLQESGPGLVKPSETLSLTCTVSGGSISGYYWSWIRQPPGKGLEWIGYIYYIGSTNYNPSLKSR
VTMSIDTSKNQFSLKLSSLTAADTAVYFCARDGSSGYYRYFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 741
14044_HC [hu anti-<huCDH19> 16C1.1 (1-469)(T92K) VH]::huIgG1z
QVQLQESGPGLVKPSETLSLTCTVSGGSISGYYWSWIRQPPGKGLEWIGYIYYIGSTNYNPSLKSR
VTMSIDTSKNQFSLKLSSLTAADTAVYFCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 742
14045_HC [hu anti-<huCDH19> 17H8.2 VH]::huIgG1z
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARDSRYRSGWYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 743
14046_HC [hu anti-<huCDH19> 17H8.2 (1-471)(D109E) VH]::huIgG1z
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARESRYRSGWYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 744
14047_HC [hu anti-<huCDH19> 17H8.2 (1-471)
(D109E,W132Y) VH]::huIgG1z
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARESRYRSGYYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 745
14048_HC [hu anti-<huCDH19> 17H8.2 (1-471)(D109E) VH]::huIgG1z
QVQLQESGPGLVKPSETLSLTCTVSGGSINSYYWSWIRQPPGKGLEWIGYIYYIGSTNYNPSLKSR
VTISVDTSKNQFSLKLSSVTAADTALYYCARESRYRSGWYDAFDIWGQGTMVTVSSASTKGPSVFP
LAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 746
14049_HC [hu anti-<huCDH19> 4F7 VH]::huIgG1z
QVQLQESGPGLVKPSETLSLTCTVSGGSISSYSWSWIRQPPGKGLEWIGYIYYSGSTNYNPSLKSR
VTISLDTSKNQFSLKLSSVTAADTAVYYCARNWAFHFDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 747
14050_HC [hu anti-<huCDH19> 4F7 VH]::huIgG1z
QVQLQESGPGLVKPSETESLTCTVSGGSISSYSWSWIRQPPGKGLEWIGYIYYSGSTNYNPSLKSR
VTISLDTSKNQFSLKLSSVTAADTAVYYCARNWAFHFDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 748
14051_HC [hu anti-<huCDH19> 4F7 (1-468)(W113Y) VH]::huIgG1z
QVQLQESGPGLVKPSETLSLTCTVSGGSISSYSWSWIRQPPGKGLEWIGYIYYSGSTNYNPSLKSR
VTISLDTSKNQFSLKLSSVTAADTAVYYCARNYAFHFDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 749
14052_HC [hu anti-<huCDH19> 4B10 (1-471)
(R17G,D61E,D72E,W134Y) VH]::huIgG1z
QVQLVESGGGVVQPGGSLRLSCAASGFTFSSYDMHWVRQAPGKGLEWVAVISYEGTNEYYAESVKG
RFTISRDTSKNTLYLQMNSLRAEDTAVYYCARERYFDYSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 750
14053_HC [hu anti-<huCDH19> 4B10 VH]::huIgG1z
QVQLVESGGGVVQPGRSLRLSCAASGFTFSSYDMHWVRQAPGKGLEWVAVISYDGTNEYYADSVKG
RFTISRDTSKNTLYLQMNSLRAEDTAVYYCARERYFDWSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 751
14054_HC [hu anti-<huCDH19> 4B10 (1-471)(R17G) VH]::huIgG1z
QVQLVESGGGVVQPGGSLRLSCAASGFTFSSYDMHWVRQAPGKGLEWVAVISYDGTNEYYADSVKG
RFTISRDTSKNTLYLQMNSLRAEDTAVYYCARERYFDWSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 752
14055_HC [hu anti-<huCDH19> 4B10 (1-471)
(R17G,D61E,D72E) VH]::huIgG1z
QVQLVESGGGVVQPGGSLRLSCAASGFTFSSYDMHWVRQAPGKGLEWVAVISYEGTNEYYAESVKG
RFTISRDTSKNTLYLQMNSLRAEDTAVYYCARERYFDWSFDYWGQGTLVSVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 753
14056_HC [hu anti-<huCDH19> 4A9 VH]::huIgG1z
QVQLQESGPGLVKPSETLSLTCTVSGGSISGYYWSWIRQPPGKGLEWFAYFSYSGSTNYNPSLKSR
VTLSVDTSKNQFSLKLSSVTAADTAVYYCARNWAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 754
14057_HC [hu anti-<huCDH19> 4A9 (1-468)(F55I,A56G) VH]::huIgG1z
QVQLQESGPGLVKPSETLSLTCTVSGGSISGYYWSWIRQPPGKGLEWIGYFSYSGSTNYNPSLKSR
VTLSVDTSKNQFSLKLSSVTAADTAVYYCARNWAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 755
14058_HC [hu anti-<huCDH19> 4A9 (1-468)(F55I,A56G) VH]::huIgG1z
QVQLQESGPGLVKPSETLSLTCTVSGGSISGYYWSWIRQPPGKGLEWIGYFSYSGSTNYNPSLKSR
VTLSVDTSKNQFSLKLSSVTAADTAVYYCARNWAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 756
14059_HC [hu anti-<huCDH19> 4A9 (1-468)
(F55I,A56G,W113Y) VH]::huIgG1z
QVQLQESGPGLVKPSETLSLTCTVSGGSISGYYWSWIRQPPGKGLEWIGYFSYSGSTNYNPSLKSR
VTLSVDTSKNQFSLKLSSVTAADTAVYYCARNYAFHFDFWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 757
14060_HC [hu anti-<huCDH19> 20D3.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 758
14061_HC [hu anti-<huCDH19> 20D3.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 759
14062_HC [hu anti-<huCDH19> 20D3.1 (1-469)(W133Y) VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSERSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 760
14063_HC [hu anti-<huCDH19> 20D3.1 (1-469)(W133Y) VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 761
14064_HC [hu anti-<huCDH19> 20D3.1 (1-469)(W133Y) VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 762
14065_HC [hu anti-<huCDH19> 22G10.1 (1-470)
(S82R,A99E) VH]::huIgG1z
EVQLLESGGGLVQPGGSLRLSCAASGFTFSSYAMNWVRQAPGKGLEWVSTISGGGANTYYADSVKG
RFTISRDNSKSTLYLQMNSLRAEDTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 763
14066_HC [hu anti-<huCDH19> 22G10.1 (1-470)
(A99E,H105Y) VH]::huIgG1z
EVQLLESGGGLVQPGGSLRLSCAASGFTFSSYAMNWVRQAPGKGLEWVSTISGGGANTYYADSVKG
RFTISSDNSKSTLYLQMNSLRAEDTAVYYCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 764
14067_HC [hu anti-<huCDH19> 22G10.1 (1-470)(A99E) VH]::huIgG1z
EVQLLESGGGLVQPGGSLRLSCAASGFTFSSYAMNWVRQAPGKGLEWVSTISGGGANTYYADSVKG
RFTISSDNSKSTLYLQMNSLRAEDTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 765
14068_HC [hu anti-<huCDH19> 22G10.1 (1-470)(A99E) VH]::huIgG1z
EVQLLESGGGLVQPGGSLRLSCAASGFTFSSYAMNWVRQAPGKGLEWVSTISGGGANTYYADSVKG
RFTISSDNSKSTLYLQMNSLRAEDTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 766
14069_HC [hu anti-<huCDH19> 22G10.1 (1-470)
(D72E,A99E) VH]::huIgG1z
EVQLLESGGGLVQPGGSLRLSCAASGFTFSSYAMNWVRQAPGKGLEWVSTISGGGANTYYAESVKG
RFTISSDNSKSTLYLQMNSLRAEDTAVYHCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 767
14070_HC [hu anti-<huCDH19> 22G10.1 (1-470)(H105Y) VH]::huIgG1z
EVQLLESGGGLVQPGGSLRLSCAASGFTFSSYAMNWVRQAPGKGLEWVSTISGGGANTYYADSVKG
RFTISSDNSKSTLYLQMNSLRAADTAVYYCAKGGMGGYYYGMDVWGQGTTVTVSSASTKGPSVFPL
APSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 768
14071_HC [hu anti-<huCDH19> 16A4.1 (1-474)(T144L) VH]::huIgG1z
QVQLQESGPGLAKPSETLSLTCTVSGDSITSYYWSWIRQPPGKGLEWIGYIYYSGSTNYNPSLKSR
VTISVDTSKNQFSLKLSSVTAADTAVYYCARDQRRIAAAGTHFYGMDVWGQGTLVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 769
14072_HC [hu anti-<huCDH19> 19B5.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 770
14073_HC [hu anti-<huCDH19> 19B5.1 (1-469)(W133Y) VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLYLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 771
14074_HC [hu anti-<huCDH19> 19B5.1 VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 772
14075_HC [hu anti-<huCDH19> 19B5.1 VH]::huIgG1z
QVQLVQSGAEVKKPGASVKVSCKVSGYTFTSYFIHWVRQAPGQGLEWMGIMPISVSTSYAQKFQGR
VTMTRDTSTSTVFMELSSLRSEDTAVYYCARGGIQLWLHLDYWGQGTLVTVSSASTKGPSVFPLAP
SSKSTSGGTAALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK
SEQ ID NO: 773
14076_HC [hu anti-<huCDH19> 19B5.1 (1-469)(W133Y) VH]::huIgG1z
QVQLVQSGAEVKKPGASVKVSCKVSGYTFTSYFIHWVRQAPGQGLEWMGIINPISVSTSYAQKFQG
RVTMTRDTSTSTVFMELSSLRSEDTAVYYCARGGIQLYLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 774
14077_HC [hu anti-<huCDH19> 23A10.3 (1-474)(L92Q) VH]::huIgG1z
QVQLVESGGGVVQPGRSLRLSCAASGFTFSRYGIHWVRQAPGKGLEWVAVIWYDGSNKYYADSVKG
RFTISRDNSKNTLYLQMNSLRAEDSAVYYCARRAGIPGTTGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 775
14078_HC [hu anti-<huCDH19> 23A10.3 (1-474)
(R17G,L92Q) VH]::huIgG1z
QVQLVESGGGVVQPGGSLRLSCAASGFTFSRYGIHWVRQAPGKGLEWVAVIWYDGSNKYYADSVKG
RFTISRDNSKNTLYLQMNSLRAEDSAVYYCARRAGIPGTTGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 776
14079_HC [hu anti-<huCDH19> 23A10.3 (1-474)
(R17G,D61E,D72E,L92Q) VH]::huIgG1z
QVQLVESGGGVVQPGGSLRLSCAASGFTFSRYGIHWVRQAPGKGLEWVAVIWYEGSNKYYAESVKG
RFTISRDNSKNTLYLQMNSLRAEDSAVYYCARRAGIPGTTGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 777
14080_HC [hu anti-<huCDH19> 23A10.3 VH]::huIgG1z
QVQLVESGGGVVQPGRSLRLSCAASGFTFSRYGIHWVRQAPGKGLEWVAVIWYDGSNKYYADSVKG
RFTISRDNSKNTLYLLMNSLRAEDSAVYYCARRAGIPGTTGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 778
14081_HC [hu anti-<huCDH19> 25G10.1 VH]::huIgG1z
QVQLQESGPGLVKPSETLSLTCTVSGGSISGYYWSWIRQPPGKGLEWIGYIYYIGSTNYNPSLKSR
VTMSVDTSKNQFSLKLSSVTAADTAVYYCARDGSSGWYRWFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 779
14082_HC [hu anti-<huCDH19> 25G10.1 (1-469)
(D109E,W132Y,W135Y) VH]::huIgG1z
QVQLQESGPGLVKPSETLSLTCTVSGGSISGYYWSWIRQPPGKGLEWIGYIYYIGSTNYNPSLKSR
VTMSVDTSKNQFSLKLSSVTAADTAVYYCAREGSSGYYRYFDPWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 780
14083_HC [hu anti-<huCDH19> 26D1.1 VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 781
14084_HC [hu anti-<huCDH19> 26D1.1 VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 782
14085_HC [hu anti-<huCDH19> 26D1.1 VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 783
14086_HC [hu anti-<huCDH19> 26D1.1 VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 784
14087_HC [hu anti-<huCDH19> 26D1.1 (1-469)(W133Y) VH]::huIgG1z
QVQLVQSGAEVKKPGASVKVSCKASRYTFTSYYMSWVRQAPGQGLEWMGIIHPSGGDTTYAQKFQG
RVTMTGDTSTSTVYMELSSLRSEDTAVYYCARGGIKLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 785
14088_HC [hu anti-<huCDH19> 26D1.1 (1-469)
(R27G,G82R) VH]::huIgG1z
QVQLVQSGAEVKKPGASVKVSCKASGYTFTSYYMSWVRQAPGQGLEWMGIIHPSGGDTTYAQKFQG
RVTMTRDTSTSTVYMELSSLRSEDTAVYYCARGGIKLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 786
14089_HC [hu anti-<huCDH19> 26F12.1 VH]::huIgG1z
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 787
14090_HC [hu anti-<huCDH19> 26F12.1 VH]::huIgG1z
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 788
14091_HC [hu anti-<huCDH19> 26F12.1 (1-469)(W133Y) VH]::huIgG1z
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 789
14092_HC [hu anti-<huCDH19> 26F12.1 (1-469)(W133Y) VH]::huIgG1z
QVQLVQSGAEVKKPGASVKVSCKASRYTFTNYYMSWVRQAPGQGLEWMGIINPSGGDSTYAQKFQG
RLTMTGDTSTSTVYMELSSLRSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 790
14093_HC [hu anti-<huCDH19> 25F8.1 VH]::huIgG1z
QVQLVQSGAEVKKPGASVKVSCKASGYTFTSYYIHWVRQAPGQGLEWMGIINPSGGSTRYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 791
14094_HC [hu anti-<huCDH19> 25F8.1 VH]::huIgG1z
QVQLVQSGAEVKKPGASVKVSCKASGYTFTSYYIHWVRQAPGQGLEWMGIINPSGGSTRYAQKFQG
RVTMTRDTSTSTVFMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 792
14095_HC [hu anti-<huCDH19> 25F8.1 (1-469)(F90Y) VH]::huIgG1z
QVQLVQSGAEVKKPGASVKVSCKASGYTFTSYYIHWVRQAPGQGLEWMGIINPSGGSTRYAQKFQG
RVTMTRDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 793
14096_HC [hu anti-<huCDH19> 25F8.1 (1-469)(F90Y) VH]::huIgG1z
QVQLVQSGAEVKKPGASVKVSCKASGYTFTSYYIHWVRQAPGQGLEWMGIINPSGGSTRYAQKFQG
RVTMTRDTSTSTVYMELSSLRSEDTAVYYCARGGIQLWLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 794
14097_HC [hu anti-<huCDH19> 25F8.1 (1-469)
(F90Y,W133Y) VH]::huIgG1z
QVQLVQSGAEVKKPGASVKVSCKASGYTFTSYYIHWVRQAPGQGLEWMGIINPSGGSTRYAQKFQG
RVTMTRDTSTSTVYMELSSLRSEDTAVYYCARGGIQLYLHFDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 795
14098_HC [hu anti-<huCDH19> 22D1.1 VH]::huIgG1z
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 796
14099_HC [hu anti-<huCDH19> 22D1.1 VH]::huIgG1z
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 797
14100_HC [hu anti-<huCDH19> 22D1.1 (1-469)(W133Y) VH]::huIgG1z
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLYLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 798
14101_HC [hu anti-<huCDH19> 22D1.1 (1-469)(W133Y) VH]::huIgG1z
QVQLVQSGAEVKKPGASVRVSCKVSGYTFTSYFIHWVRQAPGQGLEWMGIINPISVSTSYAQKFQG
RVTMTRDTSTSTVFMELSSLRSEDTAVYYCARGGIQLYLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 799
14102_HC [hu anti-<huCDH19> 22D1.1 (1-469)(F90Y) VH]::huIgG1z
QVQLVQSGAEVKKPGASVRVSCKVSGYTFTSYFIHWVRQAPGQGLEWMGIINPISVSTSYAQKFQG
RVTMTRDTSTSTVYMELSSLRSEDTAVYYCARGGIQLWLHLDYWGQGTLVTVSSASTKGPSVFPLA
PSSKSTSGGTAALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 800
13591_HC [hu anti-<huCDH19> 4F7 VH]::huIgG1z
QVQLQESGPGLVKPSETLSLTCTVSGGSISSYSWSWIRQPPGKGLEWIGYIYYSGSTNYNPSLKSR
VTISLDTSKNQFSLKLSSVTAADTAVYYCARNWAFHFDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 801
14301_HC [hu anti-<huCDH19> 2G6 VH]::huIgG1z
QVQLVESGGGVVQPGRSLRLSCAASGFTFSSYGMHWVRQAPGKGLEWVAFIWYDGSNKYYADSVKD
RFTISRDNSKNTLYLQMKSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 802
14302_HC [hu anti-<huCDH19> 2G6 (1-477)(R17G,K94N) VH]::huIgG1z
QVQLVESGGGVVQPGGSLRLSCAASGFTFSSYGMHWVRQAPGKGLEWVAFIWYDGSNKYYADSVKD
RFTISRDNSKNTLYLQMNSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 803
14303_HC [hu anti-<huCDH19> 2G6 (1-477)(D61E,D72E) VH]::huIgG1z
QVQLVESGGGVVQPGRSLRLSCAASGFTFSSYGMHWVRQAPGKGLEWVAFIWYEGSNKYYAESVKD
RFTISRDNSKNTLYLQMKSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 804
14304_HC [hu anti-<huCDH19> 2G6 (1-477)(R17G) VH]::huIgG1z
QVQLVESGGGVVQPGGSLRLSCAASGFTFSSYGMHWVRQAPGKGLEWVAFIWYDGSNKYYADSVKD
RFTISRDNSKNTLYLQMKSLRAEDTAVYYCARRAGIIGTIGYYYGMDVWGQGTTVTVSSASTKGPS
VFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 805

TABLE IIId
Light Chain Variable and Contant Region
Polynucleotide and Amino
acid Sequences

13586 LC [hu anti-<huCDH19> 4F3
VL]::huKLC
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 806
13589 LC [hu anti-<huCDH19> 4A9
VL]::huLLC-C1
QSVLTQPPSVSGAPGQRVTISCTGSSSNIGTGYAV
HWYQQFPGTAPKLLIYGNNNRPSGVPDRFSGSKSG
TSASLAITGLQAEDEADYYCQSYDSRLSGWVFGGG
TKLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 807
13590 LC [hu anti-<huCDH19> 4B10
VL]::huKLC
EIVLTQSPGTLSLSPGERATLSCRASQSVSNTYLA
WYHQRPGQAPRLLIYGASSRATGIPDRFSGSGSGT
DFALTISSLEPEDFAVYYCQQYSNSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 808
13874 LC [hu anti-<huCDH19> 17118.2
VL]::huKLC
DIVLTQSPGTLSLSPGERATLSCRASQSVAGSYLA
WYQQKPGQAPRLLISGASSRATGIPDRFSGSGSGT
DFTLTISRLEPEDFAVYYCQQYGKSPITFGQGTRL
EMKGTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 809
13875 LC [hu anti-<huCDH19> 16C1.1
VL]::huKLC
EIVLTQSPGTLSLSPGERATLSCRASQSVSSSYLA
WYQQKPGQAPRLLIFGASSRATGIPDRFSGSGSGT
DFTLTISGLEPEDFAVYHCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 810
13876 LC [hu anti-<huCDH19> 16A4.1
VL]::huKLC
EIVLTQSPGTLSLSPGERATLSCRASQSVSSSYLA
WYQQKPGQAPRLLIYGTSSRATGIPDRFSGSGSGT
DFTLTISRLEPEDFAVYYCQQYGSSPFTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 811
13877 LC [hu anti-<huCDH19> 22G10.1
VL]::huKLC
EIVMTQSPVTLSLSLGERATLSCRASQSISSNLAW
FQQKPGQAPRLLIYGAFTRATGIPARVSGSGSGTE
FTLTISSLQSEDFAVYYCQQYNYWPLTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 812
13878 LC [hu anti-<huCDH19> 20D3.1
VL]::huLLC-C2
QSALTQPPSATGTPGQRVTISCSGSSSNIGSNFVN
WYKQLPGTAPKVLIYTNNQRPSGVPDRFSGSKSGT
SASLAISGLQSEDESDYYCATWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 813
13879 LC [hu anti-<huCDH19> 22D1.1
VL]::huLLC-C2
QSALTQPPSATGTPGQRVTISCSGSSSNIGSNFVN
WYKQLPGTAPKVLIYTNNQRPSGVPDRFSGSKSGT
SASLAISGLQSEDESDYYCATWDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 814
13880 LC [hu anti-<huCDH19> 25F8.1
VL]::huLLC-C2
QSALTQPPSATGTPGQRVTISCSGSSSNIGRNFVN
WYKQLPGTAPKVLIYTNNQRPSGVPDRFSGSKSGT
SASLAISGLQSEDESDYYCAAWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 815
13881 LC [hu anti-<huCDH19> 26F12.1
VL]::huLLC-C2
QSVLTQSPSASGTPGQKVTISCSGSRSNIGSNFVN
WYQQLPGTAPKLLIYTNYQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 816
13882 LC [hu anti-<huCDH19> 26D1.1
VL]::huLLC-C2
HSVLTQSPSASGTPGQRVTISCSGSRSNIGSNFVN
WYQQLPGTAPKLLIYTNNQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 817
13883 LC [hu anti-<huCDH19> 25G10.1
VL]::huKLC
EIVLTQSPGTLSLSPGERATLSCRASQSVSSSYLA
WYQQKPGQAPRLLIFGASSRATGIPDRFSGSGSGT
DFTLTISRLEPEDFAVYHCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 818
13885 LC [hu anti-<huCDH19> 19B5.1
VL]::huLLC-C2
QSALTQPPSTTGTPGQRVTISCSGSRSNIGSNFVN
WYKQLPGTAPKVLIYTNNQRPSGVPDRFSGSKSGT
SASLAISGLQSEDESDYYCATWDDSMNGWVFGGGT
ICLTVLGQPICAAPSVTLFPPSSEELQANKATLVC
LISDFYPGAVTVAWKADSSPVKAGVETTTPSKQSN
NKYAASSYLSLTPEQWKSHRSYSCQVTHEGSTVEK
TVAPTECS
SEQ ID NO: 819
14022 LC [hu anti-<huCDH19> 4A2
(1-236)(N30Q) VL]::huKLC
EIVLTQSPGTLSLSPGERATLSCRASRQISSSYLAWY
QQKPGQAPRLLIYGPSSRATGIPDRFSGSGSGTDFTL
TISRLEPEDFTVYYCQQYGSSFTFGPGTKVDIKRT
VAAPSVFIFPPSDEQLKSGTASVVCLLNNFYPREA
KVQWKVDNALQSGNSQESVTEQDSKDSTYSLSSTL
TLSKADYEKHKVYACEVTHQGLSSPVTKSFNRGEC
SEQ ID NO: 820
14024 LC [hu anti-<huCDH19> 4A2
(1-236)(N30Q, T102A, P141Q)
VL]::huKLC
EIVLTQSPGTLSLSPGERATLSCRASRQISSSYLA
WYQQKPGQAPRLLIYGPSSRATGIPDRFSGSGSGT
DFTLTISRLEPEDFAVYYCQQYGSSFTFGQGTKVD
IKRTVAAPSVFIFPPSDEQLKSGTASVVCLLNNFY
PREAKVQWKVDNALQSGNSQESVTEQDSKDSTYSL
SSTLTLSICADYEKHKVYACEVTHQGLSSPVTKSF
NRGEC
SEQ ID NO: 821
14025 LC [hu anti-<huCDH19> 4A2
(1-236)(N30Q, T102A) VL]::huKLC
EIVLTQSPGTLSLSPGERATLSCRASRQISSSYLA
WYQQKPGQAPRLLIYGPSSRATGIPDRFSGSGSGT
DFTLTISRLEPEDFAVYYCQQYGSSFTFGPGTKVD
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 822
14026 LC [hu anti-<huCDH19> 4A2
(1-236)(N30Q, T102A) VL]::huKLC
EIVLTQSPGTLSLSPGERATLSCRASRQISSSYLA
WYQQKPGQAPRLLIYGPSSRATGIPDRFSGSGSGT
DFTLTISRLEPEDFAVYYCQQYGSSFTFGPGTKVD
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 823
14027 LC [hu anti-<huCDH19> 4A2
(1-236)(N30Q, T102A, P141Q) VL]::huKLC
EIVLTQSPGTLSLSPGERATLSCRASRQISSSYLA
WYQQKPGQAPRLLIYGPSSRATGIPDRFSGSGSGT
DFTLTISRLEPEDFAVYYCQQYGSSFTFGQGTKVD
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 824
14028 LC [hu anti-<huCDH19> 4A2
(1-236)(N30Q, T102A, P141Q) VL]::huKLC
EIVLTQSPGTLSLSPGERATLSCRASRQISSSYLAW
YQQKPGQAPRLLIYGPSSRATGIPDRFSGSGSGTD
FTLTISRLEPEDFAVYYCQQYGSSFTFGQGTKVDI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC
SEQ ID NO: 825
14029 LC [hu anti-<huCDH19> 4A2
(1-236)(R290, N30S) VL]::huKLC
EIVLTQSPGTLSLSPGERATLSCRASQSISSSYLA
WYQQKPGQAPRLLIYGPSSRATGIPDRFSGSGSGT
DFTLTISRLEPEDFTVYYCQQYGSSFTFGPGTKVD
IKRTVAAPSVFIFPPSDEQLKSGTASVVCLLNNFY
PREAKVQWKVDNALQSONSQESVTEQDSKDSTYSL
SSTLTLSKADYEKHKVYACEVTHQGLSSPVTKSFN
RGEC
SEQ ID NO: 826
14030 LC [hu anti-<huCDH19> 4F3
VL]::huKLC
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKEKVYACEVTHQGLSSPVTKSFN
RGEC
SEQ ID NO: 827
14031 LC [hu anti-<huCDH19> 4F3
VL]::huKLC
EIVLTQSPGTLSLSPGERATLSCRASQSVSSSYLA
WYQQICPGQAPRLLIYGASSRATGIPDRFSGSGSG
TDFTLTISRLEPEDFAVYYCQQYGSSWTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 828
14032 LC [hu anti-<huCDH19> 4F3
VL]::huKLC
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSWTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC
SEQ ID NO: 829
14033 LC [hu anti-<huCDH19> 4F3
VL]::huKLC
EIVLTQSPGTLSLSPGERATLSCRASQSVSSSYLAWY
QQKPGQAPRLLIYGASSRATGIPDRFSGSGSGTDFTL
TISRLEPEDFAVYYCQQYGSSWTFGQGTKVEIKRT
VAAPSVFIFPPSDEQLKSGTASVVCLLNNFYPREA
KVQWKVDNALQSONSQESVTEQDSKDSTYSLSSTL
TLSKADYEKHKVYACEVTHQGLSSPVTKSFNRGEC
SEQ ID NO: 830
14034 LC [hu anti-<huCDH19> 4F3
VL]::huKLC
EIVLTQSPGTLSLSPGERATLSCRASQSVSSSYLA
WYQQKPGQAPRLLIYGASSRATGIPDRFSGSGSGT
DFTLTISRLEPEDFAVYYCQQYGSSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 831
14039 LC [hu anti-<huCDH19> 2G6
(1-234)(C42S, D110E) VL]::huLLC-C1
SYELTQPPSVSVSPGQTASITCSGDRLGEKYTSWY
QQRPGQSPLLVIYQDTKRPSGIPERFSGSNSGNTA
TLTISGTQAMDEADYYCQAWESSTVVFGGGTKLTV
LGQPKANPTVTLFPPSSEELQANKATLVCLISDFY
PGAVTVAWKADGSPVKAGVETTKPSKQSNNKYAAS
SYLSLTPEQWKSHRSYSCQVTHEGSTVEKTVAPTE
CS
SEQ ID NO: 832
14040 LC [hu anti-<huCDH19> 16C1.1
(1-235)(H105Y) VL]::huKLC
EIVLTQSPGTLSLSPGERATLSCRASQSVSSSYLA
WYQQKPGQAPRLLIFGASSRATGIPDRFSGSGSGT
DFTLTISGLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 833
14041 LC [hu anti-<huCDH19> 16C1.1
(1-235)(H105Y) VL]::hul(LC
EIVLTQSPGTLSLSPGERATLSCRASQSVSSSYLA
WYQQKPGQAPRLLIFGASSRATGIPDRFSGSGSGT
DFTLTISGLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 834
14042 LC [hu anti-<huCDH19> 16C1.1
(1-235)(H105Y) VL]::hul(LC
EIVLTQSPGTLSLSPGERATLSCRASQSVSSSYLA
WYQQKPGQAPRLLIFGASSRATGIPDRFSGSGSGT
DFTLTISGLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 835
14043 LC [hu anti-<huCDH19> 16C1.1
(1-235)(H105Y) VL]::huKLC
EIVLTQSPGTLSLSPGERATLSCRASQSVSSSYLA
WYQQKPGQAPRLLIFGASSRATGIPDRFSGSGSGT
DFTLTISGLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 836
14044 LC [hu anti-<huCDH19> 16C1.1
(1-235)(G95R, H105Y, G141Q)
VL]::hul(LC
EIVLTQSPGTLSLSPGERATLSCRASQSVSSSYLA
WYQQKPGQAPRLLIFGASSRATGIPDRFSGSGSGT
DFTLTISRLEPEDFAVYYCQQYGNSPLTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 837
14045 LC [hu anti-<huCDH19> 17H8.2
(1-235)(G149R) VL]::huKLC
DIVLTQSPGTLSLSPGERATLSCRASQSVAGSYLA
WYQQKPGQAPRLLISGASSRATGIPDRFSGSGSGT
DFTLTISRLEPEDFAVYYCQQYGKSPITFGQGTRL
EMKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 838
14046 LC [hu anti-<huCDH19> 17H8.2
(1-235)(G149R) VL]::huKLC
DIVLTQSPGTLSLSPGERATLSCRASQSVAGSYLA
WYQQKPGQAPRLLISGASSRATGIPDRFSGSGSGT
DFTLTISRLEPEDFAVYYCQQYGKSPITFGQGTRL
EMKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 839
14047 LC [hu anti-<huCDH19> 17118.2
(1-235)(G149R) VL]::huKLC
DIVLTQSPGTLSLSPGERATLSCRASQSVAGSYLA
WYQQKPGQAPRLLISGASSRATGIPDRFSGSGSGT
DFTLTISRLEPEDFAVYYCQQYGKSPITFGQGTRL
EMKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 840
14048 LC [hu anti-<huCDH19> 17118.2
(1-235)(S57Y, G149R) VL]::huKLC
DIVLTQSPOTLSLSPGERATLSCRASQSVAGSYLA
WYQQKPGQAPRLLIYGASSRATGIPDRFSGSGSGT
DFTLTISRLEPEDFAVYYCQQYGKSPITFGQGTRL
EMKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 841
14049 LC [hu anti-<huCDH19> 4F7
(1-239)(H57Y) VL]::huLLC-C2
QSVLTQPPSVSGAPGQRVTISCTGSSSNIGTGYDV
HWYQQLPGTAPKLLIYGNSNRPSGVPDRFSGSKSG
TSASLAITGLQAEDEADYYCQSYDSSLSGWVFGGG
TRLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 842
14050 LC [hu anti-<huCDH19> 4F7
(1-239)(1157Y, D110E) VL]::huLLC-C2
QSVLTQPPSVSGAPGQRVTISCTGSSSNIGTGYDV
HWYQQLPGTAPKLLIYGNSNRPSGVPDRFSGSKSG
TSASLAITGLQAEDEADYYCQSYESSLSGWVFGGG
TRLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 843
14051 LC [hu anti-<huCDH19> 4F7
(1-239)(D110E) VL]::huLLC-C2
QSVLTQPPSVSGAPGQRVTISCTGSSSNIGTGYDV
HWYQQLPGTAPKLLIHGNSNRPSGVPDRFSGSKSG
TSASLAITGLQAEDEADYYCQSYESSLSGWVFGGG
TRLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 844
14052 LC [hu anti-<huCDH19> 4B10
(1-236)(H45Q, A90T) VL]::huKLC
EIVLTQSPGTLSLSPGERATLSCRASQSVSNTYLA
WYQQRPGQAPRLLIYGASSRATGIPDRFSGSGSGT
DFTLTISSLEPEDFAVYYCQQYSNSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 845
14053 LC [hu anti-<huCDH19> 4B10
(1-236)(11450, A90T) VL]::huKLC
EIVLTQSPOTLSLSPGERATLSCRASQSVSNTYLA
WYQQRPGQAPRLLIYGASSRATGIPDRFSGSGSGT
DFTLTISSLEPEDFAVYYCQQYSNSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 846
14054 LC [hu anti-<huCDH19> 4B10
(1-236)(11450, A90T) VL]::huKLC
EIVLTQSPGTLSLSPGERATLSCRASQSVSNTYLA
WYQQRPGQAPRLLIYGASSRATGIPDRFSGSGSGT
DFTLTISSLEPEDFAVYYCQQYSNSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 847
14055 LC [hu anti-<huCDH19> 4B10
(1-236)(H45Q, A90T) VL]::huKLC
EIVLTQSPGTLSLSPGERATLSCRASQSVSNTYLA
WYQQRPGQAPRLLIYGASSRATGIPDRFSGSGSGT
DFTLTISSLEPEDFAVYYCQQYSNSWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 848
14056 LC [hu anti-<huCDH19> 4A9
(1-239)(F47L) VL]::huLLC-C1
QSVLTQPPSVSGAPGQRVTISCTGSSSNIGTGYAV
HWYQQLPGTAPKLLIYGNNNRPSGVPDRFSGSKSG
TSASLAITGLQAEDEADYYCQSYDSRLSGWVFGGG
TKLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 849
14057 LC [hu anti-<huCDH19> 4A9
(1-239)(F47L) VL]::huLLC-C1
QSVLTQPPSVSGAPGQRVTISCTGSSSNIGTGYAV
HWYQQLPGTAPKLLIYGNNNRPSGVPDRFSGSKSG
TSASLAITGLQAEDEADYYCQSYDSRLSGWVFGGG
TKLTVLGQPKANPTVTLFPPSSEELQANKATLVCL
ISDFYPGAVTVAWKADOSPVKAGVETTKPSKQSNN
KYAASSYLSLTPEQWKSHRSYSCQVTHEGSTVEKT
VAPTECS
SEQ ID NO: 850
14058 LC [hu anti-<huCDH19> 4A9
(1-239)(F47L, D110E) VL]::huLLC-C1
QSVLTQPPSVSGAPGQRVTISCTGSSSNIGTGYAV
HWYQQLPGTAPKLLIYGNNNRPSGVPDRFSGSKSG
TSASLAITGLQAEDEADYYCQSYESRLSGWVEGGG
TKLTVLGQPKANPTVTLEPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 851
14059 LC [hu anti-<huCDH19> 4A9
(1-239)(F47L, D110E) VL]::huLLC-C1
QSVLTQPPSVSGAPGQRVTISCTGSSSNIGTGYAV
HWYQQLPGTAPKWYGNNNRPSGVPDRFSGSKSGTS
ASLAITGLQAEDEADYYCQSYESRLSGWVFGGGTK
LTVLGQPKANPTVTLFPPSSEELQANKATLVCLIS
DFYPGAVTVAWKADGSPVKAGVETTKPSKQSNNKY
AASSYLSLTPEQWKSHRSYSCQVTHEGSTVEKTVA
PTECS
SEQ ID NO: 852
14060 LC [hu anti-<huCDH19> 20D3.1
(1-235)(S102A) VL]::huLLC-C2
QSALTQPPSATGTPGQRVTISCSGSSSNIGSNFVN
WYKQLPGTAPKVLIYTNNQRPSGVPDRFSGSKSGT
SASLAISGLQSEDEADYYCATWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 853
14061 LC [hu anti-<huCDH19> 20D3.1
(1-235)(K45Q, S102A) VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDDSLNGWVFGGGT
KLTVLGQPICAAPSVTLFPPSSEELQANKATLVCL
ISDFYPGAVTVAWKADSSPVKAGVETTTPSKQSNN
KYAASSYLSLTPEQWKSHRSYSCQVTHEGSTVEKT
VAPTECS
SEQ ID NO: 854
14062 LC [hu anti-<huCDH19> 20D3.1
(1-235)(K45Q, S102A) VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDDSLNGWVEGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 855
14063 LC [hu anti-<huCDH19> 20D3.1
(1-235)(K45Q, S102A, D111E, N135Q)
VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDESLQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 856
14064 LC [hu anti-<huCDH19> 20D3.1
(1-235)(W109Y) VL]::huLLC-C2
QSALTQPPSATGTPGQRVTISCSGSSSNIGSNFVN
WYKQLPGTAPKVLIYTNNQRPSGVPDRFSGSKSGT
SASLAISGLQSEDESDYYCATYDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 857
14065 LC [hu anti-<huCDH19> 22G10.1
VL]::huKLC
EIVMTQSPVTLSLSLGERATLSCRASQSISSNLAW
FQQKPGQAPRLLIYGAFTRATGIPARVSGSGSGTE
FTLTISSLQSEDFAVYYCQQYNYWPLTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 858
14066 LC [hu anti-<huCDH19> 22G10.1
VL]::huKLC
EIVMTQSPVTLSLSLGERATLSCRASQSISSNLAW
FQQKPGQAPRLLIYGAFTRATGIPARVSGSGSGTE
FTLTISSLQSEDFAVYYCQQYNYWPLTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 859
14067 LC [hu anti-<huCDH19> 22G10.1
(1-234)(097E, S981P) VL]::huKLC
EIVMTQSPVTLSLSLGERATLSCRASQSISSNLAW
FQQKPGQAPRLLIYGAFTRATGIPARVSGSGSGTE
FTLTISSLEPEDFAVYYCQQYNYWPLTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 860
14068 LC [hu anti-<huCDH19> 22G10.1
(1-234)(V78F, 097E, S98P) VL]::huKLC
EIVMTQSPVTLSLSLGERATLSCRASQSISSNLAW
FQQKPGQAPRLLIYGAFTRATGIPARFSGSGSGTE
FTLTISSLEPEDFAVYYCQQYNYWPLTFGGGTKVE
IKRTVAAPSVFIFPPSDEQLKSGTASVVCLLNNEY
PREAKVQWKVDNALQSGNSQESVTEQDSKDSTYSL
SSTLTLSKADYEKHKVYACEVTHQGLSSPVTKSFN
RGEC
SEQ ID NO: 861
14069 LC [hu anti-<huCDH19> 22G10.1
(1-234)(V78F, 097E, S98P) VL]::huKLC
EIVMTQSPVTLSLSLGERATLSCRASQSISSNLAW
FQQKPGQAPRLLIYGAFTRATGIPARFSGSGSGTE
FTLTISSLEPEDFAVYYCQQYNYWPLTFGGGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 862
14070 LC [hu anti-<huCDH19> 22G10.1
VL]::huKLC
EIVMTQSPVTLSLSLGERATLSCRASQSISSNLAW
FQQKPGQAPRLLIYGAFTRATGIPARVSGSGSGTE
FTLTISSLQSEDFAVYYCQQYNYWPLTEGGOTKVE
IKRTVAAPSVFIFPPSDEQLKSGTASVVCLLNNEY
PREAKVQWKVDNALQSGNSQESVTEQDSKDSTYSL
SSTLTLSKADYEKHKVYACEVTHQGLSSPVTKSFN
RGEC
SEQ ID NO: 863
14071 LC [hu anti-<huCDH19> 16A4.1
(1-235)(G141Q) VL]::huKLC
EIVLTQSPGTLSLSPGERATLSCRASQSVSSSYLA
WYQQKPGQAPRLLIYGTSSRATGIPDRFSGSGSGT
DFTLTISRLEPEDFAVYYCQQYGSSPFTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 864
14072 LC [hu anti-<huCDH19> 19B5.1
(1-235)(K45Q, S102A) VL]::huLLC-C2
QSALTQPPSTTGTPGQRVTISCSGSRSNIGSNFVN
WYQQLPGTAPKVLIYTNNQRPSGVPDRFSGSKSGT
SASLAISGLQSEDEADYYCATWDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 865
14073 LC [hu anti-<huCDH19> 19B5.1
(1-235)(K450, S102A) VL]::huLLC-C2
QSALTQPPSTTGTPGQRVTISCSGSRSNIGSNEVN
WYQQLPGTAPKVLIYTNNQRPSGVPDRFSGSKSGT
SASLAISGLQSEDEADYYCATWDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 866
14074 LC [hu anti-<huCDH19> 19B5.1
(1-235)(T11V, K450, S102A)
VL]::huLLC-C2
QSALTQPPSVTGTPGQRVTISCSGSRSNIGSNFVN
WYQQLPGTAPKVLIYTNNQRPSGVPDRFSGSKSGT
SASLAISGLQSEDEADYYCATWDDSMNGWVFGGOT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 867
14075 LC [hu anti-<huCDH19> 19B5.1
(1-235)(T11V, K45Q, S102A, D111E,
N135Q) VL]::huLLC-C2
QSALTQPPSVTGTPGQRVTISCSGSRSNIGSNFVN
WYQQLPGTAPKVLIYTNNQRPSGVPDRFSGSKSGT
SASLAISGLQSEDEADYYCATWDESMQGWVEGGGT
KLTVLGQPKAAPSVTLEPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 868
14076 LC [hu anti-<huCDH19> 19B5.1
(1-235)(T11V, K45Q, S102A, W109Y,
D111E, N135Q) VL]::huLLC-C2
QSALTQPPSVTGTPGQRVTISCSGSRSNIGSNFVN
WYQQLPGTAPKVLIYTNNQRPSGVPDRFSGSKSGT
SASLAISGLQSEDEADYYCATYDESMQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 869
14077 LC [hu anti-<huCDH19> 23A10.3
(1-231)(C42S) VL]::huLLC-C2
SYELTQPPSVSVSPGQTASITCSGDRLGEKYVSWY
QQKPGQSPILVIYQDNKWPSGIPERFSGSNSGNTA
TLTISGTQAMDEADYYCQAWDSSTVVFGGGTKLTV
LGQPKAAPSVTLFPPSSEELQANKATLVCLISDFY
PGAVTVAWKADSSPVKAGVETTTPSKQSNNKYAAS
SYLSLTPEQWKSHRSYSCQVTHEGSTVEKTVAPTE
CS
SEQ ID NO: 870
14078 LC [hu anti-<huCDH19> 23A10.3
(1-231)(C42S) VL]::huLLC-C2
SYELTQPPSVSVSPGQTASITCSGDRLGEKYVSWY
QQKPGQSPILVIYQDNKWPSGIPERFSGSNSGNTA
TLTISGTQAMDEADYYCQAWDSSTVVFGGGTKLTV
LGQPKAAPSVTLFPPSSEELQANKATLVCLISDFY
PGAVTVAWKADSSPVKAGVETTTPSKQSNNKYAAS
SYLSLTPEQWKSHRSYSCQVTHEGSTVEKTVAPTE
CS
SEQ ID NO: 871
14079 LC [hu anti-<huCDH19> 23A10.3
(1-231)(C42S, D110E) VL]::huLLC-C2
SYELTQPPSVSVSPGQTASITCSGDRLGEKYVSWY
QQKPGQSPILVIYQDNKWPSGIPERFSGSNSGNTA
TLTISGTQAMDEADYYCQAWESSTVVFGGGTKLTV
LGQPKAAPSVTLFPPSSEELQANKATLVCLISDFY
PGAVTVAWKADSSPVKAGVETTTPSKQSNNKYAAS
SYLSLTPEQWKSHRSYSCQVTHEGSTVEKTVAPTE
CS
SEQ ID NO: 872
14080 LC [hu anti-<huCDH19> 23A10.3
(1-231)(C42Y) VL]::huLLC-C2
SYELTQPPSVSVSPGQTASITCSGDRLGEKYVYWY
QQKPGQSPILVIYQDNKWPSGIPERFSGSNSGNTA
TLTISGTQAMDEADYYCQAWDSSTVVFOGGTKLTV
LGQPKAAPSVTLFPPSSEELQANKATLVCLISDFY
PGAVTVAWKADSSPVKAGVETTTPSKQSNNKYAAS
SYLSLTPEQWKSHRSYSCQVTHEGSTVEKTVAPTE
CS
SEQ ID NO: 873
14081 LC [hu anti-<huCDH19> 25G10.1
(1-235)(H105Y) VL]::hul(LC
EIVLTQSPGTLSLSPGERATLSCRASQSVSSSYLA
WYQQKPGQAPRLLIFGASSRATGIPDRFSGSGSGT
DFTLTISRLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 874
14082 LC [hu anti-<huCDH19> 25G10.1
(1-235)(H105Y) VL]::hul(LC
EIVLTQSPGTLSLSPGERATLSCRASQSVSSSYLA
WYQQKPGQAPRLLIFGASSRATGIPDRFSGSGSGT
DFTLTISRLEPEDFAVYYCQQYGNSPLTFGGGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC
SEQ ID NO: 875
14083 LC [hu anti-<huCDH19> 26D1.1
(1-235)(S7P) VL]::huLLC-C2
HSVLTQPPSASGTPGQRVTISCSGSRSNIGSNFVN
WYQQLPGTAPKLLIYTNNQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 876
14084 LC [hu anti-<huCDH19> 26D1.1
(1-235)(H1Q, S7P) VL]::huLLC-C2
QSVLTQPPSASGTPGQRVTISCSGSRSNIGSNFVN
WYQQLPGTAPKLLIYTNNQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 877
14085 LC [hu anti-<huCDH19> 26D1.1
(1-235)(H1Q, S7P, W109Y)
VL]::huLLC-C2
QSVLTQPPSASGTPGQRVTISCSGSRSNIGSNFVN
WYQQLPGTAPKLLIYTNNQRPSGVPDRFSGSKSGT
SASLAISGLQSEDEADYYCAVYDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 878
14086 LC [hu anti-<huCDH19> 26D1.1
(1-235)(H1Q, S7P, W109Y, D111E,
N135Q) VL]::huLLC-C2
QSVLTQPPSASGTPGQRVTISCSGSRSNIGSNFVN
WYQQLPGTAPKLLIYTNNQRPSGVPDRFSGSKSGT
SASLAISGLQSEDEADYYCAVYDESLQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 879
14087 LC [hu anti-<huCDH19> 26D1.1
(1-235)(H1Q, S7P, W109Y, D111E, N1350)
VL]::huLLC-C2
QSVLTQPPSASGTPGQRVTISCSGSRSNIGSNFVN
WYQQLPGTAPKLLIYTNNQRPSGVPDRFSGSKSGT
SASLAISGLQSEDEADYYCAVYDESLQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 880
14088 LC [hu anti-<huCDH19> 26D1.1
(1-235)(H1Q, S7P) VL]::huLLC-C2
QSVLTQPPSASGTPGQRVTISCSGSRSNIGSNFVN
WYQQLPGTAPKLLIYTNNQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 881
14089 LC [hu anti-<huCDH19> 26E12.1
(1-235)(S7P) VL]::huLLC-C2
QSVLTQPPSASGTPGQKVTISCSGSRSNIGSNFVN
WYQQLPGTAPKLLIYTNYQRPSGVPDRFSGSKSGT
SASLAISGLQSEDEADYYCAVWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 882
14090 LC [hu anti-<huCDH19> 26F12.1
(1-235)(S7P, D111E) VL]::huLLC-C2
QSVLTQPPSASGTPGQKVTISCSGSRSNIGSNFVN
WYQQLPGTAPKLLIYTNYQRPSGVPDRFSGSKSGT
SASLAISGLQSEDEADYYCAVWDESLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 883
14091 LC [hu anti-<huCDH19> 26E12.1
(1-235)(S7P, D111E) VL]::huLLC-C2
QSVLTQPPSASGTPGQKVTISCSGSRSNIGSNFVN
WYQQLPGTAPKLLIYTNYQRPSGVPDRFSGSKSGT
SASLAISGLQSEDEADYYCAVWDESLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 884
14092 LC [hu anti-<huCDH19> 26E12.1
(1-235)(S7P, W109Y, D111E, N1350)
VL]::huLLC-C2
QSVLTQPPSASGTPGQKVTISCSGSRSNIGSNFVN
WYQQLPGTAPKLLIYTNYQRPSGVPDRFSGSKSGT
SASLAISGLQSEDEADYYCAVYDESLQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 885
14093 LC [hu anti-<huCDH19> 25E8.1
(1-235)(K45Q) VL]::huLLC-C2
QSALTQPPSATGTPGQRVTISCSGSSSNIGRNFVN
WYQQLPGTAPKVLIYTNNQRPSGVPDRFSGSKSGT
SASLAISGLQSEDESDYYCAAWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 886
14094 LC [hu anti-<huCDH19> 25E8.1
(1-235)(K45Q, S102A) VL]::huLLC-C2
QSALTQPPSATGTPGQRVTISCSGSSSNIGRNFVN
WYQQLPGTAPKVLIYTNNQRPSGVPDRFSGSKSGT
SASLAISGLQSEDEADYYCAAWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 887
14095 LC [hu anti-<huCDH19> 25F8.1
(1-235)(K450, S102A) VL]::huLLC-C2
QSALTQPPSATGTPGQRVTISCSGSSSNIGRNFVN
WYQQLPGTAPKVLIYTNNQRPSGVPDRFSGSKSGT
SASLAISGLQSEDEADYYCAAWDDSLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 888
14096 LC [hu anti-<huCDH19> 25E8.1
(1-235)(K450, S102A, D111E)
VL]::huLLC-C2
QSALTQPPSATGTPGQRVTISCSGSSSNIGRNFVN
WYQQLPGTAPKVLIYTNNQRPSGVPDRFSGSKSGT
SASLAISGLQSEDEADYYCAAWDESLNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 889
14097 LC [hu anti-<huCDH19> 25E8.1
(1-235)(K45Q, S102A, D111E, N135Q)
VL]::huLLC-C2
QSALTQPPSATGTPGQRVTISCSGSSSNIGRNFVN
WYQQLPGTAPKVLIYTNNQRPSGVPDRFSGSKSGT
SASLAISGLQSEDEADYYCAAWDESLQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 890
14098 LC [hu anti-<huCDH19> 22D1.1
(1-235)(K450, S102A)
VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 891
14099 LC [hu anti-<huCDH19> 22D1.1
(1-235)(K45Q, S102A, D111E, N135Q)
VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDESMQGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 892
14100 LC [hu anti-<huCDH19> 22D1.1
(1-235)(K45Q, S102A, W109Y, D111E,
N1350) VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATYDESMQGWVFOGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 893
14101 LC [hu anti-<huCDH19> 22D1.1
(1-235)(K450, S102A, W109Y)
VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATYDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 894
14102 LC [hu anti-<huCDH19> 22D1.1
(1-235)(K45Q, S102A) VL]::huLLC-C2
QSALTQPPSATGTPGQRVTISCSGSSSNIGSNFVN
WYQQLPGTAPKVLIYTNNQRPSGVPDRFSGSKSGT
SASLAISGLQSEDEADYYCATWDDSMNGWVFGGGT
KLTVLGQPKAAPSVTLFPPSSEELQANKATLVCLI
SDFYPGAVTVAWKADSSPVKAGVETTTPSKQSNNK
YAASSYLSLTPEQWKSHRSYSCQVTHEGSTVEKTV
APTECS
SEQ ID NO: 895
13591 LC [hu anti-<huCDH19> 4F7
VL]::huLLC-C1
QSVLTQPPSVSGAPGQRVTISCTGSSSNIGTGYDV
HWYQQLPGTAPKLLIHGNSNRPSGVPDRFSGSKSG
TSASLAITGLQAEDEADYYCQSYDSSLSGWVFGGG
TRLTVLGQPKANPTVTLFPPSSEELQANKATLVCL
ISDFYPGAVTVAWKADGSPVKAGVETTKPSKQSNN
KYAASSYLSLTPEQWKSHRSYSCQVTHEGSTVEKT
VAPTECS
SEQ ID NO: 896
14301 LC [hu anti-<huCDH19> 2G6
(1-234)(D110E) VL]::huLLC-C1
SYELTQPPSVSVSPGQTASITCSGDRLGEKYTCWY
QQRPGQSPLLVIYQDTKRPSGIPERFSGSNSGNTA
TLTISGTQAMDEADYYCQAWESSTVVFGGGTKLTV
LGQPKANPTVTLFPPSSEELQANKATLVCLISDFY
PGAVTVAWKADGSPVKAGVETTKPSKQSNNKYAAS
SYLSLTPEQWKSHRSYSCQVTHEGSTVEKTVAPIE
CS
SEQ ID NO: 897
14302 LC [hu anti-<huCDH19> 2G6
(1-234)(C42S, D110E) VL]::huLLC-C1
SYELTQPPSVSVSPGQTASITCSGDRLGEKYTSWY
QQRPGQSPLLVIYQDTKRPSGIPERFSGSNSGNTA
TLTISGTQAMDEADYYCQAWESSTVVFGGGTKLTV
LGQPKANPTVTLFPPSSEELQANKATLVCLISDFY
PGAVTVAWKADGSPVKAGVETTKPSKQSNNKYAAS
SYLSLTPEQWKSHRSYSCQVTHEGSTVEKTVAPIE
CS
SEQ ID NO: 898
14303 LC [hu anti-<huCDH19> 2G6
(1-234)(C42S, D110E)
VL]::huLLC-C1
SYELTQPPSVSVSPGQTASITCSGDRLGEKYTSWY
QQRPGQSPLLVIYQDTKRPSGIPERFSGSNSGNTA
TLTISGTQAMDEADYYCQAWESSTVVFGGGTKLTV
LGQPKANPTVTLFPPSSEELQANKATLVCLISDFY
PGAVTVAWKADGSPVKAGVETTKPSKQSNNKYAAS
SYLSLTPEQWKSHRSYSCQVTHEGSTVEKTVAPIE
CS
SEQ ID NO: 899
14304 LC [hu anti-<huCDH19> 23A10.3
(1-231)(C42S) VL]::huLLC-C2
SYELTQPPSVSVSPGQTASITCSGDRLGEKYVSWY
QQKPGQSPILVIYQDNKWPSGIPERFSGSNSGNTA
TLTISGTQAMDEADYYCQAWDSSTVVFGGGTKLTV
LGQPKAAPSVTLFPPSSEELQANKATLVCLISDFY
PGAVTVAWKADSSPVKAGVETTTPSKQSNNKYAAS
SYLSLTPEQWKSHRSYSCQVTHEGSTVEKTVAPTE
CS
SEQ ID NO: 900

TABLE IVa
HEAVY CHAIN CDRs

| Ab | Type | CDR 1 | CDR 2 | CDR 3 |
|---|---|---|---|---|
| 14039 | AA | SYGMH | FIWYE | RAGIIGT |
| 14303 | | SEQ ID | GSNKY | IGYYYGM |
| | | NO: 28 | YAESV | DV |
| | | | KD | SEQ ID |
| | | | SEQ ID | NO: 30 |
| | | | NO: 901 | |
| 14027 | AA | SSGYY | YIYYT | EGSSGW |
| | | WS | GSAYY | YFQY |
| | | SEQ ID | NPSLK | SEQ ID |
| | | NO: 46 | S | NO: 902 |
| | | | SEQ ID | |
| | | | NO: 47 | |
| 14028 | AA | SSGYY | YIYYT | EGSSGY |
| | | WS | GSAYY | YFQY |
| | | SEQ ID | NPSLK | SEQ ID |
| | | NO: 46 | S | NO: 903 |
| | | | SEQ ID | |
| | | | NO: 47 | |
| 14059 | AA | GYYWS | YFSYS | NYAFHF |
| | | SEQ ID | GSTNY | DF |
| | | NO: 52 | NPSLK | SEQ ID |
| | | | S | NO: 904 |
| | | | SEQ ID | |
| | | | NO: 53 | |
| 14052 | AA | SYDMH | VISYE | ERYFD |
| | | SEQ ID | GTNEY | YSFDY |
| | | NO: 58 | YAESV | SEQ ID |
| | | | KG | NO: 906 |
| | | | SEQ ID | |
| | | | NO: 905 | |
| 14055 | AA | SYDMH | VISYE | ERYFDW |
| | | SEQ ID | GTNEY | SFDY |
| | | NO: 58 | YAESV | SEQ ID |
| | | | KG | NO: 60 |
| | | | SEQ ID | |
| | | | NO: 905 | |
| 14033 | AA | SYDMD | VIWYE | ETGEGW |
| | | SEQ ID | GSNKY | YFDL |
| | | NO: 70 | YAESV | SEQ ID |
| | | | RG | NO: 72 |
| | | | SEQ ID | |
| | | | NO: 907 | |
| 14034 | AA | SYDMD | VIWYE | ETGEGY |
| | | SEQ ID | GSNKY | YFDL |
| | | NO: 70 | YAESV | SEQ ID |
| | | | RG | NO: 908 |
| | | | SEQ ID | |
| | | | NO: 907 | |
| 14051 | AA | SYSWS | YIYYS | NYAFH |
| | | SEQ ID | GSTNY | FDY |
| | | NO: 82 | NPSLK | SEQ ID |
| | | | S | NO: 909 |
| | | | SEQ ID | |
| | | | NO: 83 | |
| 14046 | AA | SYYWS | YIYYI | ESRYRS |
| 14048 | | SEQ ID | GSTNY | GWYDAF |
| | | NO: 94 | NPSLK | DI |
| | | | S | SEQ ID |
| | | | SEQ ID | NO: 910 |
| | | | NO: 95 | |
| 14047 | AA | SYYWS | YIYYI | ESRYRS |
| | | SEQ ID | GSTNY | GYYDAF |
| | | NO: 94 | NPSLK | DI |
| | | | S | SEQ ID |
| | | | SEQ ID | NO: 911 |
| | | | NO: 95 | |
| 14042 | AA | GYYWS | YIYYI | EGSSGW |
| | | SEQ ID | GSTNY | YRWFDP |
| | | NO: 100 | NPSLK | SEQ ID |
| | | | S | NO: 912 |
| | | | SEQ ID | |
| | | | NO: 101 | |
| 14043 | AA | GYYWS | YIYYI | DGSSGY |
| | | SEQ ID | GSTNY | YRYFDP |
| | | NO: 100 | NPSLK | SEQ ID |
| | | | S | NO: 913 |
| | | | SEQ ID | |
| | | | NO: 101 | |
| 14069 | AA | SYAMN | TISGG | GGMGGY |
| | | SEQ ID | GANTY | YYGMDV |
| | | NO: 118 | YAESV | SEQ ID |
| | | | KG | NO: 120 |
| | | | SEQ ID | |
| | | | NO: 914 | |
| 14062 | AA | SYFIH | IINPI | GGIQLY |
| 14063 | | SEQ ID | SVSTS | LHFDY |
| 14064 | | NO: 124 | YAQKF | SEQ ID |
| | | | QG | NO: 915 |
| | | | SEQ ID | |
| | | | NO: 125 | |
| 14100 | AA | SYFIH | IINPI | GGIQLY |
| 14101 | | SEQ ID | SVSTS | LHLDY |
| | | NO: 130 | YAQKF | SEQ ID |
| | | | QG | NO: 916 |
| | | | SEQ ID | |
| | | | NO: 131 | |
| 14097 | AA | SYYIH | IINPS | GGIQLY |
| | | SEQ ID | GGSTR | LHFDY |
| | | NO: 136 | YAQKF | SEQ ID |
| | | | QG | NO: 917 |
| | | | SEQ ID | |
| | | | NO: 137 | |
| 14091 | AA | NYYMS | IINPS | GGIQL |
| 14092 | | SEQ ID | GGDST | YLHFDY |
| | | NO: 142 | YAQKF | SEQ ID |
| | | | QG | NO: 918 |
| | | | SEQ ID | |
| | | | NO: 143 | |
| 14087 | AA | SYYMS | IIHPS | GGIKLY |
| | | SEQ ID | GGDTT | LHFDY |
| | | NO: 148 | YAQKF | SEQ ID |
| | | | QG | NO: 919 |
| | | | SEQ ID | |
| | | | NO: 149 | |
| 14082 | AA | GYYWS | YIYYI | EGSSGY |
| | | SEQ ID | GSTNY | YRYFDP |
| | | NO: 154 | NPSLK | SEQ ID |
| | | | S | NO: 920 |
| | | | SEQ ID | |
| | | | NO: 155 | |
| 14079 | AA | RYGIH | VIWYE | RAGIPGT |
| | | SEQ ID | GSNKY | TGYYYGM |
| | | NO: 160 | YAESV | DV |
| | | | KG | SEQ ID |
| | | | SEQ ID | NO: 162 |
| | | | NO: 921 | |
| 14073 | AA | SYFIH | IINPI | GGIQLY |
| 14076 | | SEQ ID | SVSTS | LHLDY |
| | | NO: 1 | YAQKF | SEQ ID |
| | | | QG | NO: 3 |
| | | | SEQ ID | |
| | | | NO: 2 | |
| | AA | SYGMH | VIWYD | RAGIIG |
| | | SEQ ID | GSNKY | TTGYYY |
| | | NO: 4 | YADSV | GMDV |
| | | | KG | SEQ ID |
| | | | SEQ ID | NO: 6 |
| | | | NO: 5 | |

TABLE IVb
LIGHT CHAIN CDRs

| Ab | Type | CDR 1 | CDR 2 | CDR 3 |
|---|---|---|---|---|
| 14039 | AA | SGDRL | QDTKR | QAWES |
| 14302 | | GEKYT | PS | STVV |
| 14303 | | S | SEQ ID | SEQ ID |
| | | SEQ ID | NO: 197 | NO: 923 |
| | | NO: 922 | | |
| 14301 | AA | SGDRL | QDTKR | QAWES |
| | | GEKYT | PS | STVV |
| | | C | SEQ ID | SEQ ID |
| | | SEQ ID | NO: 197 | NO: 923 |
| | | NO: 196 | | |
| 14022 | AA | RASRQ | GPSSR | QQYGS |
| 14024 | | ISSSY | AT | SFT |
| 14025 | | LA | SEQ ID | SEQ ID |
| 14026 | | SEQ ID | NO: 215 | NO: 216 |
| 14027 | | NO: 924 | | |
| 14028 | | | | |
| 14029 | AA | RASQS | GPSSR | QQYGS |
| | | ISSSY | AT | SFT |
| | | LA | SEQ ID | SEQ ID |
| | | SEQ ID | NO: 215 | NO: 216 |
| | | NO: 925 | | |
| 14058 | AA | TGSSS | GNNNR | QSYES |
| 14059 | | NIGTG | PS | RLSGW |
| | | YAVH | SEQ ID | V |
| | | SEQ ID | NO: 221 | SEQ ID |
| | | NO: 220 | | NO: 926 |
| 14050 | AA | TGSSS | GNSNR | QSYES |
| 14051 | | NIGTG | PS | SLSGW |
| | | YDVH | SEQ ID | V |
| | | SEQ ID | NO: 251 | SEQ ID |
| | | NO: 250 | | NO: 927 |
| 14063 | AA | SGSSS | TNNQR | ATWDE |
| | | NIGSN | PS | SLQGW |
| | | FVN | SEQ ID | V |
| | | SEQ ID | NO: 293 | SEQ ID |
| | | NO: 292 | | NO: 928 |
| 14064 | AA | SGSSS | TNNQR | ATYDD |
| | | NIGSN | PS | SLNGW |
| | | FVN | SEQ ID | V |
| | | SEQ ID | NO: 293 | SEQ ID |
| | | NO: 292 | | NO: 929 |
| 14099 | AA | SGSSS | TNNQR | ATWDE |
| | | NIGSN | PS | SMQGW |
| | | FVN | SEQ ID | V |
| | | SEQ ID | NO: 299 | SEQ ID |
| | | NO: 298 | | NO: 930 |
| 14100 | AA | SGSSS | TNNQR | ATYDE |
| | | NIGSN | PS | SMQGW |
| | | FVN | SEQ ID | V |
| | | SEQ ID | NO: 299 | SEQ ID |
| | | NO: 298 | | NO: 931 |
| 14101 | AA | SGSSS | TNNQR | ATYDD |
| | | NIGSN | PS | SMNGW |
| | | FVN | SEQ ID | V |
| | | SEQ ID | NO: 299 | SEQ ID |
| | | NO: 298 | | NO: 932 |
| 14096 | AA | SGSSS | TNNQR | AAWDE |
| | | NIGRN | PS | SLNGW |
| | | FVN | SEQ ID | V |
| | | SEQ ID | NO: 305 | SEQ ID |
| | | NO: 304 | | NO: 933 |
| 14097 | AA | SGSSS | TNNQR | AAWDE |
| | | NIGRN | PS | SLQGW |
| | | FVN | SEQ ID | V |
| | | SEQ ID | NO: 305 | SEQ ID |
| | | NO: 304 | | NO: 934 |
| 14090 | AA | SGSRS | TNYQR | AVWDE |
| 14091 | | NIGSN | PS | SLNGW |
| | | FVN | SEQ ID | V |
| | | SEQ ID | NO: 311 | SEQ ID |
| | | NO: 310 | | NO: 935 |
| 14092 | AA | SGSRS | TNYQR | AVYDE |
| | | NIGSN | PS | SLQGW |
| | | FVN | SEQ ID | V |
| | | SEQ ID | NO: 311 | SEQ ID |
| | | NO: 310 | | NO: 936 |
| 14085 | AA | SGSRS | TNNQR | AVYDD |
| | | NIGSN | PS | SLNGW |
| | | FVN | SEQ ID | V |
| | | SEQ ID | NO: 317 | SEQ ID |
| | | NO: 316 | | NO: 937 |
| 14086 | AA | SGSRS | TNNQR | AVYDE |
| 14087 | | NIGSN | PS | SLQGW |
| | | FVN | SEQ ID | V |
| | | SEQ ID | NO: 317 | SEQ ID |
| | | NO: 316 | | NO: 938 |
| 14077 | AA | SGDRL | QDNKW | QAWDS |
| 14078 | | GEKYV | PS | STVV |
| 14304 | | S | SEQ ID | SEQ ID |
| | | SEQ ID | NO: 329 | NO: 330 |
| | | NO: 939 | | |
| 14079 | AA | SGDRL | QDNKW | QAWES |
| | | GEKYV | PS | STVV |
| | | S | SEQ ID | SEQ ID |
| | | SEQ ID | NO: 329 | NO: 940 |
| | | NO: 939 | | |
| 14080 | AA | SGDRL | QDNKW | QAWDS |
| | | GEKYV | PS | STVV |
| | | Y | SEQ ID | SEQ ID |
| | | SEQ ID | NO: 329 | NO: 330 |
| | | NO: 941 | | |
| 14075 | AA | SGSRS | TNNQR | ATWDE |
| | | NIGSN | PS | SMQGW |
| | | FVN | SEQ ID | V |
| | | SEQ ID | NO: 335 | SEQ ID |
| | | NO: 334 | | NO: 942 |
| 14076 | AA | SGSRS | TNNQR | ATYDE |
| | | NIGSN | PS | SMQGW |
| | | FVN | SEQ ID | V |
| | | SEQ ID | NO: 335 | SEQ ID |
| | | NO: 334 | | NO: 943 |

### Human and Cynomologous Monkey Cadherin-19 Sequences

TABLE V

| SEQ | | | | |
|---|---|---|---|---|
| ID | DESIG- | | | |
| NO. | NATION | SOURCE | TYPE | SEQUENCE |
| 944 | Human | Human | aa | MNCYLLLRFMLGIPLLWPCL |
| | Cadherin- | | | GATENSQTKKVKQPVRSHLR |
| | 19 | | | VKRGWVWNQFFVPEEMNTTS |
| | | | | HHIGQLRSDLDNGNNSFQYK |
| | | | | LLGAGAGSTFIIDERTGDIY |
| | | | | AIQKLDREERSLYILRAQVI |
| | | | | DIATGRAVEPESEFVIKVSD |
| | | | | INDNEPKFLDEPYEAIVPEM |
| | | | | SPEGTLVIQVTASDADDPSS |
| | | | | GNNARLLYSLLQGQPYFSVE |
| | | | | PTTGVIRISSKMDRELQDEY |
| | | | | WVIIQAKDMIGQPGALSGTT |
| | | | | SVLIKLSDVNDNKPIFKESL |
| | | | | YRLTVSESAPTGTSIGTIMA |
| | | | | YDNDIGENAEMDYSIEEDDS |
| | | | | QTFDIITNHETQEGIVILKK |
| | | | | KVDFEHQNHYGIRAKVKNHH |
| | | | | VPEQLMKYHTEASTTFIKIQ |
| | | | | VEDVDEPPLFLLPYYVFEVF |
| | | | | EETPQGSFVGVVSATDPDNR |
| | | | | KSPIRYSITRSKVFNINDNG |
| | | | | TITTSNSLDREISAWYNLSI |
| | | | | TATEKYNIEQISSIPLYVQV |
| | | | | LNINDHAPEFSQYYETYVCE |
| | | | | NAGSGQVIQTISAVDRDESI |
| | | | | EEHHFYFNLSVEDTNNSSFT |
| | | | | IIDNQDNTAVILTNRTGFNL |
| | | | | QEEPVFYISILIADNGIPSL |
| | | | | TSTNTLTIHVCDCGDSGSTQ |
| | | | | TCQYQELVLSMGFKTEVIIA |
| | | | | ILICIMIIFGFIFLTLGLKQ |
| | | | | RRKQILFPEKSEDFRENIFQ |
| | | | | YDDEGGGEEDTEAFDIAELR |
| | | | | SSTIMRERKTRKTTSAEIRS |
| | | | | LYRQSLQVGPDSAIFRKFIL |
| | | | | EKLEEANTDPCAPPFDSLQT |
| | | | | YAFEGTGSLAGSLSSLESAV |
| | | | | SDQDESYDYLNELGPRFKRL |
| | | | | ACMFGSAVQSNN |
| 945 | Human | Human | nt | atgaactgttatttactgct |
| | Cadherin- | | | gcgttttatgttgggaattc |
| | 19 | | | ctctcctatggccttgtctt |
| | | | | ggagcaacagaaaactctca |
| | | | | aacaaagaaagtcaagcagc |
| | | | | cagtgcgatctcatttgaga |
| | | | | gtgaagcgtggctgggtgtg |
| | | | | gaaccaattttttgtaccag |
| | | | | aggaaatgaatacgactagt |
| | | | | catcacatcggccagctaag |
| | | | | atctgatttagacaatggaa |
| | | | | acaattctttccagtacaag |
| | | | | cttttgggagctggagctgg |
| | | | | aagtacttttatcattgatg |
| | | | | aaagaacaggtgacatatat |
| | | | | gccatacagaagcttgatag |
| | | | | agaggagcgatccctctaca |
| | | | | tcttaagagcccaggtaata |
| | | | | gacatcgctactggaagggc |
| | | | | tgtggaacctgagtctgagt |
| | | | | ttgtcatcaaagtttcggat |
| | | | | atcaatgacaatgaaccaaa |
| | | | | attcctagatgaaccttatg |
| | | | | aggccattgtaccagagatg |
| | | | | tctccagaaggaacattagt |
| | | | | tatccaggtgacagcaagtg |
| | | | | atgctgacgatccctcaagt |
| | | | | ggtaataatgctcgtctcct |
| | | | | ctacagcttacttcaaggcc |
| | | | | agccatatttttctgttgaa |
| | | | | ccaacaacaggagtcataag |
| | | | | aatatcttctaaaatggata |
| | | | | gagaactgcaagatgagtat |
| | | | | tgggtaatcattcaagccaa |
| | | | | ggacatgattggtcagccag |
| | | | | gagcgttgtctggaacaaca |
| | | | | agtgtattaattaaactttc |
| | | | | agatgttaatgacaataagc |
| | | | | ctatatttaaagaaagttta |
| | | | | taccgcttgactgtctctga |
| | | | | atctgcacccactgggactt |
| | | | | ctacaggaacaatcatggca |
| | | | | tatgataatgacataggaga |
| | | | | gaatgcagaaatggattaca |
| | | | | gcattgaagaggatgattcg |
| | | | | caaacatttgacattattac |
| | | | | taatcatgaaactcaagaag |
| | | | | gaatagttatattaaaaaag |
| | | | | aaagtggatcttgagcacca |
| | | | | gaaccactacggtattagag |
| | | | | caaaagttaaaaaccatcat |
| | | | | gttcctgagcagctcatgaa |
| | | | | gtaccacactgaggcttcca |
| | | | | ccactttcattaagatccag |
| | | | | gtggaagatgttgatgagcc |
| | | | | tcctcttttcctccttccat |
| | | | | attatgtatttgaagttttt |
| | | | | gaagaaaccccacagggatc |
| | | | | atttgtaggcgtggtgtctg |
| | | | | ccacagacccagacaatagg |
| | | | | aaatctcctatcaggtattc |
| | | | | tattactaggagcaaagtgt |
| | | | | tcaatatcaatgataatggt |
| | | | | acaatcactacaagtaactc |
| | | | | actgcatcgtgaaatcagtg |
| | | | | cttggtacaacctaagtatt |
| | | | | acagccacagaaaaatacaa |
| | | | | tatagaacagatctcttcga |
| | | | | tcccactgtatgtgcaagtt |
| | | | | cttaacatcaatgatcatgc |
| | | | | tcctgagttctctcaatact |
| | | | | atgagacttatgtttgtgaa |
| | | | | aatgcaggctccggtcaggt |
| | | | | aattcagactatcagtgcag |
| | | | | tggatagagatgaatccata |
| | | | | gaagagcaccatttttactt |
| | | | | taatctatctgtagaagaca |
| | | | | ctaacaattcaagttttaca |
| | | | | atcatagataaccaagataa |
| | | | | cacagctgtcattttgacta |
| | | | | atagaactggttttaacctt |
| | | | | caagaagaacctgtcttcta |
| | | | | catctccatcttaattgccg |
| | | | | acaatggaatcccgtcactt |
| | | | | acaagtacaaacacccttac |
| | | | | catccatgtctgtgactgtg |
| | | | | gtgacagtgggagcacacag |
| | | | | acctgccagtaccaggagct |
| | | | | tgtgctttccatgggattca |
| | | | | agacagaagtcatcattgct |
| | | | | attctcatttgcattatgat |
| | | | | catatttgggtttatttttt |
| | | | | tgactttgggtttaaaaca |
| | | | | acggagaaaacagattcta |
| | | | | tttcctgagaaaagtgaag |
| | | | | atttcagagagaatatat |
| | | | | tccaatatgatgatgaagg |
| | | | | gggtggagaagaagatac |
| | | | | agaggcctttgatatagcag |
| | | | | agctgaggagtagtaccata |
| | | | | atgcgggaacgcaagactcg |
| | | | | gaaaaccacaagcgctgaga |
| | | | | tcaggagcctatacaggcag |
| | | | | tctttgcaagttggccccga |
| | | | | cagtgccatattcaggaaat |
| | | | | tcattctggaaaagctcgaa |
| | | | | gaagctaatactgatccgtg |
| | | | | tgcccctccttttgattccc |
| | | | | tccagacctacgcttttgag |
| | | | | ggaacagggtcattagctgg |
| | | | | atccctgagctccttagaat |
| | | | | cagcagtctctgatcacgat |
| | | | | gaaagctatgattaccttaa |
| | | | | tgagttgggacctcgcttta |
| | | | | aaagattagcatgcatgttt |
| | | | | ggttctgcagtgcagtcaaa |
| | | | | taattag |
| 946 | Cyno | Macaca | aa | MNCYLLLPFMLGIPLLWPCL |
| | Cadherin- | fasci- | | GATENSQTKKVQQPVGSHLR |
| | 19 | cularis | | VKRGWVWNQFFVPEEMNTTS |
| | | | | HHVGRLRSDLDNGNNSFQYK |
| | | | | LLGAGAGSTFIIDERTGDIY |
| | | | | AIEKLDREERSLYILRAQVI |
| | | | | DITTGRAVEPESEFVIKVSD |
| | | | | INDNEPKFLDEPYEAIVPEM |
| | | | | SPEGTLVIQVTASDADDPSS |
| | | | | GNNARLLYSLLQGQPYFSVE |
| | | | | PTTGVIRISSKMDRELQDEY |
| | | | | WVIIQAKDMIGQPGALSGTT |
| | | | | SVLIKLSDVNDNKPIFKESL |
| | | | | YRLTVSESAPTGTSIGTIMA |
| | | | | YDNDIGENAEMDYSIEEDDS |
| | | | | QTFDIITNHETQEGIVILKK |
| | | | | KVNFEHQNHYGIRAKVKNHH |
| | | | | VDEQLMKYHTEASTTFIKIQ |
| | | | | VEDVDEPPLFLLPYYIFEIF |
| | | | | EETPQGSFVGVVSATDPDNR |
| | | | | KSPIRYSITRSKVFNIDDNG |
| | | | | TITTTNSLDREISAWYNLSI |
| | | | | TATEKYNIEQISSIPVYVQV |
| | | | | LNINDHAPEFSQYYESYVCE |
| | | | | NAGSGQVIQTISAVDRDESI |
| | | | | EEHHFYFNLSVEDTNSSSFT |
| | | | | IIDNQDNTAVILTNRTGFNL |
| | | | | QEEPIFYISILIADNGIPSL |
| | | | | TSTNTLTIHVCDCDDSGSTQ |
| | | | | TCQYQELMLSMGFKTEVIIA |
| | | | | ILICIMVIFGFIFLTLGLKQ |
| | | | | RRKQILFPEKSEDFRENIFR |
| | | | | YDDEGGGEEDTEAFDVAALR |
| | | | | SSTIMRERKTRKTTSAEIRS |
| | | | | LYRQSLQVGPDSAIFRKFIL |
| | | | | EKLEEADTDPCAPPFDSLQT |
| | | | | YAFEGTGSLAGSLSSLESAV |
| | | | | SDQDESYDYLNELGPRFKRL |
| | | | | ACMFGSAVQSNN |
| 947 | Cyno | Macaca | nt | ATGAATTGTTATTTACTGCT |
| | Cadherin- | fasci- | | GCCTTTTATGTTGGGAATTC |
| | 19 | cularis | | CTCTCCTATGGCCTTGTCTT |
| | | | | GGAGCAACAGAAAACTCTCA |
| | | | | AACAAAGAAAGTCCAGCAGC |
| | | | | CAGTAGGATCTCATCTGAGA |
| | | | | GTGAAGCGTGGCTGGGTGTG |
| | | | | GAACCAATTTTTTGTACCAG |
| | | | | AGGAAATGAATACGACTAGT |
| | | | | CATCACGTTGGCCGGCTAAG |
| | | | | ATCTGATTTAGACAATGGAA |
| | | | | ACAATTCTTTCCAGTACAAG |
| | | | | CTTTTGGGAGCTGGAGCTGG |
| | | | | AAGTACTTTTATCATTGATG |
| | | | | AAAGAACAGGTGACATATAT |
| | | | | GCCATAGAGAAGCTTGATAG |
| | | | | AGAGGAGCGATCCCTCTACA |
| | | | | TCTTAAGAGCCCAGGTAATA |
| | | | | GACATCACTACTGGAAGGGC |
| | | | | TGTGGAACCTGAGTCTGAGT |
| | | | | TTGTCATCAAAGTTTCGGAT |
| | | | | ATCAATGACAATGAACCAAA |
| | | | | ATTCCTAGATGAACCTTATG |
| | | | | AGGCCATTGTACCAGAGATG |
| | | | | TCTCCAGAAGGAACATTAGT |
| | | | | CATCCAGGTGACAGCAAGTG |
| | | | | ATGCTGATGACCCTTCAAGT |
| | | | | GGTAATAATGCTCGTCTCCT |
| | | | | CTACAGCTTATTACAAGGCC |
| | | | | AGCCATATTTTTCTGTTGAA |
| | | | | CCAACAACAGGAGTCATAAG |
| | | | | AATATCTTCTAAAATGGATA |
| | | | | GAGAACTGCAAGATGAGTAT |
| | | | | TGGGTAATCATTCAAGCCAA |
| | | | | GGACATGATTGGTCAGCCAG |
| | | | | GAGCGTTGTCTGGAACAACG |
| | | | | AGTGTATTAATTAAACTTTC |
| | | | | AGATGTTAATGACAATAAGC |
| | | | | CTATATTTAAAGAAAGTTTA |
| | | | | TACCGCCTGACGGTCTCTGA |
| | | | | ATCTGCACCCACTGGGACTT |
| | | | | CTATAGGAACAATCATGGCA |
| | | | | TATGATAATGACATAGGAGA |
| | | | | GAATGCAGAAATGGATTACA |
| | | | | GCATTGAAGAGGATGATTCA |
| | | | | CAGACATTTGACATTATTAC |
| | | | | TAATCATGAAACTCAAGAAG |
| | | | | GAATAGTTATATTAAAAAAG |
| | | | | AAAGTGAATTTTGAGCACCA |
| | | | | GAACCACTATGGTATTAGAG |
| | | | | CAAAAGTTAAAAACCATCAT |
| | | | | GTTGATGAGCAGCTCATGAA |
| | | | | ATACCACACTGAAGCTTCCA |
| | | | | CCACTTTCATTAAGATCCAG |
| | | | | GTGGAAGATGTTGATGAGCC |
| | | | | TCCTCTTTTCCTCCTTCCGT |
| | | | | ATTACATATTTGAAATTTTT |
| | | | | GAAGAAACCCCACAAGGATC |
| | | | | ATTTGTAGGCGTGGTGTCTG |
| | | | | CCACAGACCCAGACAATAGG |
| | | | | AAATCTCCTATCAGGTATTC |
| | | | | TATTACTAGGAGCAAAGTGT |
| | | | | TCAATATCGATGATAATGGT |
| | | | | ACAATCACTACAACTAACTC |
| | | | | ACTGGATCGGGAAATCAGTG |
| | | | | CTTGGTACAACCTAAGTATT |
| | | | | ACAGCCACAGAAAAATACAA |
| | | | | TATAGAGCAGATCTCTTCGA |
| | | | | TCCCAGTGTATGTGCAAGTT |
| | | | | CTTAATATCAATGATCATGC |
| | | | | TCCTGAGTTCTCTCAATACT |
| | | | | ATGAGAGTTATGTTTGTGAA |
| | | | | AATGCAGGCTCTGGTCAGGT |
| | | | | AATTCAGACTATCAGTGCAG |
| | | | | TGGATAGAGATGAATCCATA |
| | | | | GAAGAGCACCATTTTTACTT |
| | | | | TAATCTATCTGTAGAAGACA |
| | | | | CTAACTCTTCAAGTTTTACA |
| | | | | ATCATAGACAATCAAGATAA |
| | | | | CACAGCTGTCATTTTGACTA |
| | | | | ATAGAACTGGTTTTAACCTT |
| | | | | CAAGAAGAGCCCATCTTCTA |
| | | | | CATCTCCATCTTAATTGCCG |
| | | | | ACAATGGAATCCCGTCACTT |
| | | | | ACAAGTACAAACACCCTTAC |
| | | | | CATCCATGTCTGTGACTGTG |
| | | | | ATGACAGTGGGAGCACACAG |
| | | | | ACCTGCCAGTACCAGGAGCT |
| | | | | TATGCTTTCCATGGGATTCA |
| | | | | AGACAGAAGTCATCATTGCT |
| | | | | ATTCTCATTTGCATTATGGT |
| | | | | AATATTTGGGTTTATTTTTT |
| | | | | TGACTTTGGGTTTAAAACA |
| | | | | ACGGAGAAAACAGATTCT |
| | | | | ATTTCCTGAGAAAAGTGA |
| | | | | AGATTTCAGAGAGAATAT |
| | | | | ATTCCGATATGATGACGAA |
| | | | | GGGGGTGGAGAAGAAGATAC |
| | | | | AGAGGCCTTTGACGTAGCAG |
| | | | | CGCTGAGGAGTAGCACCATA |
| | | | | ATGCGGGAACGCAAGACTCG |
| | | | | GAAAACCACCAGCGCTGAGA |
| | | | | TCAGGAGCCTATACAGGCAG |
| | | | | TCTTTGCAAGTTGGCCCCGA |
| | | | | CAGTGCCATATTCAGGAAGT |
| | | | | TCATCCTGGAAAAGCTCGAA |
| | | | | GAAGCTGATACTGATCCGTG |
| | | | | TGCCCCTCCTTTTGATTCCC |
| | | | | TCCAGACCTACGCTTTTGAG |
| | | | | GGAACAGGGTCATTAGCTGG |
| | | | | ATCCCTGAGCTCCTTAGAAT |
| | | | | CAGCTGTCTCTGATCAGGAT |
| | | | | GAAAGCTATGATTACCTTAA |
| | | | | CGAGTTGGGACCTCGCTTTA |
| | | | | AAAGATTAGCATGCATGTTT |
| | | | | GGTTCTGCAGTGCAGTCAAA |
| | | | | TAATTAG |
| 948 | secreted | Human | aa | MNCYLLLRFMLGIPLLWPCL |
| | Cadherin- | | | GATENSQTKKVKQPVRSHLR |
| | 19 | | | VKRGWVWNQFFVPEEMNTTS |
| | ecto- | | | HHIGQLRSDLDNGNNSFQYK |
| | domain | | | LLGAGAGSTFIIDERTGDIY |
| | (amino | | | AIQKLDREERSLYILRAQVI |
| | acids | | | DIATGRAVEPESEFVIKVSD |
| | 1-596) | | | INDNEPKFLDEPYEAIVPEM |
| | | | | SPEGTLVIQVTASDADDPSS |
| | | | | GNNARLLYSLLQGQPYFSVE |
| | | | | PTTGVIRISSKMDRELQDEY |
| | | | | WVIIQAKDMIGQPGALSGTT |
| | | | | SVLIKLSDVNDNKPIFKESL |
| | | | | YRLTVSESAPTGTSIGTIMA |
| | | | | YDNDIGENAEMDYSIEEDDS |
| | | | | QTFDIITNHETQEGIVILKK |
| | | | | KVDFEHQNHYGIRAKVKNHH |
| | | | | VPEQLMKYHTEASTTFIKIQ |
| | | | | VEDVDEPPLFLLPYYVFEVF |
| | | | | EETPQGSFVGVVSATDPDNR |
| | | | | KSPIRYSITRSKVFNINDNG |
| | | | | TITTSNSLDREISAWYNLSI |
| | | | | TATEKYNIEQISSIPLYVQV |
| | | | | LNINDHAPEFSQYYETYVCE |
| | | | | NAGSGQVIQTISAVDRDESI |
| | | | | EEHHFYFNLSVEDTNNSSFT |
| | | | | IIDNQDNTAVILTNRTGFNL |
| | | | | QEEPVFYISILIADNGIPSL |
| | | | | TSTNTLTIHVCDCGDSGSTQ |
| | | | | TCQYQELVLSMGFKTE |
| 949 | secreted | Human | nt | atgaactgttatttactgct |
| | Cadherin- | | | gcgttttatgttgggaattc |
| | 19 | | | ctctcctatggccttgtctt |
| | ecto- | | | ggagcaacagaaaactctca |
| | domain | | | aacaaagaaagtcaagcagc |
| | (amino | | | cagtgcgatctcatttgaga |
| | acids | | | gtgaagcgtggctgggtgtg |
| | 1-596) | | | gaaccaattttttgtaccag |
| | | | | aggaaatgaatacgactagt |
| | | | | catcacatcggccagctaag |
| | | | | atctgatttagacaatggaa |
| | | | | acaattctttccagtacaag |
| | | | | cttttgggagctggagctgg |
| | | | | aagtacttttatcattgatg |
| | | | | aaagaacaggtgacatatat |
| | | | | gccatacagaagcttgatag |
| | | | | agaggagcgatccctctaca |
| | | | | tcttaagagcccaggtaata |
| | | | | gacatcgctactggaagggc |
| | | | | tgtggaacctgagtctgagt |
| | | | | ttgtcatcaaagtttcggat |
| | | | | atcaatgacaatgaaccaaa |
| | | | | attcctagatgaaccttatg |
| | | | | aggccattgtaccagagatg |
| | | | | tctccagaaggaacattagt |
| | | | | tatccaggtgacagcaagtg |
| | | | | atgctgacgatccctcaagt |
| | | | | ggtaataatgctcgtctcct |
| | | | | ctacagcttacttcaaggcc |
| | | | | agccatatttttctcttgaa |
| | | | | ccaacaacaggagtcataag |
| | | | | aatatcttctaaaatggata |
| | | | | gagaactgcaagatgagtat |
| | | | | tgggtaatcattcaagccaa |
| | | | | ggacatgattggtcagccag |
| | | | | gagcgttgtctggaacaaca |
| | | | | agtgtattaattaaactttc |
| | | | | agatgttaatgacaataagc |
| | | | | ctatttttaaagaaagttta |
| | | | | taccgcttgactgtctctga |
| | | | | atctgcacccactgggactt |
| | | | | ctataggaacaatcatggca |
| | | | | tatgataatgacataggaga |
| | | | | gaatgcagaaatggattaca |
| | | | | gcattgaagaggatgattcg |
| | | | | caaacatttgacattattac |
| | | | | taatcatgaaactcaagaag |
| | | | | gaatagttatattaaaaaag |
| | | | | aaagtggattttgagcacca |
| | | | | gaaccactacggtattagag |
| | | | | caaaagttaaaaaccatcat |
| | | | | gttcctgagcagctcatgaa |
| | | | | gtaccacactgaggcttcca |
| | | | | ccactttcattaagatccag |
| | | | | gcggaagatgttgatgagcc |
| | | | | tcctcttttcctccttccat |
| | | | | attatgtatttgaagttttt |
| | | | | gaagaaaccccacagggatc |
| | | | | atttgtaggcgtggtgtctg |
| | | | | ccacagacccagacaatagg |
| | | | | aaatctcctatcaggtattc |
| | | | | tattactaggagcaaagtgt |
| | | | | tcaatatcaatgacaatggt |
| | | | | acaatcactacaagtaactc |
| | | | | actggatcgtgaaatcagtg |
| | | | | cttggtacaacctaagtatt |
| | | | | acagccacagaaaaatacaa |
| | | | | tatagaacagatctcttcga |
| | | | | tcccactgtatgtgcaagtt |
| | | | | cttaacatcaatgatcatgc |
| | | | | tcctgagttctctcaatact |
| | | | | atgagacttatgtttgtgaa |
| | | | | aatgcaggctctggtcaggt |
| | | | | aattcagactatcagtgcag |
| | | | | tggatagagatgaatccata |
| | | | | gaagagcaccatttttactt |
| | | | | taatctatctgtagaagaca |
| | | | | ctaacaattcaagttttaca |
| | | | | atcatagataatcaagataa |
| | | | | cacagctgtcattttgacta |
| | | | | atagaactggttttaacctt |
| | | | | caagaagaacctgtcttcta |
| | | | | catctccatcttaattgccg |
| | | | | acaatggaatcccgtcactt |
| | | | | acaagtacaaacacccttac |
| | | | | catccatgtctgtgactgtg |
| | | | | gtgacagtgggagcacacag |
| | | | | acctgccagtaccaggagct |
| | | | | tgtgctttccatgggattca |
| | | | | agacagaa |
| 950 | truncated | Human | aa | MNCYLLLRFMLGIPLLWPCL |
| | membrane | | | GATENSQTKKVKQPVRSHLR |
| | bound | | | VKRGWVWNQFFVPEEMNTTS |
| | form | | | HHIGQLRSDLDNGNNSFQYK |
| | of human | | | LLGAGAGSTFIIDERTGDIY |
| | cadherin- | | | AIQKLDREERSLYILRAQVI |
| | 19 | | | DIATGRAVEPESEFVIKVSD |
| | (amino | | | INDNEPKFLDEPYEAIVPEM |
| | acids | | | SPEGTLVIQVTASDADDPSS |
| | 1-624) | | | GNNARLLYSLLQGQPYFSVE |
| | | | | PTTGVIRISSKMDRELQDEY |
| | | | | WVIIQAKDMIGQPGALSGTT |
| | | | | SVLIKLSDVNDNKPIFKE |
| | | | | SLYRLTVSESAPTGTSIGTI |
| | | | | MAYDNDIGENAEMDYSIEED |
| | | | | DSQTFDIITNHETQEGIVIL |
| | | | | KKKVDFEHQNHYGIRAKVKN |
| | | | | KHVPEQLMKYHTEASTTFIK |
| | | | | IQVEDVDEPPLFLLPYYVFE |
| | | | | VFEETPQGSFVGVVSATDPD |
| | | | | NRKSPIRYSITRSKVFNIND |
| | | | | NGTITTSNSLDREISAWYNL |
| | | | | SITATEKYNIEQISSIPLYV |
| | | | | QVLNINDHAPEFSQYYETYV |
| | | | | CENAGSGQVIQTISAVDRDE |
| | | | | SIEEHHFYFNLSVEDTNNSS |
| | | | | FTIIDNQDNTAVILTNRTGF |
| | | | | NLQEEPVFYISILIADNGIP |
| | | | | SLTSTNTLTIHVCDCGDSGS |
| | | | | TQTCQYQELVLSMGFKTEVI |
| | | | | IAILICIMIIFGFIFLTLGL |
| | | | | KQRRKQ |
| 951 | truncated | Humanl | nt | atgaactgttatttactgct |
| | membrane | | | gcgttttatgttgggaattc |
| | bound | | | ctctcctatggccttgtctt |
| | form of | | | ggagcaacagaaaactctca |
| | human | | | aacaaagaaagtcaagcagc |
| | cadherin- | | | cagtgcgatctcatttgaga |
| | 19 | | | gtgaagcgtggctgggtgtg |
| | (amino | | | gaaccaattttttgtaccag |
| | acids | | | aggaaatgaatacgactagt |
| | 1-624) | | | catcacatcggccagctaag |
| | | | | atctgatttagacaatggaa |
| | | | | acaattctttccagtacaag |
| | | | | cttttgggagctggagctgg |
| | | | | aagtacttttatcattgatg |
| | | | | aaagaacaggtgacatatat |
| | | | | gccatacagaagcttgatag |
| | | | | agaggagcgatccctctaca |
| | | | | tcttaagagcccaggtaata |
| | | | | gacatcgctactggaagggc |
| | | | | tgtggaacctgagtctgagt |
| | | | | ttgtcatcaaagtttcggat |
| | | | | atcaatgacaatgaaccaaa |
| | | | | attcctagatgaaccttatg |
| | | | | aggccattgtaccagagatg |
| | | | | tctccagaaggaacattagt |
| | | | | tatccaggtgacagcaagtg |
| | | | | atgctgacgatccctcaagt |
| | | | | ggtaataatgctcgtctcct |
| | | | | ctacagcttacttcaaggcc |
| | | | | agccatatttttctgttgaa |
| | | | | ccaacaacaggagtcataag |
| | | | | aatatcttctaaaatggata |
| | | | | gagaactgcaagatgagtat |
| | | | | tgggtaatcattcaagccaa |
| | | | | ggacatgattggtcagccag |
| | | | | gagcgttgtctggaacaaca |
| | | | | agtgtattaattaaactttc |
| | | | | agatgttaatgacaataagc |
| | | | | ctatatttaaagaaagttta |
| | | | | taccgcttgactgtctctga |
| | | | | atctgcacccactgggactt |
| | | | | ctataggaacaatcatggca |
| | | | | tatgataatgacataggaga |
| | | | | gaatgcagaaatggattaca |
| | | | | gcattgaagaggatgattcg |
| | | | | caaacatttgacattattac |
| | | | | taatcatgaaactcaagaag |
| | | | | gaatagttatattaaaaaag |
| | | | | aaagtggattttgagcacca |
| | | | | gaaccactacggtattagag |
| | | | | caaaagttaaaaaccatcat |
| | | | | gttcctgagcagctcatgaa |
| | | | | gtaccacactgaggcttcca |
| | | | | ccactttcattaagatccag |
| | | | | gtggaagatgttgatgagcc |
| | | | | tcctcttttcctccttccat |
| | | | | attatgtatttgaagttttt |
| | | | | gaagaaaccccacagggatc |
| | | | | atttgtaggcgtggtgtctg |
| | | | | ccacagacccagacaatagg |
| | | | | aaatctcctatcaggtattc |
| | | | | tattactaggagcaaagtgt |
| | | | | tcaatatcaatgataatggt |
| | | | | acaatcactacaagtaactc |
| | | | | actggatcgtgaaatcagtg |
| | | | | cttggtacaacctaagtatt |
| | | | | acagccacagaaaaatacaa |
| | | | | tatagaacagatctcttcga |
| | | | | tcccactgtatgtgcaagtt |
| | | | | cttaacatcaatgatcatgc |
| | | | | tcctgagttctctcaatact |
| | | | | atgagacttatgtttgtgaa |
| | | | | aatgcaggctctggtcaggt |
| | | | | aattcagactatcagtgcag |
| | | | | tggatagacatgaatccaLa |
| | | | | gaagagcaccatttttactt |
| | | | | taatctatctgtagaagaca |
| | | | | ctaacaattcaagttttaca |
| | | | | atcatagataatcaagataa |
| | | | | cacagctgtcattttgacta |
| | | | | atagaactggttttaacctt |
| | | | | caagaagaacctgtcttcta |
| | | | | catctccatcttaattgccg |
| | | | | acaatggaatcccgtcactt |
| | | | | acaagtacaaacacccttac |
| | | | | catccatgtctgtgactgtg |
| | | | | gtgacagtgggagcacacag |
| | | | | acctgccagtaccaggagct |
| | | | | tgtgctttccatgggattca |
| | | | | agacagaagtcatcattgct |
| | | | | attctcatttgcattatgat |
| | | | | catatttgggtttatttttt |
| | | | | tgactttgggtttaaaacaa |
| | | | | cggagaaaacag |
| 952 | C137897 | artifi- | aa | GWVWNQFFVPEEMNTTSHHI |
| | huCDH19 | cial | | GQLRSDLDNGNNSFQYKLLG |
| | (44-141) | | | AGAGSTFIIDERTGDIYAIQ |
| | muCDH19 | | | KLDREERSLYILRAQVIDIA |
| | (140-770) | | | TGRAVEPESEFVIKVSDIND |
| | | | | NEPRFLDEPYEAIVPEMSPE |
| | | | | GTFVIKVTANDADDPSTGYH |
| | | | | ARILYNLERGQPYFSVEPTT |
| | | | | GVIRISSKMDRELQDTYCVI |
| | | | | IQAKDMLGQPGALSGTTTVS |
| | | | | IKLSDINDNKPIFKESFYRF |
| | | | | TISESAPIGTSIGKIMAYDD |
| | | | | DIGENAEMEYSIEDDDSKIF |
| | | | | DIIIDMDTQEGIVILKKKVD |
| | | | | FEQQSYYGIRAKVKNCHVDE |
| | | | | ELAPAHVNASTTYIKVQVED |
| | | | | EDEPPVFLLPYYILEIPEGK |
| | | | | PYGTIVGTVSATDPDRRQSP |
| | | | | MRYYLTGSKMFDINDNGTII |
| | | | | TTNMLDREVSAWYNLTVTAT |
| | | | | ETYNVQQISSAHVYVQVFNI |
| | | | | NDNAPEFSQFYETYVCENAE |
| | | | | SGEIVQIISAIDRDESIEDH |
| | | | | HFYFNHSLEDTNNSSFMLTD |
| | | | | NQDNTAVILSNRTGFNLKEE |
| | | | | PVFYMIILIADNGIPSLTST |
| | | | | NTLTIQVCDCGDSRNTETCA |
| | | | | NKGLLFIMGFRTEAIIAIMI |
| | | | | CVMVIFGFFFLILALKQRRK |
| | | | | ETLFPEKTEDFRENIFCYDD |
| | | | | EGGGEEDSEAFDIVELRQST |
| | | | | VMRSRKPQRSKSAEIRSLYR |
| | | | | QSLQVGPDSAIFRKFILEKL |
| | | | | SEAMTDPCAPPFDSLQTFAY |
| | | | | EGTGSSAGSLSSLASRDTDQ |
| | | | | EDDFDYLNDLGPRFKRLASM |
| | | | | FGSAVQPNN |
| 953 | C137897 | artifi- | nt | ggctgggtgtggaaccaatt |
| | huCDH19 | cial | | ttttgtaccagaggaaatga |
| | (44-141) | | | atacgactagtcatcacatc |
| | muCDH19 | | | ggccagctaagatctgattt |
| | (140-770) | | | agacaatggaaacaattctt |
| | | | | tccagtacaagcttttggga |
| | | | | gctggagctggaagtacttt |
| | | | | tatcattgatgaaagaacag |
| | | | | gtgacatatatgccatacag |
| | | | | aagcttgatagagaggagcg |
| | | | | atccctctacatcttaagag |
| | | | | cccaggtaatagacatcgct |
| | | | | actggaagggctgtggaacc |
| | | | | tgagtctgagtttgtcatca |
| | | | | aagtttcggatatcaatgac |
| | | | | aatgaacccagattcctaga |
| | | | | tgaaccatatgaggccattg |
| | | | | tacctgagatgtctccagaa |
| | | | | ggaacatttgtcatcaaggt |
| | | | | gacagccaatgacgcagatg |
| | | | | atccttcaactggctatcat |
| | | | | gctcgcatcctatacaactt |
| | | | | agaacgaggtcaaccatact |
| | | | | tttctgttgagccaacaaca |
| | | | | ggagtcataaggatatcttc |
| | | | | taagatggatagagagttgc |
| | | | | aagatacatactgtgtaatt |
| | | | | attcaagccaaggacatgct |
| | | | | cggtcagcctggagccttgt |
| | | | | ctggaacaacaaccgtatca |
| | | | | attaagctgtcagatattaa |
| | | | | tgacaacaagccaatattca |
| | | | | aagaaagtttctaccgcttc |
| | | | | actatatctgaatctgcacc |
| | | | | cattggaacatcaataggga |
| | | | | aaattatggcatatgatgat |
| | | | | gacataggggagaatgcaga |
| | | | | gatggagtacagcattgaag |
| | | | | atgatgattcaaaaatattt |
| | | | | gacataatcattgacaatga |
| | | | | cacccaagaagggatagtta |
| | | | | tacttaaaaagaaagttgat |
| | | | | tttgagcagcagagctatta |
| | | | | tggcattagagctaaggtta |
| | | | | aaaactgccatgtggatgaa |
| | | | | gagcttgcacctgcccatgt |
| | | | | taacgcttccacaacctaca |
| | | | | ttaaagttcaagtagaagat |
| | | | | gaagatgaacctcctgtttt |
| | | | | cctcttaccatattacatac |
| | | | | ttgaaattcctgaaggaaaa |
| | | | | ccatatggaacaattgtggg |
| | | | | gacggtttctgccacagacc |
| | | | | cagatcgaagacaatctcct |
| | | | | atgagatattatctcactgg |
| | | | | aagcaaaatgtttgatatca |
| | | | | atgacaatggaacaataatc |
| | | | | accactaacatgcttgacag |
| | | | | agaggtcagtgcttggtaca |
| | | | | acttgactgtcacagctact |
| | | | | gaaacatacaatgtacaaca |
| | | | | gatctcttcagcccatgttt |
| | | | | atgtacaagtctttaacatt |
| | | | | aacgacaatgctccagagtt |
| | | | | ctctcaattctatgagactt |
| | | | | atgtttgtgaaaatgctgaa |
| | | | | tctggtgagatagttcagat |
| | | | | catcagtgcaattgatagag |
| | | | | atgagtccatagaagatcac |
| | | | | catttttactttaatcactc |
| | | | | tctggaagacacaaacaact |
| | | | | caagttttatgctaacagac |
| | | | | aatcaagataacacagctgt |
| | | | | aattctgagtaatagaactg |
| | | | | gtttcaatcttaaagaagag |
| | | | | cctgtcttctacattgatca |
| | | | | tcttgattgctgataacggga |
| | | | | tcccatctctcacaagcaca |
| | | | | aacactctcactatccaagt |
| | | | | ctgtgactgtggagacagta |
| | | | | gaaacacagaaacttgtgct |
| | | | | aacaagggacttctctttat |
| | | | | catgggattcagaacagagg |
| | | | | caataattgccatcatgata |
| | | | | tgtgttatggtaatatttgg |
| | | | | gtttttctttttgattcttg |
| | | | | ctctgaaacagcgaagaaag |
| | | | | gagactctatttccagagaa |
| | | | | gactgaagactttagggaga |
| | | | | atatattttgctatgatgat |
| | | | | gaaggcggcggggaagaaga |
| | | | | ctcggaagcctttgacatcg |
| | | | | tagagctgagacaaagtaca |
| | | | | gtaatgagagaaagaaagcc |
| | | | | tcagagaagcaagagtgcgg |
| | | | | agatcaggagcttgtacagg |
| | | | | cagtccctgcaggtgggccc |
| | | | | agacagtgccatatttcgaa |
| | | | | aatttatcccagagaagctt |
| | | | | gaagaagccaacacagaccc |
| | | | | atgtgctcccccctttgatt |
| | | | | cactacagacgtttgcctat |
| | | | | gagggaacagggtcaicagc |
| | | | | tggctctctgagctccttgg |
| | | | | catccagagacactgatcag |
| | | | | gaggatgacttcgactacct |
| | | | | taatgacctgggacctcgtt |
| | | | | ttaaaagattagcaagcatg |
| | | | | tttggctctgcagtacaacc |
| | | | | caacaattag |
| 954 | C137896 | artifi- | aa | GWVWNQFFVPEEMNTTSHHI |
| | huCDH19 | cial | | GQLRSDLDNGNNSFQYKLLG |
| | (44-249) | | | AGAGSTFIIDERTGDIYAIQ |
| | muCDH19 | | | KLDREERSLYILRAQVIDIA |
| | (248-770) | | | TGRAVEPESEFVIKVSDIND |
| | | | | NEPKFLDSPYEAIVPEMSPE |
| | | | | GTLVIQVTASDADDPSSGNN |
| | | | | ARLLYSLLQGQPYFSVEPTT |
| | | | | GVIRISSKMDRELQDEYWVI |
| | | | | IQAKDMIGQPGALSGTTSVL |
| | | | | IKLSDVNDNKPIFKESFYRF |
| | | | | TISESAPIGTSIGKIMAYDD |
| | | | | DIGENAEMEYSIEDDDSKIF |
| | | | | DIIIDNDTQEGIVILKKKVD |
| | | | | FEQQSYYGIRAKVKNCHVDE |
| | | | | ELAPAHVNASTTYIKVQVED |
| | | | | EDEPPVFLLPYYILEIPEGK |
| | | | | PYGTIVGTVSATDPDRRQSP |
| | | | | MRYYLTGSKMFDINDNGTII |
| | | | | TTNMLDREVSAWYNLTVTAT |
| | | | | ETYNVQQISSAHVYVQVFNI |
| | | | | NDNAPEFSQFYETYVCENAE |
| | | | | SGEIVQIISAIDRDESIEDH |
| | | | | HFYFNHSLEDTNNSSFMLTD |
| | | | | NQDNTAVILSNRTGFNLKEE |
| | | | | PVFYMIILIADNGIPSLTST |
| | | | | NTLTIQVCDCGDSRNTETCA |
| | | | | NKGLLFIMGFRTEAIIAIMI |
| | | | | CVMVIFGFFFLILALKQRRK |
| | | | | ETLFPEKTEDFRENIFCYDD |
| | | | | EGGGEEDSEAFDIVELRQST |
| | | | | VMRERKPQRSKSAEIRSLYR |
| | | | | QSLQVGPDSAIFRKFILEKL |
| | | | | SEANTDPCAPPFDSLQTFAY |
| | | | | EGTGSSAGSLSSLASRDTDQ |
| | | | | EDDFDYLNDLGPRFKRLASM |
| | | | | FGSAVQPNN |
| 955 | C137896 | artifi- | nt | ggctgggtgtggaaccaa |
| | huCDH19 | cial | | ttttttgtaccagaggaa |
| | (44-249) | | | atgaatacgactagtcat |
| | muCDH19 | | | cacatcggccagctaaga |
| | (248-770) | | | tctgatttagacaatgg |
| | | | | aaacaattctttccagt |
| | | | | acaagcttttgggagctgga |
| | | | | gctggaagtacttttatcat |
| | | | | tgatgaaagaacaggttgac |
| | | | | atatatgccatacagaagct |
| | | | | tgatagagaggagcgatccc |
| | | | | tctacatcttaagagcccag |
| | | | | gtaatagacatcgctactgg |
| | | | | aagggctgtggaacctgagt |
| | | | | ctgagtttgtcatcaaagtt |
| | | | | tcggatatcaatgacaatga |
| | | | | accaaaattcctagatgaac |
| | | | | cttatgaggccattgtacca |
| | | | | gagatgtctccagaaggaac |
| | | | | attagttatccaggtgacag |
| | | | | caagtgatgctgacgatccc |
| | | | | tcaagtggtaataatgctcg |
| | | | | tctcctctacagcttacttc |
| | | | | aaggccagccatatttttct |
| | | | | gttgaaccaacaacaggagt |
| | | | | cataagaatatcttctaaaa |
| | | | | tggatagagaactgcaagat |
| | | | | gagtattgggtaatcattca |
| | | | | agccaaggacatgattggtc |
| | | | | agccaggagcgttgtctgga |
| | | | | acaacaagtgtattaattaa |
| | | | | actttcagatgttaatgaca |
| | | | | acaagccaatattcaaagaa |
| | | | | agtttctaccgcttcactat |
| | | | | atctgaatctgcacccattg |
| | | | | gaacatcaatagggaaaatt |
| | | | | atggcatatgatgatgacat |
| | | | | aggggagaatgcagagatgg |
| | | | | agtacagcattgaagatgat |
| | | | | gattcaaaaatatttgacat |
| | | | | aatcattgacaatgacaccc |
| | | | | aagaagggatagttatactt |
| | | | | aaaaagaaagttgattttga |
| | | | | gcagcagagctattatggca |
| | | | | ttagagctaaggttaaaaac |
| | | | | tgccatgtggatgaagagct |
| | | | | tgcacctgcccatgttaacg |
| | | | | cttccacaacctacattaaa |
| | | | | gttcaagtagaagatgaaga |
| | | | | tgaacctcctgttttcctct |
| | | | | taccatattacatacttgaa |
| | | | | attcctgaaggaaaacca~a |
| | | | | tggaacaattgtggggacgg |
| | | | | tttctgccacagacccagat |
| | | | | cgaagacaatctcctatgag |
| | | | | atattatctcactggaagca |
| | | | | aaatgtttgatatcaatgac |
| | | | | aatggaacaataatcaccac |
| | | | | taacatgcttgacagagagg |
| | | | | tcagtgcttggtacaacttg |
| | | | | actgtcacagctactgaaac |
| | | | | atacaatgtacaacagatct |
| | | | | cttcagcccatgtttatgta |
| | | | | caagtctttaacattaacga |
| | | | | caatgctccagagttctctc |
| | | | | aattctatgagacttatgtt |
| | | | | tgtgaaaatgctgaatctgg |
| | | | | tgagatagttcagatcatca |
| | | | | gtgcaattgatagagatgag |
| | | | | tccatagaagatcaccattt |
| | | | | ttactttaatcactctctgg |
| | | | | aagacacaaacaactcaagt |
| | | | | tttatgctaacagacaatca |
| | | | | agataacacagctgtaattc |
| | | | | tgagtaatagaactggtttc |
| | | | | aatcttaaagaagagcctgt |
| | | | | cttctacatgatcatcttga |
| | | | | ttgctgataacgggatccca |
| | | | | tctctcacaagcacaaacac |
| | | | | tctcactatccaagtctgtg |
| | | | | actgtggagacagtacaaac |
| | | | | acagaaacttgtgctaacaa |
| | | | | gcgacttctctttatcatgg |
| | | | | gattcagaacagaggcaata |
| | | | | attgccatcatgatatgtgt |
| | | | | tatggtaatatttgggtttt |
| | | | | tctttttgattcttgctctg |
| | | | | aaacagcgaagaaaggagac |
| | | | | tctatttccagagaagactg |
| | | | | aagactttagggagaatata |
| | | | | ttttgctatgatgatgaagg |
| | | | | cggcggggaagaagactcgg |
| | | | | aagcctttgacatcgtagag |
| | | | | ctgagacaaagtacagtaat |
| | | | | gagagaaagaaagcctcaga |
| | | | | gaagcaagagtgcggagatc |
| | | | | aggagcttgtacaggcagtc |
| | | | | cctgcaggtgggcccagaca |
| | | | | gtgccatatttcgaaaattt |
| | | | | atcctagagaagcttgaaga |
| | | | | agccaacacagacccatgtg |
| | | | | ctcccccctttgattcacta |
| | | | | cagacgtttgcctatgaggg |
| | | | | aacagggtcatcagctggct |
| | | | | ctctgagctccttggcatcc |
| | | | | agagacactgatcaggagga |
| | | | | tgacttcgactaccttaatg |
| | | | | acctgggacctcgttttaaa |
| | | | | agattagcaagcatgtttgg |
| | | | | ctctgcagtacaacccaaca |
| | | | | attag |
| 956 | C137913 | artifi- | aa | AWVWRPFVVLEEMDDIQCVG |
| | muCDH19 | cial | | KLRSDLDNGNNSFQYKLLGI |
| | (44-139) | | | GAGSFSINERTGEICAIQKL |
| | huCDH19 | | | DREEKSLYILRAQVIDTTIG |
| | (142-249) | | | KAVETESEFVIRVLDINDNE |
| | muCDH19 | | | PKFLDEPYEAIVPEMSPEGT |
| | (248-770) | | | LVIQVTASDADDPSSGNNAR |
| | | | | LLYSLLQGQPYFSVEPTTGV |
| | | | | IRISSKMDRELQDEYWVIIQ |
| | | | | AKDMIGQPGALSGTTSVLIK |
| | | | | LSDVNDNKPIFKESFYRFTI |
| | | | | SESAPIGTSIGKIMAYDDDI |
| | | | | GENAEMEYSIEDDDSKIFDI |
| | | | | IIDNDTQEGIVILKKKVDFE |
| | | | | QQSYYGIRAKVKNCHVDEEL |
| | | | | APAHVNASTTYIKVQVEDED |
| | | | | EPPVFLLPYYILEIPEGKPY |
| | | | | GTIVGTVSATDPDRRQSPMR |
| | | | | YYLTGSKMFDINDNGTIITT |
| | | | | NMLDREVSAWYNLTVTATET |
| | | | | YNVQQISSAHVYVQVFNIND |
| | | | | NAPEFSQFYETYVCENAESG |
| | | | | EIVQIISAIDRDESIEDHHF |
| | | | | YFNHSLEDTNNSSFMLTDNQ |
| | | | | DNTAVILSNRTGFNLKEEPV |
| | | | | FYMIILIADNGIPSLTSTNT |
| | | | | LTIQVCDCGDSRNTETCANK |
| | | | | GLLFIMGFRTEAIIAIMICV |
| | | | | MVIFGFFFLILALKQRRKET |
| | | | | LFPEKTEDFRENIFCYDDEG |
| | | | | GGEEDSEAFDIVELRQSTVM |
| | | | | RERKPQRSKSAEIRSLYRQS |
| | | | | LQVGPDSAIFRKFILEKLEE |
| | | | | ANTDPCAPPFDSLQTFAYEG |
| | | | | TGSSAGSLSSLASRDTDQED |
| | | | | DFDYLNDLGPRFKRLASMFG |
| | | | | SAVQPNN |
| 957 | C137913 | artifi- | nt | gcctgggtgtggagaccatt |
| | muCDH19 | cial | | tgttgttctagaagaaatgg |
| | (44-139) | | | atgatatacaatgtgttgga |
| | huCDH19 | | | aagctaagatctgacttaga |
| | (142-249) | | | caatggaaacaactctttcc |
| | muCDH19 | | | agtacaagctactggggatt |
| | (248-770) | | | ggcgctggaagctttagc |
| | | | | attaatgaaagaacaggt |
| | | | | gaaatatgtgccatacag |
| | | | | aagcttgatagagaggaa |
| | | | | aaatccctctacattctgag |
| | | | | agcccaggtaatagacacca |
| | | | | ctattgggaaggctgtggaa |
| | | | | actgaatccgagtttgtcat |
| | | | | cagagttttggatatcaatg |
| | | | | acaatgaaccaaaattccta |
| | | | | gatgaaccttatgaggccat |
| | | | | tgtaccagagatgtctccag |
| | | | | aaggaacattagttatccag |
| | | | | gtgacagcaagtgatgctga |
| | | | | cgatccctcaagtggtaata |
| | | | | atgctcgtctcctctacagc |
| | | | | ttacttcaaggccagccata |
| | | | | tttttctgttgaaccaacaa |
| | | | | caggagtcataagaatatct |
| | | | | tctaaaatggatagagaact |
| | | | | gcaagatgagtattgggtaa |
| | | | | tcattcaagccaaggacatg |
| | | | | attggtcagccaggagcgtt |
| | | | | gtctggaacaacaagtgtat |
| | | | | taattaaactttcagatgtt |
| | | | | aatgacaacaagccaatatt |
| | | | | caaagaaagtttctaccgct |
| | | | | tcactatatctgaatctgca |
| | | | | cccattggaacatcaatagg |
| | | | | gaaaattatggcatatgatg |
| | | | | atgacataggggagaatgca |
| | | | | gagatggagtacagcattga |
| | | | | agatgatgattcaaaaatat |
| | | | | ttgacataatcattgacaat |
| | | | | gacacccaagaagggatagt |
| | | | | tatacttaaaaagaaagttg |
| | | | | attttgagcagcagagctat |
| | | | | tatggcattagagctaaggt |
| | | | | taaaaactgccatgtggatg |
| | | | | aagagcttgcacctgcccat |
| | | | | gttaacgcttccacaaccta |
| | | | | cattaaagttcaagtagaag |
| | | | | atgaagatgaacctcctgtt |
| | | | | ttcctcttaccatattacat |
| | | | | acttgaaattcctgaaggaa |
| | | | | aaccatatggaacaattgtg |
| | | | | gggacggtttctgccacaga |
| | | | | cccagatcgaagacaatctc |
| | | | | ctatgagatattatctcact |
| | | | | ggaagcaaaatgtttgatat |
| | | | | caatgacaatggaacaataa |
| | | | | tcaccactaacatgcttgac |
| | | | | agagaggtcagtgcttggta |
| | | | | caacttgactgtcacagcta |
| | | | | ctgaaacatacaatgtacaa |
| | | | | cagatctcttcagcccatgt |
| | | | | ttatgtacaagtctttaaca |
| | | | | ttaacgacaatgctccagag |
| | | | | ttctctcaa-tctatgagac |
| | | | | ttatgtttgtgaaaatgctg |
| | | | | aatctggtgagatagttcag |
| | | | | atcatcagtgcaattgatag |
| | | | | agatgagtccatagaagatc |
| | | | | accatttttactttaatcac |
| | | | | tctctggaagacacaaacaa |
| | | | | ctcaagttttatgctaacag |
| | | | | acaatcaagataacacagct |
| | | | | gtaattctgagtaatagaac |
| | | | | tggtttcaatcttaaagaag |
| | | | | agcctgtcttctacatgatc |
| | | | | atcttgattgctgataacgg |
| | | | | gatcccatctctcacaagca |
| | | | | caaacactctcactatccaa |
| | | | | gtctgtgactgtggagacag |
| | | | | tagaaacacagaaacttgtg |
| | | | | ctaacaagggacttctcttt |
| | | | | atcatgggattcagaacaga |
| | | | | ggcaacaattgccatcatga |
| | | | | tatgtgttatggtaatattt |
| | | | | gggtttttctttttgattct |
| | | | | tgctctgaaacagcgaagaa |
| | | | | aggagactctacttccagag |
| | | | | aagactgaagactttaggga |
| | | | | gaatatattttgctatgatg |
| | | | | atgaaggcggcggggaagaa |
| | | | | gactcggaagcctttgacat |
| | | | | cgtagagctgagacaaagta |
| | | | | cagcaatgagagaaagaaag |
| | | | | cctcagagaagcaagagtgc |
| | | | | ggagatcaggagcttgtaca |
| | | | | ggcagtccctgcaggtgggc |
| | | | | ccagacagtgccatatttcg |
| | | | | aaaatttatcctagagaagc |
| | | | | ttgaagaagccaacacagac |
| | | | | ccatgtgctcccccctttga |
| | | | | ttcactacagacgtttgcct |
| | | | | atgagggaacagggtcatca |
| | | | | gctggctctctgagctcctt |
| | | | | ggcatccagagacactgatc |
| | | | | aggaggatgacttcgactac |
| | | | | cttaatgacctgggacctcg |
| | | | | ttttaaaagattagcaagca |
| | | | | tgtttggctctgcagtacaa |
| | | | | cccaacaattag |
| 958 | C137847 | artifi- | aa | AWVWRPFVVLEEMDDIQCVG |
| | muCDH19 | cial | | KLRSDLDNGNNSFQYKLLGI |
| | (44-139) | | | GAGSFSINERTGEICAIQKL |
| | huCDH19 | | | DREEKSLYILRAQVIOTTIG |
| | (142-364) | | | KAVETESEFVIRVLDINDNE |
| | muCDH19 | | | PKFLDEPYEAIVPEMSPEGT |
| | (363-770) | | | LVIQVTASDADDPSSGNNAR |
| | | | | LLYSLLQGQPYFSVEPTTGV |
| | | | | IRISSKMDRELQDEYWVIIQ |
| | | | | AKDMIGQPGALSGTTSVLIK |
| | | | | LSDVNDNKPIFKESLYRLTV |
| | | | | SESAPTGTSIGTIMAYDNDI |
| | | | | GENAEMDYSIEEDDSQTFDI |
| | | | | ITNHETQEGIVILKKKVDFE |
| | | | | HQNHYGIRAKVKNHHVPEQL |
| | | | | MKYHTEASTTFIKIQVEDVD |
| | | | | EPPVFLLPYYILEIPEGKPY |
| | | | | GTIVGTVSATDPDRRQSPMR |
| | | | | YYLTGSKMFDINDNGTIITT |
| | | | | NMLDREVSAWYNLTVTATET |
| | | | | YNVQQISSAHVYVQVFNIND |
| | | | | NAPEFSQFYETYVCENAESG |
| | | | | EIVQIISAIDRDESIEDHHF |
| | | | | YFNHSLEDTNNSSFMLTDNQ |
| | | | | DNTAVILSNRTGFNLKEEPV |
| | | | | FYMIILIADNGIPSLTSTNT |
| | | | | LTIQVCDCGDSRNTETCANK |
| | | | | GLLFIMGFRTEAIIAIMICV |
| | | | | MVIFGFFFLILALKQRRKET |
| | | | | LFPEKTEDFRENIFCYDDEG |
| | | | | GGEEDSEAFDIVELRQSTVM |
| | | | | RERKPQRSKSAEIRSLYRQS |
| | | | | LQVGPDSAIFRKFILEKLEE |
| | | | | ANTDPCAPPFDSLQTFAYEG |
| | | | | TGSSAGSLSSLASRDTDQED |
| | | | | DFDYLNDLGPRFKRLASMFG |
| | | | | SAVQPNN |
| 959 | C137847 | artifi- | nt | gcctgggtgtggagaccatt |
| | muCDH19 | cial | | tgttgttctagaagaaatgg |
| | (44-139) | | | atgatatacaatgtgttgga |
| | huCDH19 | | | aagctaagatctgacttaga |
| | (142-364) | | | caatggaaacaactctttcc |
| | muCDH19 | | | agtacaagctactggggatt |
| | (363-770) | | | ggcgctggaagctttagcat |
| | | | | taatgaaagaacaggtgaaa |
| | | | | tatgtgccatacagaagctt |
| | | | | gatagagaggaaaaatccct |
| | | | | ctacattctgagagcccagg |
| | | | | taatagacaccactattggg |
| | | | | aaggctgtggaaactgaatc |
| | | | | cgagtttgtcatcagagttt |
| | | | | tggatatcaatgacaatgaa |
| | | | | ccaaaattcctagatgaacc |
| | | | | ttatgaggccattgtaccag |
| | | | | agatgtctccagaaggaaca |
| | | | | ttagttatccaggtgacagc |
| | | | | aagtgatgctgacgatccct |
| | | | | caagtggtaataatgctcgt |
| | | | | ctcctctacagcttacttca |
| | | | | aggccagccatatttttctg |
| | | | | ttgaaccaacaacaggagtc |
| | | | | ataagaatatcttctaaaat |
| | | | | ggatagagaactgcaagatg |
| | | | | agtattgggtaatcattcaa |
| | | | | gccaaggacatgattggtca |
| | | | | gccaggagcgttgtctggaa |
| | | | | caacaagtgtattaattaaa |
| | | | | ctttcagatgttaatgacaa |
| | | | | taagcctatatttaaagaaa |
| | | | | gtttataccgcttgactgtc |
| | | | | tctgaatctgcacccactgg |
| | | | | gacttctataggaacaatca |
| | | | | tggcatatgataatgacata |
| | | | | ggagagaatgcagaaatgga |
| | | | | ttacagcattgaagaggatg |
| | | | | attcgcaaacatttgacatt |
| | | | | attactaatcatgaaactca |
| | | | | agaaggaatagttatattaa |
| | | | | aaaagaaagtggattttgag |
| | | | | caccagaaccactacggtat |
| | | | | tagagcaaaagttaaaaacc |
| | | | | atcatgttcctgagcagctc |
| | | | | atgaagtaccacactgaggc |
| | | | | ttccaccactttcattaaga |
| | | | | tccaggtggaagatgttgat |
| | | | | gaacctcctgttttcctctt |
| | | | | accatattacatacttgaaa |
| | | | | ttcctgaaggaaaaccatat |
| | | | | ggaacaattgtggggacggt |
| | | | | ttctgccacagacccagatc |
| | | | | gaagacaatctcctatgaga |
| | | | | tattatctcactggaagcaa |
| | | | | aatgtttgatatcaatgaca |
| | | | | atggaacaataatcaccact |
| | | | | aacatgcttgacagagaggt |
| | | | | cagtgcttggtacaacttga |
| | | | | ctgtcacagctactgaaaca |
| | | | | tacaatgtacaacagatctc |
| | | | | ttcagcccatgtttatgtac |
| | | | | aagtctttaacattaacgac |
| | | | | aatgctccagagttctctca |
| | | | | attctatgagacttatgttt |
| | | | | gtgaaaatgctgaatctggt |
| | | | | gagatagttcagatcatcag |
| | | | | tgcaattgatagagatgagt |
| | | | | ccatagaagatcaccatttt |
| | | | | tactttaatcactctctgga |
| | | | | agacacaaacaactcaagtt |
| | | | | ttatgctaacagacaatcaa |
| | | | | gataacacagctgtaattct |
| | | | | gagtaatagaactggtttca |
| | | | | atcttaaagaagagcctgtc |
| | | | | ttctacatgatcatcttgat |
| | | | | tgctgataacgggatcccat |
| | | | | ctctcacaagcacaaacact |
| | | | | ctcactatccaagtctgtga |
| | | | | ctgtggagacagtagaaaca |
| | | | | cagaaacttgtgctaacaag |
| | | | | ggacttctctttatcatggg |
| | | | | attcagaacagaggcaataa |
| | | | | ttgccatcatgatatgtgtt |
| | | | | atggtaatatttgggttttt |
| | | | | ctttttgattcttgctctga |
| | | | | aacagcgaagaaaggagact |
| | | | | ctatttccagagaagactga |
| | | | | agactttagggagaatatat |
| | | | | tttgctatgatgatgaaggc |
| | | | | ggcggggaagaagactcgga |
| | | | | agcctttgacatcgtagagc |
| | | | | tgagacaaagtacagtaatg |
| | | | | agagaaagaaagcctcagag |
| | | | | aagcaagagtgcggagatca |
| | | | | ggagcttgtacaggcagtcc |
| | | | | ctgcaggtgggcccagacag |
| | | | | tgccatatttcgaaaattta |
| | | | | tcctagagaagcttgaagaa |
| | | | | gccaacacagacccatgtgc |
| | | | | tcccccctttgattcactac |
| | | | | agacgtttgcctatgaggga |
| | | | | acagggtcatcagctggctc |
| | | | | tctgagctccttggcatcca |
| | | | | gagacactgatcaggaggat |
| | | | | gacttcgactaccttaatga |
| | | | | cctgggacctcgttttaaaa |
| | | | | gattagcaagcatgtttggc |
| | | | | tctgcagtacaacccaacaa |
| | | | | ttag |
| 960 | C137911 | artifi- | aa | AWVWRPFVVLEEMDDIQCVG |
| | muCDH19 | cial | | KLRSDLDNGNNSFQYKLLGI |
| | (44-247) | | | GAGSFSINERTGEICAIQKL |
| | huCDH19 | | | DREEKSLYILRAQVIDTTIG |
| | (250-364) | | | KAVETESEFVIRVLDINDNE |
| | muCDH19 | | | PRFLDEPYEAIVPEMSPEGT |
| | (363-770) | | | FVIKVTANDADDPSTGYHAR |
| | | | | ILYNLERGQPYFSVEPTTGV |
| | | | | IRISSKMDRELQDTYCVIIQ |
| | | | | AKDMLGQPGALSGTTTVSIK |
| | | | | LSDINDNKPIFKESLYRLTV |
| | | | | SESAPTGTSIGTIMAYDNDI |
| | | | | GENAEMDYSIEEDDSQTFDI |
| | | | | ITNHETQEGIVILKKKVDFE |
| | | | | HQNHYGIRAKVKNHHVPEQL |
| | | | | MKYHTEASTTFIKIQVEDVD |
| | | | | EPPVFLLPYYILEIPEGKPY |
| | | | | GTIVGTVSATDPDRRQSPMR |
| | | | | YYLTGSKMFDINDNGTIITT |
| | | | | NMLDREVSAWYNLTVTATET |
| | | | | YNVQQISSAHVYVQVFNIND |
| | | | | NAPEFSQFYETYVCENAESG |
| | | | | EIVQIISAIDRDESIEDHHF |
| | | | | YFNHSLEDTNNSSFMLTDNQ |
| | | | | DNTAVILSNRTGFNLKEEPV |
| | | | | FYMIILIADNGIPSLTSTNT |
| | | | | LTIQVCDCGDSRNTETCANK |
| | | | | GLLFIMGFRTEAIIAIMICV |
| | | | | MVIFGFFFLILALKQRRKET |
| | | | | LFPEKTEDFRENIFCYDDEG |
| | | | | GGEEDSEAFDIVELRQSTVM |
| | | | | RERKPQRSKSAEIRSLYRQS |
| | | | | LQVGPDSAIFRKFILEKLEE |
| | | | | ANTDPCAPPFDSLQTFAYEG |
| | | | | TGSSAGSLSSLASRDTDQED |
| | | | | DFDYLNDLGPRF |
| 961 | C137911 | artifi- | nt | gcctgggtgtggagaccatt |
| | muCDH19 | cial | | tgttgttctagaagaaatgg |
| | (44-247) | | | atgatatacaatgtgttgga |
| | huCDH19 | | | aagctaagatctgacttaga |
| | (250-364) | | | caatggaaacaactctttcc |
| | muCDH19 | | | agtacaagctactggggatt |
| | (363-770) | | | ggcgctggaagctttagcat |
| | | | | taatgaaagaacaggtgaaa |
| | | | | tatgtgccatacagaagctt |
| | | | | gatagagaggaaaaatccct |
| | | | | ctacattctgagagcccagg |
| | | | | taatagacaccactattggg |
| | | | | aaggctgtggaaactgaatc |
| | | | | cgagtttgtcatcagagttt |
| | | | | tggatatcaatgacaatgaa |
| | | | | cccagattcctagatgaacc |
| | | | | atatgaggccattgtacctg |
| | | | | agat |
| | | | | gtctccagaaggaacatttg |
| | | | | tcatcaaggtgacagccaat |
| | | | | gacgcagatgatccttcaac |
| | | | | tggctatcatgctcgcatcc |
| | | | | tatacaacttagaacgaggt |
| | | | | caaccatacttttctgttga |
| | | | | gccaacaacaggagtcataa |
| | | | | ggatatcttctaagatggat |
| | | | | agagagttgcaagatacata |
| | | | | ctgtgtaattattcaagcca |
| | | | | aggacatgctcggtcagcct |
| | | | | ggagccttgtctggaacaac |
| | | | | aaccgtatcaattaagctgt |
| | | | | cagatattaatgacaataag |
| | | | | cctatatttaaagaaagttt |
| | | | | ataccgcttgactgtctctg |
| | | | | aatctgcacccactgggact |
| | | | | tctataggaacaatcatggc |
| | | | | atatgataatgacataggag |
| | | | | agaatgcagaaatggattac |
| | | | | agcattgaagaggatgattc |
| | | | | gcaaacatttgacattatta |
| | | | | ctaatcatgaaactcaagaa |
| | | | | ggaatagttatattaaaaaa |
| | | | | gaaagtggattttgagcacc |
| | | | | agaaccactacggtattaga |
| | | | | gcaaaagttaaaaaccatca |
| | | | | tgttcctgagcagctcatga |
| | | | | agtaccacactgaggcttcc |
| | | | | accactttcattaagatcca |
| | | | | ggtggaagatgttgatgaac |
| | | | | ctcctgttttcctcttacca |
| | | | | tattacatacttgaaattcc |
| | | | | tgaaggaaaaccatatggaa |
| | | | | caattgtggggacggtttct |
| | | | | gccacagacccagatcgaag |
| | | | | acaatctcctatgagatatt |
| | | | | atctcactggaagcaaaatg |
| | | | | tttgatatcaatgacaatgg |
| | | | | aacaataatcaccactaaca |
| | | | | tgcttgacagagaggtcagt |
| | | | | gcttggtacaacttgactgt |
| | | | | cacagctactgaaacataca |
| | | | | atgtacaacagatctcttca |
| | | | | gcccatgtttatgtacaagt |
| | | | | ctttaacattaacgacaatg |
| | | | | ctccagagttctctcaattc |
| | | | | tatgagacttatgtttgtga |
| | | | | aaatgctgaatctggtgaga |
| | | | | tagttcagatcatcagtgca |
| | | | | attgatagagatgagtccat |
| | | | | agaagatcaccatttttact |
| | | | | ttaatctatctgtagaagac |
| | | | | actaacaattcaagttttac |
| | | | | aatcatagataatcaagata |
| | | | | acacagctgtcattttgagt |
| | | | | aatagaactggtttcaatct |
| | | | | taaagaagagcctgtcttct |
| | | | | acatgatcatcttgattgct |
| | | | | gataacgggatcccatctct |
| | | | | cacaagcacaaacactctca |
| | | | | ctatccaagtctgtgactgt |
| | | | | ggagacagtagaaacacaga |
| | | | | aacttgtgctaacaagggac |
| | | | | ttctctttatcatgggattc |
| | | | | agaacagaggcaataattgc |
| | | | | catcatgatatgtgttatgg |
| | | | | taatatttgggtttttcttt |
| | | | | ttgattcttgctctgaaaca |
| | | | | gcgaagaaaggagactctat |
| | | | | ttccagagaagactgaagac |
| | | | | tttagggagaatatattttg |
| | | | | ctatgatgatgaaggcggcg |
| | | | | gggaagaagatacagaggcc |
| | | | | tttgatatagcagagctgag |
| | | | | gagtagtaccataatgcggg |
| | | | | aacgcaagactcggaaaacc |
| | | | | acaagcgcggagatcaggag |
| | | | | cttgtacaggcagtccctgc |
| | | | | aggtgggcccagacagtgcc |
| | | | | atatttcgaaaatttatcct |
| | | | | agagaagcttgaagaagcca |
| | | | | acacagacccatgtgctccc |
| | | | | ccctttgattcactacagac |
| | | | | gtttgcctatgagggaacag |
| | | | | ggtcatcagctggctctctg |
| | | | | agctccttagaatcagcagt |
| | | | | ctctgatcaggatgaaagct |
| | | | | atgattaccttaatgagttg |
| | | | | ggacctcgctttaaaagatt |
| | | | | agcatgcatgtttggctctg |
| | | | | cagtacaacccaacaattag |
| 962 | C137917 | artifi- | aa | AWVWRPFVVLEEMDDIQCVG |
| | muCDH19 | cial | | KLRSDLDNGNNSFQYKLLGI |
| | (44-362) | | | GAGSFSINERTGEICAIQKL |
| | huCDH19 | | | DREEKSLYILRAQVIDTTIG |
| | (365-772) | | | KAVETESEFVIRVLDINDNE |
| | | | | PRFLDEPYEAIVPEMSPEGT |
| | | | | FVIKVTANDADDPSTGYHAR |
| | | | | ILYNLERGQPYFSVEPTTGV |
| | | | | IRISSKMDRELQDTYCVIIQ |
| | | | | AKDMLGQPGALSGTTTVSIK |
| | | | | LSDINDNKPIFKESFYRFTI |
| | | | | SESAPIGTSIGKIMAYDDDI |
| | | | | GENAEMEYSIEDDDSKIFDI |
| | | | | IIDNDTQEGIVILKKKVDFE |
| | | | | QQSYYGIRAKVKNCHVDEEL |
| | | | | APAHVNASTTYIKVQVEDED |
| | | | | EPPLFLLPYYVFEVFEETPQ |
| | | | | GSFVGVVSATDPDNRKSPIR |
| | | | | YSITRSKVFNINDNGTITTS |
| | | | | NSLDREISAWYNLSITATEK |
| | | | | YNIEQISSIPLYVQVLNIND |
| | | | | HAPEFSQYYETYVCENAGSG |
| | | | | QVIQTISAVDRDESIEEHHF |
| | | | | YFNLSVEDTNNSSFTIIDNQ |
| | | | | DNTAVILTNRTGFNLQEEPV |
| | | | | FYISILIADNGIPSLTSTNT |
| | | | | LTIHVCDCGDSGSTQTCQYQ |
| | | | | ELVLSMGFKTEVIIAILICI |
| | | | | MIIFGFIFLTLGLKQRRKQI |
| | | | | LFPEKSEDFRENIFQYDDEG |
| | | | | GGEEDTEAFDIAELRSSTIM |
| | | | | RERKTRKTTSAEIRSLYRQS |
| | | | | LQVGPDSAIFRKFILEKLEE |
| | | | | ANTDPCAPPFDSLQTYAFEG |
| | | | | TGSLAGSLSSLESAVSDQDE |
| | | | | SYDYLNELGPRFKRLACMFG |
| | | | | SAVQSNN |
| 963 | C137917 | artifi- | nt | gcctgggtgtggagaccatt |
| | muCDH19 | cial | | tgtcgttctagaagaaatgg |
| | (44-362) | | | atgatatacaatgtgttgga |
| | huCDH19 | | | aagctaagatctgacttaga |
| | (365-772) | | | caatggaaacaactctttcc |
| | | | | agtacaagctactggggatt |
| | | | | ggcgctggaagctttagcat |
| | | | | taatgaaagaacaggtgaaa |
| | | | | tatgtgccatacagaagctt |
| | | | | gatagagaggaaaaatccct |
| | | | | ctacattctgagagcccagg |
| | | | | taatagacaccactattggg |
| | | | | aaggctgtggaaactgaatc |
| | | | | cgagtttgtcatcagagttt |
| | | | | tggatatcaatgacaatgaa |
| | | | | cccagattcctagatgaacc |
| | | | | atatgaggccattgtacctg |
| | | | | agatgtctccagaaggaaca |
| | | | | tttgtcatcaaggtgacagc |
| | | | | caatgacgcagatgatcctt |
| | | | | caactggctatcatgctcgc |
| | | | | atcctataca |
| | | | | acttagaacgaggtcaacca |
| | | | | tacttttctgttgagccaac |
| | | | | aacaggagtcataaggatat |
| | | | | cttctaagatggatagagag |
| | | | | ttgcaagatacatactgtgt |
| | | | | aattattcaagccaaggaca |
| | | | | tgctcggtcagcctggagcc |
| | | | | ttgtctggaacaacaaccgt |
| | | | | atcaattaagctgtcagata |
| | | | | ttaatgacaacaagccaata |
| | | | | ttcaaagaaagtttctaccg |
| | | | | cttcactatatctgaatctg |
| | | | | cacccattggaacatcaata |
| | | | | gggaaaattatggcatatga |
| | | | | tgatgacataggggagaatg |
| | | | | cagagatggagtacagcatt |
| | | | | gaagatgatgattcaaaaat |
| | | | | atttgacataatcattgaca |
| | | | | atgacacccaagaagggata |
| | | | | gttatacttaaaaagaaagt |
| | | | | tgattttgagcagcagagct |
| | | | | attatggcattagagctaag |
| | | | | gttaaaaactgccatgtgga |
| | | | | tgaagagcttgcacctgccc |
| | | | | atgttaacgcttccacaacc |
| | | | | tacattaaagttcaagtaga |
| | | | | agatgaagatgagcctcctc |
| | | | | ttttcctccttccatattat |
| | | | | gtatttgaagtttttgaaga |
| | | | | aaccccacagggaLcatttg |
| | | | | taggcgtggtgtctgccaca |
| | | | | gacccagacaataggaaatc |
| | | | | tcctatcaggtattctatta |
| | | | | ctaggagcaaagtgttcaat |
| | | | | atcaatgataatggtacaat |
| | | | | cactacaagtaactcactgg |
| | | | | atcgtgaaatcagtgct~gg |
| | | | | tacaacctaagtattacagc |
| | | | | cacagaaaaatacaatatag |
| | | | | aacagatctcttcgatccca |
| | | | | ctgtatgtgcaagttcttaa |
| | | | | catcaatgatcatgctcctg |
| | | | | agttctctcaatactatgag |
| | | | | acttatgtttgtgaaaatgc |
| | | | | aggctctggtcaggtaattc |
| | | | | agactatcagtgcagtggat |
| | | | | agagatgaatccatagaaga |
| | | | | gcaccatttttactttaatc |
| | | | | tatctgtagaagacactaac |
| | | | | aattcaagttttacaatcat |
| | | | | agataatcaagataacacag |
| | | | | ctgtcattttgactaataga |
| | | | | actggttttaaccttcaaga |
| | | | | agaacctgtcttctacatct |
| | | | | ccattcttaattgccgacaat |
| | | | | ggaatcccgtcacttacaag |
| | | | | tacaaacacccttaccatcc |
| | | | | atgtctgtgactgtggtgac |
| | | | | agtgggagcacacagacctg |
| | | | | ccagtaccaggagcttgtgc |
| | | | | tttccatgggattcaagaca |
| | | | | gaagtcatcattgctattct |
| | | | | catttgcattatgatcatat |
| | | | | ttgggtttatttttttgact |
| | | | | ttgggtttaaaacaacggag |
| | | | | aaaacagattctatttcctg |
| | | | | agaaaagtgaagatttcaga |
| | | | | gagaatatattccaatatga |
| | | | | tgatgaagggggtggagaag |
| | | | | aagatacagaggcctttgat |
| | | | | atagcagagctgaggagtag |
| | | | | taccataatgcgggaacgca |
| | | | | agactcggaaaaccacaagc |
| | | | | gctgagatcaggagcctata |
| | | | | caggcagtctttgcaagttg |
| | | | | gccccgacagtgccatattc |
| | | | | aggaaattcattctggaaaa |
| | | | | gctcgaagaagctaatactg |
| | | | | atccgtgtgcccctcctttt |
| | | | | gattccctccagacctacgc |
| | | | | ttttgagggaacagggtcat |
| | | | | tagctggatccctgagctcc |
| | | | | ttagaatcagcagtctctga |
| | | | | tcaggatgaaagctatgatt |
| | | | | accttaatgagttgggacct |
| | | | | cgctttaaaagattagcatg |
| | | | | catgtttggttctgcagtgc |
| | | | | agtcaaataattag |
| 964 | C137915 | artifi- | aa | AWVWRPFWLEEMDDIQCVGK |
| | muCDH19 | cial | | LRSDLDNGNNSFQYKLLGIG |
| | (44-461) | | | AGSFSINERTGEICAIQKLD |
| | huCDH19 | | | REEKSLYILRAQVIDTTIGK |
| | (464-772) | | | AVETESEFVIRVLDINDNEP |
| | | | | RFLDSPYEAIVPEMSPEGTF |
| | | | | VIKVTANDADDPSTGYHARI |
| | | | | LYNLERGQPYFSVEPTTGVI |
| | | | | RISSKMDRELQDTYCVIIQA |
| | | | | KDMLGQPGALSGTTTVSIKL |
| | | | | SDINDNKPIFKESFYRFTIS |
| | | | | ESAPIGTSIGKIMAYDDDIG |
| | | | | ENAEMEYSIEDDDSKIFDII |
| | | | | IDNDTQEGIVILKKKVDFSQ |
| | | | | QSYYGIRAKVKNCHVDEELA |
| | | | | PAHVNASTTYIKVQVEDEDE |
| | | | | PPVFLLPYYILEIPEGKPYG |
| | | | | TIVGTVSATDPDRRQSPMRY |
| | | | | YLTGSKMFDINDNGTIITTN |
| | | | | MLDREVSAWYNLTVTATETY |
| | | | | NVQQISSAHVYVQVFNINDH |
| | | | | APEFSQYYETYVCENAGSGQ |
| | | | | VIQTISAVDRDESIEEHHFY |
| | | | | FNLSVEDTNNSSFTIIDNQD |
| | | | | NTAVILTNRTGFNLQEEPVF |
| | | | | YISILIADNGIPSLTSTNTL |
| | | | | TIHVCDCGDSGSTQTCQYQE |
| | | | | LVLSMGFKTEVIIAILICIM |
| | | | | IIFGFIFLTLGLKQRRKQIL |
| | | | | FPEKSEDFRENIFQYDDEGG |
| | | | | GEEDTEAFDIAELRSSTIMR |
| | | | | ERKTRKTTSAEIRSLYRQSL |
| | | | | QVGPDSAIFRKFILEKLEEA |
| | | | | NTDPCAPPFDSLQTYAFEGT |
| | | | | GSLAGSLSSLESAVSDQDES |
| | | | | YDYLNELGPRFKRLACMFGS |
| | | | | AVQSNN |
| 965 | C137915 | artifi- | nt | gcctgggtgtggagaccatt |
| | muCDH19 | cial | | tgttgttctagaagaaatgg |
| | (44-461) | | | atgatatacaatgtgttgga |
| | huCDH19 | | | aagctaagatctgacttaga |
| | (464-772) | | | caatggaaacaactctttcc |
| | | | | agtacaagctactggggatt |
| | | | | ggcgctggaagctttagcat |
| | | | | taatgaaagaacaggtgaaa |
| | | | | tatgtgccatacagaagctt |
| | | | | gatagagaggaaaaatccct |
| | | | | ctacattctgagagcccagg |
| | | | | taatagacaccactattggg |
| | | | | aaggctgtggaaactgaatc |
| | | | | cgagtttgtcatcagagttt |
| | | | | tggatatcaatgacaatgaa |
| | | | | cccagattcctagatgaacc |
| | | | | atatgaggccattgtacctg |
| | | | | agatgtctccagaaggaaca |
| | | | | tttgtcatcaaggtgacagc |
| | | | | caatgacgcagatgatcctt |
| | | | | caactggctatcatgctcgc |
| | | | | atcctatacaacttagaacg |
| | | | | aggtcaaccatacttttctg |
| | | | | ttgagccaacaacaggagtc |
| | | | | ataaggatatcttctaagat |
| | | | | ggatagagagttgcaa |
| | | | | gatacatactgtgtaattat |
| | | | | tcaagccaaggacatgctcg |
| | | | | gtcagcctggagccttgtct |
| | | | | ggaacaacaaccgtatcaat |
| | | | | taagctgtcagatattaatg |
| | | | | acaacaagccaatattcaaa |
| | | | | gaaagtttctaccgcttcac |
| | | | | tatatctgaatctgcaccca |
| | | | | ttggaacatcaatagggaaa |
| | | | | attatggcatatgatgatga |
| | | | | cataggggagaatgcagaga |
| | | | | tggactacagcattgaagat |
| | | | | gatgattcaaaaatatttga |
| | | | | cataatcattgacaatgaca |
| | | | | cccaagaagggatagttata |
| | | | | cttaaaaagaaagttgattt |
| | | | | tgagcagcagagctattatg |
| | | | | gcattagagctaaggttaaa |
| | | | | aactgccatgtggatgaaga |
| | | | | gcttgcacctgcccatgtta |
| | | | | acgcttccacaacctacatt |
| | | | | aaagttcaagtagaagatga |
| | | | | agatgaacctcctgttttcc |
| | | | | tcttaccatattacatactt |
| | | | | gaaattcctgaaggaaaacc |
| | | | | atatggaacaattgtgggga |
| | | | | cggtttctgccacagaccca |
| | | | | gatcgaagacaatctcctat |
| | | | | gagatattatctcactggaa |
| | | | | gcaaaatgtttgaLatcaat |
| | | | | gacaatggaacaataatcac |
| | | | | cactaacatgcttgacagag |
| | | | | aggtcagtgcttggtacaac |
| | | | | ttgactgtcacagctactga |
| | | | | aacatacaatgtacaacaga |
| | | | | tctcttcagcccatgtttat |
| | | | | gtacaagtctttaacattaa |
| | | | | tgatcatgctcctgagttct |
| | | | | ctcaatactatgagacttat |
| | | | | gtttgtgaaaatgcaggctc |
| | | | | tggtcaggtaattcagacta |
| | | | | tcagtgcagtggatagagat |
| | | | | gaatccatagaacagcacca |
| | | | | tttttactttaatctatctg |
| | | | | tagaagacactaacaattca |
| | | | | agttttacaatcatagataa |
| | | | | tcaagataacacagctgtca |
| | | | | ttttgactaatagaactggt |
| | | | | tttaaccttcaagaagaacc |
| | | | | tgtcttctacatctccatct |
| | | | | taattgccgacaatggaatc |
| | | | | ccgtcacttacaagtacaaa |
| | | | | cacccttaccatccaLgtct |
| | | | | gtgactgtggtgacagtggg |
| | | | | agcacacagacctgccagta |
| | | | | ccaggagctttgtgctttcca |
| | | | | tgggattcaagacagaagtc |
| | | | | atcattgctattctcatttg |
| | | | | cattatgatcatatttgggt |
| | | | | ttatttttttgactttgggt |
| | | | | ttaaaacaacggagaaaaca |
| | | | | gattctatttcctgagaaaa |
| | | | | gtgaagatttcagagagaat |
| | | | | atattccaatatgatgatga |
| | | | | agggcgtggagaagaagata |
| | | | | cagaggcctttgatatagca |
| | | | | gagctgaggagtagtaccat |
| | | | | aatgcgggaacgcaagactc |
| | | | | ggaaaaccacaagcgctgag |
| | | | | atcaggagcctatacaggca |
| | | | | gtctttgcaagttggccccg |
| | | | | acagtgccatattcaggaaa |
| | | | | ttcattctggaaaagctcga |
| | | | | agaagctaatactgatccgt |
| | | | | gtgcccctccttttgattcc |
| | | | | ctccagacctacgcttttga |
| | | | | gggaacagggtcattagctg |
| | | | | catccctgagctccttagaa |
| | | | | tcagcagtctctgatcagga |
| | | | | tgaaagctatgattacctta |
| | | | | atgagttgggacctcgcttt |
| | | | | aaaagattagcatgcatgtt |
| | | | | tggttctgcagtgcagtcaa |
| | | | | ataattag |
| 966 | C71144 | artifi- | aa | AWVWRPFVVLEEMDDIQCVG |
| | muCDH19 | cial | | KLRSDLDNGNNSFQYKLLGI |
| | (44-770) | | | GAGSFSINERTGEICAIQKL |
| | | | | DREEKSLYILRAQVIDTTIG |
| | | | | KAVETESEFVIRVLDINDNE |
| | | | | PRFLDEPYEAIVPEMSPEGT |
| | | | | FVIKVTANDADDPSTGYHAR |
| | | | | ILYNLERGQPYFSVEPTTGV |
| | | | | IRISSKMDRELQDTYCVIIQ |
| | | | | AKDMLGQPGALSGTTTVSIK |
| | | | | LSDINDNKPIFKESFYRFTI |
| | | | | SESAPIGTSIGKIMAYDDDI |
| | | | | GENAEMEYSIEDDDSKIFDI |
| | | | | IIDNDTQEGIVILKKKVDFE |
| | | | | QQSYYGIRAKVKNCHVDEEL |
| | | | | APAHVNASTTYIKVQVEDED |
| | | | | EPPVFLLPYYILEIPEGKPY |
| | | | | GTIVGTVSATDPDRRQSPMR |
| | | | | YYLTGSKMFDINDNSTIITT |
| | | | | NMLDREVSAWYNLTVTATET |
| | | | | YNVQQISSAHVYVQVFNIND |
| | | | | NAPEFSQFYETYVCENAESG |
| | | | | EIVQIISAIDRDESIEDHHF |
| | | | | YFNHSLEDTNNSSFMLTDNQ |
| | | | | DNTAVILSNRTGFNLKEEPV |
| | | | | FYMIILIADNGIPSLTSTNT |
| | | | | LTIQVCDCGDSRNTETCANK |
| | | | | GLLFIMGFRTEAIIAIMICV |
| | | | | MVIFGFFFLILALKQRRKET |
| | | | | LFPEKTEDFRENIFCYDDEG |
| | | | | GGEEDSEAFDIVELRQSTVM |
| | | | | RERKPQRSKSAEIRSLYRQS |
| | | | | LQVGPDSAIFRKFILEKLEE |
| | | | | ANTDPCAPPFDSLQTFAYEG |
| | | | | TGSSAGSLSSLASRDTDQED |
| | | | | DFDYLNDLGPRFKRLASMFG |
| | | | | SAVQPNN |
| 967 | C71144 | artifi- | nt | gcctgggtgtggagaccatt |
| | muCDH19 | cial | | tgttgttctagaagaaatgg |
| | (44-770) | | | atgatatacaatgtgttgga |
| | | | | aagctaagatctgacttaga |
| | | | | caatggaaacaactctttcc |
| | | | | agtacaagctactggggatt |
| | | | | ggcgctggaagctttagcat |
| | | | | taatgaaagaacaggtgaaa |
| | | | | tatgtgccatacagaagctt |
| | | | | gatagagaggaaaaatccct |
| | | | | ctacattctgagagcccagg |
| | | | | taatagacaccactattggg |
| | | | | aaggctgcggaaactgaatc |
| | | | | cgagtttgtcatcagagttt |
| | | | | tggatatcaatgacaatgaa |
| | | | | cccagattcctagatgaacc |
| | | | | atatgaggccattgtacctg |
| | | | | agatgtctccagaaggaaca |
| | | | | tttgtcatcaaggtgacagc |
| | | | | caatgacgcagatgatcctt |
| | | | | caactggctatcatgctcgc |
| | | | | atcctatacaacttagaacg |
| | | | | aggtcaaccatacttttctg |
| | | | | ttgagccaacaacaggagtc |
| | | | | ataaggatatcttctaagat |
| | | | | ggatagagagttgcaagata |
| | | | | catactgtgtaattattcaa |
| | | | | gccaaggacatgctcggtca |
| | | | | gcctggagccttgtctggaa |
| | | | | caacaaccgtatcaattaag |
| | | | | ctgtcagatattaatgacaa |
| | | | | caagccaatattcaaagaaa |
| | | | | gtttctaccgcttcactata |
| | | | | tctgaatctgcacccattgg |
| | | | | aacatcaatagggaaaatta |
| | | | | tggcatatgatgatgacata |
| | | | | ggggagaatgcagagatgga |
| | | | | gtacagcattgaagatgatg |
| | | | | attcaaaaatatttgacata |
| | | | | atcattgacaatgacaccca |
| | | | | agaagggatagttatactta |
| | | | | aaaagaaagttgattttgag |
| | | | | cagcagagccattatggcat |
| | | | | tagagctaaggttaaaaact |
| | | | | gccatgtggatgaagagctt |
| | | | | gcacctgcccatgttaacgc |
| | | | | ttccacaacctacattaaag |
| | | | | ttcaagtagaagatgaagat |
| | | | | gaacctcctgttttcctctt |
| | | | | accatattacatacttgaaa |
| | | | | ttcctgaaggaaaaccatat |
| | | | | ggaacaattgtggggacggt |
| | | | | ttctgccacagacccagatc |
| | | | | gaagacaatctcctatgaga |
| | | | | tattatctcactggaagcaa |
| | | | | aatgtttgatatcaatgaca |
| | | | | atggaacaataatcaccact |
| | | | | aacatgcttgacagagaggt |
| | | | | cagtgcttggtacaacttga |
| | | | | ctgtcacagctactgaaaca |
| | | | | tacaatgtacaacagatctc |
| | | | | ttcagcccatgtttatgtac |
| | | | | aagtctttaacattaacgac |
| | | | | aatgctccagagttctctca |
| | | | | attctatgagacttatgttt |
| | | | | gtgaaaatgctgaatctggt |
| | | | | gagatagttcagatcatcag |
| | | | | tgcaattgatagagatgagt |
| | | | | ccatagaagatcaccatttt |
| | | | | tactttaatcactctctgga |
| | | | | agacacaaacaactcaagtt |
| | | | | ttatgctaacagacaatcaa |
| | | | | gataacacagctgtaattct |
| | | | | gagtaatagaactggtttca |
| | | | | atcttaaagaagagcctgtc |
| | | | | ttctacatgatcatcttgat |
| | | | | tgctgataacgggatcccat |
| | | | | ctctcacaagcacaaacact |
| | | | | ctcactatccaagtctgtga |
| | | | | ctgtggagacagtagaaaca |
| | | | | cagaaacttgtgctaacaag |
| | | | | ggacttctctttatcatggg |
| | | | | attcagaacagaggcaataa |
| | | | | ttgccatcatgatatgtgtt |
| | | | | atggtaatatttgggttttt |
| | | | | ctttttgatttctttgctctg |
| | | | | aaacagcgaagaaaggagact |
| | | | | ctatttccagagaagactga |
| | | | | agactttagggagaatatat |
| | | | | tttgctatgatgatgaaggc |
| | | | | ggcggggaagaagactcgga |
| | | | | agcctttgacatcgtagagc |
| | | | | tgagacaaagtacagtaatg |
| | | | | agagaaagaaagcctcagag |
| | | | | aagcaagagtgcggagatca |
| | | | | ggagcttgtacaggcagtcc |
| | | | | ctgcaggtgggcccagacag |
| | | | | tgccatatttcgaaaattta |
| | | | | tcctagagaagcttgaagaa |
| | | | | gccaacacagacccatgtgc |
| | | | | tcccccctttgattcactac |
| | | | | agacgtttgcctatgaggga |
| | | | | acagggtcatcagctggctc |
| | | | | tctgagctccttggcatcca |
| | | | | gagacaccgatcaggaggat |
| | | | | gacttcgactaccttaatga |
| | | | | cctgggacctcgttttaaaa |
| | | | | gattagcaagcatgtttggc |
| | | | | tctgcagtacaacccaacaa |
| | | | | ttag |
| 968 | Flag Tag | artifi- | aa | DYKDDDDK |
| | | cial | | |
| 969 | Flag Tag | artifi- | nt | gactacaaagacgatgacga |
| | | cial | | caag |
| 970 | ckCDH19 | artifi- | aa | MNCSTFLSLVLALVQLQLCS |
| | (1-43):: | cial | | PTTQIFSAQKTDQSYTTIRR |
| | FLAG::ckC | | | VKRDYKDDDDKGWVWEPLFV |
| | DH19 | | | TEEETSTMPMYVGQLKSDLD |
| | (44-776) | | | KEDGSLQYILTGEGADSIFF |
| | | | | INEHGKIYVRQKLDREKKSF |
| | | | | YILRAQVINRKTRHPIEPDS |
| | | | | EFIIKVRDINDHEPQFLDGP |
| | | | | YVATVPEMSPEGTSVTQVTA |
| | | | | TDGDDPSYGNNARLLYSLIQ |
| | | | | GQPYFSVEPKTGVIRMTSQM |
| | | | | DRETKDQYLVVIQAKDMVGQ |
| | | | | AGAFSATATVTINLSDVNDN |
| | | | | PPKFQQRLYYLNVSEEAPVG |
| | | | | TTVGRLLAEDSDIGENAAMN |
| | | | | YFIEEDSSDVFGIITDRETQ |
| | | | | EGIIILKKRVDYESKRKHSV |
| | | | | RVKAVNRYIDDRFLKEGPFE |
| | | | | DITIVQISVVDADEPPVFTL |
| | | | | ESYVMEIAEGVVSGSLVGTV |
| | | | | SARDLDNDDSSVRYSIVQGL |
| | | | | HLKRLFSINEHNGTIITTEP |
| | | | | LDREKASWHNITVTATETRN |
| | | | | PEKISEANVYIQVLDVNDHA |
| | | | | PEFSKYYETFVCENAVPGQL |
| | | | | IQNISAVDKDDSAENHRFYF |
| | | | | SLAQATNSSHFTVKDNQDNT |
| | | | | AGIFTA3SGFSRKEQFYFFL |
| | | | | PILILDNGSPPLTSTNTLTV |
| | | | | TVCDCDTEVNTLYCRYGAFL |
| | | | | YSIGLSTEALVAVLACLLIL |
| | | | | LVFFLAIIGIRQQRKKTLFS |
| | | | | EKVEEFRENIVRYDDEGGGE |
| | | | | EDTEAFDISALRTRAVLRTH |
| | | | | KPRKKITTEIHSLYRQSLQV |
| | | | | GPDSAIFRQFISEKLEEANT |
| | | | | DPSVPPYDSLQTYAFEGTGS |
| | | | | LAGSLSSLGSNTSDVDQNYE |
| | | | | YLVGWGPPFKQLAGMYTSQR |
| | | | | STRD |
| 971 | huCDH19(1- | | | MNCYLLLRFMLGIPLLWPCL |
| | 43)::FLAG:: | | | GATENSQTKKVKQPVRSHLR |
| | hu(44- | | | VKRDYKDDDDKGWVWNQFFV |
| | 141):: | | | PEEMNTTSHHIGQLRSDLDN |
| | ckCDH19 | | | GNNSFQYKLLGAGAGSTFII |
| | (142-776) | | | DERTGDIYAIQKLDREERSL |
| | | | | YILRAQVIDIATGRAVEPES |
| | | | | EFVIKVSDINDHEPQFLDGP |
| | | | | YVATVPEMSPEGTSVTQVTA |
| | | | | TDGDDPSYGKNARLLYSLIQ |
| | | | | GQPYFSVEPKTGVIRKTSQM |
| | | | | DRETKDQYLVVIQAKDMVGQ |
| | | | | AGAFSATATVTIKLSDVNDN |
| | | | | PPKFQQRLYYLNVSEEAPVG |
| | | | | TTVGRLLAEDSDIGENAAMN |
| | | | | YFIEEDSSDVFGIITDRETQ |
| | | | | EGIIILKKRVDYESKRKHSV |
| | | | | RVKAVNRYIDDRFLKEGPFE |
| | | | | DITIVQISVVDADEPPVFTL |
| | | | | ESYVMEIAEGVVSGSLVGTV |
| | | | | SARDLDNDDSSVRYSIVQGL |
| | | | | HLKRLFSINE |
| | | | | HNGTIITTEPLDREKASWHN |
| | | | | ITVTATETRNPEKISEANVY |
| | | | | IQVLDVNDHAPEFSKYYETF |
| | | | | VCENAVPGQLIQNISAVDKD |
| | | | | DSAENHRFYFSLAQATNSSH |
| | | | | FTVKDNQDNTAGIFTAGSGF |
| | | | | SRKEQFYFFLPILILDNGSP |
| | | | | PLTSTNTLTVTVCDCDTEVN |
| | | | | TLYCRYGAFLYSIGLSTEAL |
| | | | | VAVLACLLILLVFFLAIIGI |
| | | | | RQQRKKTLFSEKVEEFRENI |
| | | | | VRYDDEGGGEEDTEAFDISA |
| | | | | LRTRAVLRTHKPRKKITTEI |
| | | | | HSLYRQSLQVGPDSAIFRQF |
| | | | | ISEKLEEANTDPSVPPYDSL |
| | | | | QTYAFEGTGSLAGSLSSLGS |
| | | | | NTSDVDQNYEYLVGWGPPFK |
| | | | | QLAGMYTSQRSTRD |
| 972 | ckCDH19(1- | | | MNCSTFLSLVLALVQLQLCS |
| | 43):: | | | PTTQIFSAQKTDQSYTTIRR |
| | FLAG::ckC | | | VKRDYKDDDDKGWVWEPLFV |
| | DH19(44- | | | TEEETSTMPMYVGQLKSDLD |
| | 141):: | | | KEDGSLQYILTGEGADSIFF |
| | huCDH19 | | | INEHGKIYVRQKLDREKKSF |
| | (142- | | | YILRAQVINRKTRHPIEPDS |
| | 249):: | | | EFIIKVRDINDNEPKFLDEP |
| | ckCDH19 | | | YEAIVPEMSPEGTLVIQVTA |
| | (250-776) | | | SDADDPSSGNNARLLYSLLQ |
| | | | | GQPYFSVEPTTGVIRISSKM |
| | | | | DRELQDEYWVIIQAKDMIGQ |
| | | | | PGALSGTTSVLIKLSDVNDN |
| | | | | PPKFQQRLYYLNVSEEAPVG |
| | | | | TTVGRLLAEDSDIGENAAMN |
| | | | | YFIEEDSSDVFGIITDRETQ |
| | | | | EGIIILKKRVDYESKRKHSV |
| | | | | RVKAVNRYIDDRFLKEGPFE |
| | | | | DITIVQISVVDADEPPVFTL |
| | | | | ESYVMEIAEGVVSGSLVGTV |
| | | | | SARDLDNDDSSVRYSIVQGL |
| | | | | HLKRLFSINEHNGTIITTEP |
| | | | | LDREKASWHNITVTATETRN |
| | | | | PEKISEANVYIQVLDVNDHA |
| | | | | PEFSKYYETFVCENAVPGQL |
| | | | | IQNISAVDKDDSAENHRFYF |
| | | | | SLAQATNSSHFTVKDNQDNT |
| | | | | AGIFTAGSGFSRKEQFYFFL |
| | | | | PILILDNGSPPLTSTNTLTV |
| | | | | TVCDCDTEVNTLYCRYGAFL |
| | | | | YSIGLSTEALVAVLACLLIL |
| | | | | LVFFLAIIGIRQQRKKTLFS |
| | | | | EKVEEFRENIVRYDDEGGGE |
| | | | | EDTEAFDISALRTRAVLRTH |
| | | | | KPRKKITTEIHSLYRQSLQV |
| | | | | GPDSAIFRQFISEKLEEANT |
| | | | | DPSVPPYDSLQTYAFEGTGS |
| | | | | LAGSLSSLGSNTSDVDQNYE |
| | | | | YLVGWGPPFKQLAGMYTSQR |
| | | | | STRD |
| 973 | ckCDH19(1- | | | MNCSTFLSLVLALVQLQLCS |
| | 43):: | | | PTTQIFSAQKTDQSYTTIRR |
| | FLAG::ckC | | | VKRDYKDDDDKGWVWEPLFV |
| | DH19(44- | | | TEEETSTMPMYVGQLKSDLD |
| | 249):: | | | KEDGSLQYILTGEGADSIFF |
| | huCDH19 | | | INEHGKIYVRQKLDREKKSF |
| | (250- | | | YILRAQVINRKTRHPIEPDS |
| | 364):: | | | EFIIKVRDINDHEPQFLDGP |
| | ckCDH19 | | | YVATVPEMSPEGTSVTQVTA |
| | (365-776) | | | TDGDDPSYGKNARLLYSLIQ |
| | | | | GQPYFSVEPKTGVIRKTSQM |
| | | | | DRETKDQYLWIQAKDMVGQA |
| | | | | GAFSATATVTINLSDVNDNK |
| | | | | PIFKESLYRLTVSESAPTGT |
| | | | | SIGTIMAYDNDIGENAEMDY |
| | | | | SIEEDDSQTFDIITNHETQE |
| | | | | GIVILKKKVDFEHQNHYGIR |
| | | | | AKVKNHHVPEQLMKYHTEAS |
| | | | | TTFIKIQVEDVDEPPVFTLE |
| | | | | SYVMEIAEGWSGSLVGTVSA |
| | | | | RDLDNDDSSVRYSIVQGLHL |
| | | | | KRLFSINEHNGTIITTEPLD |
| | | | | REKASWHNITVTATETRNPE |
| | | | | KISEANVYIQVLDVNDHAPE |
| | | | | FSKYYETFVCENAVPGQLIQ |
| | | | | NISAVDKDDSAENHRFYFSL |
| | | | | AQATNSSHFTVKDNQDNTAG |
| | | | | IFTAGSGFSRKEQFYFFLPI |
| | | | | LILDNGSPPLTSTNTLTVTV |
| | | | | CDCDTEVNTLYCRYGAFLYS |
| | | | | IGLSTEALVAVLACLLILLV |
| | | | | FFLAIIGIRQQRKKTLFSEK |
| | | | | VEEFRENIVRYDDEGGGEED |
| | | | | TEAFDISALRTRAVLRTHKP |
| | | | | RKKITTEIHSLYRQSLQVGP |
| | | | | DSAIFRQFISEKLEEANTDP |
| | | | | SVPPYDSLQTYAFEGTGSLA |
| | | | | GSLSSLGSNTSDVDQNYEYL |
| | | | | VGWGPPFKQLAGMYTSQRST |
| | | | | RD |
| 974 | ckCDH19(1- | | | MNCSTFLSLVLALVQLQLCS |
| | 43):: | | | PTTQIFSAQKTDQSYTTIRR |
| | FLAG::ckC | | | VKRDYKDDDDKGWVWEPLFV |
| | DH19(44- | | | TEEETSTMPMYVGQLKSDLD |
| | 364):: | | | KEDGSLQYILTGEGADSIFF |
| | huCDH19 | | | INEHGKIYVRQKLDREKKSF |
| | (365- | | | YILRAQVINRKTRHPIEPDS |
| | 463):: | | | EFIIKVRDINDHEPQFLDGP |
| | ckCDH19 | | | YVATVPEMSPEGTSVTQVTA |
| | (469-776) | | | TDGDDPSYGNNARLLYSLIQ |
| | | | | GQPYFSVEPKTGVIRMTSQM |
| | | | | DRETKDQYLVVIQAKDMVGQ |
| | | | | AGAFSATATVTINLSDVNDN |
| | | | | PPKFQQRLYYLNVSEEAPVG |
| | | | | TTVGRLLAEDSDIGENAAMN |
| | | | | YFIEEDSSDVFGIITDRETQ |
| | | | | EGIIILKKRVDYESKRKHSV |
| | | | | RVKAVNRYIDDRFLKEGPFE |
| | | | | DITIVQISVVDADEPPLFLL |
| | | | | PYYVFEVFEETPQGSFVGVV |
| | | | | SATDPDNRKSPIRYSITRSK |
| | | | | VFNINDNGTITTSNSLDREI |
| | | | | SAWYNLSITATEKYNIEQIS |
| | | | | SIPLYVQVLNINDHAPEFSK |
| | | | | YYETFVCENAVPGQLIQNIS |
| | | | | AVDKDDSAENHRFYFSLAQA |
| | | | | TNSSHFTVKDNQDNTAGIFT |
| | | | | AGSGFSRKEQFYFFLPILIL |
| | | | | DNGSPPLTSTNTLTVTVCDC |
| | | | | DTEVNTLYCRYGAFLYSIGL |
| | | | | STEALVAVLACLLILLVFFL |
| | | | | AIIGIRQQRKKTLFSEKVEE |
| | | | | FRENIVRYDDEGGGEEDTEA |
| | | | | FDISALRTRAVLRTHKPRKK |
| | | | | ITTEIHSLYRQSLQVGPDSA |
| | | | | IFRQFISEKLEEANTDPSVP |
| | | | | PYDSLQTYAFEGTGSLAGSL |
| | | | | SSLGSNTSDVDQNYEYLVGW |
| | | | | GPPFKQLAGMYTSQRSTRD |
| 975 | (1- | | | MNCSTFLSLVLALVQLQLCS |
| | 43):: | | | PTTQIFSAQKTDQSYTTIRR |
| | FLAG::ckC | | | VKRDYKDDDDKGWVWEPLFV |
| | DH19(44- | | | TEEETSTMPMYVGQLKSDLD |
| | 468):: | | | KEDGSL |
| | huCDH19 | | | QYILTGEGADSIFFIKEHGK |
| | (464-772) | | | IYVRQKLDREKKSFYILRAQ |
| | | | | VINRKTRHPIEPDSEFIIKV |
| | | | | RDINDHEPQFLEGPYVATVP |
| | | | | EMSPEGTSVTQVTATDGDDP |
| | | | | SYGNNARLLYSLIQGQPYFS |
| | | | | VEPKTGVIRMTSQMDRETKD |
| | | | | QYLVVIQAKDMVGQAGAFSA |
| | | | | TATVTINLSDVNDNPPKFQQ |
| | | | | RLYYLNVSEEAPVGTTVGRL |
| | | | | LAEDSDIGENAAMNYFIEED |
| | | | | SSDVFGIITDRETQEGIIIL |
| | | | | KKRVDYESKRKHSVRVKAVN |
| | | | | RYIDDRFLKEGPFEDITIVQ |
| | | | | ISVVDADEPPVFTLESYVME |
| | | | | IAEGVVSGSLVGTVSARDLD |
| | | | | MDDSSVRYSIVQGLHLKRLF |
| | | | | SINEHNGTIITTEPLDREKA |
| | | | | SWHNITVTATETRNPEKISE |
| | | | | ANVYIQVLDVNDHAPEFSQY |
| | | | | YETYVCENAGSGQVIQTISA |
| | | | | VDRDESIEEHHFYFNLSVED |
| | | | | TNNSSFTIIDNQDNTAVILT |
| | | | | NRTGFNLQEEPVFYISILIA |
| | | | | DNGIPSLTSTNTLTIHVCDC |
| | | | | GDSGSTQTCQYQELVLSMGF |
| | | | | KTEVIIAILICIMIIFGFIF |
| | | | | LTLGLKQRRKQILFPEKSED |
| | | | | FRENIFQYDDEGGGEEDTEA |
| | | | | FDIAELRSSTIMRERKTRKT |
| | | | | TSAEIRSLYRQSLQVGPDSA |
| | | | | IFRKFILEKLEEANTDPCAP |
| | | | | PFDSLQTYAFEGTGSLAGSL |
| | | | | SSLESAVSDQDESYDYLNEL |
| | | | | GPRFKRLACMFGSAVQSNN |
| 976 | rhCDH19(1- | | | MNCYLLLPFMLGIPLLKPCL |
| | 43):: | | | GATENSQTKKVQQPVGSHLR |
| | FLAG::rhC | | | VKRDYKDDDDKGWVWNQFFV |
| | DH19 | | | PEEMNTTSHHVGRLRSDIDN |
| | (44-772) | | | GNNSFQYKLLGAGAGSTFII |
| | | | | DERTGDIYAIEKLDREERSL |
| | | | | YILRAQVIDITTGRAVEPES |
| | | | | EFVIKVSDINDNEPKFLDEP |
| | | | | YEAIVPEMSPEGTLVIQVTA |
| | | | | SDADDPSSGNNARLLYSLLQ |
| | | | | GQPYFSVEPTTGVIRISSKM |
| | | | | DRELQDEYWVIIQAKDMIGQ |
| | | | | PGALSGTTSVLIKLSDVNDN |
| | | | | KPIFKESLYRLTVSESAPTG |
| | | | | TSIGTIMAYDNDIGENAEMD |
| | | | | YSIEEDDSQTFDIITNHETQ |
| | | | | EGIVILKKKVNFEHQNHYGI |
| | | | | RAKVKNHHVDEQLMKYHTEA |
| | | | | STTFIKIQVEDVDEPPLFLL |
| | | | | PYYIFEIFEETPQGSFVGVV |
| | | | | SATDPDNRKSPIRYSITRSK |
| | | | | VFNIDDNGTITTTNSLDREI |
| | | | | SAWYNLSITATEKYNIEQIS |
| | | | | SIPVYVQVLNINDHAPEFSQ |
| | | | | YYESYVCENAGSGQVIQTIS |
| | | | | AVDRDESIEEHHFYFNLSVE |
| | | | | DTNSSSFTIIDNQDNTAVIL |
| | | | | TNRTGFNLQEEPIFYISILI |
| | | | | ADNGIPSLTSTNTLTIHVCD |
| | | | | CDDSGSTQTCQYQELMLSMG |
| | | | | FKTEVIIAILICIMVIFGFI |
| | | | | FLTLGLKQRRKQILFPEKSE |
| | | | | DFRENIFRYDDEGGGEEDTE |
| | | | | AFDVAALRSSTIMRERKTRK |
| | | | | TTSAEIRSLYRQSLQVGPDS |
| | | | | AIFRKFILEKLEEADTDPCA |
| | | | | PPFDSLQTYAFEGTGSLAGS |
| | | | | LSSLESAVSDQDESYDYLNE |
| | | | | LGPRFKRLACMFGSAVQSNM |
| 977 | caCDH19(1- | | | QFFVPEEMNKTDYHIGQLRS |
| | 42):: | | | DLDNGNNSFQYKLLGAGAGS |
| | FLAG::caC | | | IFVIDERTGDIYAIQKLDRE |
| | DH19 | | | ERSLYTLRAQVIDSTTGRAV |
| | (43-770) | | | EPESEFVIRVSDINDNEPKF |
| | | | | LDEPYEAIVPEMSPEGTLVI |
| | | | | QVTATDADDPASGNNARLLY |
| | | | | SLLQGQPYFSIEPTTGVIRI |
| | | | | SSKMDRELQDEYWVIIQAKD |
| | | | | MIGLPGALSGTTSVLIKLSD |
| | | | | VNDNKPIFKERLYRLTVSES |
| | | | | APTGTSIGRIMAYDNDIGEN |
| | | | | AEMDYSIEDDSQTFDIITNN |
| | | | | ETQEGIVILKKKVDFEHQNH |
| | | | | YLIRANVKNRHVAEHLMEYH |
| | | | | VEASTTFVRVQVEDEDEPPV |
| | | | | FLLPYYLFEILEESPHGSFV |
| | | | | GMVSATDPDQRKSPIRYSIT |
| | | | | RSKVFSIDDNGTIITTNPLD |
| | | | | REISAWYNLSITATEKYNVQ |
| | | | | QISAVPVYVQVLNINDHAPE |
| | | | | FSEYYDSYVCENAGSGQVIQ |
| | | | | TISAVDRDESVEDHHFYFNL |
| | | | | SVEDTKNSSFIIIDNEDNTA |
| | | | | VILTNRTGFSLQEEPVFYIS |
| | | | | VLIADNGIPSLTSTNTLTIH |
| | | | | ICDCDDYGSTQTCRDKDLLL |
| | | | | SMGFRTEVILAILISIMIIF |
| | | | | GFIFLILGLKQRRKPTLFPE |
| | | | | KGEDFRENIFRYDDEGGGEE |
| | | | | DTEAFDIVQLRSSTIMRERK |
| | | | | TRKTAAAEIRSLYRQSLQVG |
| | | | | PDSAIFRKFILEKLEEANTD |
| | | | | PCAPPFDSLQTYAFEGTGSL |
| | | | | AGSLSSLGSAVSDQDENYDY |
| | | | | LNELGPRFKRLACMFGSAMQ |
| | | | | SNN |
| 978 | rhCDH19(1- | | | MNCYLLLPFMLGIPLLWPCL |
| | 43): | | | GATENSQTKKVQQPVGSHLR |
| | FLAG::rhC | | | VKRDYKDDDDKGWVWNQFFV |
| | DH19(44- | | | PEEMNTTSHHVGRLRSDLDN |
| | 141):: | | | GNNSFQYKLLGAGAGSTFII |
| | caCDH19 | | | DERTGDIYAIEKLDREERSL |
| | (141-770) | | | YILRAQVIDITTGRAVEPES |
| | | | | EFVIKVSDINDNEPKFLDEP |
| | | | | YEAIVPEMSPEGTLVIQVTA |
| | | | | TDADDPASGNNARLLYSLLQ |
| | | | | GQPYFSIEPTTGVIRISSKM |
| | | | | DRELQDEYKVIIQAKDMIGL |
| | | | | PGALSGTTSVLIKLSDVNDN |
| | | | | KPIFKERLYRITVSESAPTG |
| | | | | TSIGRIMAYDNDIGENAEMD |
| | | | | YSIEDDSQTFDIITNNETQE |
| | | | | GIVILKKKVDFEHQNHYLIR |
| | | | | ANVKNRHVAEHLMEYHVEAS |
| | | | | TTFVRVQVEDEDEPPVFLLP |
| | | | | YYLFEILEESPHGSFVGMVS |
| | | | | ATDPDQRKSPIRYSITRSKV |
| | | | | FSIDDNGTIITTNPLDREIS |
| | | | | AWYNLSITATEKYNVQQISA |
| | | | | VPVYVQVLNINDHAPEFSEY |
| | | | | YDSYVCENAGSGQVIQTISA |
| | | | | VDRDESVEDHHFYFNLSVED |
| | | | | TKNSSFIIIDNEDNTAVILT |
| | | | | NRTGFSLQEEPVFYISVLIA |
| | | | | DNGIPSLTSTNTLTIHICDC |
| | | | | DDYGSTQTCRDKDLLLSMGF |
| | | | | RTEVILAILISIMIIFGFIF |
| | | | | LILGLKQRRKPTLFPEKGED |
| | | | | FRENIFRYDDEGGGEEDTEA |
| | | | | FDIVQLRSSTIMRERKTRKT |
| | | | | AAAEIRSL |
| | | | | YRQSLQVGPDSAIFRKFILE |
| | | | | KLEEANTDPCAPPFDSLQTY |
| | | | | AFEGTGSLAGSLSSLGSAVS |
| | | | | DQDENYDYLNELGPRFKRLA |
| | | | | CMFGSAMQSNN |
| 979 | rhCDH19(1- | | | MNCYLLLPFMLGIPLLWPCL |
| | 43):: | | | GATENSQTKKVQQPVGSHLR |
| | FLAG::rhC | | | VKRDYKDDDOKGWVWNQFFV |
| | DH19(44- | | | PEEMNTTSHHVGRLRSDLDN |
| | 65):: | | | GNNSFQYKLLGAGAGSIFVI |
| | caCDH19 | | | DERTGDIYAIQKLDREERSL |
| | (65-770) | | | YTLRAQVIDSTTGRAVEPES |
| | | | | EFVIRVSDINDNEPKFLDEP |
| | | | | YEAIVPEMSPEGTLVIQVTA |
| | | | | TDADDPASGNNARLLYSLLQ |
| | | | | GQPYFSIEPTTGVIRISSKM |
| | | | | DRELQDEYWVIIQAKDMIGL |
| | | | | PGALSGTTSVLIKLSDVNDN |
| | | | | KPIFKERLYRLTVSESAPTG |
| | | | | TSIGRIMAYDNDIGENAEMD |
| | | | | YSIEDDSQTFDIITNNETQE |
| | | | | GIVILKKKVDFEHQNHYLIR |
| | | | | ANVKNRHVAEHLMEYHVEAS |
| | | | | TTFVRVQVEDEDEPPVFLLP |
| | | | | YYLFEILEESPHGSFVGMVS |
| | | | | ATDPDQRKSPIRYSITRSKV |
| | | | | FSIDDNGTIITTNPLDREIS |
| | | | | AWYNLSITATEKYNVQQISA |
| | | | | VPVYVQVLNINDHAPEFSEY |
| | | | | YDSYVCENAGSGQVIQTISA |
| | | | | VDRDESVEDHKFYFNLSVED |
| | | | | TKNSSFIIIDNEDNTAVILT |
| | | | | NRTGFSLQEEPVFYISVLIA |
| | | | | DNGIPSLTSTNTLTIHICDC |
| | | | | DDYGSTQTCRDKDLLLSMGF |
| | | | | RTEVILAILISIMIIFGFIF |
| | | | | LILGLKQRRKPTLFPEKGED |
| | | | | FRENIFRYDDEGGGEEDTEA |
| | | | | FDIVQLRSSTIMRERKTRKT |
| | | | | AAAEIRSLYRQSLQVGPDSA |
| | | | | IFRKFILEKLEEANTDPCAP |
| | | | | PFDSLQTYAFEGTGSLAGSL |
| | | | | SSLGSAVSDQDENYDYLNEL |
| | | | | GPRFKRLACMFGSAMQSNN |
| 980 | caCDH19(1- | | | MNYCFLLPLMLGIPLIWPCF |
| | 43):: | | | TASESSKTEVKHQAGSHLRV |
| | FLAG::caC | | | KRDYKDDDDKGWMWNQFFVP |
| | DH19(44- | | | EEMNKTDYHIGQLRSDLDNG |
| | 87):: | | | NNSFQYKLLGAGAGSTFIID |
| | rhCDH19(89- | | | ERTGDIYAIEKLDREERSLY |
| | 114):: | | | ILRAQVIDSTTGRAVEPESE |
| | caCDH19 | | | FVIRVSDINDNEPKFLDEPY |
| | (115-770) | | | EAIVPEMSPEGTLVIQVTAT |
| | | | | DADDPASGNNARLLYSLLQG |
| | | | | QPYFSIEPTTGVIRISSKMD |
| | | | | RELQDEYWVIIQAKDMIGLP |
| | | | | GALSGTTSVLIKLSDVNDNK |
| | | | | PIFKERLYRLTVSESAPTGT |
| | | | | SIGRIMAYDNDIGENAEMDY |
| | | | | SIEDDSQTFDIITNNETQEG |
| | | | | IVILKKKVDFEHQNHYLIRA |
| | | | | KVKNRHVAEHLMEYHVEAST |
| | | | | TFVRVQVEDEDEPPVFLLPY |
| | | | | YLFEILEESPHGSFVGMVSA |
| | | | | TDPDQRKSPIRYSITRSKVF |
| | | | | SIDDNGTIITTNPLDREISA |
| | | | | WYNLSITATEKYNVQQISAV |
| | | | | PVYVQVLNINDHAPEFSEYY |
| | | | | DSYVCENAGSGQVIQTISAV |
| | | | | DRDESVEDHHFYFNLSVEDT |
| | | | | KNSSFIIIDNEDNTAVILTN |
| | | | | RTGFSLQEEPVFYISVLIAD |
| | | | | NGIPSLTSTNTLTIHICDCD |
| | | | | DYGSTQTCRDKDLLLSMGFR |
| | | | | TEVILAILISIMIIFGFIFL |
| | | | | ILGLKQRRKPTLFPEKGEDF |
| | | | | RENIFRYDDEGGGEEDTEAF |
| | | | | DIVQLRSSTIMRERKTRKTA |
| | | | | AAEIRSLYRQSLQVGPDSAI |
| | | | | FRKFILEKLEEANTDPCAPP |
| | | | | FDSLQTYAFEGTGSLAGSLS |
| | | | | SLGSAVSDQDENYDYLNELG |
| | | | | PRFKRLACMFGSAMQSNN |
| 981 | caCDH19(1- | | | MNYCFLLPLMLGIPLIWPCF |
| | 43):: | | | TASESSKTEVKHQAGSHLRV |
| | FLAG::caC | | | KRDYKDDDDKGWMWNQFFVP |
| | DH19(44- | | | EEMNKTDYHIGQLRSDLDNG |
| | 120):: | | | NNSFQYKLLGAGAGSIFVID |
| | rhCDH19 | | | ERTGDIYAIQKLDREERSLY |
| | (122- | | | TLRAQVIDITTGRAVEPESE |
| | 137):: | | | FVIKVSDINDNEPKFLDEPY |
| | caCDH19 | | | EAIVPEMSPEGTLVIQVTAT |
| | (137-770) | | | DADDPASGNNARLLYSLLQG |
| | | | | QPYFSIEPTTGVIRISSKMD |
| | | | | RELQDEYWVIIQAKDMIGLP |
| | | | | GALSGTTSVLIKLSDVNDNK |
| | | | | PIFKERLYRLTVSESAPTGT |
| | | | | SIGRIMAYDNDIGENAEMDY |
| | | | | SIEDDSQTFDIITNNETQEG |
| | | | | IVILKKKVDFEHQNHYLIRA |
| | | | | NVKNRHVAEHLMEYHVEAST |
| | | | | TFVRVQVEDEDEPPVFLLPY |
| | | | | YLFEILEESPHGSFVGMVSA |
| | | | | TDPDQRKSPIRYSITRSKVF |
| | | | | SIDDNGTIITTNPLDREISA |
| | | | | WYNLSITATEKYNVQQISAV |
| | | | | PVYVQVLNINDHAPEFSEYY |
| | | | | DSYVCENAGSGQVIQTISAV |
| | | | | DRDESVEDEHFYFNLSVEDT |
| | | | | KNSSFIIIDNEDNTAVILTN |
| | | | | RTGFSLQEEPVFYISVLIAD |
| | | | | NGIPSLTSTNTLTIHICDCD |
| | | | | DYGSTQTCRDKDLLLSMGFR |
| | | | | TEVILAILISIMIIFGFIFL |
| | | | | ILGLKQRRKPTLFPEKGEDF |
| | | | | RENIFRYDDEGGGEEDTEAF |
| | | | | DIVQLRSSTIMRERKTRKTA |
| | | | | AAEIRSLYRQSLQVGPDSAI |
| | | | | FRKFILEKLEEANTDPCAPP |
| | | | | FDSLQTYAFEGTGSLAGSLS |
| | | | | SLGSAVSDQDENYDYLNELG |
| | | | | PRFKRLACMFGSAMQSNN |
| 982 | rhCDH19(1- | | | MNCYLLLPFMLGIPLLWPCL |
| | 43):: | | | GATENSQTKKVQQPVGSHLR |
| | FLAG::rhC | | | VKRDYKDDDDKGWVWNQFFV |
| | DH19(44- | | | PEEMNTTSHHVGRLRSDLDN |
| | 141):: | | | GNNSFQYKLLGAGAGSTFII |
| | raCDH19 | | | DERTGDIYAIEKLDREERSL |
| | (140- | | | YILRAQVIDITTGRAVEPES |
| | 247):: | | | EFVIKVSDINDNEPRFLDEP |
| | rhCDH19 | | | YEAIVPEMSPEGTFVIKVTA |
| | (250-772) | | | NDADDPTSGYHARILYNLEQ |
| | | | | GQPYFSVEPTTGVIRISSKM |
| | | | | DRELQDTYCVIIQAKDMLGQ |
| | | | | PGALSGTTTISIKLSDINDN |
| | | | | KPIFKESLYRLTVSESAPTG |
| | | | | TSIGTIMAYDNDIGENAEMD |
| | | | | YSIEEDDSQTFDIITNHETQ |
| | | | | EGIVILKKKVNFEHQNHYGI |
| | | | | RAKV |
| | | | | KNHHVDEQLMKYHTEASTTF |
| | | | | IKIQVEDVDEPPLFLLPYYI |
| | | | | FEIFEETPQGSFVGVVSATD |
| | | | | PDNRKSPIRYSITRSKVFNI |
| | | | | DDNGTITTTNSLDREISAWY |
| | | | | NLSITATEKYNIEQISSIPV |
| | | | | YVQVLNINDHAPEFSQYYES |
| | | | | YVCENAGSGQVIQTISAVDR |
| | | | | DESIEEKHFYFNLSVEDTNS |
| | | | | SSFTIIDNQDNTAVILTNRT |
| | | | | GFNLQEEPIFYISILIADNG |
| | | | | IPSLTSTNTLTIHVCDCDDS |
| | | | | GSTQTCQYQELMLSMGFKTE |
| | | | | VIIAILICIMVIFGFIFLTL |
| | | | | GLKQRRKQILFPEKSEDFRE |
| | | | | NIFRYDDEGGGEEDTEAFDV |
| | | | | AALRSSTIMRERKTRKTTSA |
| | | | | EIRSLYRQSLQVGPDSAIFR |
| | | | | KFILEKLEEADTDPCAPPFD |
| | | | | SLQTYAFEGTGSLAGSLSSL |
| | | | | ESAVSDQDESYDYLNELGPR |
| | | | | FKRLACMFGSAVQSNN |
| 983 | raCDH19(1- | | | MNHYFLKYWILMVPLIWPCL |
| | 43):: | | | KVAETLKIEKAQRAVPSLGR |
| | FLAG::raC | | | AKRDYKDDDDKGWVWKQFVV |
| | DH19 | | | PEEMDTIQHVGRLRSDLDNG |
| | (44-770) | | | NNSFQYKLLGTGDGSFSIDE |
| | | | | KTGDIFAMQKLDREKQSLYI |
| | | | | LRAQVIDTTIGKAVEPESEF |
| | | | | VIRVSDVNDNEPRFLDEPYE |
| | | | | AIVPEMSPEGTFVIKVTAND |
| | | | | ADDPTSGYHARILYNLEQGQ |
| | | | | PYFSVEPTTGVIRISSKMDR |
| | | | | ELQDTYCVIIQAKDMLGQPG |
| | | | | ALSGTTTISIKLSDINDNKP |
| | | | | IFKESFYRFTISESAPSGTT |
| | | | | IGKIMAYDDDIGENAEMDYS |
| | | | | IEDDESQIFDIVIDNETQEG |
| | | | | IVILKKKVDFEHQNHYGIRV |
| | | | | KVKNCHVDEELAPAHVNAST |
| | | | | TYIKVQVEDEDEPPTFLLPY |
| | | | | YIFEIPEGKPYGTMVGTVSA |
| | | | | VDPDRRQSPMRYSLIGSKMF |
| | | | | DINGNGTIVTTNLLDREVSA |
| | | | | WYNLSVTATETYNVQQISSA |
| | | | | HVYVQVLNINDHAPEFSQLY |
| | | | | ETYVCENAESGEIIQTISAI |
| | | | | DRDESIEDHHFYFNHSVEDT |
| | | | | NNSSFILTDNQDNTAVILSN |
| | | | | RAGFSLKEETVFYMIILIAD |
| | | | | NGIPPLTSTNTLTIQVCDCG |
| | | | | DSRSTETCTSKELLFIMGFK |
| | | | | AEAIIAIVICVMVIFGFIFL |
| | | | | ILALKQRRKETLFPEKTEDF |
| | | | | RENIFCYDDEGGGEEDSEAF |
| | | | | DIIELRQSTVMRERKPRKSR |
| | | | | SAEIRSLYRQSLQVGPDSAI |
| | | | | FRKFILEKLEEANTDSSAPP |
| | | | | FDSLQTFAYEGTGSSAGSLS |
| | | | | SLGSSVTDQEDDFDYLNDLG |
| | | | | PCFKRLANMFGSAVQPDN |
| 984 | (1- | | | MNYCFLKHWILMIPLLWPCL |
| | 43):: | | | KVSETLKAEKARRTVPSTWR |
| | FLAG::mu | | | AKRDYKDDDDKAWVWRPFVV |
| | CDH19 | | | LEEMDDIQCVGKLRSDLDNG |
| | (44-323):: | | | NNSFQYKLLGIGAGSFSINE |
| | raCDH19 | | | RTGEICAIQKLDREEKSLYI |
| | (324- | | | LRAQVIDTTIGKAVETESEF |
| | 327):: | | | VIRVLDINDNEPRFLDEPYE |
| | muCDH19 | | | AIVPEMSPEGTFVIKVTAND |
| | (328-770) | | | ADDPSTGYHARILYNLERGQ |
| | | | | PYFSVEPTTGVIRISSKMDR |
| | | | | ELQDTYCVIIQAKDMLGQPG |
| | | | | ALSGTTTVSIKLSDINDNKP |
| | | | | IFKESFYRFTISESAPIGTS |
| | | | | IGKIMAYDDDIGENAEMEYS |
| | | | | IEDDDSKIFDIIIDNDTQEG |
| | | | | IVILKKKVDFEHQNHYGIRA |
| | | | | KVKNCHVDEELAPAHVNAST |
| | | | | TYIKVQVEDEDEPPVFLLPY |
| | | | | YILEIPEGKPYGTIVGTVSA |
| | | | | TDPDRRQSPMRYYLTGSKMF |
| | | | | DINDNGTIITTNMLDREVSA |
| | | | | WYNLTVTATETYNVQQISSA |
| | | | | HVYVQVFNINDNAPEFSQFY |
| | | | | ETYVCENAESGEIVQIISAI |
| | | | | DRDESIEDEHFYFNHSLEDT |
| | | | | NNSSFMLTDNQDNTAVILSN |
| | | | | RTGFNLKEEPVFYMIILIAD |
| | | | | NGIPSLTSTNTLTIQVCDCG |
| | | | | DSRNTETCANKGLLFIMGFR |
| | | | | TEAIIAIMICVMVIFGFFFL |
| | | | | ILALKQRRKETLFPEKTEDF |
| | | | | RENIFCYDDEGGGEEDSEAF |
| | | | | DIVELRQSTVMRERKPQRSK |
| | | | | SAEIRSLYRQSLQVGPDSAI |
| | | | | FRKFILEKLEEANTDPCAPP |
| | | | | FDSLQTFAYEGTGSSAGSLS |
| | | | | SLASRDTDQEDDFDYLNDLG |
| | | | | PRFKRLASMFGSAVQPNN |
| 985 | muCDH19(1- | | | MNYCFLKHWILMIPLLWPCL |
| | 43):: | | | KVSETLKAEKARRTVPSTWR |
| | FLAG::mu | | | AKRDYKDDDDKAWVWRPFVV |
| | CDH19(44- | | | LEEMDDIQCVGKLRSDLDNG |
| | 770):: | | | NNSFQYKLLGIGAGSFSINE |
| | raCDH19 | | | RTGEICAIQKLDREEKSLYI |
| | (290,299, | | | LRAQYIDTTIGKAVETESEF |
| | 308) | | | VIRVLDINDNEPRFLDEPYE |
| | | | | AIVPEMSPEGTFVIKVTAND |
| | | | | ADDPSTGYHARILYNLERGQ |
| | | | | PYFSVEPTTGVIRISSKMDR |
| | | | | ELQDTYCVIIQAKDMLGQPG |
| | | | | ALSGTTTVSIKLSDINDNKP |
| | | | | IFKESFYRFTISESAPIGTS |
| | | | | IGKIMAYDDDIGENAEMEYS |
| | | | | IEDDDSKIFDIIIDNDTQEG |
| | | | | IVILKKKVDFEQQSYYGIRA |
| | | | | KVKNCHVDEELAPAHVNAST |
| | | | | TYIKVQVEDEDEPFVFLLPY |
| | | | | YILEIPEGKPYGTIVGTVSA |
| | | | | TDPDRRQSPMRYYLTGSKMF |
| | | | | DINDNGTIITTNMLDREVSA |
| | | | | WYNLTVTATETYNVQQISSA |
| | | | | HVYVQVFNINDNAPEFSQFY |
| | | | | ETYVCENAESGEIVQIISAI |
| | | | | DRDESIEDKHFYFNHSLEDT |
| | | | | NNSSFMLTDNQDNIAVILSN |
| | | | | RTGFNLKEEPVFYMIILIAD |
| | | | | NGIPSLTSTNTLTIQVCDCG |
| | | | | DSRNTETCANKGLLFIMGFR |
| | | | | TEAIIAIMICVMVIFGFFFL |
| | | | | ILALKQRRKETLFPEKTEDF |
| | | | | RENIFCYDDEGGGEEDSEAF |
| | | | | DIVELRQSTVMRERKPQRSK |
| | | | | SAEIRSLYRQSLQVGPDSAI |
| | | | | FRKFILEKLEEANTDPCAPP |
| | | | | FDSLQTFAYEGTGSSAGSLS |
| | | | | SLASRDTDQEDDFDYLNDLG |
| | | | | PRFKRLASMFGSAVQPNN |
| 986 | muCDH19(1- | | | MNYCFLKHWILMIPLLWPCL |
| | 43):: | | | KVSETLKAEKARRTVPSTWR |
| | FLAG::mu | | | AKRDYKDDDDKAWVWRPFVV |
| | CDH19(44- | | | LEEMDDIQCVGKLRSDLDNG |
| | 770):: | | | NNSFQYKLLGIGAGSFSINE |
| | huCDH19 | | | RTGEICAIQKLDREEKSLYI |
| | (271) | | | LRAQVIDTTIGKAVETESEF |
| | | | | VIRVLDINDNEPRFLDEPYE |
| | | | | AIVPEMSPEGTFVIKVTAND |
| | | | | ADDPSTGYHARILYNLERGQ |
| | | | | PYFSVEPTTGVIRISSKMDR |
| | | | | ELQDTYCVIIQAKDMLGQPG |
| | | | | ALSGTTTVSIKLSDINDNKP |
| | | | | IFKESFYRFTISESAPTGTS |
| | | | | IGKIMAYDDDIGENAEMEYS |
| | | | | IEDDDSKIFDIIIDNDTQEG |
| | | | | IVILKKKVDFEQQSYYGIRA |
| | | | | KVKNCHVDEELAPAHVNAST |
| | | | | TYIKVQVEDEDEPPVFLLPY |
| | | | | YILEIPEGKPYGTIVGTVSA |
| | | | | TDPDRRQSPMRYYLTGSKMF |
| | | | | DINDNGTIITTNMLDREVSA |
| | | | | WYNLTVTATETYNVQQISSA |
| | | | | HVYVQVFNINDNAPEFSQFY |
| | | | | ETYVCENAESGEIVQIISAI |
| | | | | DRDESIEDHHFYFNHSLEDT |
| | | | | NNSSFMLTDNQDNTAVILSN |
| | | | | RTGFNLKEEPVFYMIILIAD |
| | | | | NGIPSLTSTNTLTIQVCDCG |
| | | | | DSRNTETCANKGLLFIMGFR |
| | | | | TEAIIAIMICVMVIFGFFFL |
| | | | | ILALKQRRKETLFPEKTEDF |
| | | | | RENIFCYDDEGGGEEDSEAF |
| | | | | DIVELRQSTVMRERKPQRSK |
| | | | | SAEIRSLYRQSLQVGPDSAI |
| | | | | FRKFILEKLEEANTDPCAPP |
| | | | | FDSLQTFAYEGTGSSAGSLS |
| | | | | SLASRDTDQEDDFDYLNDLG |
| | | | | PRFKRLASMFGSAVQPNN |

1. An isolated human antibody or antigen binding fragment thereof capable of binding to human cadherin 19 (CDH19) on the surface of a target cell, comprising a VH region comprising CDR-H1, CDR-H2 and CDR-H3 and a VL region comprising CDR-L1, CDR-L2 and CDR-L3 selected from the group consisting of:
(a) a VH region comprising CDR-H1 as set forth in SEQ ID NO: 94, CDR-H2 as set forth in SEQ ID NO: 95, CDR-H3 as set forth in SEQ ID NO: 96, and a VL region comprising CDR-L1 as set forth in SEQ ID NO: 262, CDR-L2 as set forth in SEQ ID NO: 263 and CDR-L3 as set forth in SEQ ID NO: 264,
(b) a VH region comprising CDR-H1 as set forth in CDR-H1 as set forth in SEQ ID NO: 100, CDR-H2 as set forth in SEQ ID NO: 101, CDR-H3 as set forth in SEQ ID NO: 102, and a VL region comprising CDR-L1 as set forth in SEQ ID NO: 268, CDR-L2 as set forth in SEQ ID NO: 269 and CDR-L3 as set forth in SEQ ID NO: 270,
(c) a VH region comprising CDR-H1 as set forth in CDR-H1 as set forth in SEQ ID NO: 118, CDR-H2 as set forth in SEQ ID NO: 119, CDR-H3 as set forth in SEQ ID NO: 120, and a VL region comprising CDR-L1 as set forth in SEQ ID NO: 286, CDR-L2 as set forth in SEQ ID NO: 287 and CDR-L3 as set forth in SEQ ID NO: 288,
(d) a VH region comprising CDR-H1 as set forth in CDR-H1 as set forth in SEQ ID NO: 154, CDR-H2 as set forth in SEQ ID NO: 155, CDR-H3 as set forth in SEQ ID NO: 156, and a VL region comprising CDR-L1 as set forth in SEQ ID NO: 322, CDR-L2 as set forth in SEQ ID NO: 323 and CDR-L3 as set forth in SEQ ID NO: 324,
(e) a VH region comprising CDR-H1 as set forth in CDR-H1 as set forth in SEQ ID NO: 100, CDR-H2 as set forth in SEQ ID NO: 101, CDR-H3 as set forth in SEQ ID NO: 912, and a VL region comprising CDR-L1 as set forth in SEQ ID NO: 268, CDR-L2 as set forth in SEQ ID NO: 269 and CDR-L3 as set forth in SEQ ID NO: 270,
(f) a VH region comprising CDR-H1 as set forth in CDR-H1 as set forth in SEQ ID NO: 100, CDR-H2 as set forth in SEQ ID NO: 101, CDR-H3 as set forth in SEQ ID NO: 913, and a VL region comprising CDR-L1 as set forth in SEQ ID NO: 268, CDR-L2 as set forth in SEQ ID NO: 269 and CDR-L3 as set forth in SEQ ID NO: 270,
(g) a VH region comprising CDR-H1 as set forth in CDR-H1 as set forth in SEQ ID NO: 94, CDR-H2 as set forth in SEQ ID NO: 95, CDR-H3 as set forth in SEQ ID NO: 910, and a VL region comprising CDR-L1 as set forth in SEQ ID NO: 262, CDR-L2 as set forth in SEQ ID NO: 263 and CDR-L3 as set forth in SEQ ID NO: 264,
(h) a VH region comprising CDR-H1 as set forth in CDR-H1 as set forth in SEQ ID NO: 94, CDR-H2 as set forth in SEQ ID NO: 95, CDR-H3 as set forth in SEQ ID NO: 911, and a VL region comprising CDR-L1 as set forth in SEQ ID NO: 262, CDR-L2 as set forth in SEQ ID NO: 263 and CDR-L3 as set forth in SEQ ID NO: 264,
(i) a VH region comprising CDR-H1 as set forth in CDR-H1 as set forth in SEQ ID NO: 118, CDR-H2 as set forth in SEQ ID NO: 119, CDR-H3 as set forth in SEQ ID NO: 120, and a VL region comprising CDR-L1 as set forth in SEQ ID NO: 286, CDR-L2 as set forth in SEQ ID NO: 287 and CDR-L3 as set forth in SEQ ID NO: 288,
(j) a VH region comprising CDR-H1 as set forth in CDR-H1 as set forth in SEQ ID NO: 118, CDR-H2 as set forth in SEQ ID NO: 914, CDR-H3 as set forth in SEQ ID NO: 120, and a VL region comprising CDR-L1 as set forth in SEQ ID NO: 286, CDR-L2 as set forth in SEQ ID NO: 287 and CDR-L3 as set forth in SEQ ID NO: 288, and
(k) a VH region comprising CDR-H1 as set forth in CDR-H1 as set forth in SEQ ID NO: 154, CDR-H2 as set forth in SEQ ID NO: 155, CDR-H3 as set forth in SEQ ID NO: 920, and a VL region comprising CDR-L1 as set forth in SEQ ID NO: 322, CDR-L2 as set forth in SEQ ID NO: 323 and CDR-L3 as set forth in SEQ ID NO: 324.

2. The human antibody or antigen binding fragment thereof according to claim 1, wherein the antibody or antigen binding fragment thereof is a monoclonal antibody or a fragment thereof.

3. (canceled)

4. The human antibody or antigen binding fragment thereof of claim 1, comprising a VH region comprising the amino acid sequence selected from the group consisting of:
SEQ ID NO: 338, SEQ ID NO: 354, SEQ ID NO: 378, SEQ ID NO: 356, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 501, SEQ ID NO: 502, SEQ ID NO: 503, SEQ ID NO: 504, SEQ ID NO: 505, SEQ ID NO: 506, SEQ ID NO: 517, and SEQ ID NO: 518.

5. The human antibody or antigen binding fragment thereof of claim 1, comprising a VL region comprising the amino acid sequence selected from the group consisting of:
SEQ ID NO: 394, SEQ ID NO: 410, SEQ ID NO: 434, SEQ ID NO: 412, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 596, SEQ ID NO: 597, SEQ ID NO: 598, SEQ ID NO: 599, SEQ ID NO: 600, SEQ ID NO: 601, SEQ ID NO: 612, and SEQ ID NO: 613.

6. The human antibody or antigen binding fragment thereof of claim 1, comprising VH region and a VL region comprising a pair of amino acid sequences selected from the group consisting of:
SEQ ID NOs: 338and 394, SEQ ID NOs: 354and 410, SEQ ID NOs: 378and 434, SEQ ID NOs: 356and 412, SEQ ID NOs: 476and 571, SEQ ID NOs: 477 and 572, SEQ ID NOs: 478and 573, SEQ ID NOs: 479and 574, SEQ ID NOs: 480and 575, SEQ ID NOs: 481 and 576, SEQ ID NOs: 482and 577, SEQ ID NOs: 483and 578, SEQ ID NOs: 484and 579, SEQ ID NOs: 501 and 596, SEQ ID NOs; 502and 597, SEQ ID NOs: 503and 598, SEQ ID NOs: 504and 599, SEQ ID NOs: 505and 600, SEQ ID NOs: 506 and 601, SEQ ID NOs: 517and 612, and SEQ ID NOs: 518and 613.

7. The human antibody or antigen binding fragment thereof of claim 6, wherein the human binding domain or antigen binding fragment thereof comprising a heavy and light chain amino acid sequence selected from the group consisting of:
SEQ ID NOs: 656and 692, SEQ ID NOs: 654and 690, SEQ ID NOs: 664and 700, SEQ ID NOs: 670 and 706, SEQ ID NOs: 738and 833, SEQ ID NOs: 739and 834, SEQ ID NOs: 740and 835, SEQ ID NOs: 741 and 836, SEQ ID NOs: 742and 837, SEQ ID NOs: 743and 838, SEQ ID NOs: 744and 839, SEQ ID NOs: 745and 840, SEQ ID NOs: 746and 841, SEQ ID NOs: 763and 858, SEQ ID NOs: 764and 859, SEQ ID NOs: 765and 860, SEQ ID NOs: 766 and 861, SEQ ID NOs: 767and 862, SEQ ID NOs: 768and 863, SEQ ID NOs: 779and 874, and SEQ ID NOs: 780and 875.

8. An antibody construct comprising the human antibody or antigen binding fragment thereof of claim 1 conjugated to a chemotherapeutic agent.

9. The antibody construct of claim 8, further comprising a linker, wherein the linker conjugates the chemotherapeutic agent to the human antibody or antigen binding fragment thereof.

10. The antibody construct of claim 9, wherein the linker is a non-cleavable linker.

11. The antibody construct of claim 10, wherein the linker comprises N-succinimidyl 4-(N-maleimidomethyl) cyclohexane-1 carboxylate (MCC).

12. The antibody construct of claim 8, wherein the chemotherapeutic agent is conjugated to one or more lysines contained in the human antibody or antigen binding fragment thereof.

13. The antibody construct of claim 8, wherein the chemotherapeutic agent is maytansinoid (DM1).

14. The antibody construct of claim 13, wherein the average number of DM1 molecules per antibody construct is between 1 and 10.

15. The antibody construct of claim 13, wherein the average number of DM1 molecules per antibody construct is between 3 and 7.

16. The antibody construct of claim 13, wherein the average number of DM1 molecules per antibody construct is between 4 and 6.

17. The antibody construct of claim 13, wherein the average number of DM1 molecules per antibody construct is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0.

18. An isolated nucleic acid molecule encoding the human antibody or antigen binding fragment thereof of claim 1.

19. A vector comprising the nucleic acid molecule of claim 18.

20. A host cell transformed or transfected with the nucleic acid molecule of claim 18.

21. A process for producing a human antibody or antigen binding fragment thereof capable of binding to human cadherin 19 (CDH19) on the surface of a target cell, said process comprising culturing the host cell of claim 21 under conditions allowing the expression of the antibody or antigen binding fragment thereof.

22. The process of claim 21 further comprising recovering the produced antibody or antigen binding fragment thereof, and conjugating a chemotherapeutic agent to the recovered antibody or antigen binding fragment thereof to produce an antibody conjugate.

23. A composition comprising the human antibody or antigen binding fragment thereof of claim 1 and a pharmaceutically acceptable carrier.

24-25. (canceled)

26. A method for treating or ameliorating a melanoma disease or metastatic melanoma disease, comprising administering to a subject in need thereof the antibody or antigen binding fragment thereof of claim 1.

27. The method of claim 26, wherein the melanoma disease or metastatic melanoma disease is selected from the group consisting of superficial spreading melanoma, lentigo maligna, lentigo maligna melanoma, acral lentiginous melanoma and nodular melanoma.

28. A kit comprising the antibody or antigen binding fragment thereof of claim 1 in a vial or a syringe.
